(19) United States
(12) Patent Application Publication
(10) Pub. No.: US 2022/0041615 A1
(43) Pub. Date: Feb. 10, 2022

(54) ORGANIC ELECTROLUMINESCENT COMPOUND AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Hong-Se OH, Gyeonggi-do (KR); Du-Yong PARK, Gyeonggi-do (KR); Doo-Hyeon MOON, Gyeonggi-do (KR); Dong-Hyung LEE, Gyeonggi-do (KR)

(21) Appl. No.: 17/373,518

(22) Filed: Jul. 12, 2021

(30) Foreign Application Priority Data
Jul. 13, 2020 (KR) 10-2020-0086006
May 12, 2021 (KR) 10-2021-0061494

(51) Int. Cl. C07D 493/10; H01L 51/00

(52) U.S. Cl. CPC C07D 493/10; H01L 51/0073; H01L 51/006; H01L 51/5056; H01L 51/0056; H01L 51/0074; H01L 51/0061

(57) ABSTRACT

The present disclosure relates to an organic electroluminescent compound and an organic electroluminescent device comprising the same. By comprising the organic electroluminescent compound according to the present disclosure, an organic electroluminescent device having low driving voltage and/or high luminous efficiency characteristics can be provided.

### TECHNICAL FIELD

The present disclosure relates to an organic electroluminescent compound and an organic electroluminescent device comprising the same.

### BACKGROUND ART

Among a display device, an electroluminescent device (EL device) is a self-light-emitting display device which has advantages in that it provides a wider viewing angle, a greater contrast ratio, and a faster response time. An organic EL device was first developed by Eastman Kodak in 1987, by using small aromatic diamine molecules and aluminum complexes as materials for forming a light-emitting layer [Appl. Phys. Lett. 51, 913, 1987].

An organic electroluminescent device (OLED) consists of a multi-layer structure including a hole injection layer, a hole transport layer, a light-emitting layer, an electron transport layer, and an electron injection layer, etc., in order to improve its efficiency and stability. In this case, the selection of a compound included in the hole transport layer or the like is recognized as one of the means for improving device properties such as the hole transport efficiency to a light-emitting layer, the luminous efficiency, and the lifespan.

In this regard, copper phthalocyanine (CuPc), 4,4″-bis[N-(1-naphthyl)-N-phenylamino]biphenyl (NPB), N,N″-diphenyl-N,N′-bis(3-methylphenyl)-(1,1″-biphenyl)-4,4′-diamine (TPD), 4,4′,4″-tris(3-methylphenylphenylamino)triphenylamine (MTDATA), etc., were used as a compound comprised in a hole injection and transport material in an OLED. However, an OLED prepared using these materials have problems of reduction in quantum efficiency and lifespan. This is due to the circumstance when an OLED is driven under high current, thermal stress occurs between an anode and a hole injection layer, thereby such thermal stress significantly reduces the lifespan of the device. Further, since the organic material used in the hole injection layer has very high hole mobility, there have been problems in that the hole-electron charge balance is broken and the quantum efficiency (cd/A) is lowered.

Therefore, the development of a material for a hole transport layer for improving the performance of an OLED is still required.

WO 2016/021989 A1 discloses an example of using a spiro[fluorene-9,9′-xanthene] derivative compound and a spiro[fluorene-9,9′-thioxanthene] derivative compound as a material of an electron buffer layer or an electron transport layer, but said compounds in the reference are not used as a material for a hole transport layer.

### Disclosure of the Invention

### Technical Problem

The object of the present disclosure is firstly, to provide an organic electroluminescent compound which can be prepared for an organic electroluminescent device having low driving voltage and/or high luminous efficiency characteristics, and secondly, to provide an organic electroluminescent device comprising the organic electroluminescent compound.

### Solution to Problem

As a result of intensive studies to solve the technical problem above, the present inventors found that the aforementioned objective can be achieved by an organic electroluminescent compound represented by the following formula 1, so that the present invention was completed.

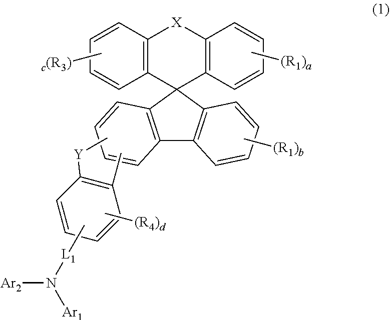

In formula 1,

X and Y each independently represent, O or S;

L₁ represents a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene;

Ar₁ and Ar₂ each independently represent, a substituted or unsubstituted (C6-C30)aryl, a substituted or unsubstituted (3- to 30-membered)heteroaryl, a substituted or unsubstituted (C3-C30)cycloalkyl, a substituted or unsubstituted fused ring of (C3-C30) aliphatic ring and (C6-C30) aromatic ring, or -L₂-N-(Ar₃)(Ar₄); or may be linked to an adjacent substituent(s) to form a ring(s);

R₁ to R₄ each independently represent, hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or -L₃-N-(Ar₅)(Ar₆); or may be linked to an adjacent substituent(s) to form a ring(s);

L₂ and L₃ each independently represent, a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene;

Ar₃ to Ar₆ each independently represent; a substituted or unsubstituted (C1-C30)alkyl, a substituted or unsubstituted (C2-C30)alkenyl, a substituted or unsubstituted (C6-C30)aryl, a substituted or unsubstituted fused ring of (C3-C30) aliphatic ring and (C6-C30) aromatic ring, or a substituted or unsubstituted (3- to 30-membered)heteroaryl;

a to c each independently represent, an integer of 1 to 4, and d represents an integer of 1 to 3; and

when a to d each independently are an integer of 2 or more, each of R₁ to R₄ may be the same or different.

### Advantageous Effects of Invention

An organic electroluminescent device having low driving voltage and/or high luminous efficiency characteristics can be manufactured by comprising an organic electroluminescent compound according to the present disclosure.

### MODE FOR THE INVENTION

Hereinafter, the present disclosure will be described in detail. However, the following description is intended to explain the invention, and is not meant in any way to restrict the scope of the invention.

The present disclosure relates to an organic electroluminescent compound represented by formula 1 above, an organic electroluminescent material comprising the organic electroluminescent compound, and an organic electroluminescent device comprising the organic electroluminescent compound.

The term “organic electroluminescent compound” in the present disclosure means a compound that may be used in an organic electroluminescent device, and may be comprised in any layer constituting an organic electroluminescent device, as necessary.

The term “organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (including host and dopant materials), an electron buffer material, a hole blocking material, an electron transport material, or an electron injection material, etc.

The “hole transport zone” in the present disclosure means a zone where holes move between the first electrode and the light-emitting layer. For example, the hole transport zone may include at least one of a hole injection layer, a hole transport layer, a hole auxiliary layer, a light-emitting auxiliary layer, and an electron blocking layer. Each of the hole injection layer, the hole transport layer, the hole auxiliary layer, the light-emitting auxiliary layer, and the electron blocking layer can be a single layer or a multi-layer of which two or more layers are stacked. According to one embodiment of the present application, the hole transport zone may include a first hole transport layer and a second hole transport layer. The second hole transport layer may be at least one layer of a plurality of hole transport layers, and may include at least one of a hole auxiliary layer, a light-emitting auxiliary layer, and an electron blocking layer. In addition, according to another embodiment of the present application, the hole transport zone may include a first hole transport layer and a second hole transport layer, and the first hole transport layer may be located between the first electrode and the light-emitting layer, and the second hole transport layer may be positioned between the first hole transport layer and the light-emitting layer, and the second hole transport layer may be a layer serving as a hole transport layer, a light-emitting auxiliary layer, a hole auxiliary layer and/or an electron blocking layer.

The term “(C1-C30)alkyl” in the present disclosure is meant to be a linear or branched alkyl having 1 to 30 carbon atoms constituting the chain, in which the number of carbon atoms is preferably 1 to 20, and more preferably 1 to 10. The above alkyl may include methyl, ethyl, n-propyl, isopropyl, n-butyl, isobutyl, Cert-butyl, sec-butyl, etc. The term “(C3-C30)cycloalkyl” in the present disclosure is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, cyclopentylmethyl, cyclohexylmethyl, etc. The term “(C6-C30)aryl(ene)” in the present disclosure is a monocyclic or fused ring radical derived from an aromatic hydrocarbon having 6 to 30 ring backbone carbon atoms, in which the number of the ring backbone carbon atoms is preferably 6 to 20, more preferably 6 to 15, and may be partially saturated. The aryl may comprise a spire structure. Examples of the aryl specifically include phenyl, biphenyl, terphenyl, quaterphenyl, naphthyl, binaphthyl, phenylnaphthyl, naphthylphenyl, fluorenyl, phenylfluorenyl, dimethylfluorenyl, diphenylfluorenyl, benzofluorenyl, diphenylbenzofluorenyl, dibenzofluorenyl, phenanthrenyl, benzophenanthrenyl, phenylphenanthrenyl, anthracenyl, benzanthracenyl, indenyl, triphenylenyl, pyrenyl, tetracenyl, perylenyl, chrysenyl, benzochrysenyl, naphthacenyl, fluoranthenyl, benzofluoranthenyl, tolyl, xylyl, mesityl, cumenyl, spiro[fluorene-fluorene]yl, spiro[fluorene-benzofluorene]yl, azulenyl, tetramethyl-dihydrophenanthrenyl, etc. More specifically, the aryl may be o-tolyl, m-tolyl, p-tolyl, 2,3-xylyl, 3,4-xylyl, 2,5-xylyl, mesityl, o-cumenyl, m-cumenyl, p-cumenyl, p-t-butylphenyl, p-(2-phenylpropyl)phenyl, 4′-methylbiphenyl, 4″-t-butyl-p-terphenyl-4-yl, o-biphenyl, m-biphenyl, p-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 1-naphthyl, 2-naphthyl, 1-fluorenyl, 2-fluorenyl, 3-fluorenyl, 4-fluorenyl, 9-fluorenyl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 1-anthryl, 2-anthryl, 9-anthryl, 1-phenanthryl, 2-phenanthryl, 3-phenanthryl, 4-phenanthryl, 9-phenanthryl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 3-fluoranthenyl, 4-fluoranthenyl, 8-fluoranthenyl, 9-fluoranthenyl, benzofluoranthenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-16-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11 11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc. The term “(3- to 30-membered)heteroaryl(ene)” in the present disclosure is an aryl having 3 to 30 ring backbone atoms including at least one, preferably 1 to 4 heteroatoms selected from the group consisting of B, N, O, S, Si, P, Se, and Ge, in which the number of ring backbone atoms is preferably 5 to 25. The above heteroaryl or heteroarylene may be a monocyclic ring, or a fused ring condensed with at least one benzene ring; and may be partially saturated. Also, the above heteroaryl or heteroarylene in the present disclosure may be one formed by linking at least one heteroaryl or aryl group to a heteroary l group via a single bond(s). Examples of the heteroaryl specifically may include a monocyclic ring-type heteroaryl including furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl including benzofuranyl, benzothiophenyl, isobenzofuranyl, dibenzofuranyl, dibenzothiophenyl, dibenzoselenophenyl, benzofuroquinolinyl, benzofuroquinazolinyl, benzofuronaphthiridinyl, benzofuropyriraidinyl, naphthofuropyrimidinyl, benzothienoquinolinyl, benzothienoquinazolinyl, benzothienonaphthiridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzoimidazolyl, benzothiazolyl, benzoisothiazolyl, benzoisoxazolyl, benzoxazolyl, imidazopyridinyl, isoindolyl, indolyl, benzoindolyl, indazolyl, benzothiadiazolyl, quinolyl, isoquinolyl, cinnolinyl, quinazolinyl, quinoxalinyl, carbazolyl, azacarbazolyl, benzocarbazolyl, dibenzocarbazolyl, phenoxazinyl, phenanthridinyl, benzodioxolyl, indolizidinyl, acridinyl, silafluorenyl, germafluorenyl, benzotriazolyl, phenazinyl, imidazopyridinyl, chromenoquinazolinyl, thiochromenoquinazolinyl, dimethylbenzoperimidinyl, indolocarbazolyl, indenocarbazolyl, etc. More specifically, the heteroaryl may be 1-pyrrolyl, 2-pyrrolyl, 3-pyrrolyl, 2-pyridinyl, 3-pyridinyl, 4-pyridinyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolizidinyl, 2-indolizidinyl, 3-indolizidinyl, 5-indolizidinyl, 6-indolizidinyl, 7-indolizidinyl, 8-indolizidinyl, 2-imidazopyridinyl, 3-imidazopyridinyl, 5-1midazopyridinyl, 6-1midazopyrldinyl, 7-imidazopyridinyl, 8-imidazopyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-t-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-t-butyl-1-indolyl, 4-t-butyl-1-indolyl, 2-t-butyl-3-indolyl, 4-t-butyl-3-indolyl. 1-dibenzofuranyl, 2-dibenzofuranyl, 3-dibenzofuranyl, 4-dibenzofuranyl, 1-dibenzothiophenyl, 2-dibenzothiophenyl, 3-dibenzothlophenyl, 4-dibenzothlophenyl, 1-naphtho-[1,2-b]-benzofuranyl, 2-naphtho-[1,2-b]-benzofuranyl, 8-naphtha-[1,2-b]-benzofuranyl, 4-naphtho-[1,2b]-benzofuranyl, 5-naphtho-[1,2-b]-benzofuranyl, 6-naphtha-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtha-[2,3-b]-benzofuranyl, 2-naphtho-[2,3-b]-benzofuranyl, 3-naphtha-[2,3-b]-benzofuranyl, 4-naphtha-[2,3-b-]benzofuranyl, 5-naphtho-[2,3-13]-benzofuranyl, 6-naphtho-[2,3-11-benzofuranyl, 7-naphtho-[2,3-13]-benzofuranyl, 8-naphtha-[2,3-b]-benzofuranyl, 9-naphtha-[2,3-b]-benzofuranyl, 10-naphtho-[2,3-b]-benzofuranyl, 1-naphtha[2,1-b]-benzofuranyl, 2-naphtha-[2,1-b]-benzofuranyl, 3-naphtho-[2,1 -b]-benzofuranyl, 4-naphtha-[2,1-b]-benzofuranyl, 5-naphtha-[2,1-b]-benzofuranyl, 6-naphtho-[2,1-b]-benzofuranyl, 7-naphtha-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-13]-benzothiophenyl, 2-naphtha-[1,2-b]-benzothiophenyl, 3-naphtho-[1,2-b]-benzothiophenyl, 4-naphtha-[1,2-b]-benzothiophenyl, 5-naphtha-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-13]-benzothiophenyl, 10-naphtha-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtha-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1 -b]-benzothiophenyl, 4-naphtho[2,1-b]-benzothiophenyl, 5-naphtha-[2,1-b]-benzothiophenyl, 6-naphtho-2,1-b]-benzothiophenyl, 7-naphtha-[2,1-b]-benzothiophenyl, 8-naphtho-[2,1-b]-benzothiophenyl, 9-naphtha-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. The term “a fused ring of (C3-C30) aliphatic ring and (C6-C30) aromatic ring” in the present disclosure means a ring formed by fusing at least one aliphatic ring having 3 to 30 ring backbone carbon atoms in which the carbon atoms number is preferably 3 to 25, more preferably 3 to 18, and at least one aromatic ring having 6 to 30 ring backbone carbon atoms in which the carbon atoms number is preferably 6 to 25, more preferably 6 to 18. For example, the fused ring may be a fused ring of at least one benzene and at least one cyclohexane, or a fused ring of at least one naphthalene and at least one cyclopentane, etc. The carbon atoms in the fused ring of (C3-C30) aliphatic ring and (C6-C30) aromatic ring of the present disclosure may be replaced with at least one heteroatoms selected from B, N, O, S, Si and P, preferably at least one heteroatoms selected from N, O and S. The term “Halogen” in the present disclosure includes F, Cl, Br, and I.

In addition, “ortho (o),” “meta (m),” and “para (p)” in the present disclosure are meant to signify the substitution position of all substituents. Ortho position is a compound with substituents, which are adjacent to each other, i.e., at the 1 and 2 positions on benzene. Meta position is the next substitution position of the immediately adjacent substitution position, i.e., a compound with substituents at the 1 and 3 positions on benzene. Para position is the next substitution position of the meta position, i.e., a compound with substituents at the 1 and 4 positions on benzene,

The term “a ring formed in linking to an adjacent substituent” in the present disclosure means a substituted or unsubstituted (3- to 30-membered) mono- or polycyclic, alicyclic, aromatic ring, or a combination thereof, formed by linking or fusing two or more adjacent substituents, preferably may be a substituted or unsubstituted (3- to 26-membered) mono- or polycyclic, alicyclic, aromatic ring, or a combination thereof. Further, the formed ring may include at least one heteroatom selected from the group consisting of B, N, O, S, Si and P, preferably, at least one heteroatom selected from N, O and S. According to one embodiment of the present disclosure, the number of atoms in the ring skeleton is 5 to 20; according to another embodiment of the present disclosure, the number of atoms in the ring skeleton is 5 to 15. In one embodiment, the fused ring may be, for example, a substituted or unsubstituted dibenzothiophene ring, a substituted or unsubstituted dibenzofuran ring, a substituted or unsubstituted naphthalene ring, a substituted or unsubstituted phenanthrene ring, a substituted or unsubstituted fluorene ring, a substituted or unsubstituted benzothiophene ring, a substituted or unsubstituted benzofuran ring, a substituted or unsubstituted indole ring, a substituted or unsubstituted indene ring, a substituted or unsubstituted benzene ring, or a substituted or unsubstituted carbazole ring, etc.

In addition, “substituted” in the expression “substituted or unsubstituted” of the present disclosure means that a hydrogen atom in a certain functional group is replaced with another atom or functional group, i.e., a substituent, and substituted with a group to which two or more substituents are connected among the substituents. For example, “a substituent to which two or more substituents are connected” may be pyridine-triazine. That is, pyridine-triazine may be heteroaryl or may be interpreted as a substituent in which two heteroaryls are connected. Preferably, the substituent of the substituted alkyl, the substituted alkylene, the substituted alkenyl, the substituted aryl, the substituted arylene, the substituted heteroaryl, the substituted heteroarylene, the substituted cycloalkyl, the substituted cycloalkylene, the substituted alkoxy, the substituted trialkylsilyl, the substituted dialkylarylsilyl, the substituted alkyldiarylsilyl, the substituted trialkylsilyl and the substituted fused ring of aliphatic ring and aromatic ring in the formulas of the present disclosure, each independently represents at least one selected from the group consisting of deuterium; halogen; cyano; carboxyl; nitro; hydroxyl; phosphine oxide; (C1-C30)alkyl; halo(C1-C30)alkyl; (C2-C30)alkenyl; (C2-C30)alkynyl; (C1-C30)alkoxy; (C1-C30)alkylthio; (C3-C30)cycloalkyl: (C3-C30)cycloalkenyl; (3- to 7-membered)heterocycloalkyl; (C6-C30)aryloxy; (C6-C30)arylthio; (5- to 50-membered)heteroaryl unsubstituted or substituted with at least one of (C1-C30)alkyl, (C6-C30)aryl and di(C6-C30)arylamino; (C6-C30)aryl unsubstituted or substituted with at least one of (C1-C30)alkyl, (3- to 50-membered)heteroaryl, and mono- or di- (C6-C30)arylamino; tri(C1-C30)alkylsilyl; tri(C6-C30)arylsilyl; di(C1-C30)alkyl(C6-C30)arylsilyl; (C1-C30)alkyldi(C6-C30)arylsilyl; amino; mono- or di- (C1-C30)alkylamino; mono- or di- (C2-C30)alkenylamino; mono- or di- (C6-C30)arylamino unsubstituted or substituted with at least one of (C1-C30)alkyl, (5- to 30-membered)heteroaryl and di(C6-C30)arylamino; mono- or di- (3- to 30-membered)heteroarylamino; (C1-C30)alkyl(C2-C30)alkenylamino; (C1-C30)alkyl(C6-C30)arylamino; (C1-C30)alkyl(3- to 30-membered)heteroarylamino; (C2-C30)alkenyl(C6-C30)arylamino; (C2-C30)alkenyl(3- to 30-membered)heteroarylamino; (C6-C30)aryl(3- to 30-membered)heteroarylamino; (C1-C30)alkylcarbonyl; (C1-C30)alkoxycarbonyl; (C6-C30)arylcarbonyl; (C6-C30)arylphosphinyl; di(C6-C30)arylboronyl; di(C1-C30)alkylboronyl; (Cl -C30)alkyl(C6-C30)arylboronyl; (C6-C30)ar(C1-C30)alkyl; and (C1-C30)alkyl(C6-C30)aryl. For example, the substituent may be unsubstituted methyl, unsubstituted phenyl, or unsubstituted naphthyl.

Hereinafter, an organic electroluminescent compound according to one embodiment will be described.

The organic electroluminescent compound according to one embodiment is represented by the following formula 1.

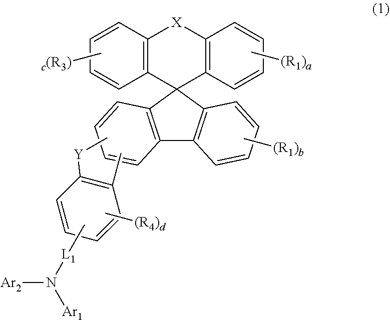

In formula 1,

X and Y each independently represent, O or S;

L₁ represents a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene;

Ar₁ and Ar₂ each independently represent, a substituted or unsubstituted (C6-C30)aryl, a substituted or unsubstituted (3- to 30-membered)heteroaryl, a substituted or unsubstituted (C3-C30)cycloalkyl, a substituted or unsubstituted fused ring of (C3-C30) aliphatic ring and (C6-C30) aromatic ring, or -L₂-N-(Ar₃)(Ar₄); or may be linked to an adjacent substituent(s) to form a ring(s);

R₁ to R₄ each independently represent, hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or -L₃-N-(Ar₅)(Ar₆); or may be linked to an adjacent substituent(s) to form a ring(s);

L₂ and L₃ each independently represent, a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene;

Ar₃ to Ar₅ each independently represent, a substituted or unsubstituted (C1-C30)alkyl, a substituted or unsubstituted (C2-C30)alkenyl, a substituted or unsubstituted (C6-C30)aryl, a substituted or unsubstituted fused ring of (C3-C30) aliphatic ring and (C6-C30) aromatic ring, or a substituted or unsubstituted (3- to 30-membered)heteroaryl;

a to c each independently represent, an integer of 1 to 4, and d represents an integer of 1 to 3; and

when a to d each independently are an integer of 2 or more, each of R₁ to R₄ may be the same or different.

In one embodiment, all of X and Y may be O, or all of X and Y may be S. For example, X may be O, and Y may be S. For further example, X may be S, and Y may be O.

In one embodiment, L₁ may be a single bond or a substituted or unsubstituted (C6-C30)arylene, preferably, a single bond or a substituted or unsubstituted (C6-C25)arylene, more preferably, a single bond or a substituted or unsubstituted (C6-C18)arylene. For example, L₁ may be a single bond or unsubstituted phenylene.

In one embodiment, Ar₁ and Ar₂ each independently may be, a substituted or unsubstituted (C6-C30)aryl, a substituted or unsubstituted (3- to 30-membered)heteroaryl, or a substituted or unsubstituted fused ring of (C3-C30) aliphatic ring and (C6-C30) aromatic ring, preferably, a substituted or unsubstituted (C6-C30)aryl, a substituted or unsubstituted (5- to 30-membered)heteroaryl, or a substituted or unsubstituted fused ring of (C3-C20) aliphatic ring and (C6-C25) aromatic ring, more preferably, a substituted or unsubstituted (C6-C25)aryl, a substituted or unsubstituted (5- to 25-membered)heteroaryl, or a substituted or unsubstituted fused ring of (C3-C10) aliphatic ring and (C6-C18) aromatic ring. For example, Ar₁ and Ar₂ each independently may be phenyl unsubstituted or substituted with naphthyl, a substituted or unsubstituted o-biphenyl, a substituted or unsubstituted m-biphenyl, a substituted or unsubstituted p-biphenyl, a substituted or unsubstituted dimethylfluorenyl, a substituted or unsubstituted diphenylfluorenyl, a substituted or unsubstituted spirobifluorenyl, a substituted or unsubstituted dibenzofuranyl, a substituted or unsubstituted dibenzothiophenyl, or a substituted or unsubstituted dihydrotetramethylphenanthrenyl.

In one embodiment, all of R₁ to R₄ may be hydrogen.

According to one embodiment, the organic electroluminescent compound of formula 1 may be represented by any one of the following formulas 1-1 to 1-4.

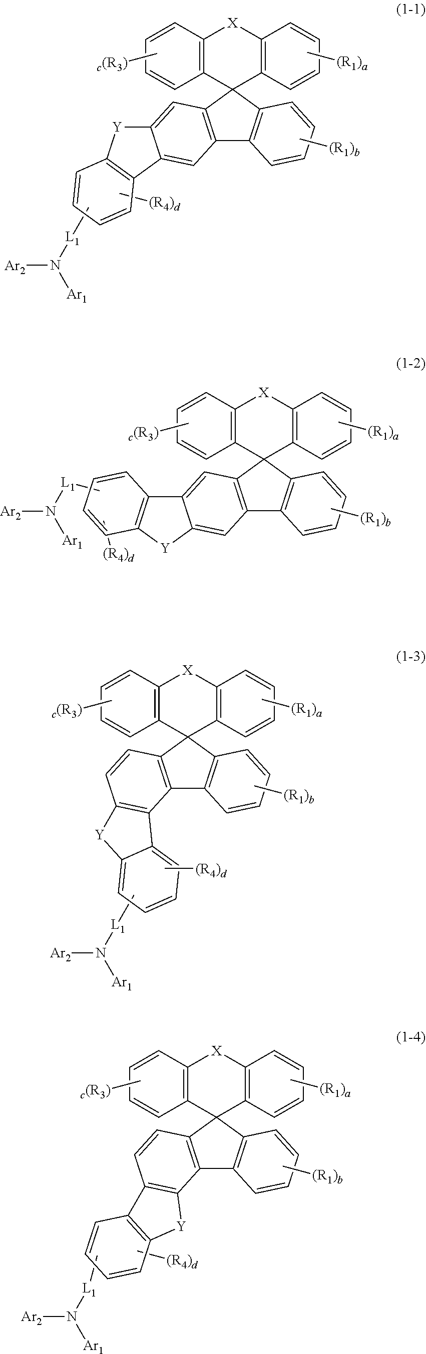

In formulas 1-1 to 1-4,

R₁ to R_{4,} X, Y, L₁, Ar₁, Ar₂, and a to d each independently are as defined in formula 1 above.

The formulas 1-1 to 1-4 according to one embodiment may be the organic electroluminescent compound where X and Y each independently represent, O or S; represents a single bond or a substituted or unsubstituted (C6-C30)arylene; Ar₁, and Ar₂ each independently represent, a substituted or unsubstituted (C6-C30)aryl, a substituted or unsubstituted (3- to 30-membered)heteroaryl, or a substituted or unsubstituted fused ring of (C3-C30) aliphatic ring and (C6-C30) aromatic ring; and all of R₁ to R₄ are hydrogen.

According to one embodiment, the organic electroluminescent compound represented by formula 1 above may be more specifically illustrated by the following compounds, but is not limited thereto.

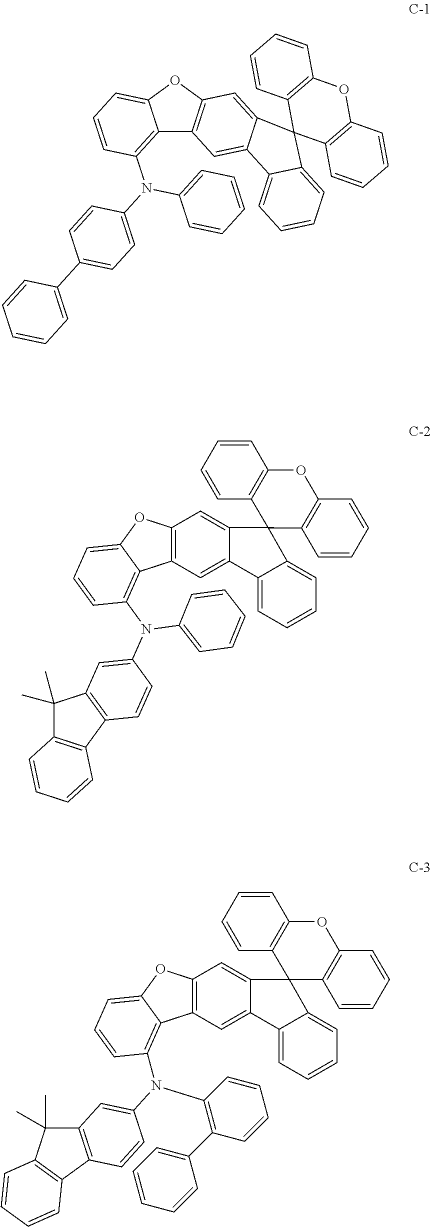 [SMILES: c1ccc(-c2ccc(N(c3ccccc3)c3cccc4oc5cc6c(cc5c34)-c3ccccc3C63c4ccccc4Oc4ccccc43)cc2)cc1 ; CC1(C)c2ccccc2-c2ccc(N(c3ccccc3)c3cccc4oc5cc6c(cc5c34)-c3ccccc3C63c4ccccc4Oc4ccccc43)cc21 ; CC1(C)c2ccccc2-c2ccc(N(c3ccccc3-c3ccccc3)c3cccc4oc5cc6c(cc5c34)-c3ccccc3C63c4ccccc4Oc4ccccc43)cc21]

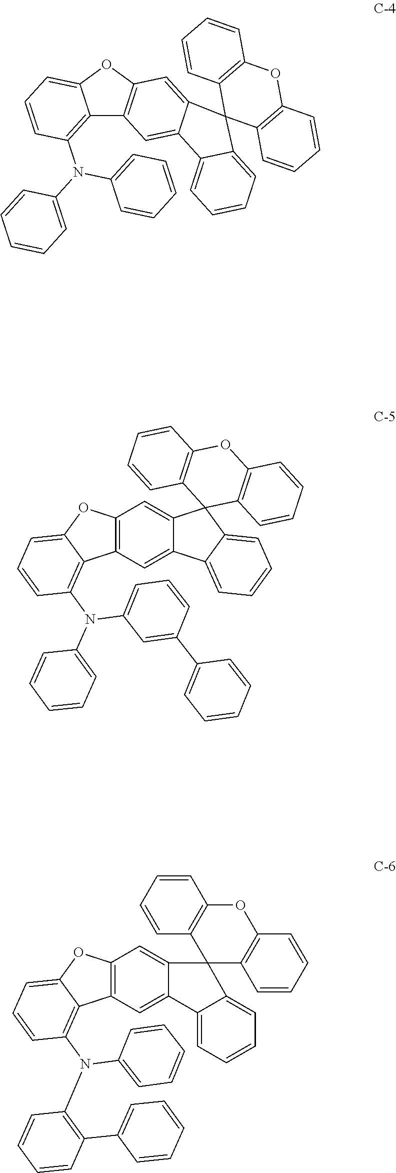 [SMILES: c1ccc(N(c2ccccc2)c2cccc3oc4cc5c(cc4c23)-c2ccccc2C52c3ccccc3Oc3ccccc32)cc1 ; c1ccc(-c2cccc(N(c3ccccc3)c3cccc4oc5cc6c(cc5c34)-c3ccccc3C63c4ccccc4Oc4ccccc43)c2)cc1 ; c1ccc(-c2ccccc2N(c2ccccc2)c2cccc3oc4cc5c(cc4c23)-c2ccccc2C52c3ccccc3Oc3ccccc32)cc1]

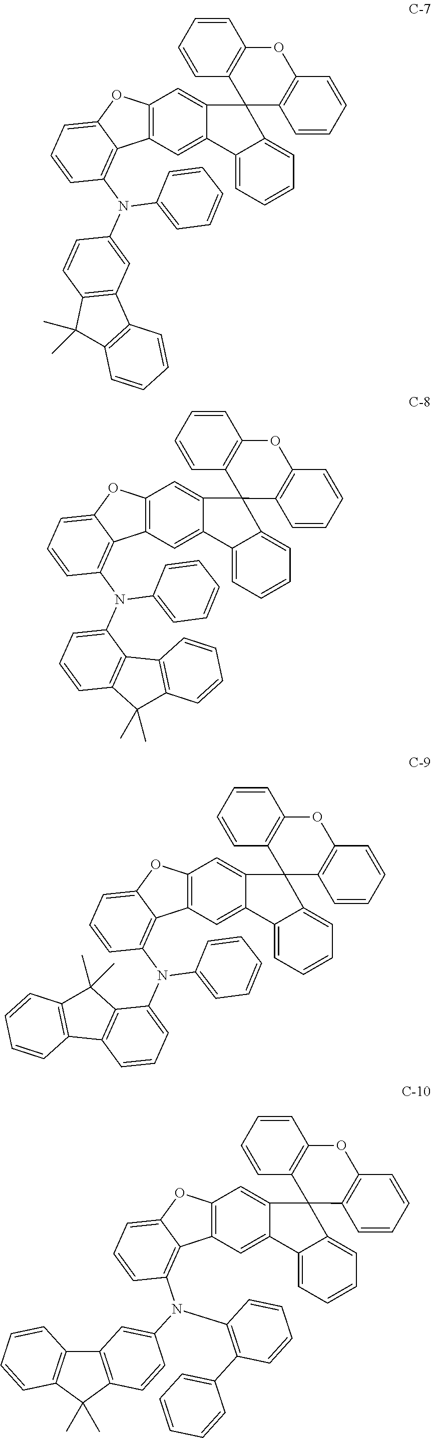 [SMILES: CC1(C)c2ccccc2-c2cc(N(c3ccccc3)c3cccc4oc5cc6c(cc5c34)-c3ccccc3C63c4ccccc4Oc4ccccc43)ccc21 ; CC1(C)c2ccccc2-c2c(N(c3ccccc3)c3cccc4oc5cc6c(cc5c34)-c3ccccc3C63c4ccccc4Oc4ccccc43)cccc21 ; CC1(C)c2ccccc2-c2cccc(N(c3ccccc3)c3cccc4oc5cc6c(cc5c34)-c3ccccc3C63c4ccccc4Oc4ccccc43)c21 ; CC1(C)c2ccccc2-c2cc(N(c3ccccc3-c3ccccc3)c3cccc4oc5cc6c(cc5c34)-c3ccccc3C63c4ccccc4Oc4ccccc43)ccc21]

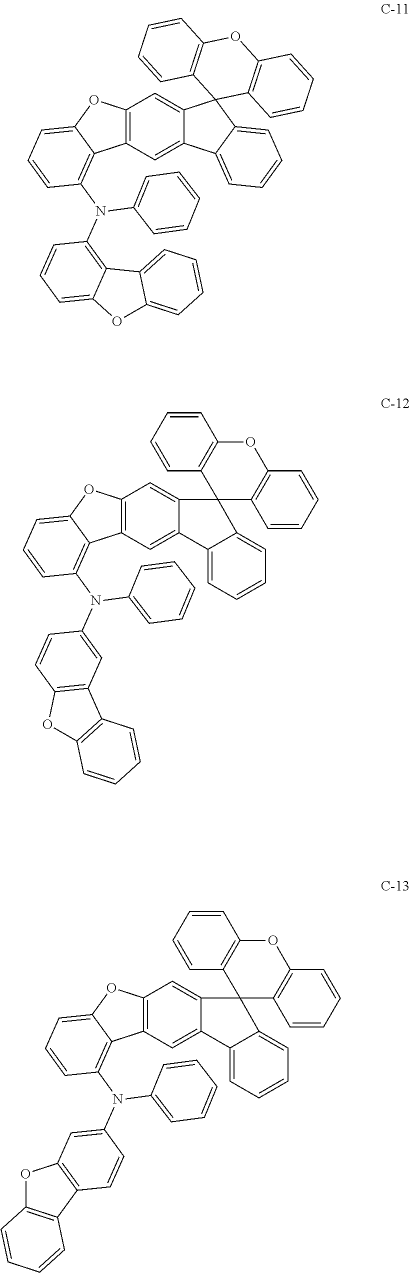 [SMILES: c1ccc(N(c2cccc3oc4ccccc4c23)c2cccc3oc4cc5c(cc4c23)-c2ccccc2C52c3ccccc3Oc3ccccc32)cc1 ; c1ccc(N(c2ccc3oc4ccccc4c3c2)c2cccc3oc4cc5c(cc4c23)-c2ccccc2C52c3ccccc3Oc3ccccc32)cc1 ; c1ccc(N(c2ccc3c(c2)oc2ccccc23)c2cccc3oc4cc5c(cc4c23)-c2ccccc2C52c3ccccc3Oc3ccccc32)cc1]

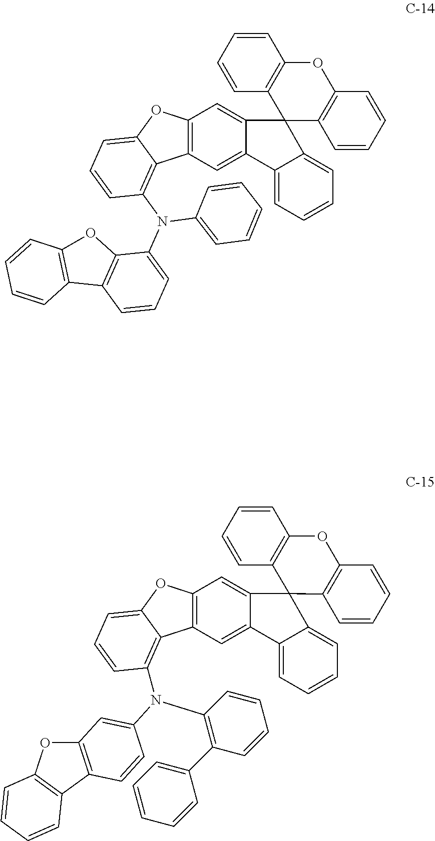 [SMILES: c1ccc(N(c2cccc3c2oc2ccccc23)c2cccc3oc4cc5c(cc4c23)-c2ccccc2C52c3ccccc3Oc3ccccc32)cc1 ; c1ccc(-c2ccccc2N(c2ccc3c(c2)oc2ccccc23)c2cccc3oc4cc5c(cc4c23)-c2ccccc2C52c3ccccc3Oc3ccccc32)cc1]

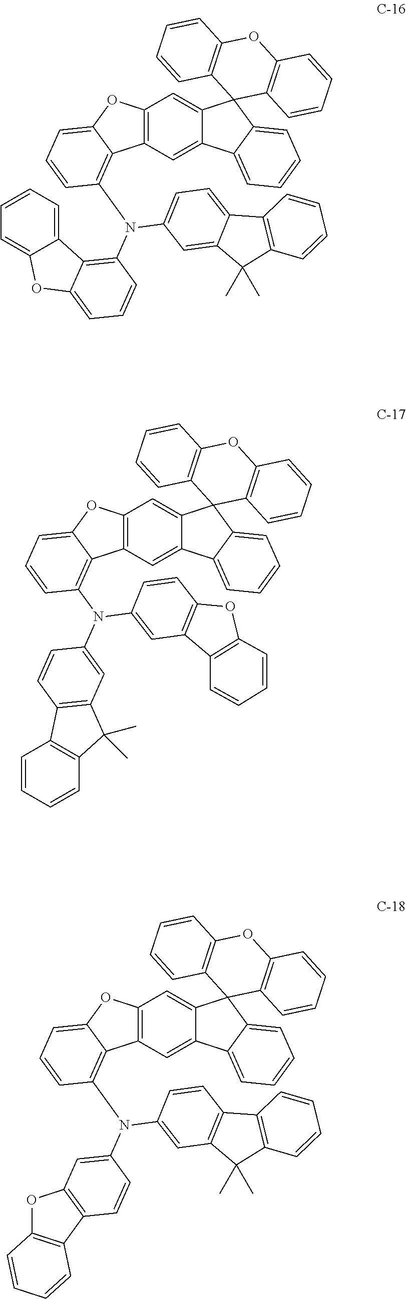 [SMILES: CC1(C)c2ccccc2-c2ccc(N(c3cccc4oc5ccccc5c34)c3cccc4oc5cc6c(cc5c34)-c3ccccc3C63c4ccccc4Oc4ccccc43)cc21 ; CC1(C)c2ccccc2-c2ccc(N(c3ccc4oc5ccccc5c4c3)c3cccc4oc5cc6c(cc5c34)-c3ccccc3C63c4ccccc4Oc4ccccc43)cc21 ; CC1(C)c2ccccc2-c2ccc(N(c3ccc4c(c3)oc3ccccc34)c3cccc4oc5cc6c(cc5c34)-c3ccccc3C63c4ccccc4Oc4ccccc43)cc21]

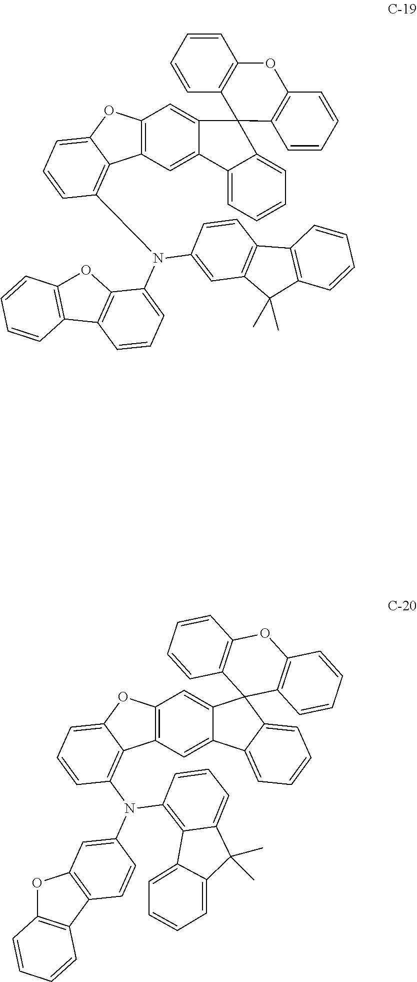 [SMILES: CC1(C)c2ccccc2-c2ccc(N(c3cccc4c3oc3ccccc34)c3cccc4oc5cc6c(cc5c34)-c3ccccc3C63c4ccccc4Oc4ccccc43)cc21 ; CC1(C)c2ccccc2-c2c(N(c3ccc4c(c3)oc3ccccc34)c3cccc4oc5cc6c(cc5c34)-c3ccccc3C63c4ccccc4Oc4ccccc43)cccc21]

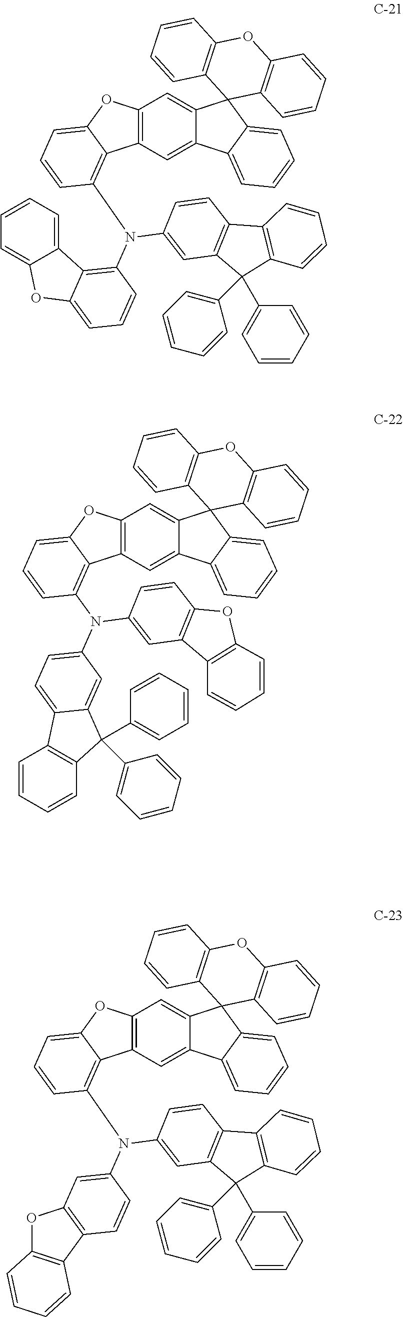 [SMILES: c1ccc(C2(c3ccccc3)c3ccccc3-c3ccc(N(c4cccc5oc6ccccc6c45)c4cccc5oc6cc7c(cc6c45)-c4ccccc4C74c5ccccc5Oc5ccccc54)cc32)cc1 ; c1ccc(C2(c3ccccc3)c3ccccc3-c3ccc(N(c4ccc5oc6ccccc6c5c4)c4cccc5oc6cc7c(cc6c45)-c4ccccc4C74c5ccccc5Oc5ccccc54)cc32)cc1 ; c1ccc(C2(c3ccccc3)c3ccccc3-c3ccc(N(c4ccc5c(c4)oc4ccccc45)c4cccc5oc6cc7c(cc6c45)-c4ccccc4C74c5ccccc5Oc5ccccc54)cc32)cc1]

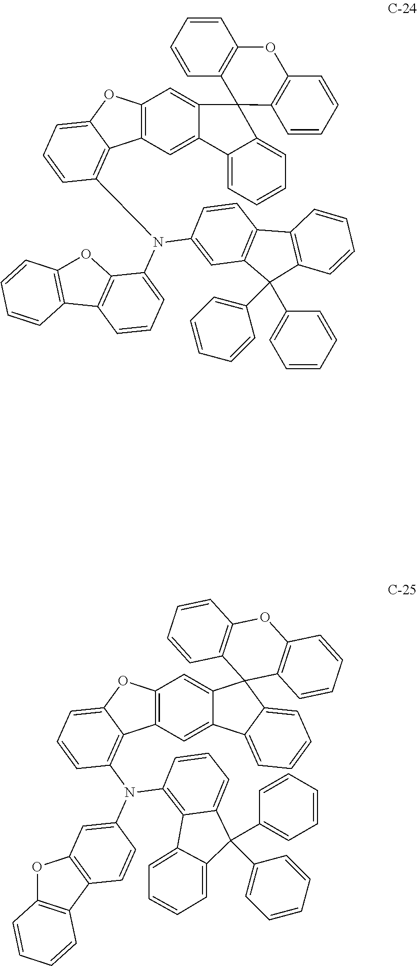 [SMILES: c1ccc(C2(c3ccccc3)c3ccccc3-c3ccc(N(c4cccc5c4oc4ccccc45)c4cccc5oc6cc7c(cc6c45)-c4ccccc4C74c5ccccc5Oc5ccccc54)cc32)cc1 ; c1ccc(C2(c3ccccc3)c3ccccc3-c3c(N(c4ccc5c(c4)oc4ccccc45)c4cccc5oc6cc7c(cc6c45)-c4ccccc4C74c5ccccc5Oc5ccccc54)cccc32)cc1]

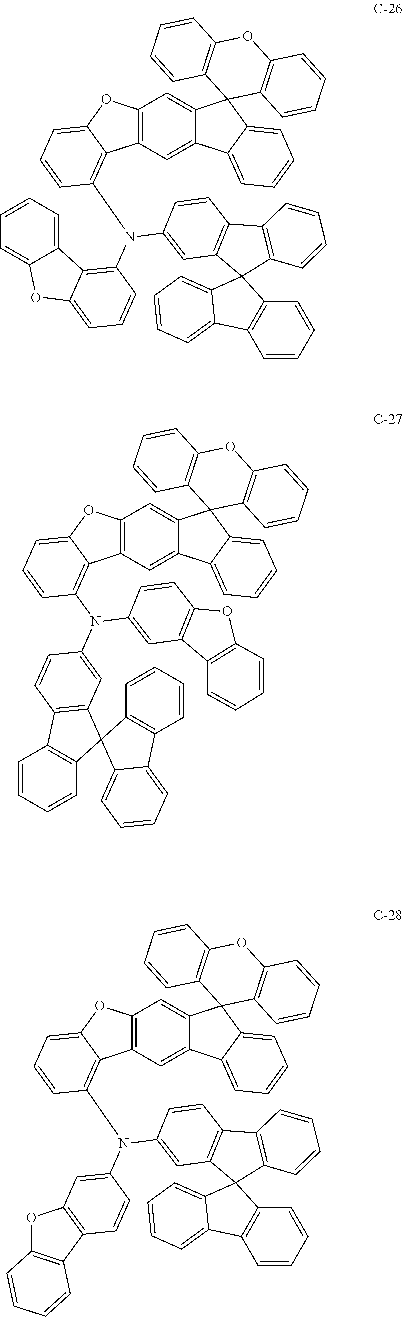 [SMILES: c1ccc2c(c1)Oc1ccccc1C21c2ccccc2-c2cc3c(cc21)oc1cccc(N(c2ccc4c(c2)C2(c5ccccc5-c5ccccc52)c2ccccc2-4)c2cccc4oc5ccccc5c24)c13 ; c1ccc2c(c1)Oc1ccccc1C21c2ccccc2-c2cc3c(cc21)oc1cccc(N(c2ccc4c(c2)C2(c5ccccc5-c5ccccc52)c2ccccc2-4)c2ccc4oc5ccccc5c4c2)c13 ; c1ccc2c(c1)Oc1ccccc1C21c2ccccc2-c2cc3c(cc21)oc1cccc(N(c2ccc4c(c2)C2(c5ccccc5-c5ccccc52)c2ccccc2-4)c2ccc4c(c2)oc2ccccc24)c13]

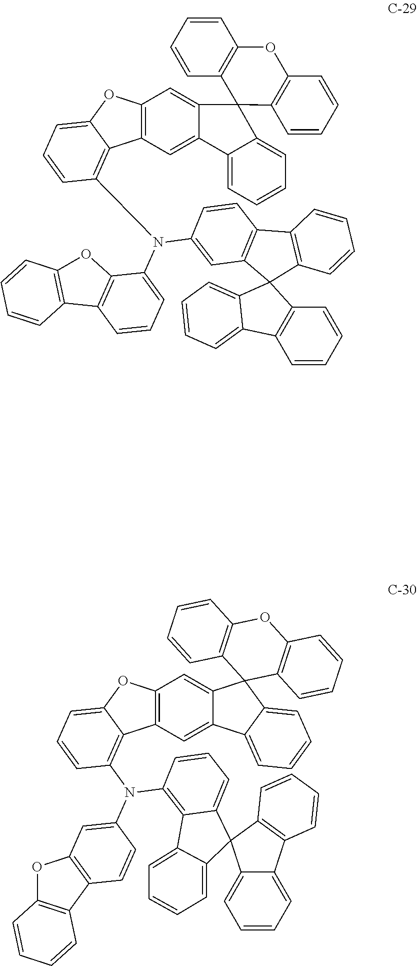 [SMILES: c1ccc2c(c1)Oc1ccccc1C21c2ccccc2-c2cc3c(cc21)oc1cccc(N(c2ccc4c(c2)C2(c5ccccc5-c5ccccc52)c2ccccc2-4)c2cccc4c2oc2ccccc24)c13 ; c1ccc2c(c1)Oc1ccccc1C21c2ccccc2-c2cc3c(cc21)oc1cccc(N(c2ccc4c(c2)oc2ccccc24)c2cccc4c2-c2ccccc2C42c4ccccc4-c4ccccc42)c13]

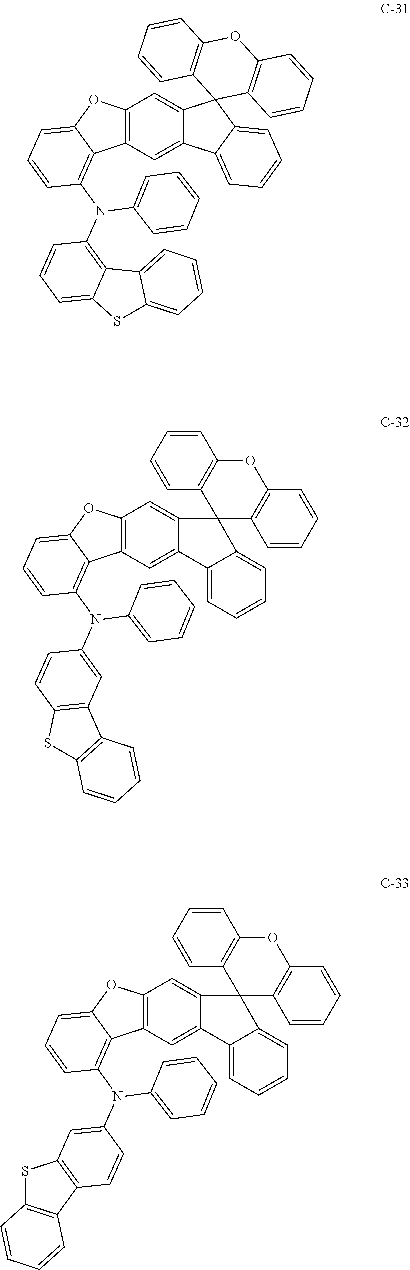 [SMILES: c1ccc(N(c2cccc3oc4cc5c(cc4c23)-c2ccccc2C52c3ccccc3Oc3ccccc32)c2cccc3sc4ccccc4c23)cc1 ; c1ccc(N(c2ccc3sc4ccccc4c3c2)c2cccc3oc4cc5c(cc4c23)-c2ccccc2C52c3ccccc3Oc3ccccc32)cc1 ; c1ccc(N(c2ccc3c(c2)sc2ccccc23)c2cccc3oc4cc5c(cc4c23)-c2ccccc2C52c3ccccc3Oc3ccccc32)cc1]

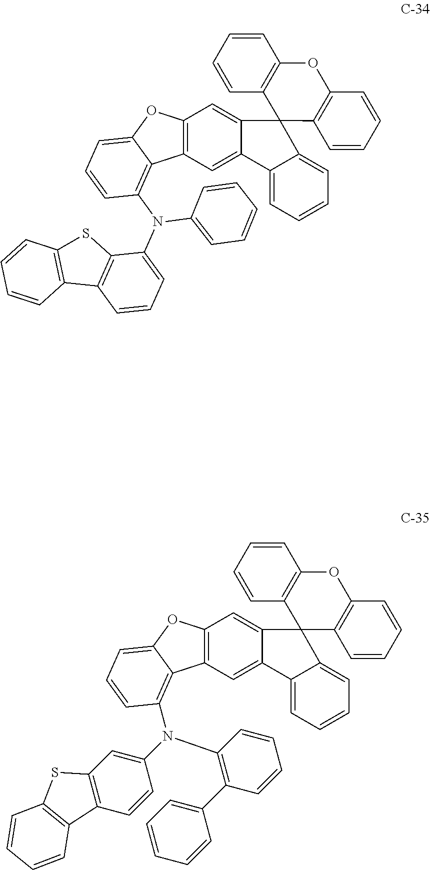 [SMILES: c1ccc(N(c2cccc3c2sc2ccccc23)c2cccc3oc4cc5c(cc4c23)-c2ccccc2C52c3ccccc3Oc3ccccc32)cc1 ; c1ccc(-c2ccccc2N(c2ccc3c(c2)sc2ccccc23)c2cccc3oc4cc5c(cc4c23)-c2ccccc2C52c3ccccc3Oc3ccccc32)cc1]

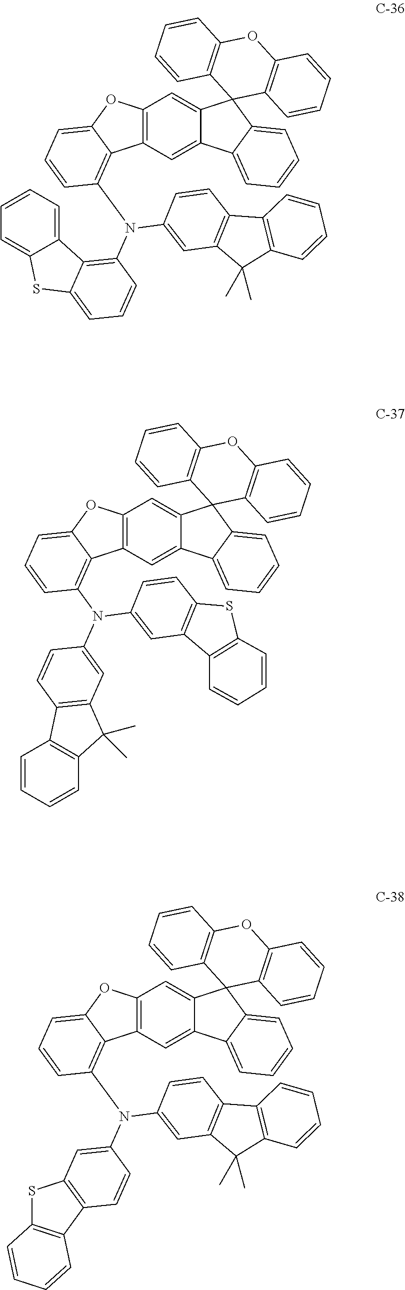 [SMILES: CC1(C)c2ccccc2-c2ccc(N(c3cccc4oc5cc6c(cc5c34)-c3ccccc3C63c4ccccc4Oc4ccccc43)c3cccc4sc5ccccc5c34)cc21 ; CC1(C)c2ccccc2-c2ccc(N(c3ccc4sc5ccccc5c4c3)c3cccc4oc5cc6c(cc5c34)-c3ccccc3C63c4ccccc4Oc4ccccc43)cc21 ; CC1(C)c2ccccc2-c2ccc(N(c3ccc4c(c3)sc3ccccc34)c3cccc4oc5cc6c(cc5c34)-c3ccccc3C63c4ccccc4Oc4ccccc43)cc21]

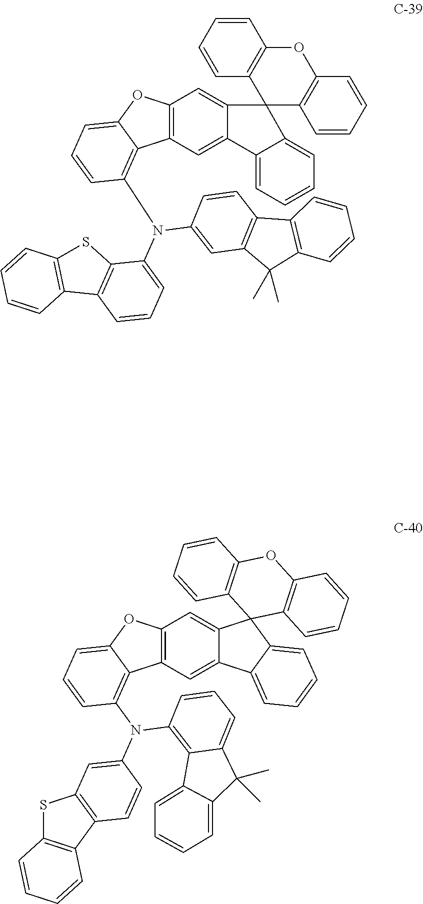 [SMILES: CC1(C)c2ccccc2-c2ccc(N(c3cccc4c3sc3ccccc34)c3cccc4oc5cc6c(cc5c34)-c3ccccc3C63c4ccccc4Oc4ccccc43)cc21 ; CC1(C)c2ccccc2-c2c(N(c3ccc4c(c3)sc3ccccc34)c3cccc4oc5cc6c(cc5c34)-c3ccccc3C63c4ccccc4Oc4ccccc43)cccc21]

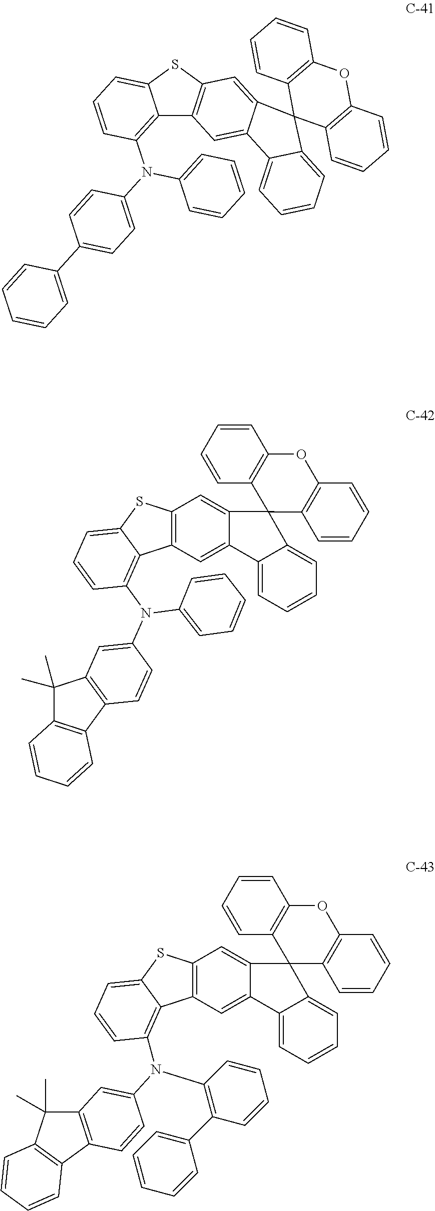 [SMILES: c1ccc(-c2ccc(N(c3ccccc3)c3cccc4sc5cc6c(cc5c34)-c3ccccc3C63c4ccccc4Oc4ccccc43)cc2)cc1 ; CC1(C)c2ccccc2-c2ccc(N(c3ccccc3)c3cccc4sc5cc6c(cc5c34)-c3ccccc3C63c4ccccc4Oc4ccccc43)cc21 ; CC1(C)c2ccccc2-c2ccc(N(c3ccccc3-c3ccccc3)c3cccc4sc5cc6c(cc5c34)-c3ccccc3C63c4ccccc4Oc4ccccc43)cc21]

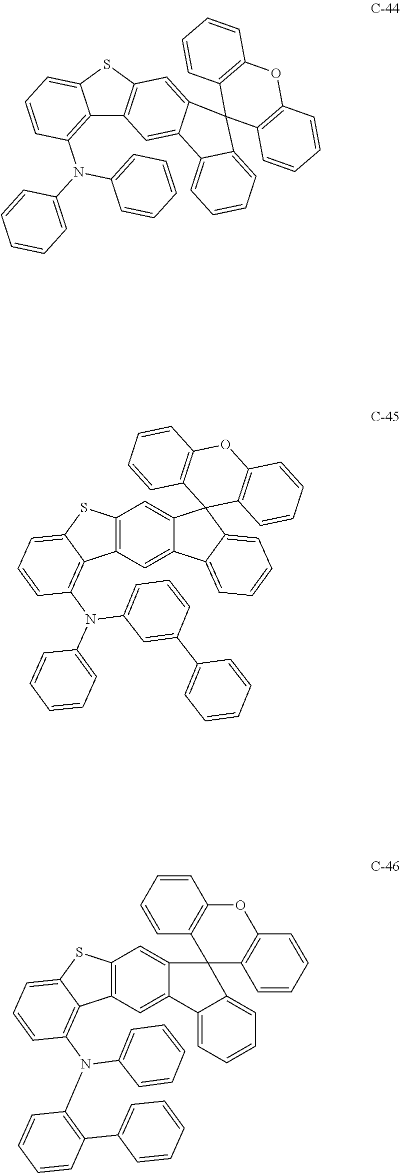 [SMILES: c1ccc(N(c2ccccc2)c2cccc3sc4cc5c(cc4c23)-c2ccccc2C52c3ccccc3Oc3ccccc32)cc1 ; c1ccc(-c2cccc(N(c3ccccc3)c3cccc4sc5cc6c(cc5c34)-c3ccccc3C63c4ccccc4Oc4ccccc43)c2)cc1 ; c1ccc(-c2ccccc2N(c2ccccc2)c2cccc3sc4cc5c(cc4c23)-c2ccccc2C52c3ccccc3Oc3ccccc32)cc1]

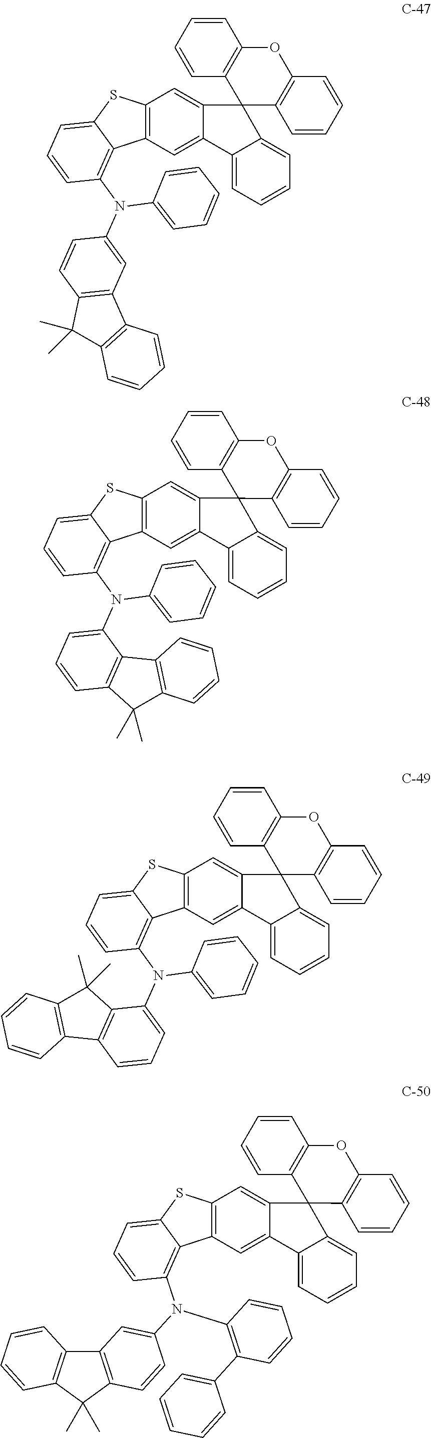 [SMILES: CC1(C)c2ccccc2-c2cc(N(c3ccccc3)c3cccc4sc5cc6c(cc5c34)-c3ccccc3C63c4ccccc4Oc4ccccc43)ccc21 ; CC1(C)c2ccccc2-c2c(N(c3ccccc3)c3cccc4sc5cc6c(cc5c34)-c3ccccc3C63c4ccccc4Oc4ccccc43)cccc21 ; CC1(C)c2ccccc2-c2cccc(N(c3ccccc3)c3cccc4sc5cc6c(cc5c34)-c3ccccc3C63c4ccccc4Oc4ccccc43)c21 ; CC1(C)c2ccccc2-c2cc(N(c3ccccc3-c3ccccc3)c3cccc4sc5cc6c(cc5c34)-c3ccccc3C63c4ccccc4Oc4ccccc43)ccc21]

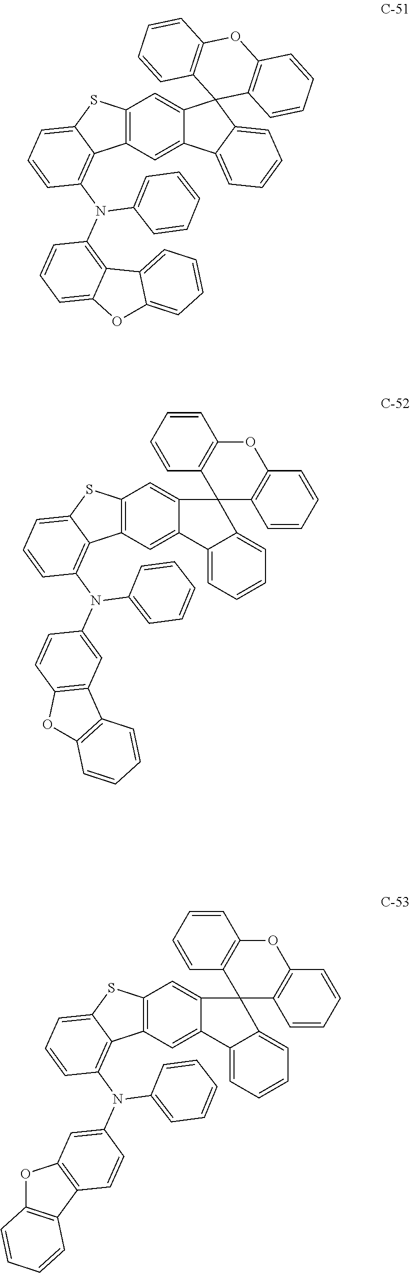 [SMILES: c1ccc(N(c2cccc3oc4ccccc4c23)c2cccc3sc4cc5c(cc4c23)-c2ccccc2C52c3ccccc3Oc3ccccc32)cc1 ; c1ccc(N(c2ccc3oc4ccccc4c3c2)c2cccc3sc4cc5c(cc4c23)-c2ccccc2C52c3ccccc3Oc3ccccc32)cc1 ; c1ccc(N(c2ccc3c(c2)oc2ccccc23)c2cccc3sc4cc5c(cc4c23)-c2ccccc2C52c3ccccc3Oc3ccccc32)cc1]

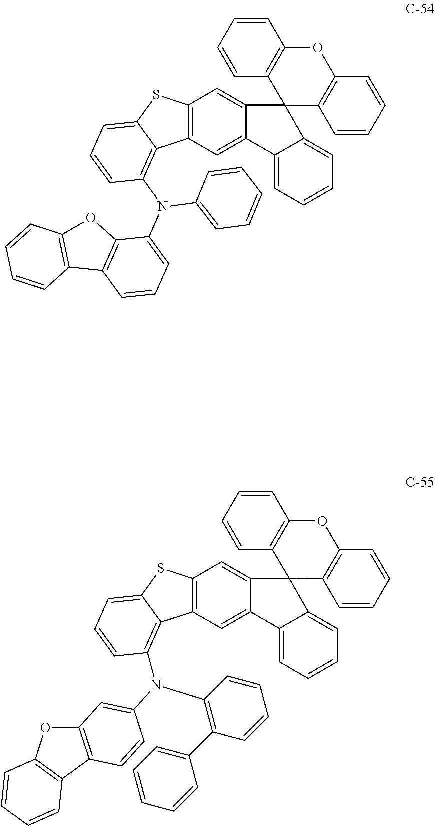 [SMILES: c1ccc(N(c2cccc3c2oc2ccccc23)c2cccc3sc4cc5c(cc4c23)-c2ccccc2C52c3ccccc3Oc3ccccc32)cc1 ; c1ccc(-c2ccccc2N(c2ccc3c(c2)oc2ccccc23)c2cccc3sc4cc5c(cc4c23)-c2ccccc2C52c3ccccc3Oc3ccccc32)cc1]

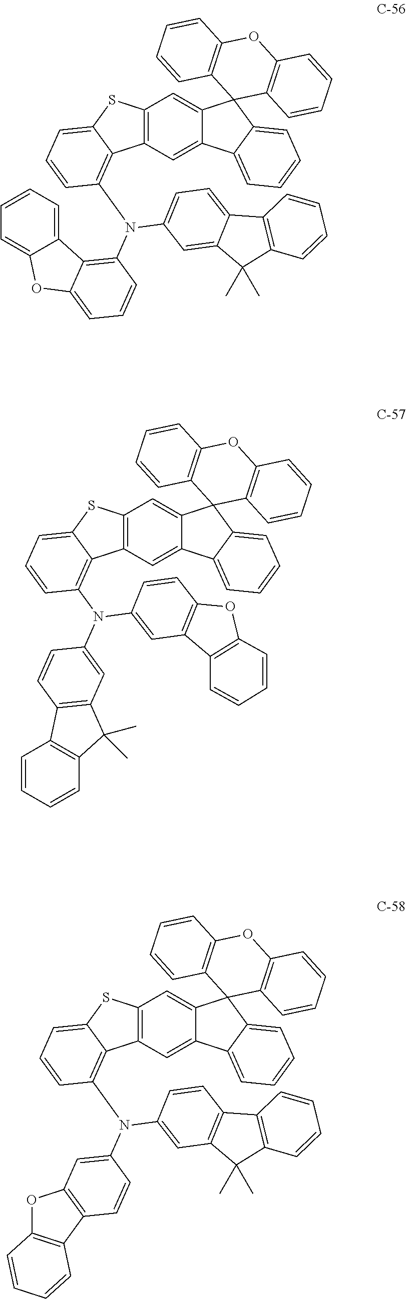 [SMILES: CC1(C)c2ccccc2-c2ccc(N(c3cccc4oc5ccccc5c34)c3cccc4sc5cc6c(cc5c34)-c3ccccc3C63c4ccccc4Oc4ccccc43)cc21 ; CC1(C)c2ccccc2-c2ccc(N(c3ccc4oc5ccccc5c4c3)c3cccc4sc5cc6c(cc5c34)-c3ccccc3C63c4ccccc4Oc4ccccc43)cc21 ; CC1(C)c2ccccc2-c2ccc(N(c3ccc4c(c3)oc3ccccc34)c3cccc4sc5cc6c(cc5c34)-c3ccccc3C63c4ccccc4Oc4ccccc43)cc21]

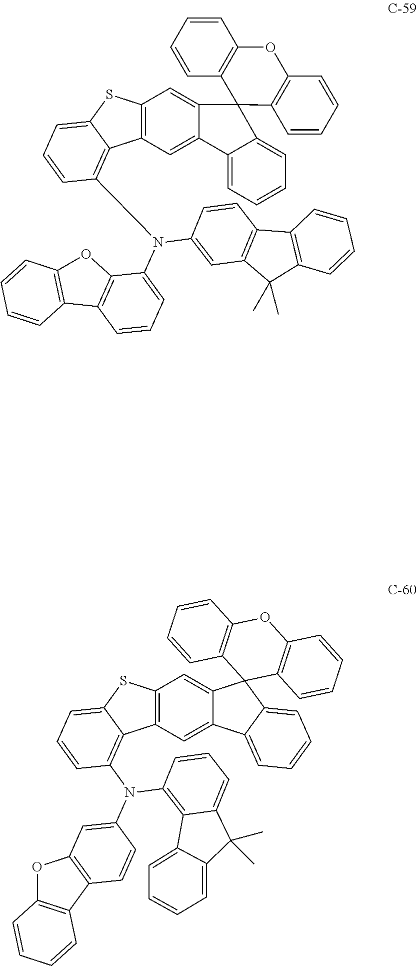 [SMILES: CC1(C)c2ccccc2-c2ccc(N(c3cccc4c3oc3ccccc34)c3cccc4sc5cc6c(cc5c34)-c3ccccc3C63c4ccccc4Oc4ccccc43)cc21 ; CC1(C)c2ccccc2-c2c(N(c3ccc4c(c3)oc3ccccc34)c3cccc4sc5cc6c(cc5c34)-c3ccccc3C63c4ccccc4Oc4ccccc43)cccc21]

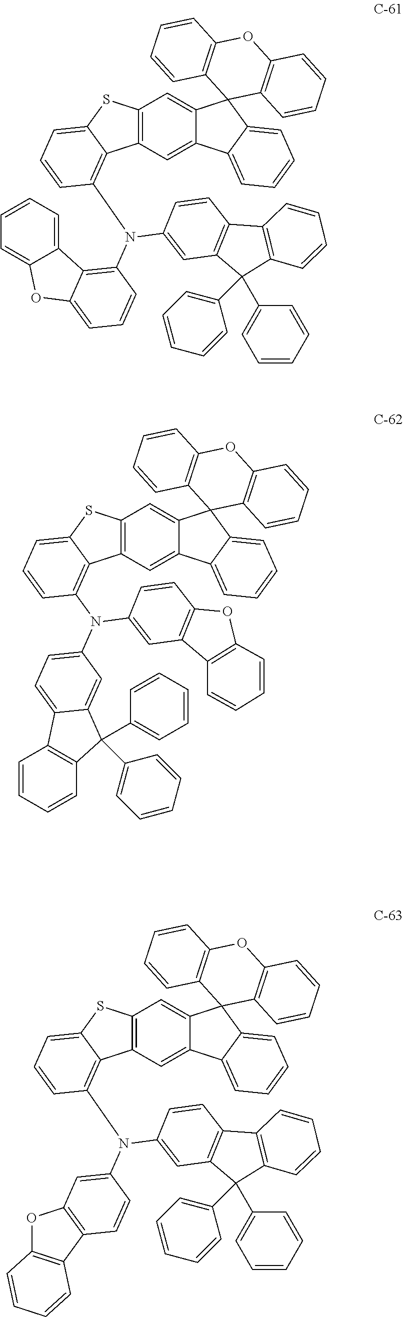 [SMILES: c1ccc(C2(c3ccccc3)c3ccccc3-c3ccc(N(c4cccc5oc6ccccc6c45)c4cccc5sc6cc7c(cc6c45)-c4ccccc4C74c5ccccc5Oc5ccccc54)cc32)cc1 ; c1ccc(C2(c3ccccc3)c3ccccc3-c3ccc(N(c4ccc5oc6ccccc6c5c4)c4cccc5sc6cc7c(cc6c45)-c4ccccc4C74c5ccccc5Oc5ccccc54)cc32)cc1 ; c1ccc(C2(c3ccccc3)c3ccccc3-c3ccc(N(c4ccc5c(c4)oc4ccccc45)c4cccc5sc6cc7c(cc6c45)-c4ccccc4C74c5ccccc5Oc5ccccc54)cc32)cc1]

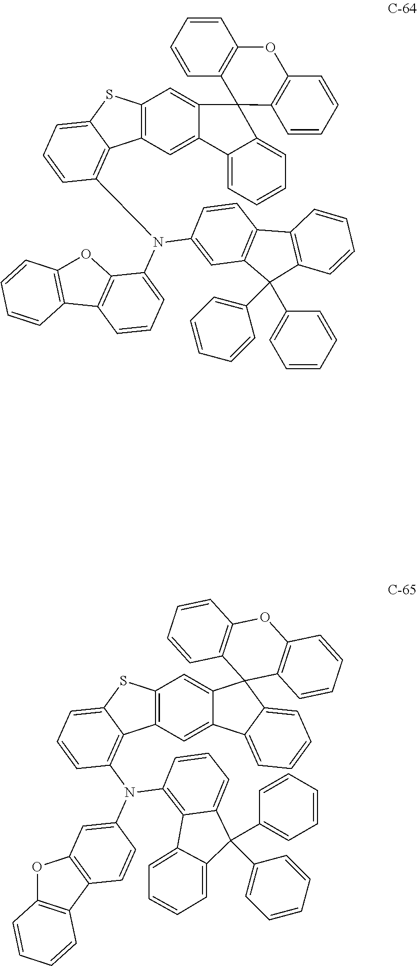 [SMILES: c1ccc(C2(c3ccccc3)c3ccccc3-c3ccc(N(c4cccc5c4oc4ccccc45)c4cccc5sc6cc7c(cc6c45)-c4ccccc4C74c5ccccc5Oc5ccccc54)cc32)cc1 ; c1ccc(C2(c3ccccc3)c3ccccc3-c3c(N(c4ccc5c(c4)oc4ccccc45)c4cccc5sc6cc7c(cc6c45)-c4ccccc4C74c5ccccc5Oc5ccccc54)cccc32)cc1]

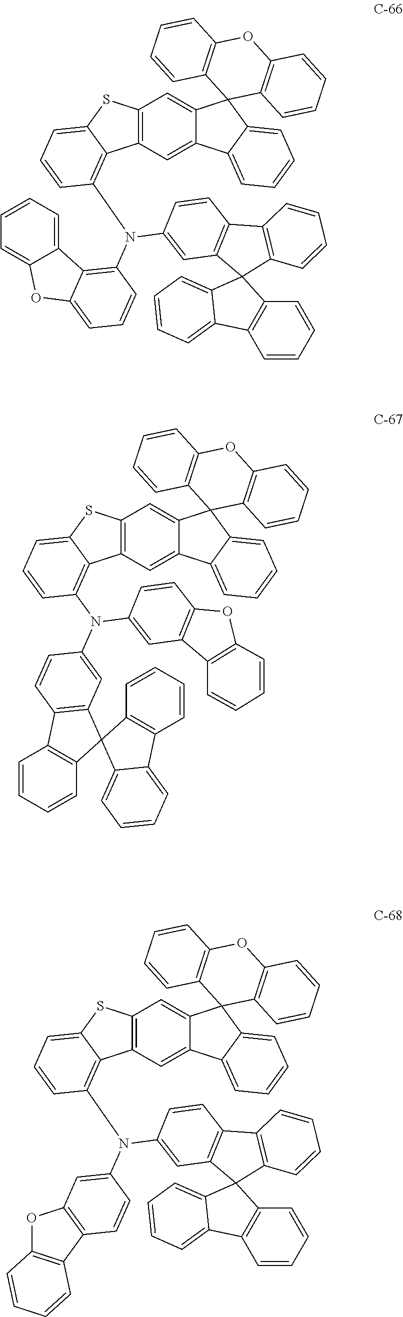 [SMILES: c1ccc2c(c1)Oc1ccccc1C21c2ccccc2-c2cc3c(cc21)sc1cccc(N(c2ccc4c(c2)C2(c5ccccc5-c5ccccc52)c2ccccc2-4)c2cccc4oc5ccccc5c24)c13 ; c1ccc2c(c1)Oc1ccccc1C21c2ccccc2-c2cc3c(cc21)sc1cccc(N(c2ccc4c(c2)C2(c5ccccc5-c5ccccc52)c2ccccc2-4)c2ccc4oc5ccccc5c4c2)c13 ; c1ccc2c(c1)Oc1ccccc1C21c2ccccc2-c2cc3c(cc21)sc1cccc(N(c2ccc4c(c2)C2(c5ccccc5-c5ccccc52)c2ccccc2-4)c2ccc4c(c2)oc2ccccc24)c13]

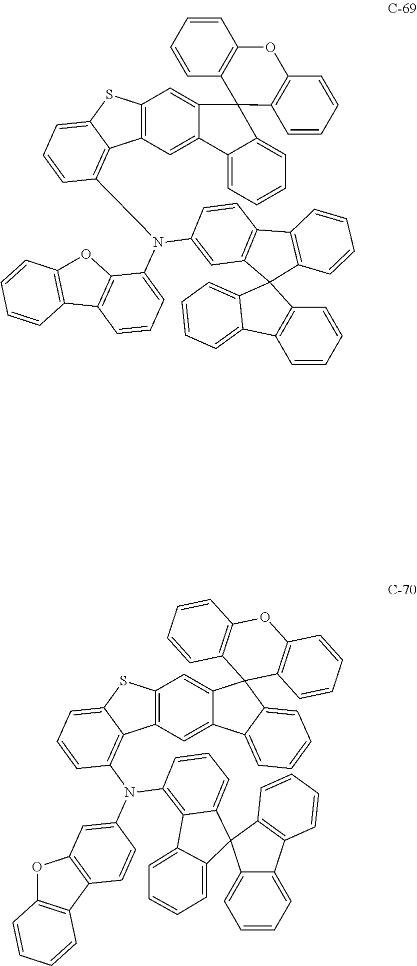 [SMILES: c1ccc2c(c1)Oc1ccccc1C21c2ccccc2-c2cc3c(cc21)sc1cccc(N(c2ccc4c(c2)C2(c5ccccc5-c5ccccc52)c2ccccc2-4)c2cccc4c2oc2ccccc24)c13 ; c1ccc2c(c1)Oc1ccccc1C21c2ccccc2-c2cc3c(cc21)sc1cccc(N(c2ccc4c(c2)oc2ccccc24)c2cccc4c2-c2ccccc2C42c4ccccc4-c4ccccc42)c13]

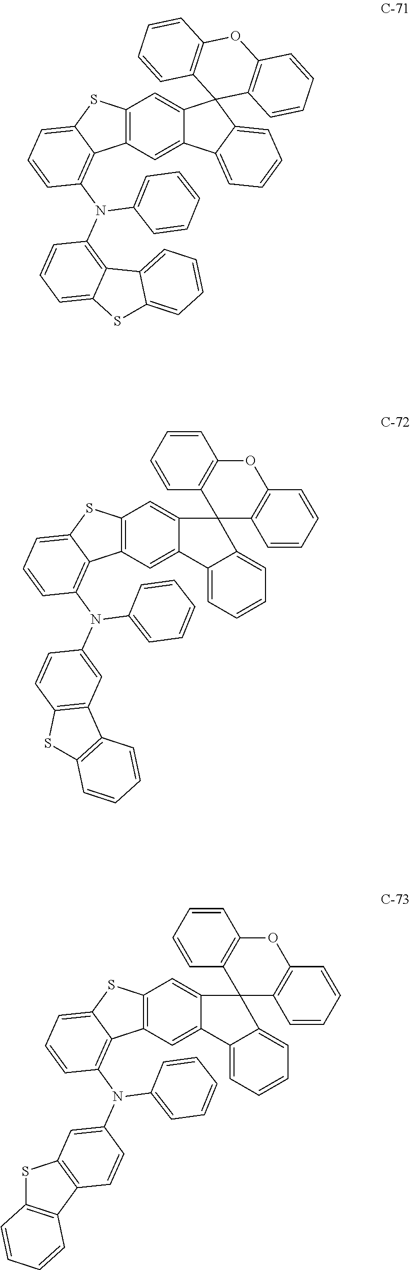 [SMILES: c1ccc(N(c2cccc3sc4ccccc4c23)c2cccc3sc4cc5c(cc4c23)-c2ccccc2C52c3ccccc3Oc3ccccc32)cc1 ; c1ccc(N(c2ccc3sc4ccccc4c3c2)c2cccc3sc4cc5c(cc4c23)-c2ccccc2C52c3ccccc3Oc3ccccc32)cc1 ; c1ccc(N(c2ccc3c(c2)sc2ccccc23)c2cccc3sc4cc5c(cc4c23)-c2ccccc2C52c3ccccc3Oc3ccccc32)cc1]

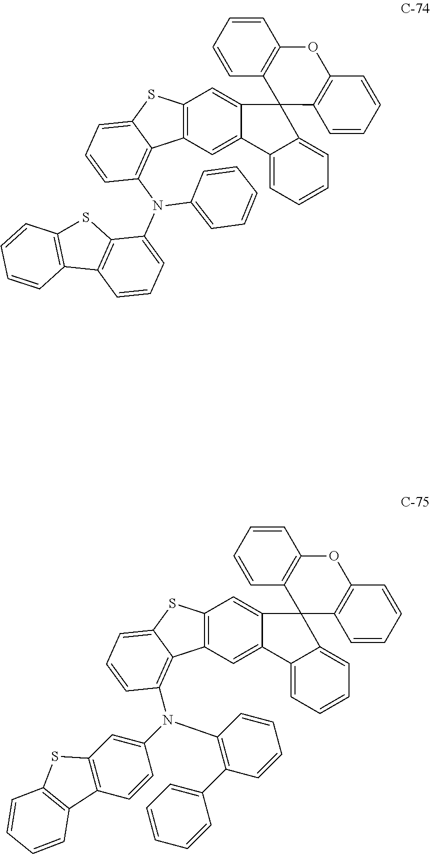 [SMILES: c1ccc(N(c2cccc3c2sc2ccccc23)c2cccc3sc4cc5c(cc4c23)-c2ccccc2C52c3ccccc3Oc3ccccc32)cc1 ; c1ccc(-c2ccccc2N(c2ccc3c(c2)sc2ccccc23)c2cccc3sc4cc5c(cc4c23)-c2ccccc2C52c3ccccc3Oc3ccccc32)cc1]

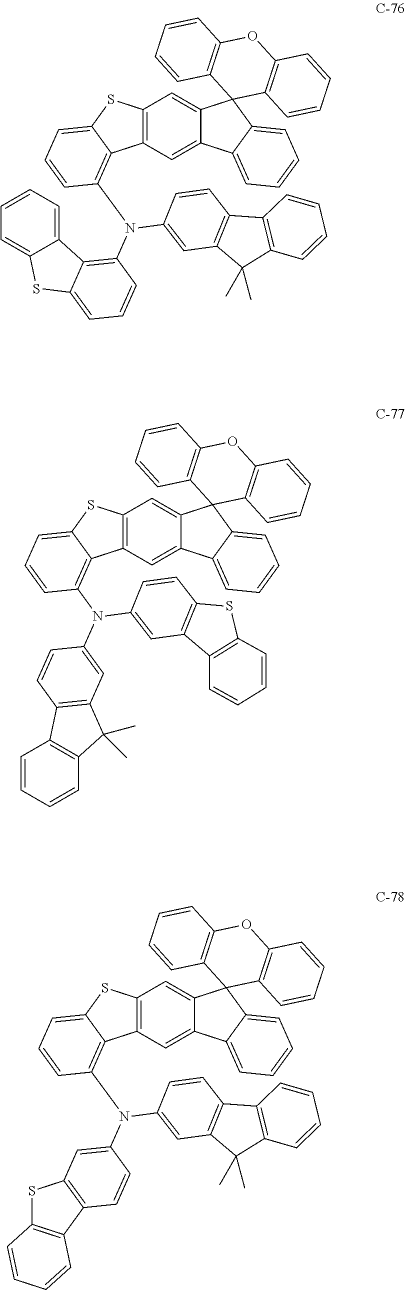 [SMILES: CC1(C)c2ccccc2-c2ccc(N(c3cccc4sc5ccccc5c34)c3cccc4sc5cc6c(cc5c34)-c3ccccc3C63c4ccccc4Oc4ccccc43)cc21 ; CC1(C)c2ccccc2-c2ccc(N(c3ccc4sc5ccccc5c4c3)c3cccc4sc5cc6c(cc5c34)-c3ccccc3C63c4ccccc4Oc4ccccc43)cc21 ; CC1(C)c2ccccc2-c2ccc(N(c3ccc4c(c3)sc3ccccc34)c3cccc4sc5cc6c(cc5c34)-c3ccccc3C63c4ccccc4Oc4ccccc43)cc21]

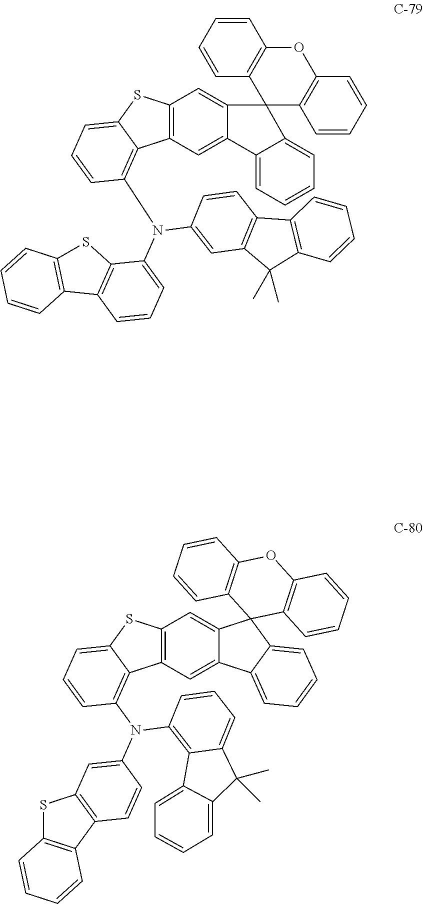 [SMILES: CC1(C)c2ccccc2-c2ccc(N(c3cccc4c3sc3ccccc34)c3cccc4sc5cc6c(cc5c34)-c3ccccc3C63c4ccccc4Oc4ccccc43)cc21 ; CC1(C)c2ccccc2-c2c(N(c3ccc4c(c3)sc3ccccc34)c3cccc4sc5cc6c(cc5c34)-c3ccccc3C63c4ccccc4Oc4ccccc43)cccc21]

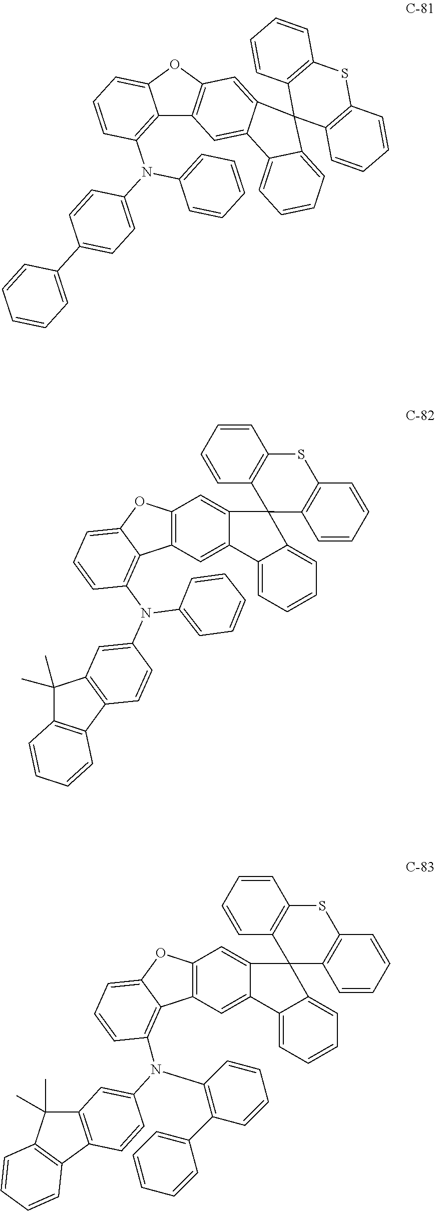 [SMILES: c1ccc(-c2ccc(N(c3ccccc3)c3cccc4oc5cc6c(cc5c34)-c3ccccc3C63c4ccccc4Sc4ccccc43)cc2)cc1 ; CC1(C)c2ccccc2-c2ccc(N(c3ccccc3)c3cccc4oc5cc6c(cc5c34)-c3ccccc3C63c4ccccc4Sc4ccccc43)cc21 ; CC1(C)c2ccccc2-c2ccc(N(c3ccccc3-c3ccccc3)c3cccc4oc5cc6c(cc5c34)-c3ccccc3C63c4ccccc4Sc4ccccc43)cc21]

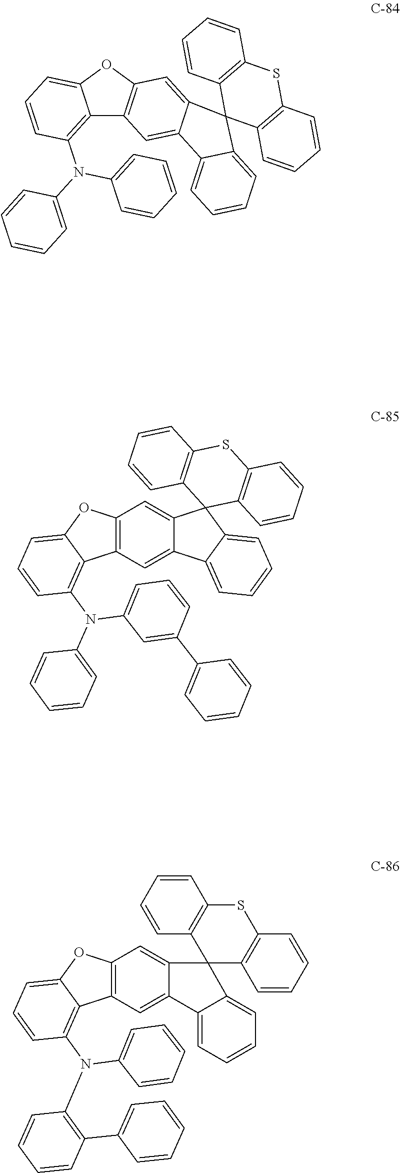 [SMILES: c1ccc(N(c2ccccc2)c2cccc3oc4cc5c(cc4c23)-c2ccccc2C52c3ccccc3Sc3ccccc32)cc1 ; c1ccc(-c2cccc(N(c3ccccc3)c3cccc4oc5cc6c(cc5c34)-c3ccccc3C63c4ccccc4Sc4ccccc43)c2)cc1 ; c1ccc(-c2ccccc2N(c2ccccc2)c2cccc3oc4cc5c(cc4c23)-c2ccccc2C52c3ccccc3Sc3ccccc32)cc1]

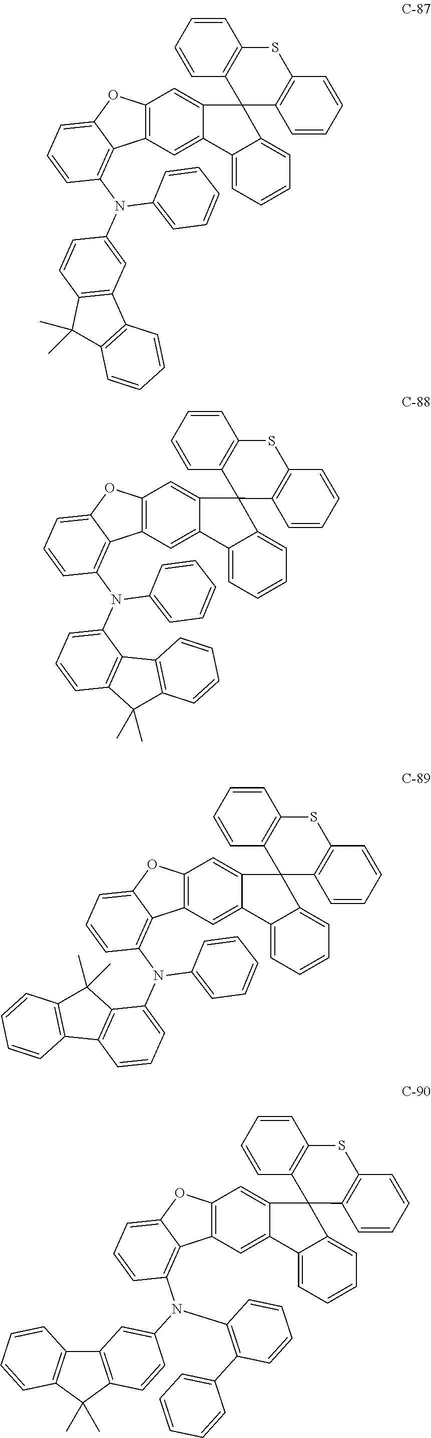 [SMILES: CC1(C)c2ccccc2-c2cc(N(c3ccccc3)c3cccc4oc5cc6c(cc5c34)-c3ccccc3C63c4ccccc4Sc4ccccc43)ccc21 ; CC1(C)c2ccccc2-c2c(N(c3ccccc3)c3cccc4oc5cc6c(cc5c34)-c3ccccc3C63c4ccccc4Sc4ccccc43)cccc21 ; CC1(C)c2ccccc2-c2cccc(N(c3ccccc3)c3cccc4oc5cc6c(cc5c34)-c3ccccc3C63c4ccccc4Sc4ccccc43)c21 ; CC1(C)c2ccccc2-c2cc(N(c3ccccc3-c3ccccc3)c3cccc4oc5cc6c(cc5c34)-c3ccccc3C63c4ccccc4Sc4ccccc43)ccc21]

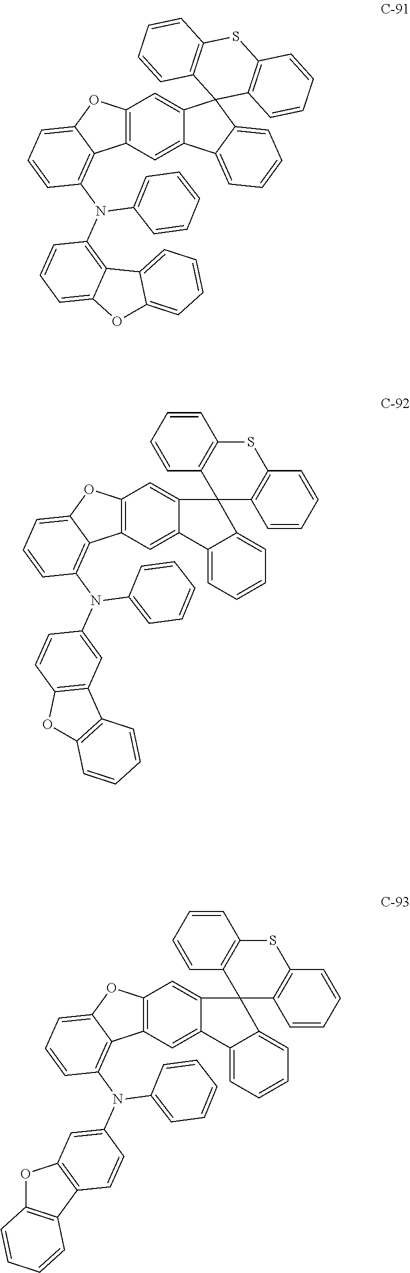 [SMILES: c1ccc(N(c2cccc3oc4ccccc4c23)c2cccc3oc4cc5c(cc4c23)-c2ccccc2C52c3ccccc3Sc3ccccc32)cc1 ; c1ccc(N(c2ccc3oc4ccccc4c3c2)c2cccc3oc4cc5c(cc4c23)-c2ccccc2C52c3ccccc3Sc3ccccc32)cc1 ; c1ccc(N(c2ccc3c(c2)oc2ccccc23)c2cccc3oc4cc5c(cc4c23)-c2ccccc2C52c3ccccc3Sc3ccccc32)cc1]

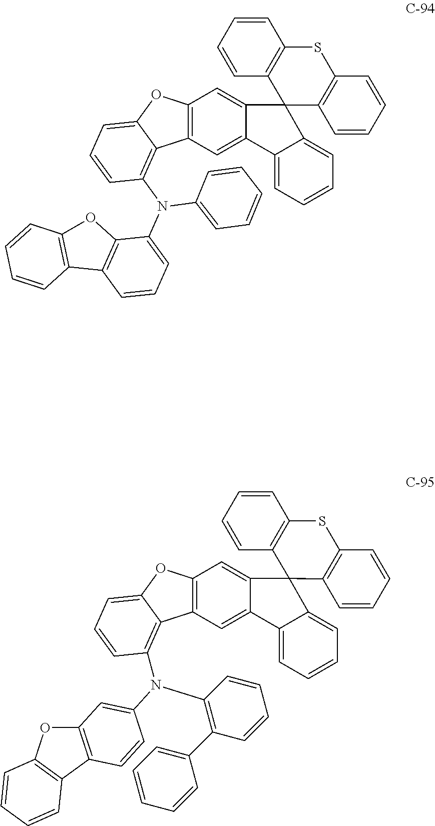 [SMILES: c1ccc(N(c2cccc3c2oc2ccccc23)c2cccc3oc4cc5c(cc4c23)-c2ccccc2C52c3ccccc3Sc3ccccc32)cc1 ; c1ccc(-c2ccccc2N(c2ccc3c(c2)oc2ccccc23)c2cccc3oc4cc5c(cc4c23)-c2ccccc2C52c3ccccc3Sc3ccccc32)cc1]

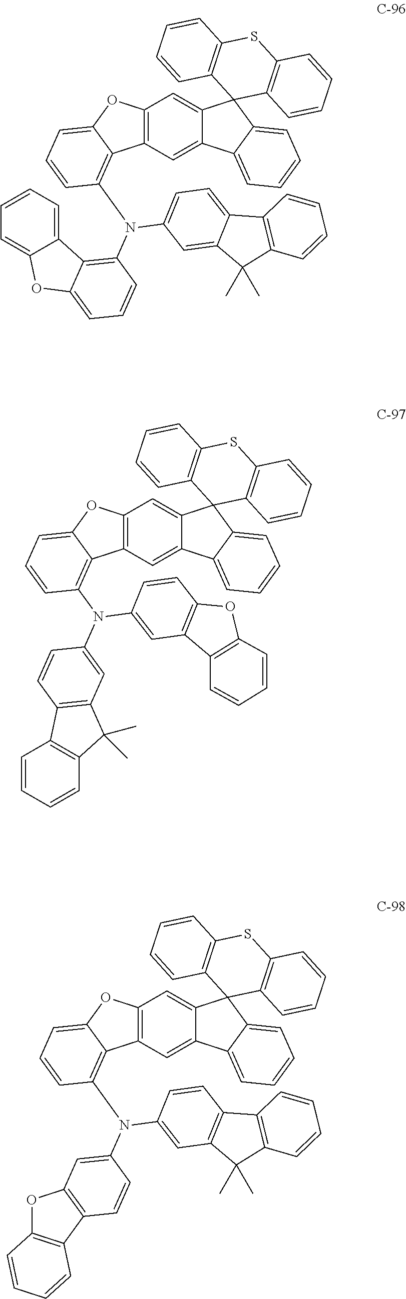 [SMILES: CC1(C)c2ccccc2-c2ccc(N(c3cccc4oc5ccccc5c34)c3cccc4oc5cc6c(cc5c34)-c3ccccc3C63c4ccccc4Sc4ccccc43)cc21 ; CC1(C)c2ccccc2-c2ccc(N(c3ccc4oc5ccccc5c4c3)c3cccc4oc5cc6c(cc5c34)-c3ccccc3C63c4ccccc4Sc4ccccc43)cc21 ; CC1(C)c2ccccc2-c2ccc(N(c3ccc4c(c3)oc3ccccc34)c3cccc4oc5cc6c(cc5c34)-c3ccccc3C63c4ccccc4Sc4ccccc43)cc21]

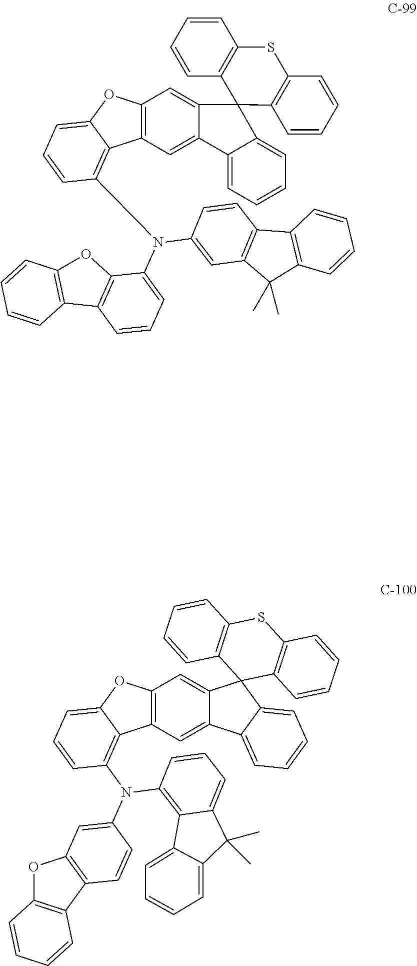 [SMILES: CC1(C)c2ccccc2-c2ccc(N(c3cccc4c3oc3ccccc34)c3cccc4oc5cc6c(cc5c34)-c3ccccc3C63c4ccccc4Sc4ccccc43)cc21 ; CC1(C)c2ccccc2-c2c(N(c3ccc4c(c3)oc3ccccc34)c3cccc4oc5cc6c(cc5c34)-c3ccccc3C63c4ccccc4Sc4ccccc43)cccc21]

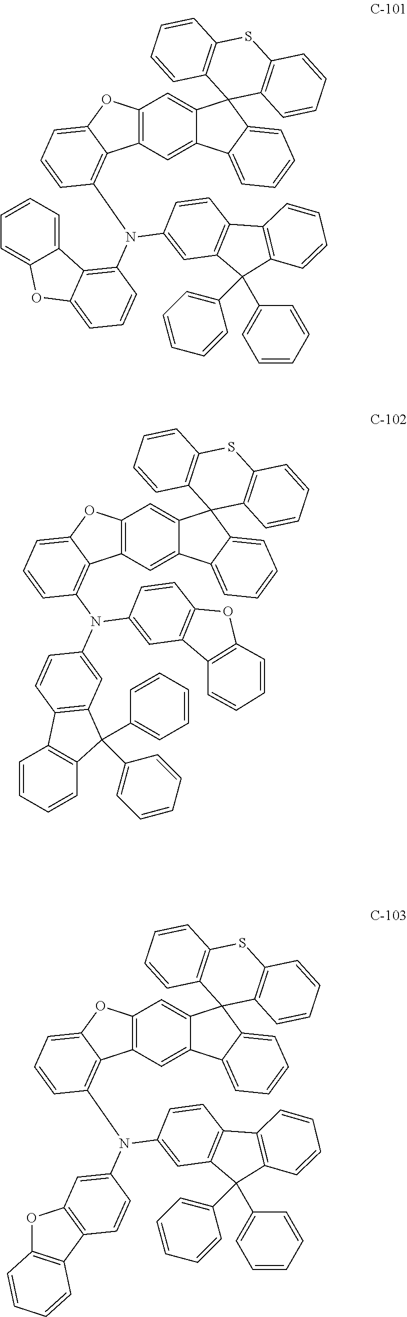 [SMILES: c1ccc(C2(c3ccccc3)c3ccccc3-c3ccc(N(c4cccc5oc6ccccc6c45)c4cccc5oc6cc7c(cc6c45)-c4ccccc4C74c5ccccc5Sc5ccccc54)cc32)cc1 ; c1ccc(C2(c3ccccc3)c3ccccc3-c3ccc(N(c4ccc5oc6ccccc6c5c4)c4cccc5oc6cc7c(cc6c45)-c4ccccc4C74c5ccccc5Sc5ccccc54)cc32)cc1 ; c1ccc(C2(c3ccccc3)c3ccccc3-c3ccc(N(c4ccc5c(c4)oc4ccccc45)c4cccc5oc6cc7c(cc6c45)-c4ccccc4C74c5ccccc5Sc5ccccc54)cc32)cc1]

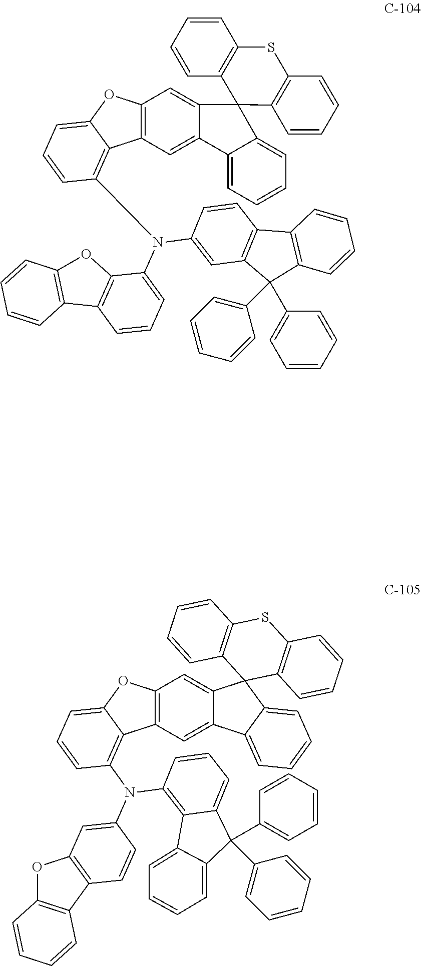 [SMILES: c1ccc(C2(c3ccccc3)c3ccccc3-c3ccc(N(c4cccc5c4oc4ccccc45)c4cccc5oc6cc7c(cc6c45)-c4ccccc4C74c5ccccc5Sc5ccccc54)cc32)cc1 ; c1ccc(C2(c3ccccc3)c3ccccc3-c3c(N(c4ccc5c(c4)oc4ccccc45)c4cccc5oc6cc7c(cc6c45)-c4ccccc4C74c5ccccc5Sc5ccccc54)cccc32)cc1]

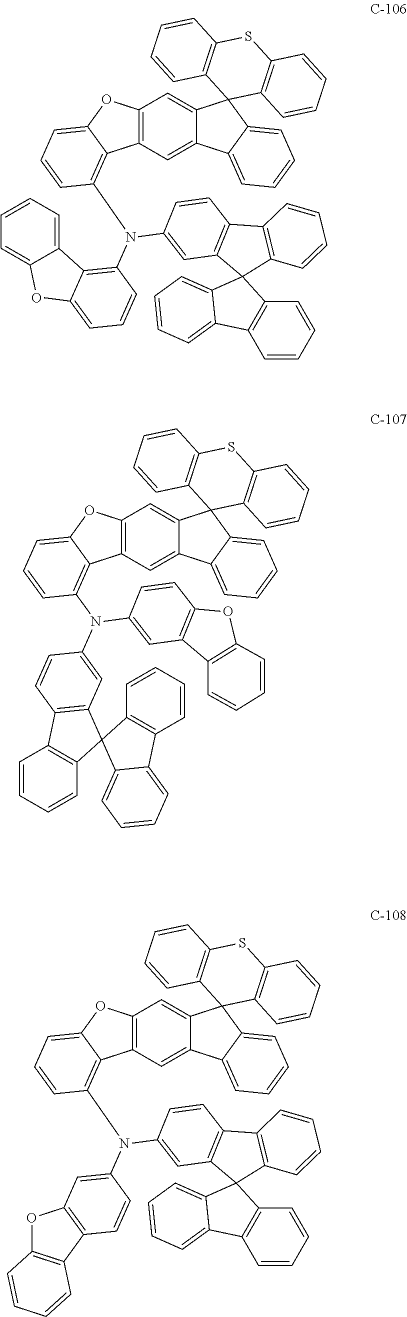 [SMILES: c1ccc2c(c1)Sc1ccccc1C21c2ccccc2-c2cc3c(cc21)oc1cccc(N(c2ccc4c(c2)C2(c5ccccc5-c5ccccc52)c2ccccc2-4)c2cccc4oc5ccccc5c24)c13 ; c1ccc2c(c1)Sc1ccccc1C21c2ccccc2-c2cc3c(cc21)oc1cccc(N(c2ccc4c(c2)C2(c5ccccc5-c5ccccc52)c2ccccc2-4)c2ccc4oc5ccccc5c4c2)c13 ; c1ccc2c(c1)Sc1ccccc1C21c2ccccc2-c2cc3c(cc21)oc1cccc(N(c2ccc4c(c2)C2(c5ccccc5-c5ccccc52)c2ccccc2-4)c2ccc4c(c2)oc2ccccc24)c13]

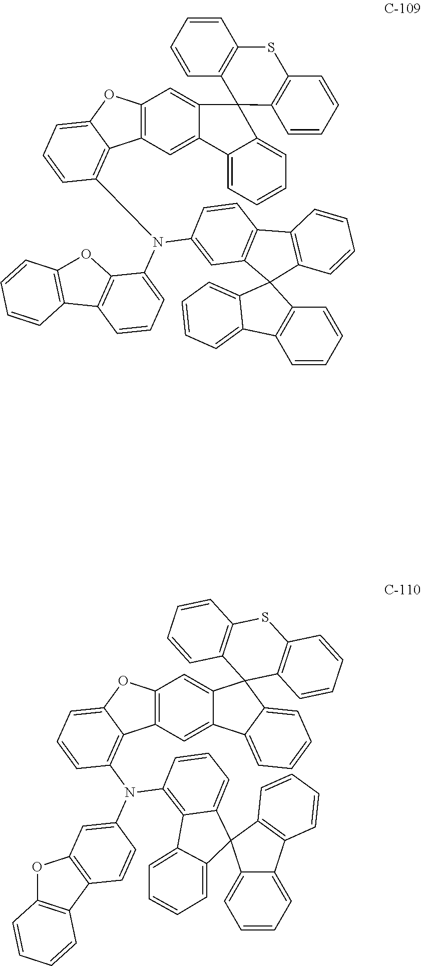 [SMILES: c1ccc2c(c1)Sc1ccccc1C21c2ccccc2-c2cc3c(cc21)oc1cccc(N(c2ccc4c(c2)C2(c5ccccc5-c5ccccc52)c2ccccc2-4)c2cccc4c2oc2ccccc24)c13 ; c1ccc2c(c1)Sc1ccccc1C21c2ccccc2-c2cc3c(cc21)oc1cccc(N(c2ccc4c(c2)oc2ccccc24)c2cccc4c2-c2ccccc2C42c4ccccc4-c4ccccc42)c13]

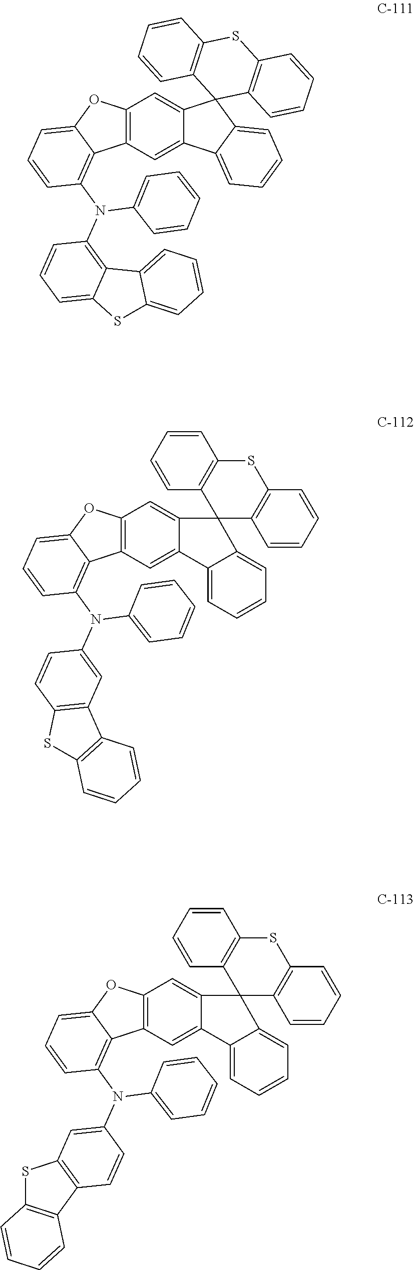 [SMILES: c1ccc(N(c2cccc3oc4cc5c(cc4c23)-c2ccccc2C52c3ccccc3Sc3ccccc32)c2cccc3sc4ccccc4c23)cc1 ; c1ccc(N(c2ccc3sc4ccccc4c3c2)c2cccc3oc4cc5c(cc4c23)-c2ccccc2C52c3ccccc3Sc3ccccc32)cc1 ; c1ccc(N(c2ccc3c(c2)sc2ccccc23)c2cccc3oc4cc5c(cc4c23)-c2ccccc2C52c3ccccc3Sc3ccccc32)cc1]

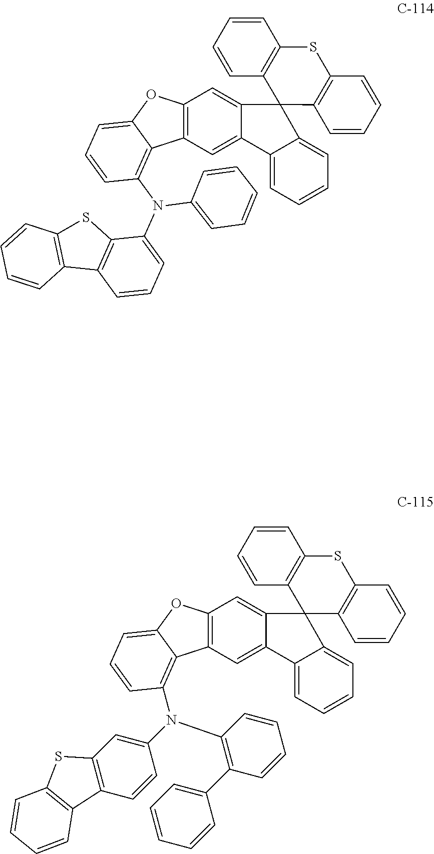 [SMILES: c1ccc(N(c2cccc3c2sc2ccccc23)c2cccc3oc4cc5c(cc4c23)-c2ccccc2C52c3ccccc3Sc3ccccc32)cc1 ; c1ccc(-c2ccccc2N(c2ccc3c(c2)sc2ccccc23)c2cccc3oc4cc5c(cc4c23)-c2ccccc2C52c3ccccc3Sc3ccccc32)cc1]

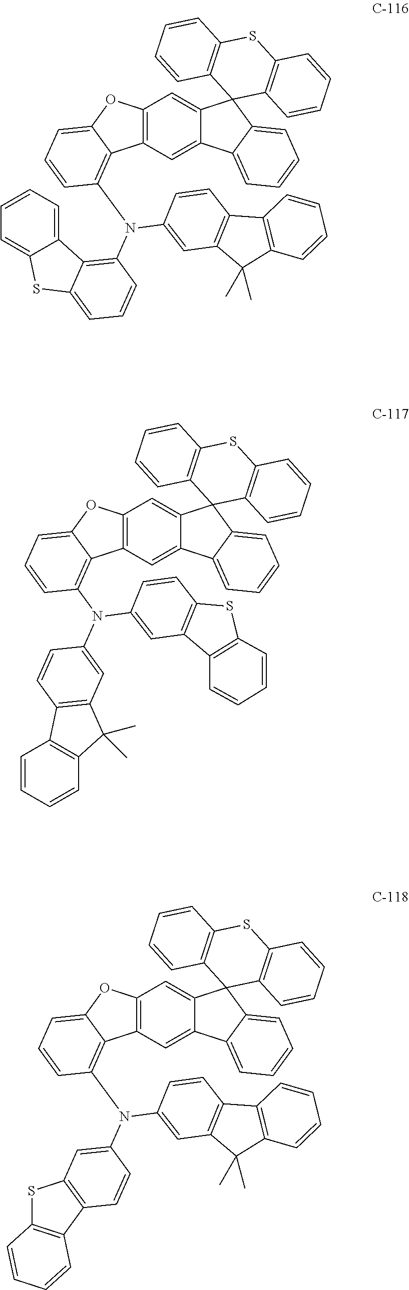 [SMILES: CC1(C)c2ccccc2-c2ccc(N(c3cccc4oc5cc6c(cc5c34)-c3ccccc3C63c4ccccc4Sc4ccccc43)c3cccc4sc5ccccc5c34)cc21 ; CC1(C)c2ccccc2-c2ccc(N(c3ccc4sc5ccccc5c4c3)c3cccc4oc5cc6c(cc5c34)-c3ccccc3C63c4ccccc4Sc4ccccc43)cc21 ; CC1(C)c2ccccc2-c2ccc(N(c3ccc4c(c3)sc3ccccc34)c3cccc4oc5cc6c(cc5c34)-c3ccccc3C63c4ccccc4Sc4ccccc43)cc21]

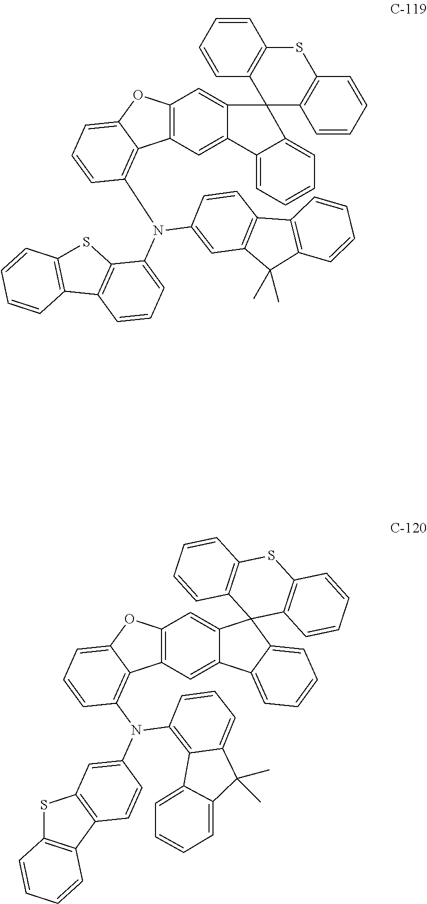 [SMILES: CC1(C)c2ccccc2-c2ccc(N(c3cccc4c3sc3ccccc34)c3cccc4oc5cc6c(cc5c34)-c3ccccc3C63c4ccccc4Sc4ccccc43)cc21 ; CC1(C)c2ccccc2-c2c(N(c3ccc4c(c3)sc3ccccc34)c3cccc4oc5cc6c(cc5c34)-c3ccccc3C63c4ccccc4Sc4ccccc43)cccc21]

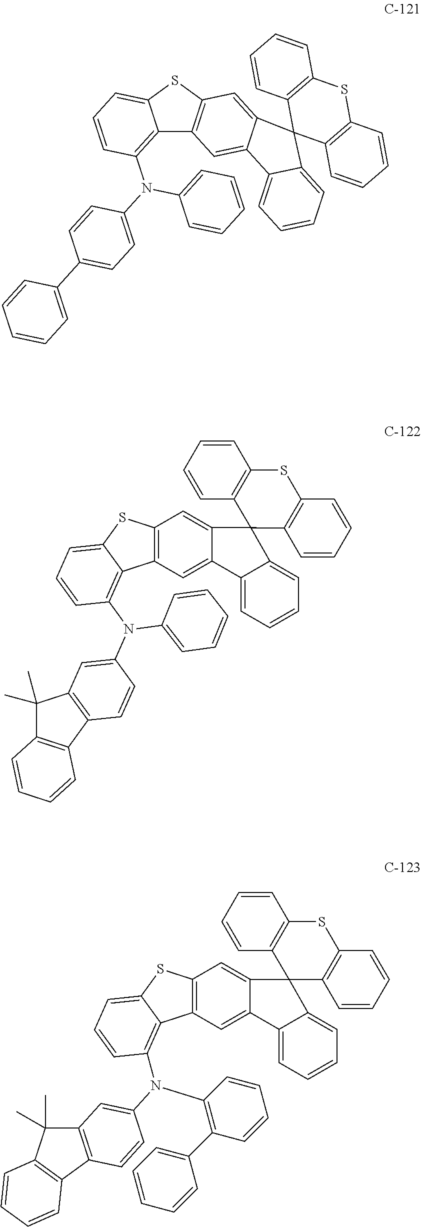 [SMILES: c1ccc(-c2ccc(N(c3ccccc3)c3cccc4sc5cc6c(cc5c34)-c3ccccc3C63c4ccccc4Sc4ccccc43)cc2)cc1 ; CC1(C)c2ccccc2-c2ccc(N(c3ccccc3)c3cccc4sc5cc6c(cc5c34)-c3ccccc3C63c4ccccc4Sc4ccccc43)cc21 ; CC1(C)c2ccccc2-c2ccc(N(c3ccccc3-c3ccccc3)c3cccc4sc5cc6c(cc5c34)-c3ccccc3C63c4ccccc4Sc4ccccc43)cc21]

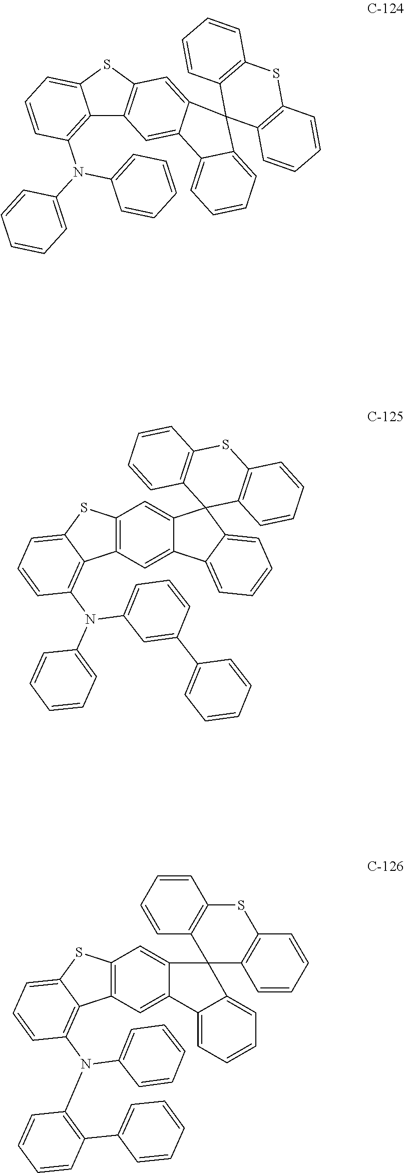 [SMILES: c1ccc(N(c2ccccc2)c2cccc3sc4cc5c(cc4c23)-c2ccccc2C52c3ccccc3Sc3ccccc32)cc1 ; c1ccc(-c2cccc(N(c3ccccc3)c3cccc4sc5cc6c(cc5c34)-c3ccccc3C63c4ccccc4Sc4ccccc43)c2)cc1 ; c1ccc(-c2ccccc2N(c2ccccc2)c2cccc3sc4cc5c(cc4c23)-c2ccccc2C52c3ccccc3Sc3ccccc32)cc1]

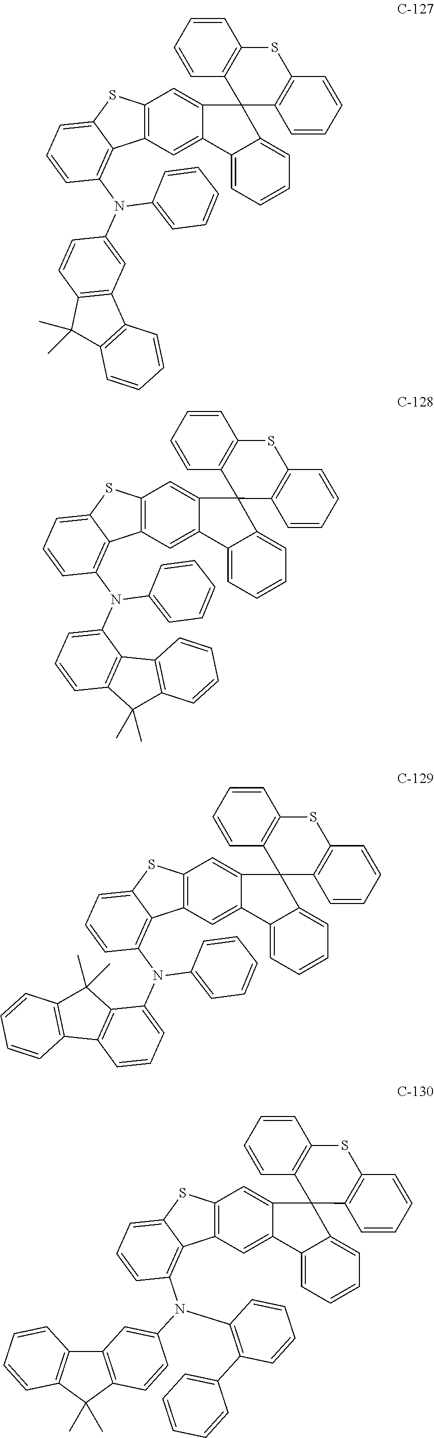 [SMILES: CC1(C)c2ccccc2-c2cc(N(c3ccccc3)c3cccc4sc5cc6c(cc5c34)-c3ccccc3C63c4ccccc4Sc4ccccc43)ccc21 ; CC1(C)c2ccccc2-c2c(N(c3ccccc3)c3cccc4sc5cc6c(cc5c34)-c3ccccc3C63c4ccccc4Sc4ccccc43)cccc21 ; CC1(C)c2ccccc2-c2cccc(N(c3ccccc3)c3cccc4sc5cc6c(cc5c34)-c3ccccc3C63c4ccccc4Sc4ccccc43)c21 ; CC1(C)c2ccccc2-c2cc(N(c3ccccc3-c3ccccc3)c3cccc4sc5cc6c(cc5c34)-c3ccccc3C63c4ccccc4Sc4ccccc43)ccc21]

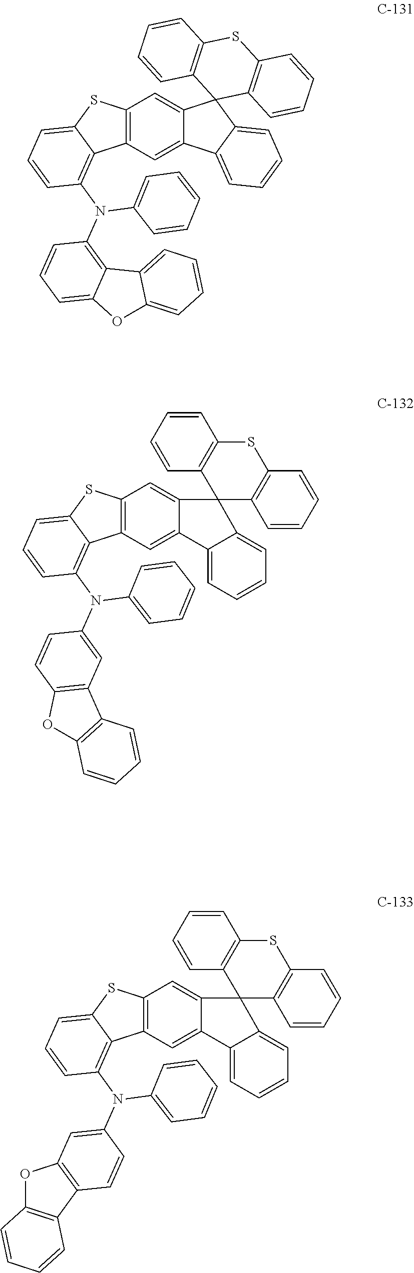 [SMILES: c1ccc(N(c2cccc3oc4ccccc4c23)c2cccc3sc4cc5c(cc4c23)-c2ccccc2C52c3ccccc3Sc3ccccc32)cc1 ; c1ccc(N(c2ccc3oc4ccccc4c3c2)c2cccc3sc4cc5c(cc4c23)-c2ccccc2C52c3ccccc3Sc3ccccc32)cc1 ; c1ccc(N(c2ccc3c(c2)oc2ccccc23)c2cccc3sc4cc5c(cc4c23)-c2ccccc2C52c3ccccc3Sc3ccccc32)cc1]

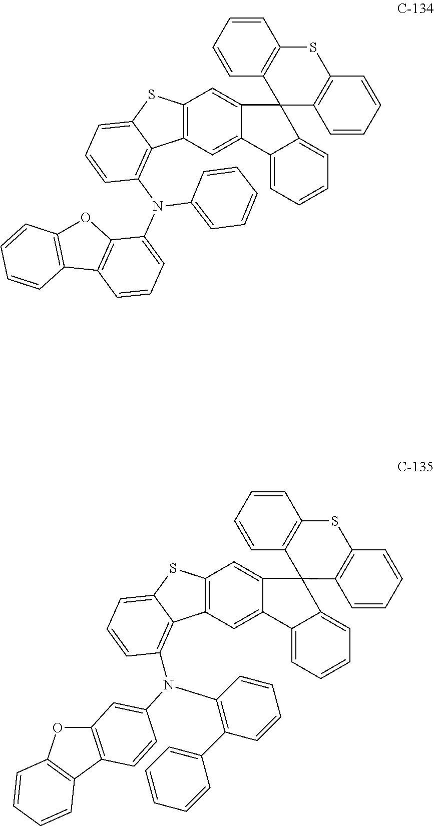 [SMILES: c1ccc(N(c2cccc3c2oc2ccccc23)c2cccc3sc4cc5c(cc4c23)-c2ccccc2C52c3ccccc3Sc3ccccc32)cc1 ; c1ccc(-c2ccccc2N(c2ccc3c(c2)oc2ccccc23)c2cccc3sc4cc5c(cc4c23)-c2ccccc2C52c3ccccc3Sc3ccccc32)cc1]

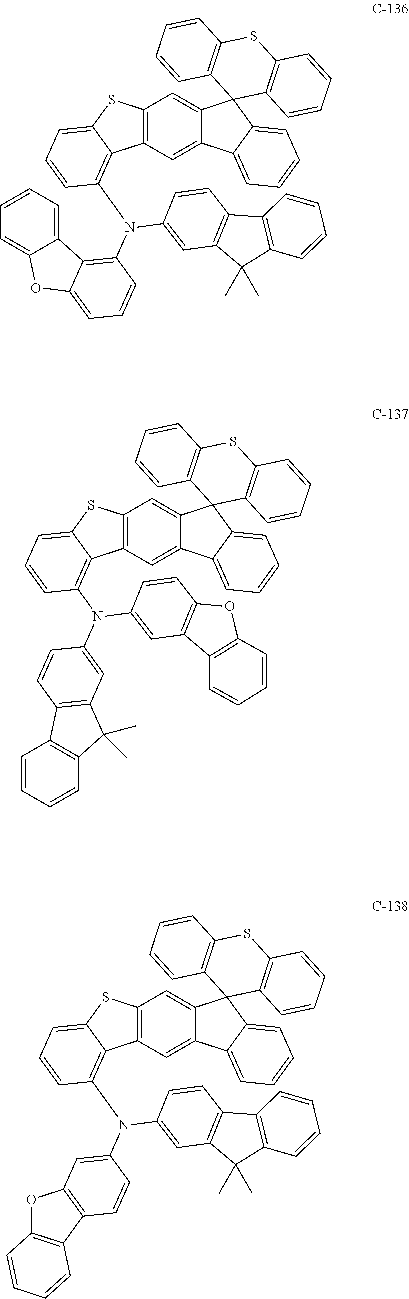 [SMILES: CC1(C)c2ccccc2-c2ccc(N(c3cccc4oc5ccccc5c34)c3cccc4sc5cc6c(cc5c34)-c3ccccc3C63c4ccccc4Sc4ccccc43)cc21 ; CC1(C)c2ccccc2-c2ccc(N(c3ccc4oc5ccccc5c4c3)c3cccc4sc5cc6c(cc5c34)-c3ccccc3C63c4ccccc4Sc4ccccc43)cc21 ; CC1(C)c2ccccc2-c2ccc(N(c3ccc4c(c3)oc3ccccc34)c3cccc4sc5cc6c(cc5c34)-c3ccccc3C63c4ccccc4Sc4ccccc43)cc21]

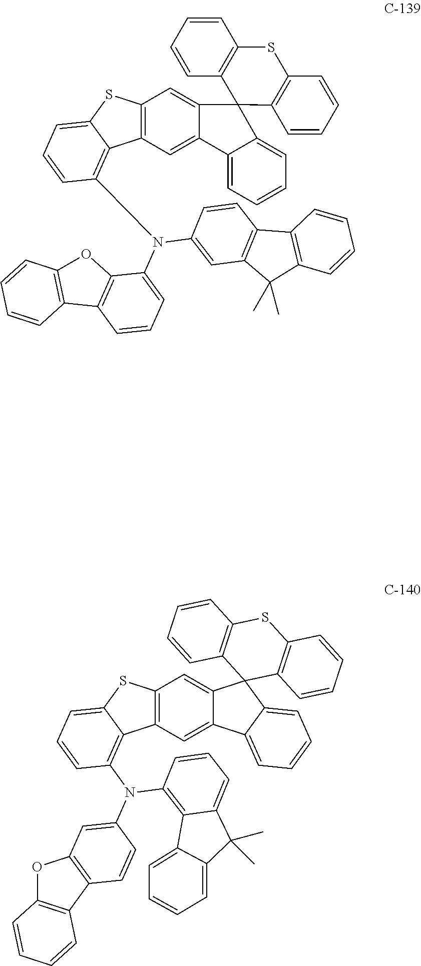 [SMILES: CC1(C)c2ccccc2-c2ccc(N(c3cccc4c3oc3ccccc34)c3cccc4sc5cc6c(cc5c34)-c3ccccc3C63c4ccccc4Sc4ccccc43)cc21 ; CC1(C)c2ccccc2-c2c(N(c3ccc4c(c3)oc3ccccc34)c3cccc4sc5cc6c(cc5c34)-c3ccccc3C63c4ccccc4Sc4ccccc43)cccc21]

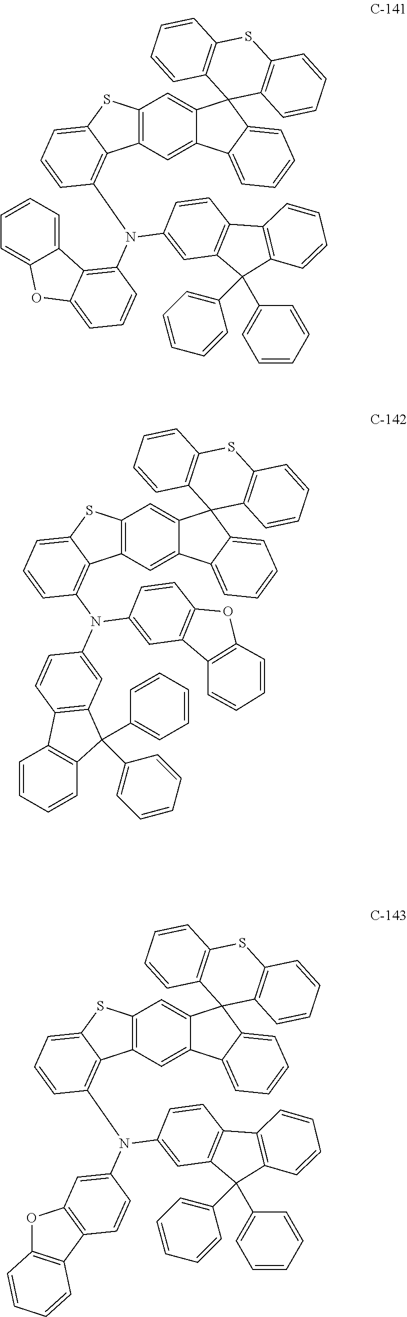 [SMILES: c1ccc(C2(c3ccccc3)c3ccccc3-c3ccc(N(c4cccc5oc6ccccc6c45)c4cccc5sc6cc7c(cc6c45)-c4ccccc4C74c5ccccc5Sc5ccccc54)cc32)cc1 ; c1ccc(C2(c3ccccc3)c3ccccc3-c3ccc(N(c4ccc5oc6ccccc6c5c4)c4cccc5sc6cc7c(cc6c45)-c4ccccc4C74c5ccccc5Sc5ccccc54)cc32)cc1 ; c1ccc(C2(c3ccccc3)c3ccccc3-c3ccc(N(c4ccc5c(c4)oc4ccccc45)c4cccc5sc6cc7c(cc6c45)-c4ccccc4C74c5ccccc5Sc5ccccc54)cc32)cc1]

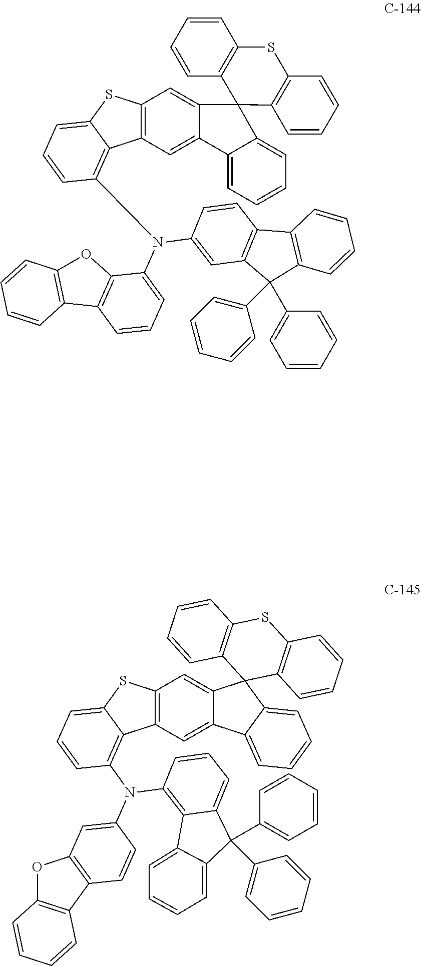 [SMILES: c1ccc(C2(c3ccccc3)c3ccccc3-c3ccc(N(c4cccc5c4oc4ccccc45)c4cccc5sc6cc7c(cc6c45)-c4ccccc4C74c5ccccc5Sc5ccccc54)cc32)cc1 ; c1ccc(C2(c3ccccc3)c3ccccc3-c3c(N(c4ccc5c(c4)oc4ccccc45)c4cccc5sc6cc7c(cc6c45)-c4ccccc4C74c5ccccc5Sc5ccccc54)cccc32)cc1]

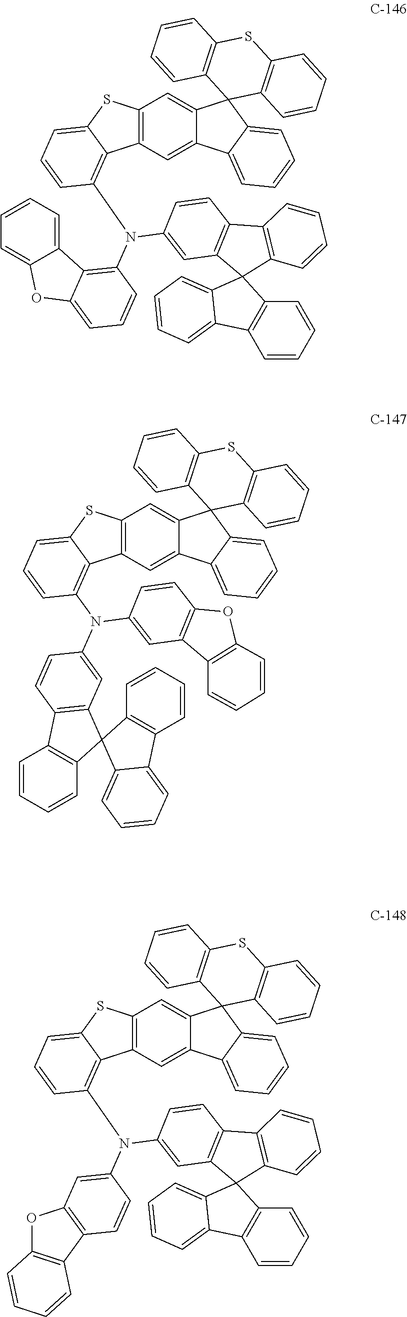 [SMILES: c1ccc2c(c1)Sc1ccccc1C21c2ccccc2-c2cc3c(cc21)sc1cccc(N(c2ccc4c(c2)C2(c5ccccc5-c5ccccc52)c2ccccc2-4)c2cccc4oc5ccccc5c24)c13 ; c1ccc2c(c1)Sc1ccccc1C21c2ccccc2-c2cc3c(cc21)sc1cccc(N(c2ccc4c(c2)C2(c5ccccc5-c5ccccc52)c2ccccc2-4)c2ccc4oc5ccccc5c4c2)c13 ; c1ccc2c(c1)Sc1ccccc1C21c2ccccc2-c2cc3c(cc21)sc1cccc(N(c2ccc4c(c2)C2(c5ccccc5-c5ccccc52)c2ccccc2-4)c2ccc4c(c2)oc2ccccc24)c13]

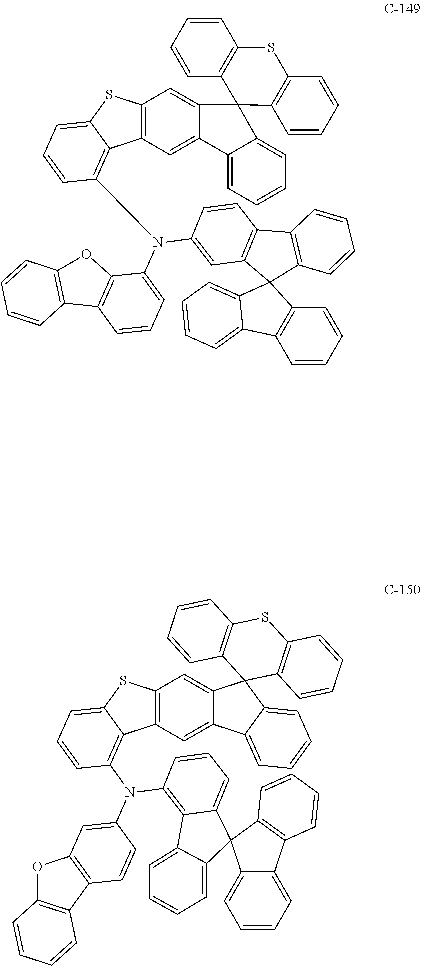 [SMILES: c1ccc2c(c1)Sc1ccccc1C21c2ccccc2-c2cc3c(cc21)sc1cccc(N(c2ccc4c(c2)C2(c5ccccc5-c5ccccc52)c2ccccc2-4)c2cccc4c2oc2ccccc24)c13 ; c1ccc2c(c1)Sc1ccccc1C21c2ccccc2-c2cc3c(cc21)sc1cccc(N(c2ccc4c(c2)oc2ccccc24)c2cccc4c2-c2ccccc2C42c4ccccc4-c4ccccc42)c13]

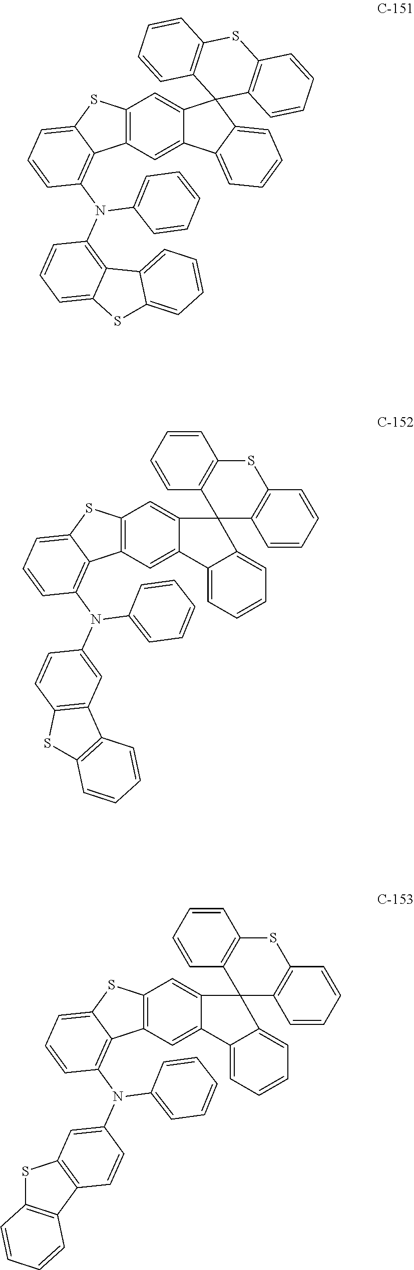 [SMILES: c1ccc(N(c2cccc3sc4ccccc4c23)c2cccc3sc4cc5c(cc4c23)-c2ccccc2C52c3ccccc3Sc3ccccc32)cc1 ; c1ccc(N(c2ccc3sc4ccccc4c3c2)c2cccc3sc4cc5c(cc4c23)-c2ccccc2C52c3ccccc3Sc3ccccc32)cc1 ; c1ccc(N(c2ccc3c(c2)sc2ccccc23)c2cccc3sc4cc5c(cc4c23)-c2ccccc2C52c3ccccc3Sc3ccccc32)cc1]

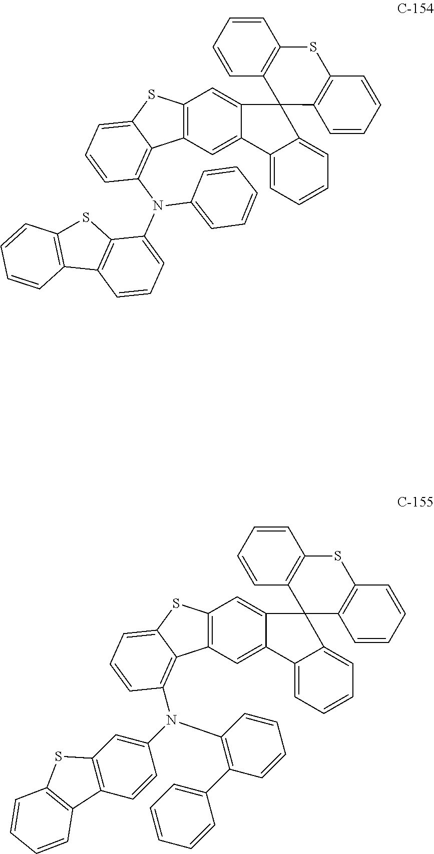 [SMILES: c1ccc(N(c2cccc3c2sc2ccccc23)c2cccc3sc4cc5c(cc4c23)-c2ccccc2C52c3ccccc3Sc3ccccc32)cc1 ; c1ccc(-c2ccccc2N(c2ccc3c(c2)sc2ccccc23)c2cccc3sc4cc5c(cc4c23)-c2ccccc2C52c3ccccc3Sc3ccccc32)cc1]

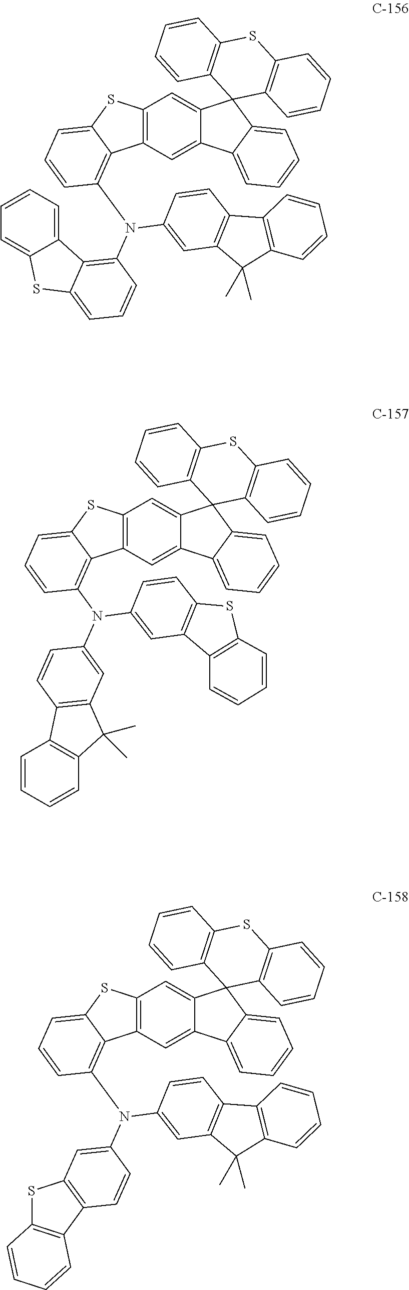 [SMILES: CC1(C)c2ccccc2-c2ccc(N(c3cccc4sc5ccccc5c34)c3cccc4sc5cc6c(cc5c34)-c3ccccc3C63c4ccccc4Sc4ccccc43)cc21 ; CC1(C)c2ccccc2-c2ccc(N(c3ccc4sc5ccccc5c4c3)c3cccc4sc5cc6c(cc5c34)-c3ccccc3C63c4ccccc4Sc4ccccc43)cc21 ; CC1(C)c2ccccc2-c2ccc(N(c3ccc4c(c3)sc3ccccc34)c3cccc4sc5cc6c(cc5c34)-c3ccccc3C63c4ccccc4Sc4ccccc43)cc21]

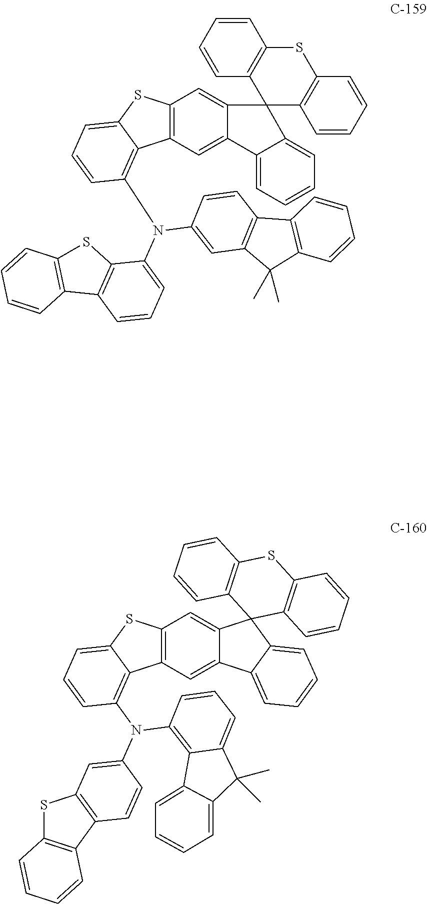 [SMILES: CC1(C)c2ccccc2-c2ccc(N(c3cccc4c3sc3ccccc34)c3cccc4sc5cc6c(cc5c34)-c3ccccc3C63c4ccccc4Sc4ccccc43)cc21 ; CC1(C)c2ccccc2-c2c(N(c3ccc4c(c3)sc3ccccc34)c3cccc4sc5cc6c(cc5c34)-c3ccccc3C63c4ccccc4Sc4ccccc43)cccc21]

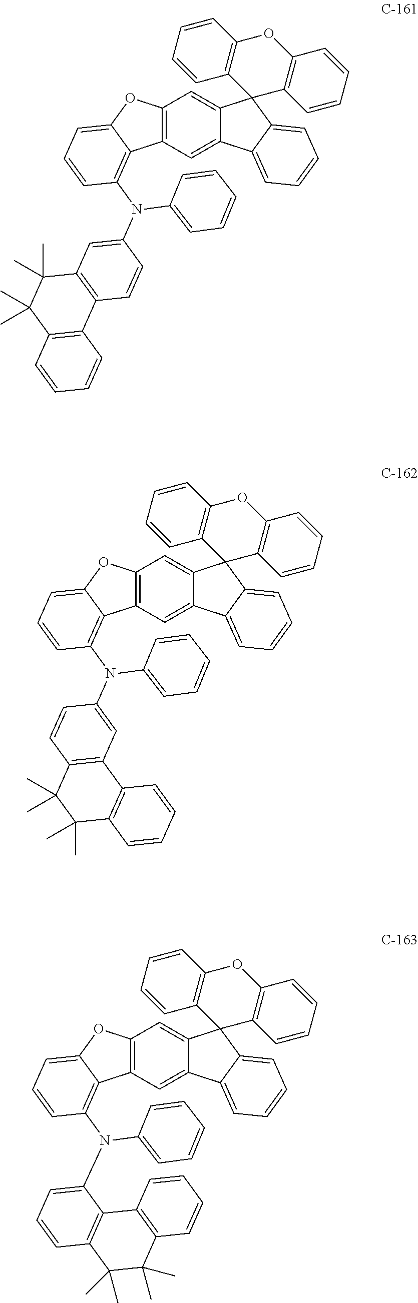 [SMILES: CC1(C)c2ccccc2-c2ccc(N(c3ccccc3)c3cccc4oc5cc6c(cc5c34)-c3ccccc3C63c4ccccc4Oc4ccccc43)cc2C1(C)C ; CC1(C)c2ccccc2-c2cc(N(c3ccccc3)c3cccc4oc5cc6c(cc5c34)-c3ccccc3C63c4ccccc4Oc4ccccc43)ccc2C1(C)C ; CC1(C)c2ccccc2-c2c(N(c3ccccc3)c3cccc4oc5cc6c(cc5c34)-c3ccccc3C63c4ccccc4Oc4ccccc43)cccc2C1(C)C]

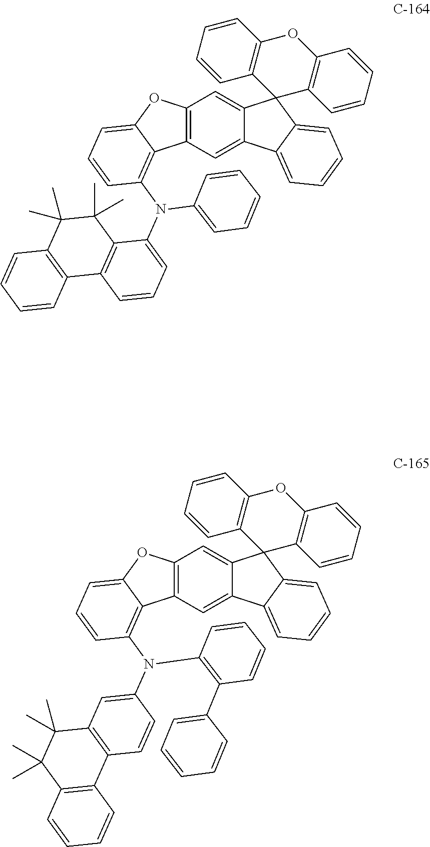 [SMILES: CC1(C)c2ccccc2-c2cccc(N(c3ccccc3)c3cccc4oc5cc6c(cc5c34)-c3ccccc3C63c4ccccc4Oc4ccccc43)c2C1(C)C ; CC1(C)c2ccccc2-c2ccc(N(c3ccccc3-c3ccccc3)c3cccc4oc5cc6c(cc5c34)-c3ccccc3C63c4ccccc4Oc4ccccc43)cc2C1(C)C]

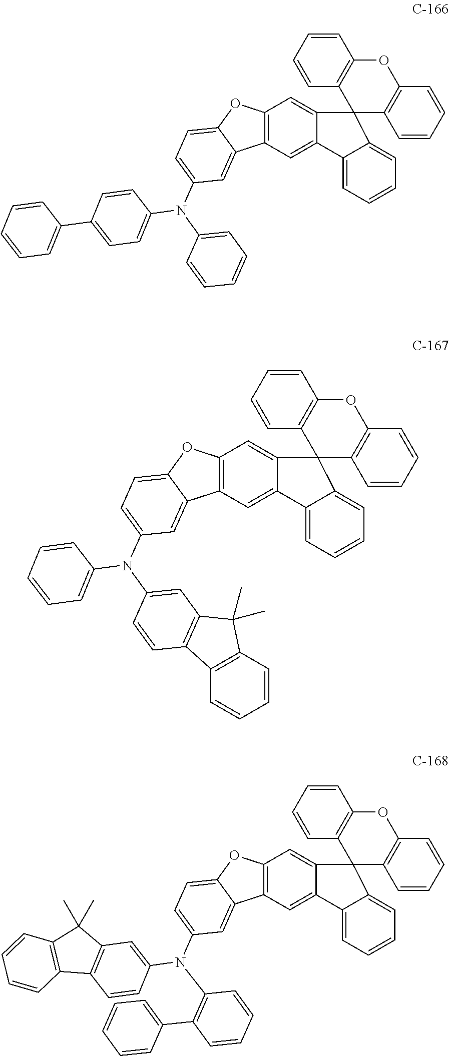 [SMILES: c1ccc(-c2ccc(N(c3ccccc3)c3ccc4oc5cc6c(cc5c4c3)-c3ccccc3C63c4ccccc4Oc4ccccc43)cc2)cc1 ; CC1(C)c2ccccc2-c2ccc(N(c3ccccc3)c3ccc4oc5cc6c(cc5c4c3)-c3ccccc3C63c4ccccc4Oc4ccccc43)cc21 ; CC1(C)c2ccccc2-c2ccc(N(c3ccc4oc5cc6c(cc5c4c3)-c3ccccc3C63c4ccccc4Oc4ccccc43)c3ccccc3-c3ccccc3)cc21]

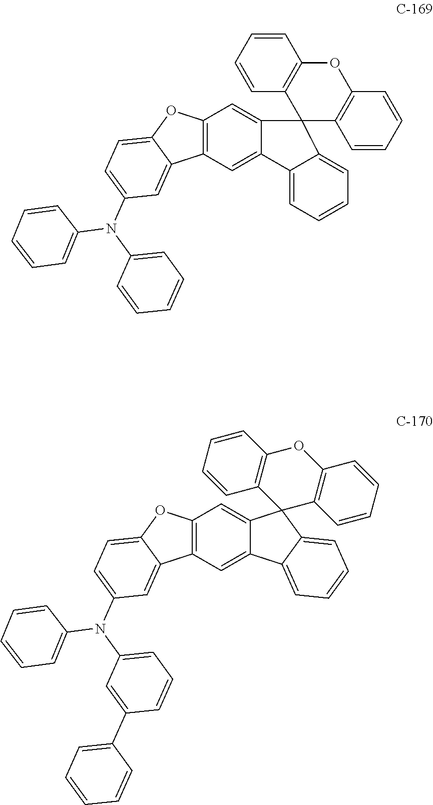 [SMILES: c1ccc(N(c2ccccc2)c2ccc3oc4cc5c(cc4c3c2)-c2ccccc2C52c3ccccc3Oc3ccccc32)cc1 ; c1ccc(-c2cccc(N(c3ccccc3)c3ccc4oc5cc6c(cc5c4c3)-c3ccccc3C63c4ccccc4Oc4ccccc43)c2)cc1]

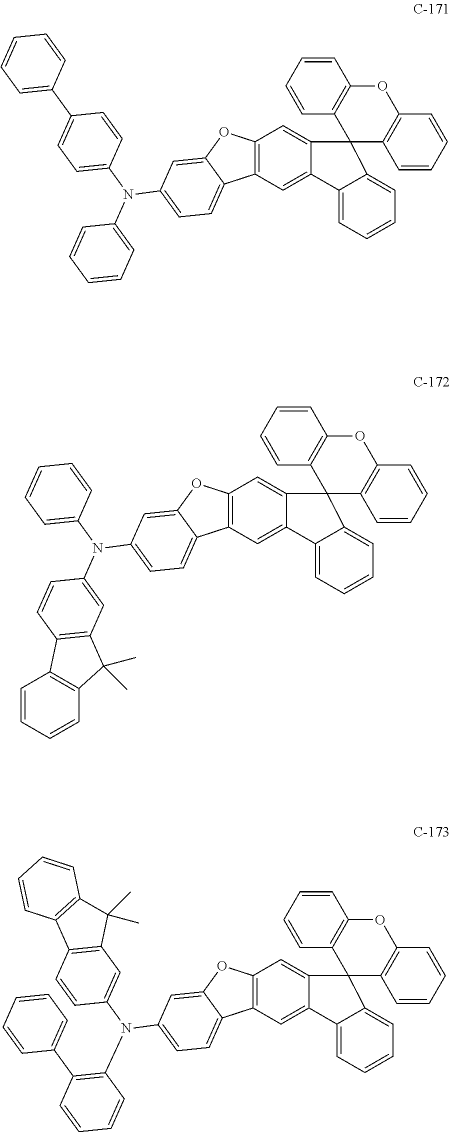 [SMILES: c1ccc(-c2ccc(N(c3ccccc3)c3ccc4c(c3)oc3cc5c(cc34)-c3ccccc3C53c4ccccc4Oc4ccccc43)cc2)cc1 ; CC1(C)c2ccccc2-c2ccc(N(c3ccccc3)c3ccc4c(c3)oc3cc5c(cc34)-c3ccccc3C53c4ccccc4Oc4ccccc43)cc21 ; CC1(C)c2ccccc2-c2ccc(N(c3ccc4c(c3)oc3cc5c(cc34)-c3ccccc3C53c4ccccc4Oc4ccccc43)c3ccccc3-c3ccccc3)cc21]

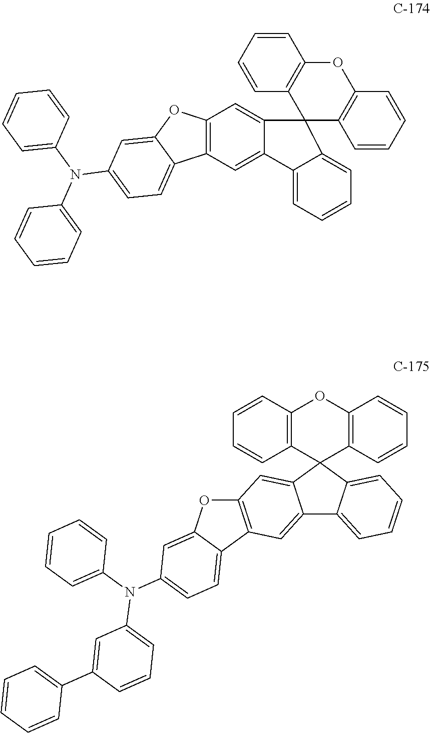 [SMILES: c1ccc(N(c2ccccc2)c2ccc3c(c2)oc2cc4c(cc23)-c2ccccc2C42c3ccccc3Oc3ccccc32)cc1 ; c1ccc(-c2cccc(N(c3ccccc3)c3ccc4c(c3)oc3cc5c(cc34)-c3ccccc3C53c4ccccc4Oc4ccccc43)c2)cc1]

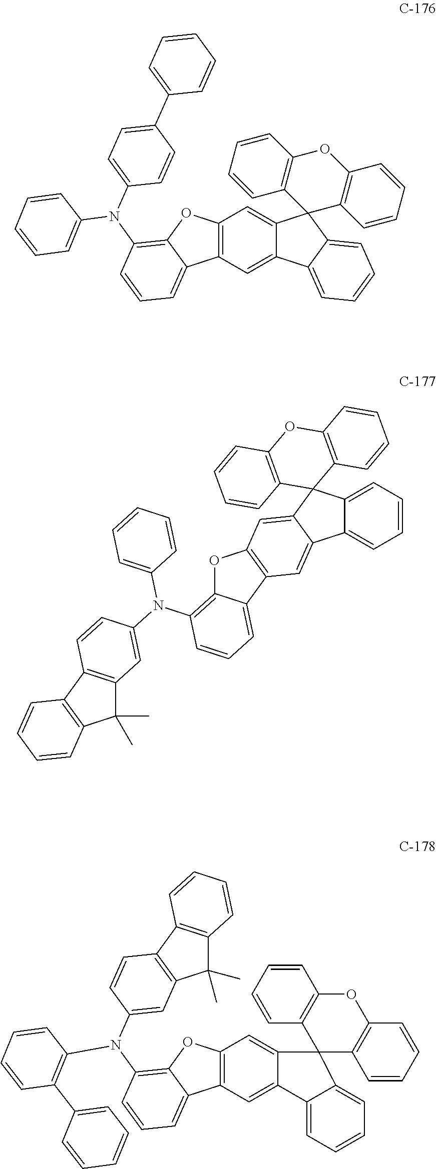 [SMILES: c1ccc(-c2ccc(N(c3ccccc3)c3cccc4c3oc3cc5c(cc34)-c3ccccc3C53c4ccccc4Oc4ccccc43)cc2)cc1 ; CC1(C)c2ccccc2-c2ccc(N(c3ccccc3)c3cccc4c3oc3cc5c(cc34)-c3ccccc3C53c4ccccc4Oc4ccccc43)cc21 ; CC1(C)c2ccccc2-c2ccc(N(c3ccccc3-c3ccccc3)c3cccc4c3oc3cc5c(cc34)-c3ccccc3C53c4ccccc4Oc4ccccc43)cc21]

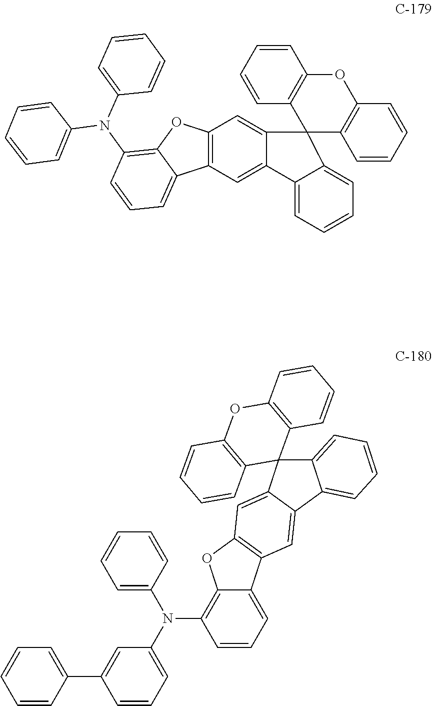 [SMILES: c1ccc(N(c2ccccc2)c2cccc3c2oc2cc4c(cc23)-c2ccccc2C42c3ccccc3Oc3ccccc32)cc1 ; c1ccc(-c2cccc(N(c3ccccc3)c3cccc4c3oc3cc5c(cc34)-c3ccccc3C53c4ccccc4Oc4ccccc43)c2)cc1]

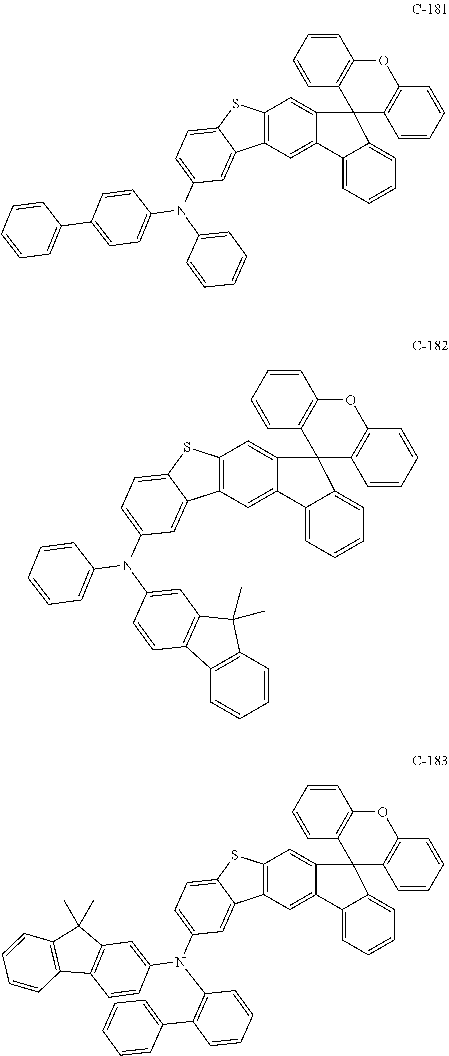 [SMILES: c1ccc(-c2ccc(N(c3ccccc3)c3ccc4sc5cc6c(cc5c4c3)-c3ccccc3C63c4ccccc4Oc4ccccc43)cc2)cc1 ; CC1(C)c2ccccc2-c2ccc(N(c3ccccc3)c3ccc4sc5cc6c(cc5c4c3)-c3ccccc3C63c4ccccc4Oc4ccccc43)cc21 ; CC1(C)c2ccccc2-c2ccc(N(c3ccc4sc5cc6c(cc5c4c3)-c3ccccc3C63c4ccccc4Oc4ccccc43)c3ccccc3-c3ccccc3)cc21]

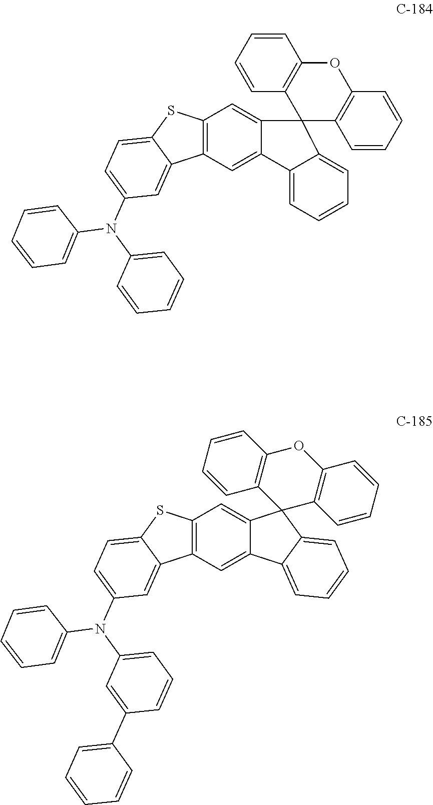 [SMILES: c1ccc(N(c2ccccc2)c2ccc3sc4cc5c(cc4c3c2)-c2ccccc2C52c3ccccc3Oc3ccccc32)cc1 ; c1ccc(-c2cccc(N(c3ccccc3)c3ccc4sc5cc6c(cc5c4c3)-c3ccccc3C63c4ccccc4Oc4ccccc43)c2)cc1]

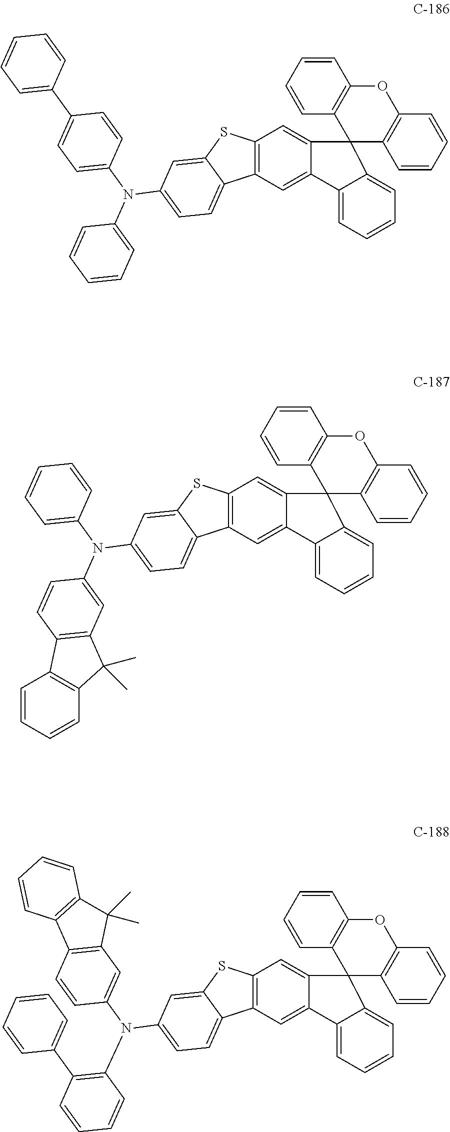 [SMILES: c1ccc(-c2ccc(N(c3ccccc3)c3ccc4c(c3)sc3cc5c(cc34)-c3ccccc3C53c4ccccc4Oc4ccccc43)cc2)cc1 ; CC1(C)c2ccccc2-c2ccc(N(c3ccccc3)c3ccc4c(c3)sc3cc5c(cc34)-c3ccccc3C53c4ccccc4Oc4ccccc43)cc21 ; CC1(C)c2ccccc2-c2ccc(N(c3ccc4c(c3)sc3cc5c(cc34)-c3ccccc3C53c4ccccc4Oc4ccccc43)c3ccccc3-c3ccccc3)cc21]

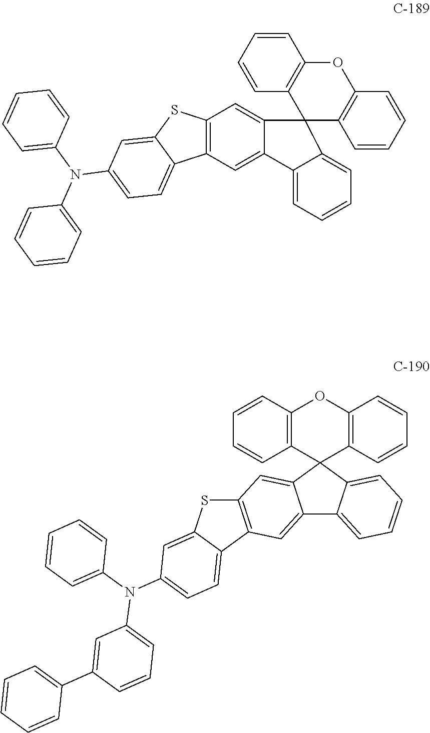 [SMILES: c1ccc(N(c2ccccc2)c2ccc3c(c2)sc2cc4c(cc23)-c2ccccc2C42c3ccccc3Oc3ccccc32)cc1 ; c1ccc(-c2cccc(N(c3ccccc3)c3ccc4c(c3)sc3cc5c(cc34)-c3ccccc3C53c4ccccc4Oc4ccccc43)c2)cc1]

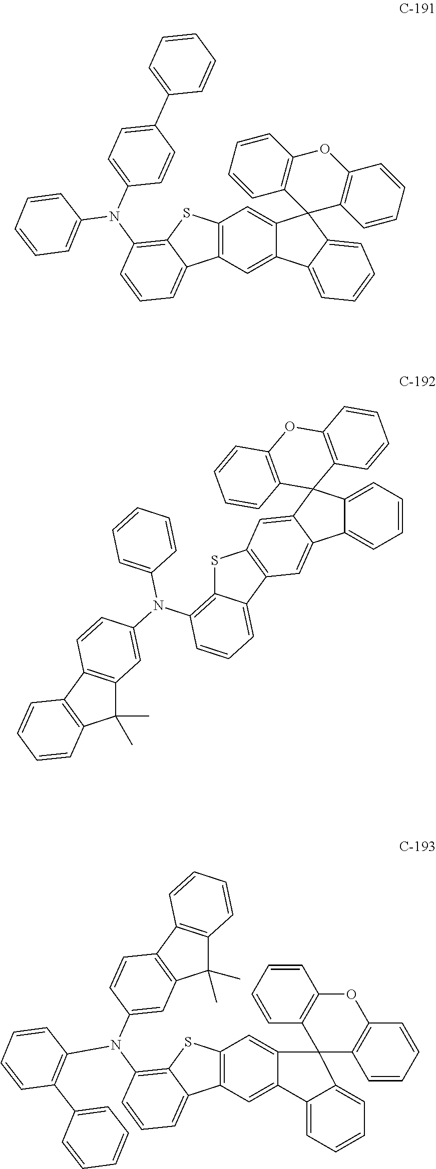 [SMILES: c1ccc(-c2ccc(N(c3ccccc3)c3cccc4c3sc3cc5c(cc34)-c3ccccc3C53c4ccccc4Oc4ccccc43)cc2)cc1 ; CC1(C)c2ccccc2-c2ccc(N(c3ccccc3)c3cccc4c3sc3cc5c(cc34)-c3ccccc3C53c4ccccc4Oc4ccccc43)cc21 ; CC1(C)c2ccccc2-c2ccc(N(c3ccccc3-c3ccccc3)c3cccc4c3sc3cc5c(cc34)-c3ccccc3C53c4ccccc4Oc4ccccc43)cc21]

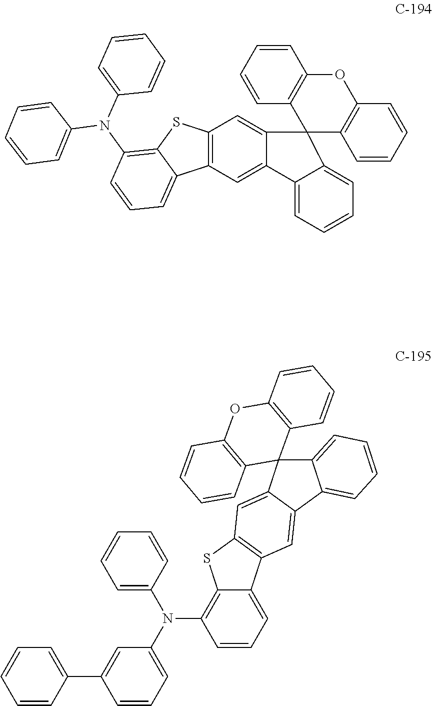 [SMILES: c1ccc(N(c2ccccc2)c2cccc3c2sc2cc4c(cc23)-c2ccccc2C42c3ccccc3Oc3ccccc32)cc1 ; c1ccc(-c2cccc(N(c3ccccc3)c3cccc4c3sc3cc5c(cc34)-c3ccccc3C53c4ccccc4Oc4ccccc43)c2)cc1]

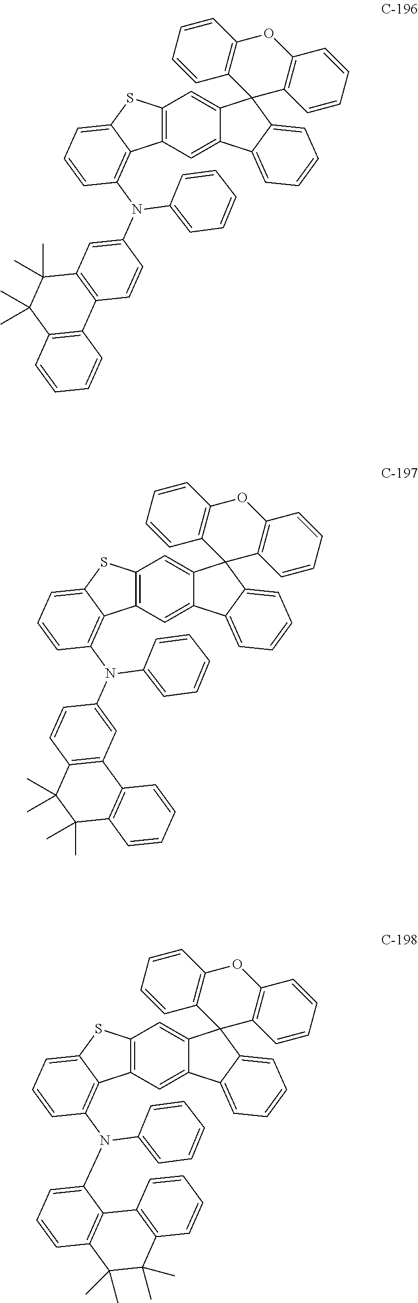 [SMILES: CC1(C)c2ccccc2-c2ccc(N(c3ccccc3)c3cccc4sc5cc6c(cc5c34)-c3ccccc3C63c4ccccc4Oc4ccccc43)cc2C1(C)C ; CC1(C)c2ccccc2-c2cc(N(c3ccccc3)c3cccc4sc5cc6c(cc5c34)-c3ccccc3C63c4ccccc4Oc4ccccc43)ccc2C1(C)C ; CC1(C)c2ccccc2-c2c(N(c3ccccc3)c3cccc4sc5cc6c(cc5c34)-c3ccccc3C63c4ccccc4Oc4ccccc43)cccc2C1(C)C]

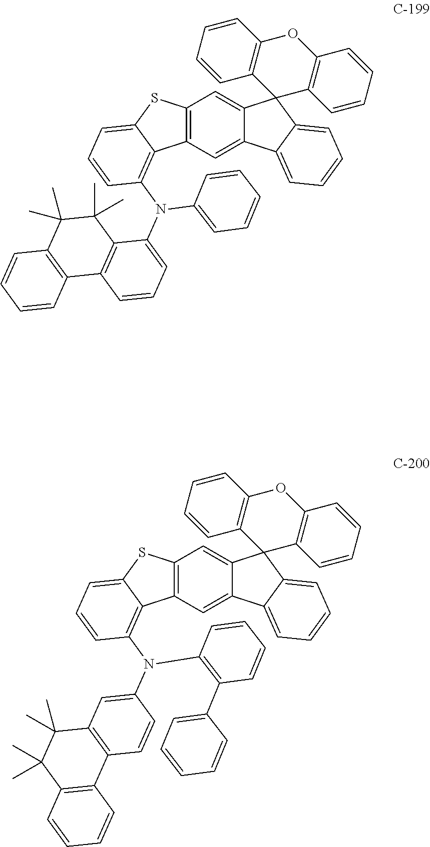 [SMILES: CC1(C)c2ccccc2-c2cccc(N(c3ccccc3)c3cccc4sc5cc6c(cc5c34)-c3ccccc3C63c4ccccc4Oc4ccccc43)c2C1(C)C ; CC1(C)c2ccccc2-c2ccc(N(c3ccccc3-c3ccccc3)c3cccc4sc5cc6c(cc5c34)-c3ccccc3C63c4ccccc4Oc4ccccc43)cc2C1(C)C]

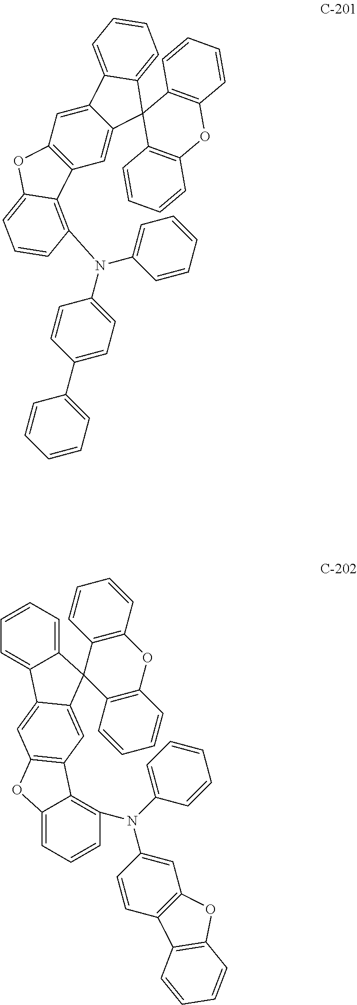 [SMILES: c1ccc(-c2ccc(N(c3ccccc3)c3cccc4oc5cc6c(cc5c34)C3(c4ccccc4Oc4ccccc43)c3ccccc3-6)cc2)cc1 ; c1ccc(N(c2ccc3c(c2)oc2ccccc23)c2cccc3oc4cc5c(cc4c23)C2(c3ccccc3Oc3ccccc32)c2ccccc2-5)cc1]

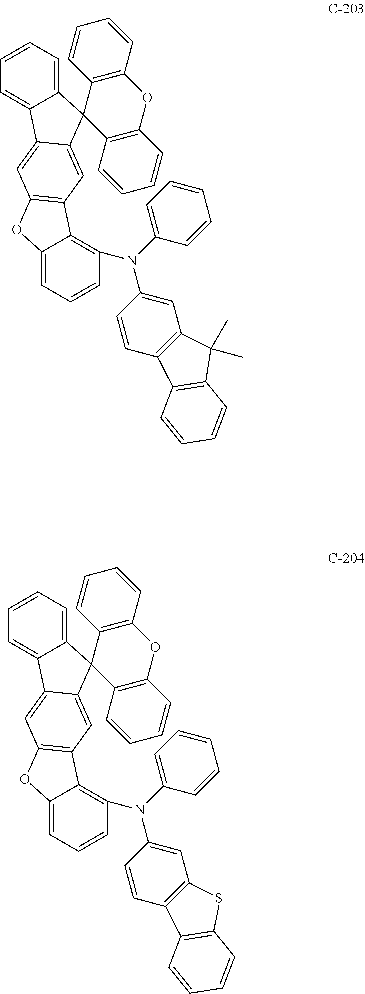 [SMILES: CC1(C)c2ccccc2-c2ccc(N(c3ccccc3)c3cccc4oc5cc6c(cc5c34)C3(c4ccccc4Oc4ccccc43)c3ccccc3-6)cc21 ; c1ccc(N(c2ccc3c(c2)sc2ccccc23)c2cccc3oc4cc5c(cc4c23)C2(c3ccccc3Oc3ccccc32)c2ccccc2-5)cc1]

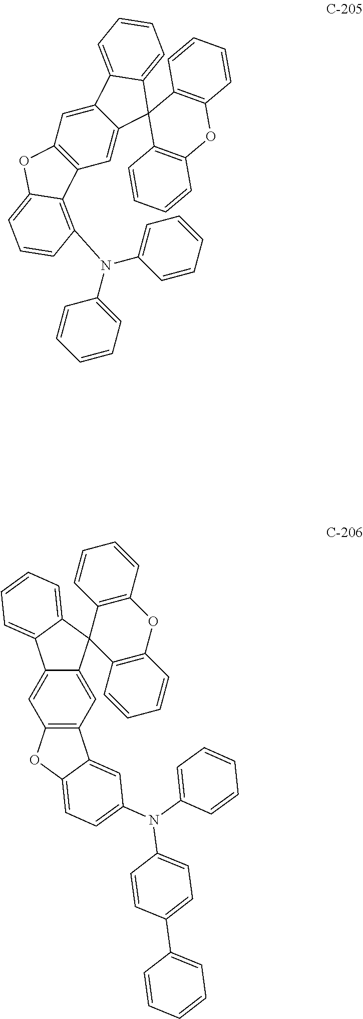 [SMILES: c1ccc(N(c2ccccc2)c2cccc3oc4cc5c(cc4c23)C2(c3ccccc3Oc3ccccc32)c2ccccc2-5)cc1 ; c1ccc(-c2ccc(N(c3ccccc3)c3ccc4oc5cc6c(cc5c4c3)C3(c4ccccc4Oc4ccccc43)c3ccccc3-6)cc2)cc1]

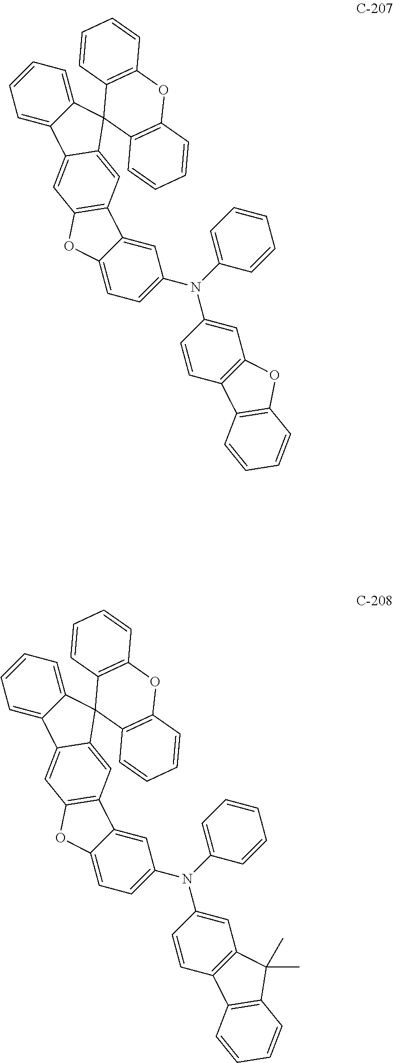 [SMILES: c1ccc(N(c2ccc3c(c2)oc2ccccc23)c2ccc3oc4cc5c(cc4c3c2)C2(c3ccccc3Oc3ccccc32)c2ccccc2-5)cc1 ; CC1(C)c2ccccc2-c2ccc(N(c3ccccc3)c3ccc4oc5cc6c(cc5c4c3)C3(c4ccccc4Oc4ccccc43)c3ccccc3-6)cc21]

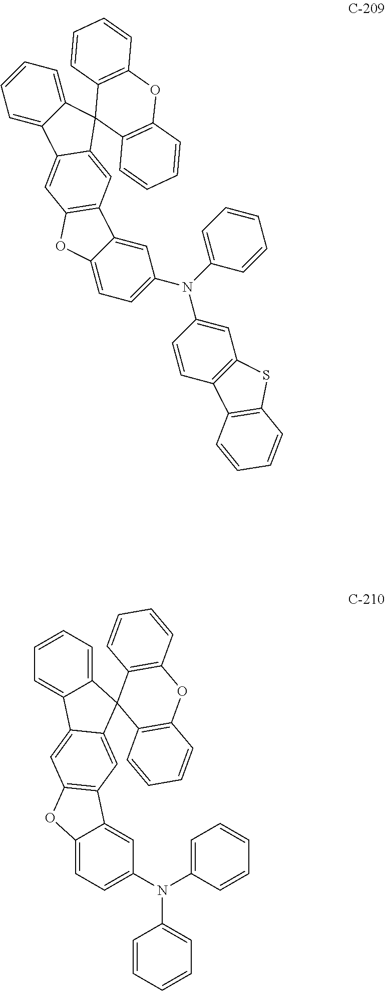 [SMILES: c1ccc(N(c2ccc3c(c2)sc2ccccc23)c2ccc3oc4cc5c(cc4c3c2)C2(c3ccccc3Oc3ccccc32)c2ccccc2-5)cc1 ; c1ccc(N(c2ccccc2)c2ccc3oc4cc5c(cc4c3c2)C2(c3ccccc3Oc3ccccc32)c2ccccc2-5)cc1]

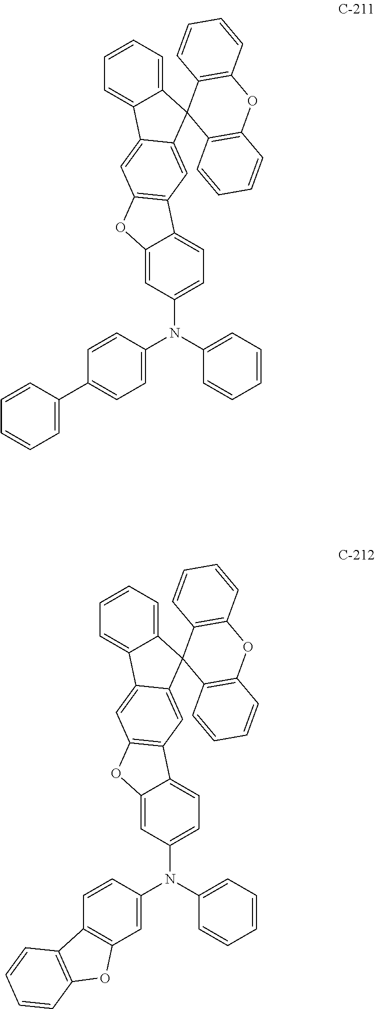 [SMILES: c1ccc(-c2ccc(N(c3ccccc3)c3ccc4c(c3)oc3cc5c(cc34)C3(c4ccccc4Oc4ccccc43)c3ccccc3-5)cc2)cc1 ; c1ccc(N(c2ccc3c(c2)oc2ccccc23)c2ccc3c(c2)oc2cc4c(cc23)C2(c3ccccc3Oc3ccccc32)c2ccccc2-4)cc1]

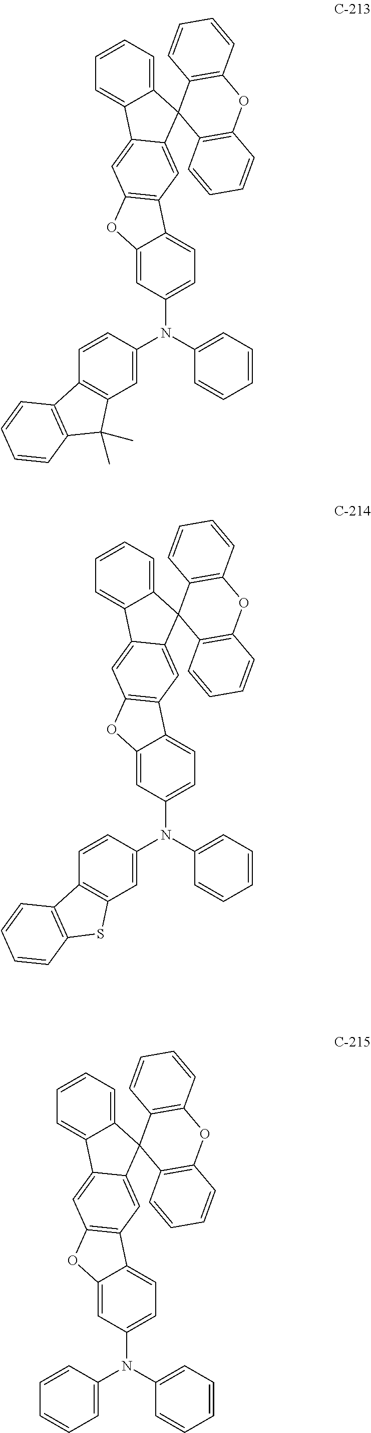 [SMILES: CC1(C)c2ccccc2-c2ccc(N(c3ccccc3)c3ccc4c(c3)oc3cc5c(cc34)C3(c4ccccc4Oc4ccccc43)c3ccccc3-5)cc21 ; c1ccc(N(c2ccc3c(c2)oc2cc4c(cc23)C2(c3ccccc3Oc3ccccc32)c2ccccc2-4)c2ccc3c(c2)sc2ccccc23)cc1 ; c1ccc(N(c2ccccc2)c2ccc3c(c2)oc2cc4c(cc23)C2(c3ccccc3Oc3ccccc32)c2ccccc2-4)cc1]

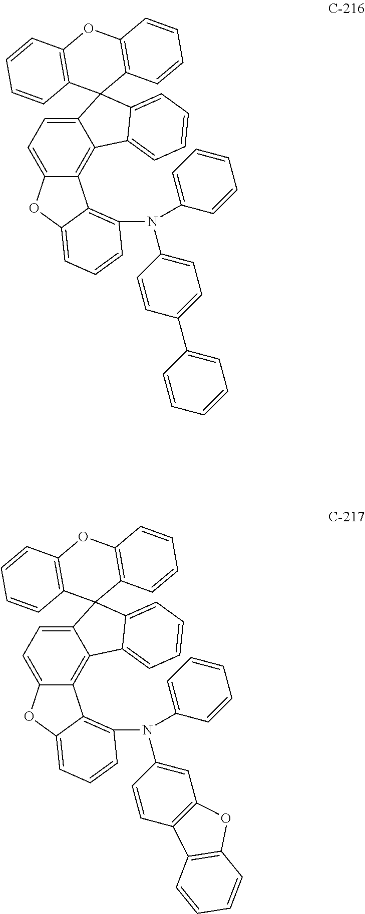 [SMILES: c1ccc(-c2ccc(N(c3ccccc3)c3cccc4oc5ccc6c(c5c34)-c3ccccc3C63c4ccccc4Oc4ccccc43)cc2)cc1 ; c1ccc(N(c2ccc3c(c2)oc2ccccc23)c2cccc3oc4ccc5c(c4c23)-c2ccccc2C52c3ccccc3Oc3ccccc32)cc1]

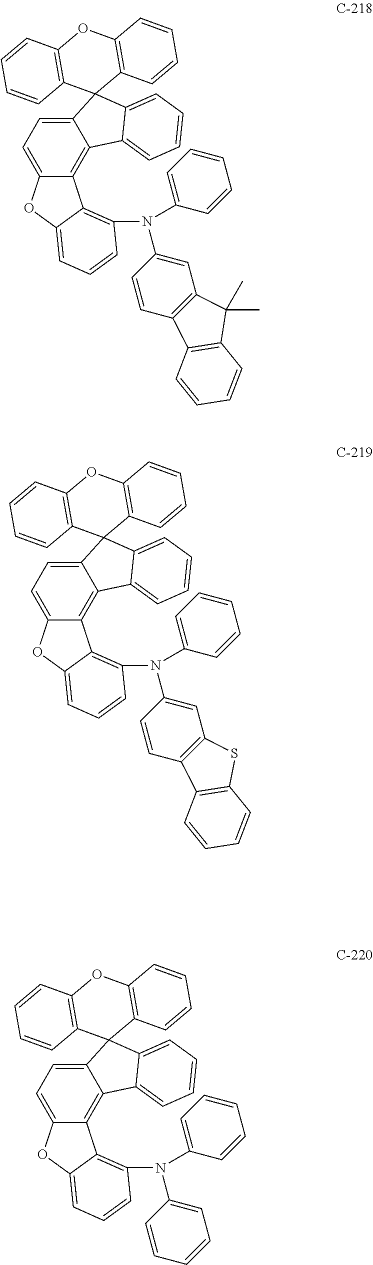 [SMILES: CC1(C)c2ccccc2-c2ccc(N(c3ccccc3)c3cccc4oc5ccc6c(c5c34)-c3ccccc3C63c4ccccc4Oc4ccccc43)cc21 ; c1ccc(N(c2ccc3c(c2)sc2ccccc23)c2cccc3oc4ccc5c(c4c23)-c2ccccc2C52c3ccccc3Oc3ccccc32)cc1 ; c1ccc(N(c2ccccc2)c2cccc3oc4ccc5c(c4c23)-c2ccccc2C52c3ccccc3Oc3ccccc32)cc1]

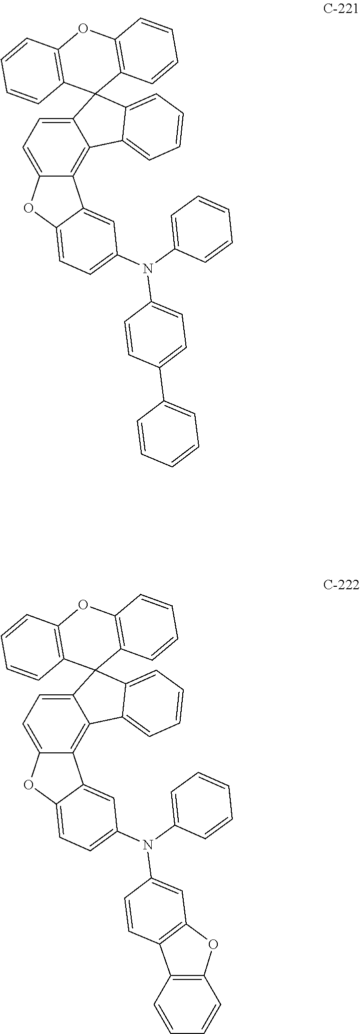 [SMILES: c1ccc(-c2ccc(N(c3ccccc3)c3ccc4oc5ccc6c(c5c4c3)-c3ccccc3C63c4ccccc4Oc4ccccc43)cc2)cc1 ; c1ccc(N(c2ccc3c(c2)oc2ccccc23)c2ccc3oc4ccc5c(c4c3c2)-c2ccccc2C52c3ccccc3Oc3ccccc32)cc1]

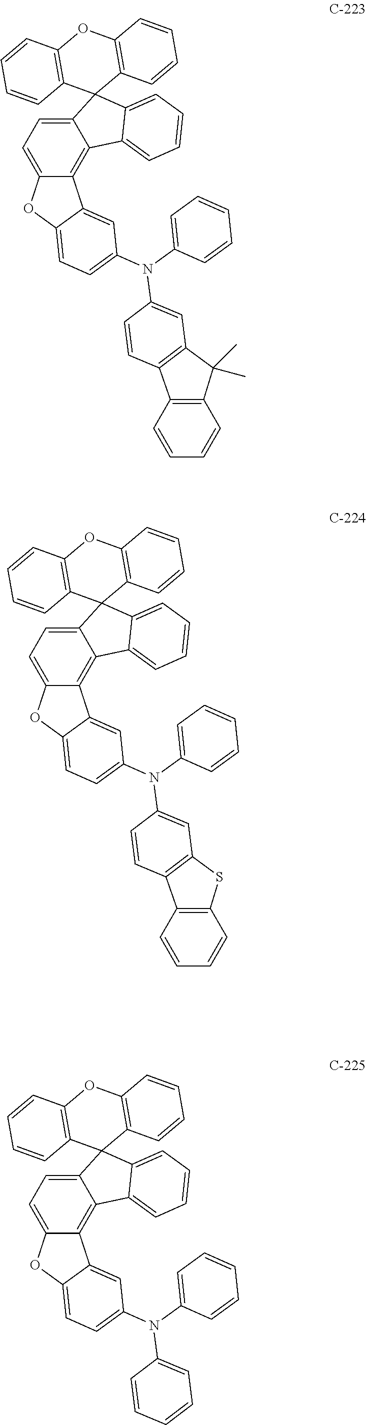 [SMILES: CC1(C)c2ccccc2-c2ccc(N(c3ccccc3)c3ccc4oc5ccc6c(c5c4c3)-c3ccccc3C63c4ccccc4Oc4ccccc43)cc21 ; c1ccc(N(c2ccc3c(c2)sc2ccccc23)c2ccc3oc4ccc5c(c4c3c2)-c2ccccc2C52c3ccccc3Oc3ccccc32)cc1 ; c1ccc(N(c2ccccc2)c2ccc3oc4ccc5c(c4c3c2)-c2ccccc2C52c3ccccc3Oc3ccccc32)cc1]

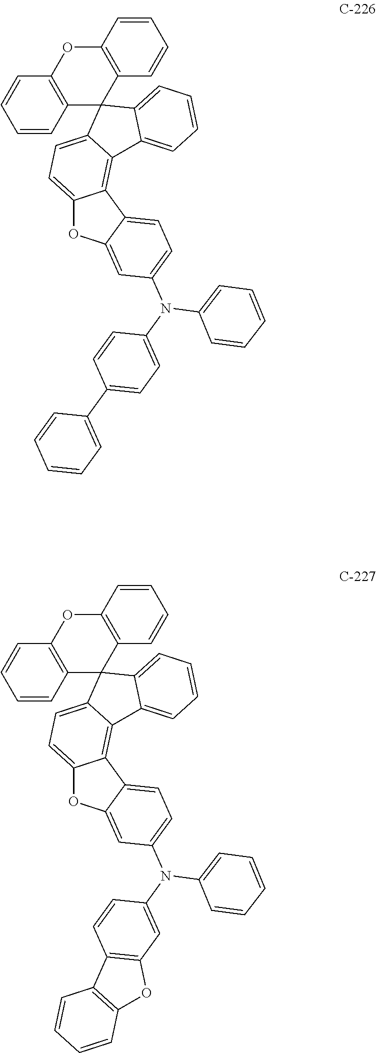 [SMILES: c1ccc(-c2ccc(N(c3ccccc3)c3ccc4c(c3)oc3ccc5c(c34)-c3ccccc3C53c4ccccc4Oc4ccccc43)cc2)cc1 ; c1ccc(N(c2ccc3c(c2)oc2ccccc23)c2ccc3c(c2)oc2ccc4c(c23)-c2ccccc2C42c3ccccc3Oc3ccccc32)cc1]

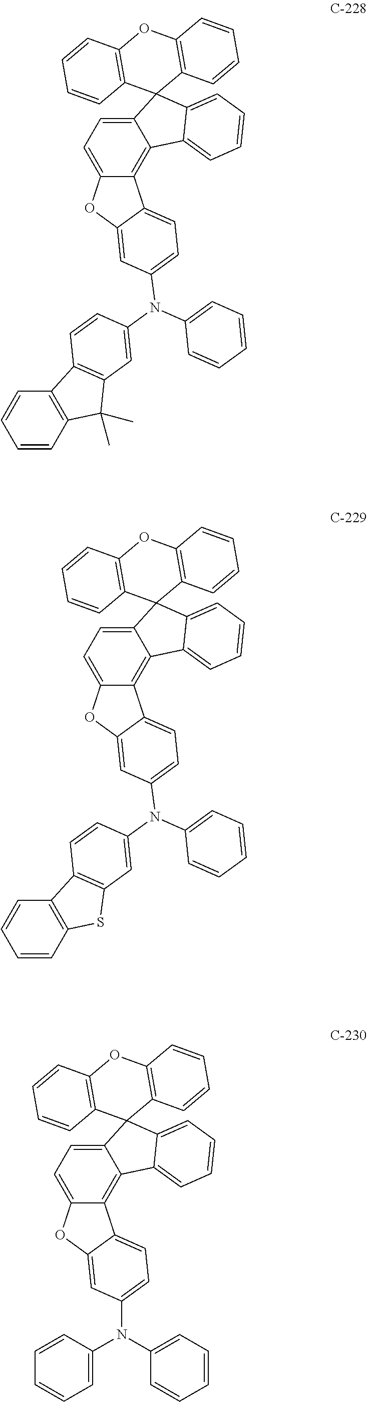 [SMILES: CC1(C)c2ccccc2-c2ccc(N(c3ccccc3)c3ccc4c(c3)oc3ccc5c(c34)-c3ccccc3C53c4ccccc4Oc4ccccc43)cc21 ; c1ccc(N(c2ccc3c(c2)oc2ccc4c(c23)-c2ccccc2C42c3ccccc3Oc3ccccc32)c2ccc3c(c2)sc2ccccc23)cc1 ; c1ccc(N(c2ccccc2)c2ccc3c(c2)oc2ccc4c(c23)-c2ccccc2C42c3ccccc3Oc3ccccc32)cc1]

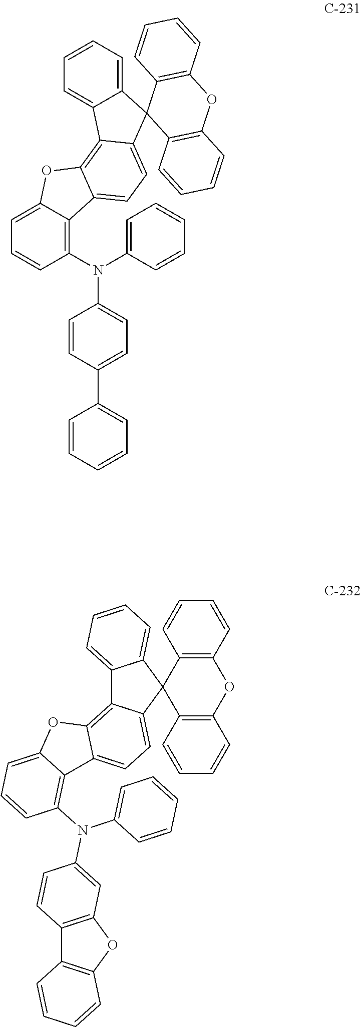 [SMILES: c1ccc(-c2ccc(N(c3ccccc3)c3cccc4oc5c6c(ccc5c34)C3(c4ccccc4Oc4ccccc43)c3ccccc3-6)cc2)cc1 ; c1ccc(N(c2ccc3c(c2)oc2ccccc23)c2cccc3oc4c5c(ccc4c23)C2(c3ccccc3Oc3ccccc32)c2ccccc2-5)cc1]

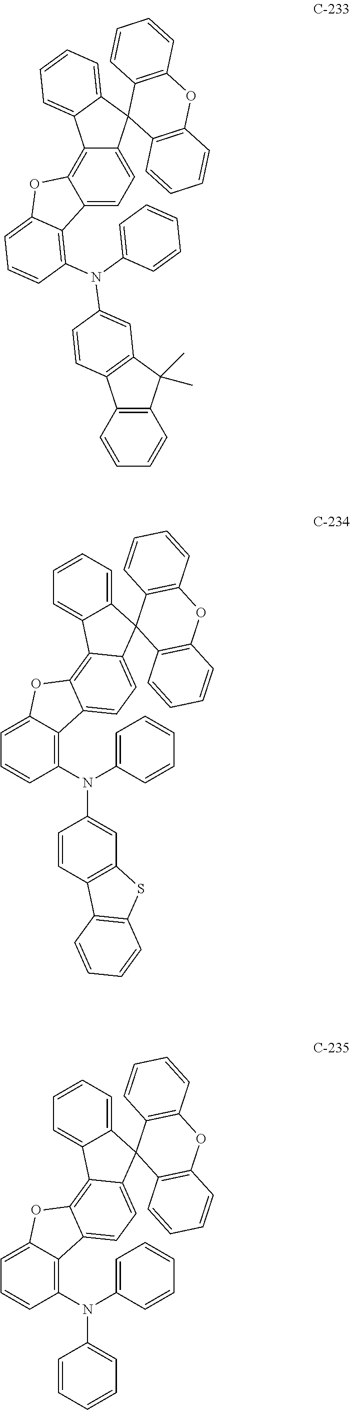 [SMILES: CC1(C)c2ccccc2-c2ccc(N(c3ccccc3)c3cccc4oc5c6c(ccc5c34)C3(c4ccccc4Oc4ccccc43)c3ccccc3-6)cc21 ; c1ccc(N(c2ccc3c(c2)sc2ccccc23)c2cccc3oc4c5c(ccc4c23)C2(c3ccccc3Oc3ccccc32)c2ccccc2-5)cc1 ; c1ccc(N(c2ccccc2)c2cccc3oc4c5c(ccc4c23)C2(c3ccccc3Oc3ccccc32)c2ccccc2-5)cc1]

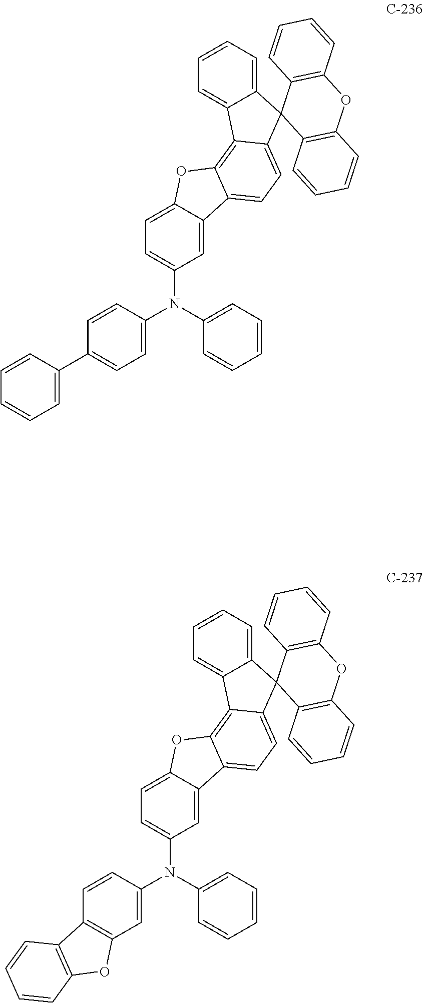 [SMILES: c1ccc(-c2ccc(N(c3ccccc3)c3ccc4oc5c6c(ccc5c4c3)C3(c4ccccc4Oc4ccccc43)c3ccccc3-6)cc2)cc1 ; c1ccc(N(c2ccc3c(c2)oc2ccccc23)c2ccc3oc4c5c(ccc4c3c2)C2(c3ccccc3Oc3ccccc32)c2ccccc2-5)cc1]

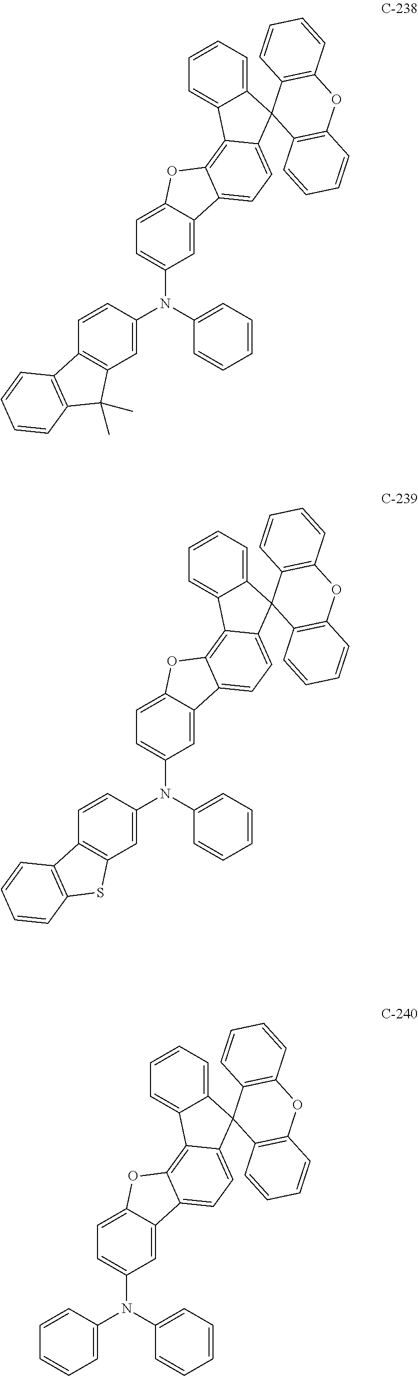 [SMILES: CC1(C)c2ccccc2-c2ccc(N(c3ccccc3)c3ccc4oc5c6c(ccc5c4c3)C3(c4ccccc4Oc4ccccc43)c3ccccc3-6)cc21 ; c1ccc(N(c2ccc3c(c2)sc2ccccc23)c2ccc3oc4c5c(ccc4c3c2)C2(c3ccccc3Oc3ccccc32)c2ccccc2-5)cc1 ; c1ccc(N(c2ccccc2)c2ccc3oc4c5c(ccc4c3c2)C2(c3ccccc3Oc3ccccc32)c2ccccc2-5)cc1]

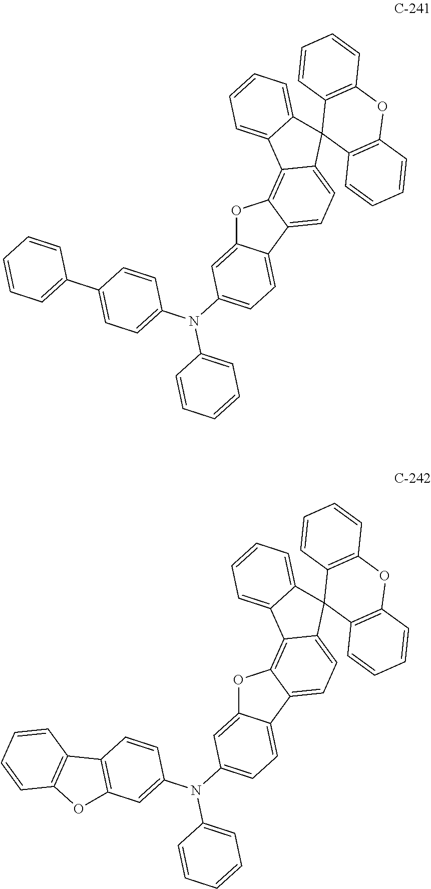 [SMILES: c1ccc(-c2ccc(N(c3ccccc3)c3ccc4c(c3)oc3c5c(ccc34)C3(c4ccccc4Oc4ccccc43)c3ccccc3-5)cc2)cc1 ; c1ccc(N(c2ccc3c(c2)oc2ccccc23)c2ccc3c(c2)oc2c4c(ccc23)C2(c3ccccc3Oc3ccccc32)c2ccccc2-4)cc1]

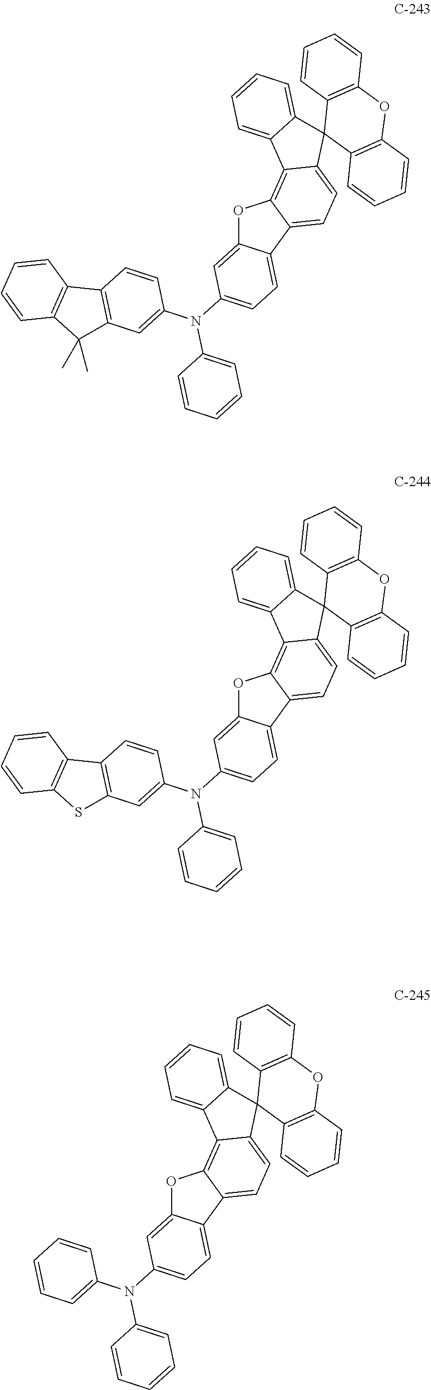 [SMILES: CC1(C)c2ccccc2-c2ccc(N(c3ccccc3)c3ccc4c(c3)oc3c5c(ccc34)C3(c4ccccc4Oc4ccccc43)c3ccccc3-5)cc21 ; c1ccc(N(c2ccc3c(c2)oc2c4c(ccc23)C2(c3ccccc3Oc3ccccc32)c2ccccc2-4)c2ccc3c(c2)sc2ccccc23)cc1 ; c1ccc(N(c2ccccc2)c2ccc3c(c2)oc2c4c(ccc23)C2(c3ccccc3Oc3ccccc32)c2ccccc2-4)cc1]

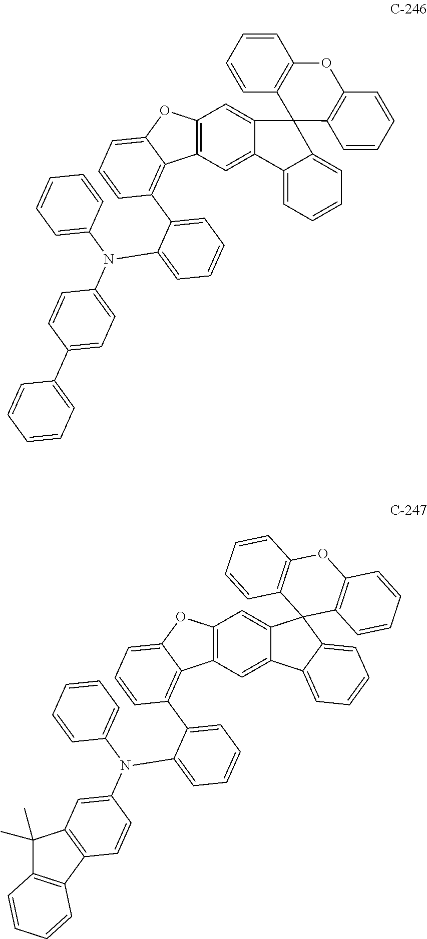 [SMILES: c1ccc(-c2ccc(N(c3ccccc3)c3ccccc3-c3cccc4oc5cc6c(cc5c34)-c3ccccc3C63c4ccccc4Oc4ccccc43)cc2)cc1 ; CC1(C)c2ccccc2-c2ccc(N(c3ccccc3)c3ccccc3-c3cccc4oc5cc6c(cc5c34)-c3ccccc3C63c4ccccc4Oc4ccccc43)cc21]

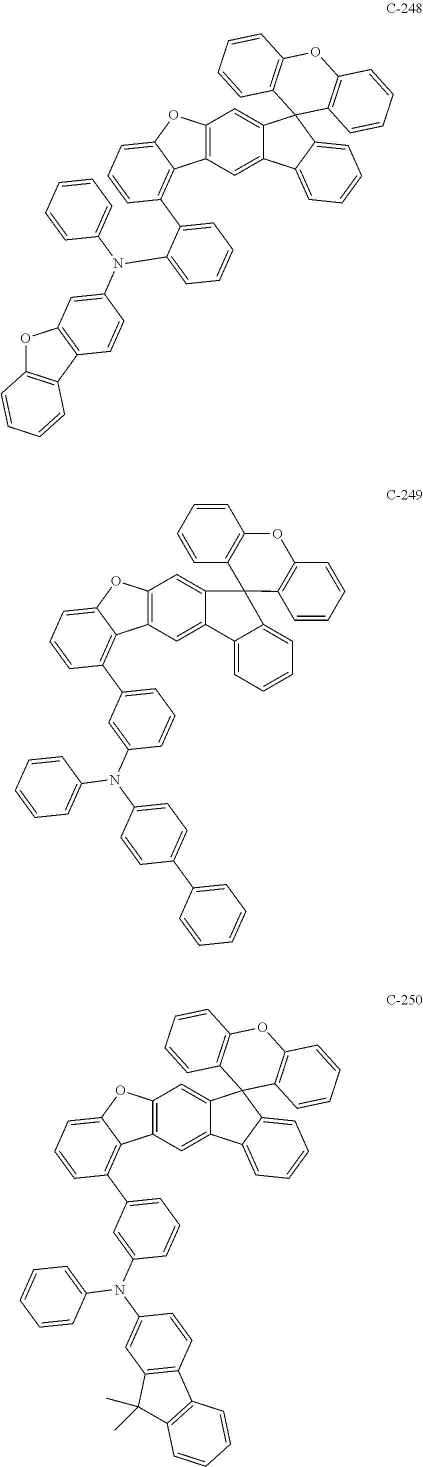 [SMILES: c1ccc(N(c2ccc3c(c2)oc2ccccc23)c2ccccc2-c2cccc3oc4cc5c(cc4c23)-c2ccccc2C52c3ccccc3Oc3ccccc32)cc1 ; c1ccc(-c2ccc(N(c3ccccc3)c3cccc(-c4cccc5oc6cc7c(cc6c45)-c4ccccc4C74c5ccccc5Oc5ccccc54)c3)cc2)cc1 ; CC1(C)c2ccccc2-c2ccc(N(c3ccccc3)c3cccc(-c4cccc5oc6cc7c(cc6c45)-c4ccccc4C74c5ccccc5Oc5ccccc54)c3)cc21]

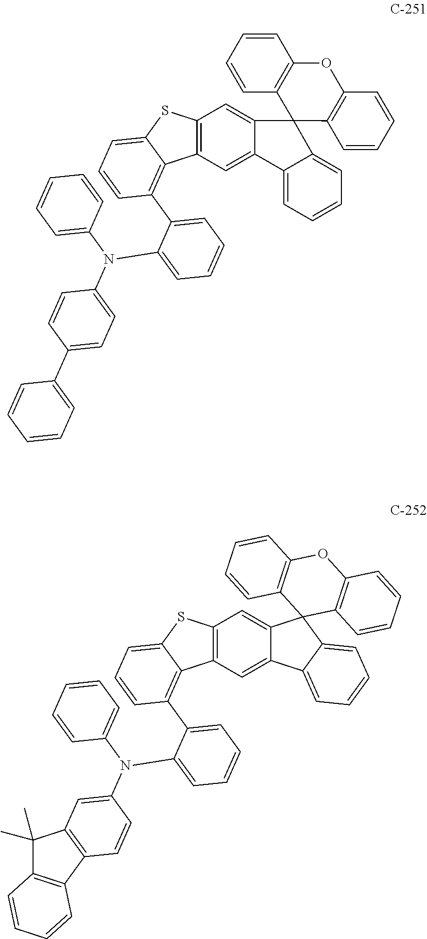 [SMILES: c1ccc(-c2ccc(N(c3ccccc3)c3ccccc3-c3cccc4sc5cc6c(cc5c34)-c3ccccc3C63c4ccccc4Oc4ccccc43)cc2)cc1 ; CC1(C)c2ccccc2-c2ccc(N(c3ccccc3)c3ccccc3-c3cccc4sc5cc6c(cc5c34)-c3ccccc3C63c4ccccc4Oc4ccccc43)cc21]

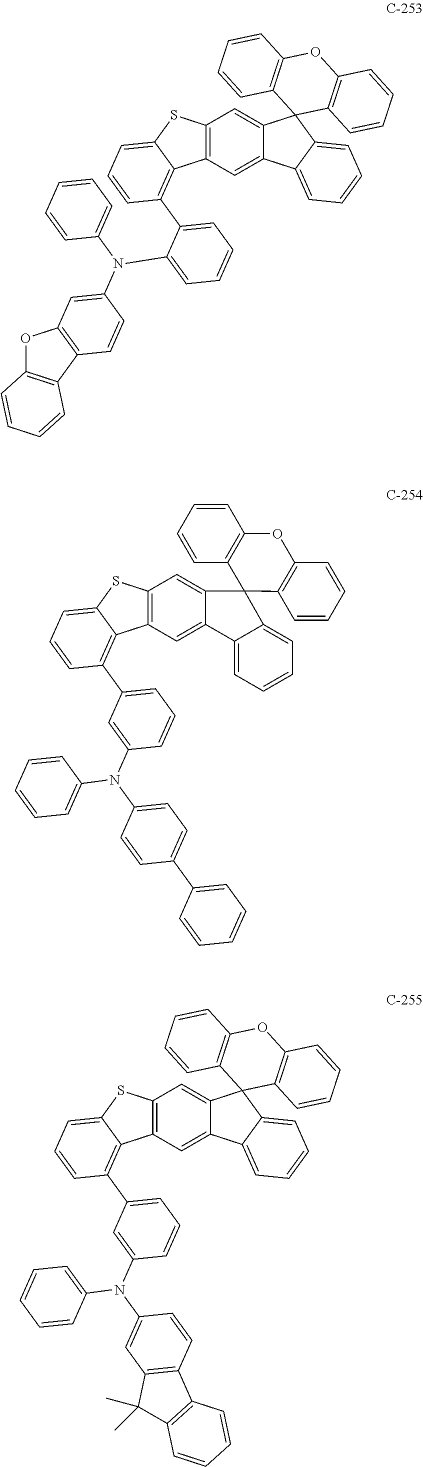 [SMILES: c1ccc(N(c2ccc3c(c2)oc2ccccc23)c2ccccc2-c2cccc3sc4cc5c(cc4c23)-c2ccccc2C52c3ccccc3Oc3ccccc32)cc1 ; c1ccc(-c2ccc(N(c3ccccc3)c3cccc(-c4cccc5sc6cc7c(cc6c45)-c4ccccc4C74c5ccccc5Oc5ccccc54)c3)cc2)cc1 ; CC1(C)c2ccccc2-c2ccc(N(c3ccccc3)c3cccc(-c4cccc5sc6cc7c(cc6c45)-c4ccccc4C74c5ccccc5Oc5ccccc54)c3)cc21]

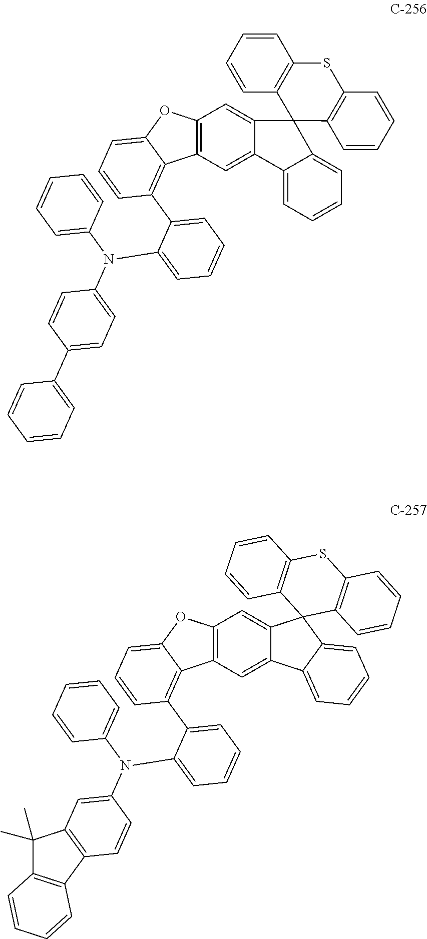 [SMILES: c1ccc(-c2ccc(N(c3ccccc3)c3ccccc3-c3cccc4oc5cc6c(cc5c34)-c3ccccc3C63c4ccccc4Sc4ccccc43)cc2)cc1 ; CC1(C)c2ccccc2-c2ccc(N(c3ccccc3)c3ccccc3-c3cccc4oc5cc6c(cc5c34)-c3ccccc3C63c4ccccc4Sc4ccccc43)cc21]

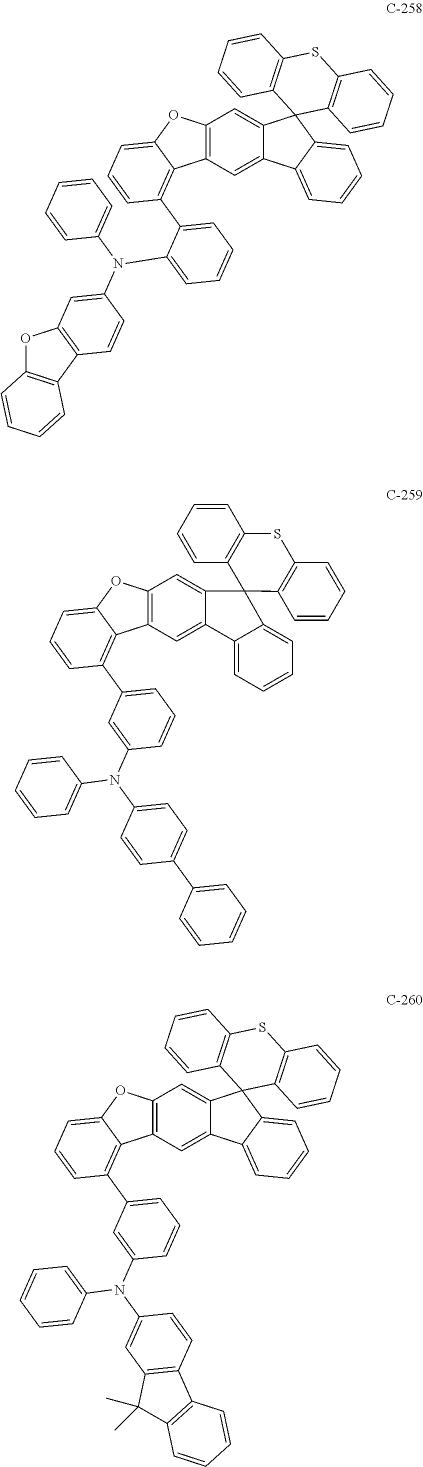 [SMILES: c1ccc(N(c2ccc3c(c2)oc2ccccc23)c2ccccc2-c2cccc3oc4cc5c(cc4c23)-c2ccccc2C52c3ccccc3Sc3ccccc32)cc1 ; c1ccc(-c2ccc(N(c3ccccc3)c3cccc(-c4cccc5oc6cc7c(cc6c45)-c4ccccc4C74c5ccccc5Sc5ccccc54)c3)cc2)cc1 ; CC1(C)c2ccccc2-c2ccc(N(c3ccccc3)c3cccc(-c4cccc5oc6cc7c(cc6c45)-c4ccccc4C74c5ccccc5Sc5ccccc54)c3)cc21]

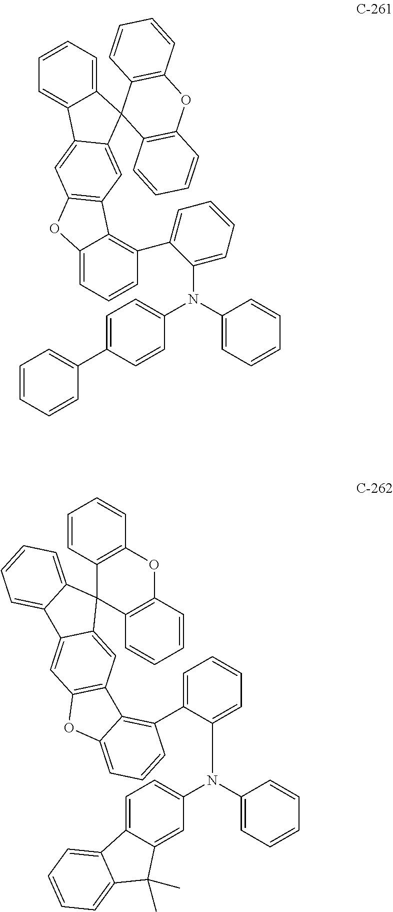 [SMILES: c1ccc(-c2ccc(N(c3ccccc3)c3ccccc3-c3cccc4oc5cc6c(cc5c34)C3(c4ccccc4Oc4ccccc43)c3ccccc3-6)cc2)cc1 ; CC1(C)c2ccccc2-c2ccc(N(c3ccccc3)c3ccccc3-c3cccc4oc5cc6c(cc5c34)C3(c4ccccc4Oc4ccccc43)c3ccccc3-6)cc21]

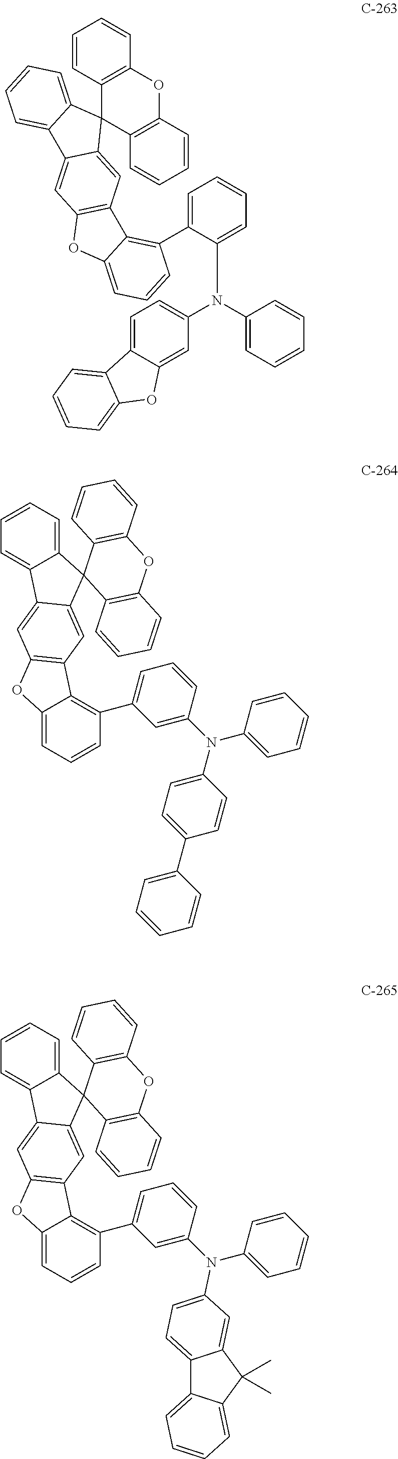 [SMILES: c1ccc(N(c2ccc3c(c2)oc2ccccc23)c2ccccc2-c2cccc3oc4cc5c(cc4c23)C2(c3ccccc3Oc3ccccc32)c2ccccc2-5)cc1 ; c1ccc(-c2ccc(N(c3ccccc3)c3cccc(-c4cccc5oc6cc7c(cc6c45)C4(c5ccccc5Oc5ccccc54)c4ccccc4-7)c3)cc2)cc1 ; CC1(C)c2ccccc2-c2ccc(N(c3ccccc3)c3cccc(-c4cccc5oc6cc7c(cc6c45)C4(c5ccccc5Oc5ccccc54)c4ccccc4-7)c3)cc21]

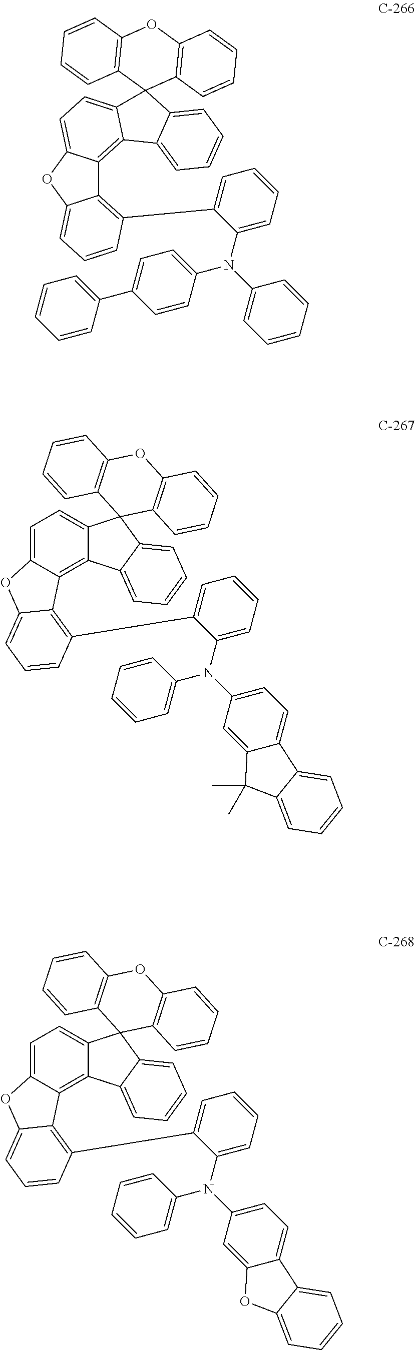 [SMILES: c1ccc(-c2ccc(N(c3ccccc3)c3ccccc3-c3cccc4oc5ccc6c(c5c34)-c3ccccc3C63c4ccccc4Oc4ccccc43)cc2)cc1 ; CC1(C)c2ccccc2-c2ccc(N(c3ccccc3)c3ccccc3-c3cccc4oc5ccc6c(c5c34)-c3ccccc3C63c4ccccc4Oc4ccccc43)cc21 ; c1ccc(N(c2ccc3c(c2)oc2ccccc23)c2ccccc2-c2cccc3oc4ccc5c(c4c23)-c2ccccc2C52c3ccccc3Oc3ccccc32)cc1]

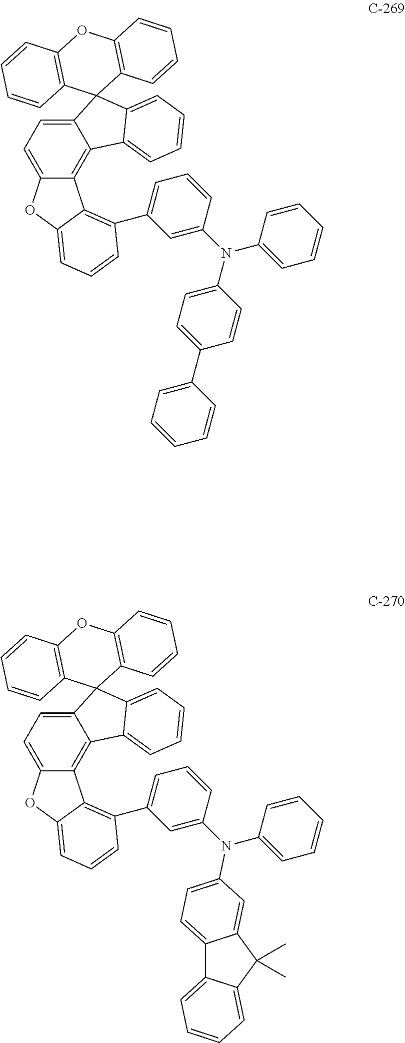 [SMILES: c1ccc(-c2ccc(N(c3ccccc3)c3cccc(-c4cccc5oc6ccc7c(c6c45)-c4ccccc4C74c5ccccc5Oc5ccccc54)c3)cc2)cc1 ; CC1(C)c2ccccc2-c2ccc(N(c3ccccc3)c3cccc(-c4cccc5oc6ccc7c(c6c45)-c4ccccc4C74c5ccccc5Oc5ccccc54)c3)cc21]

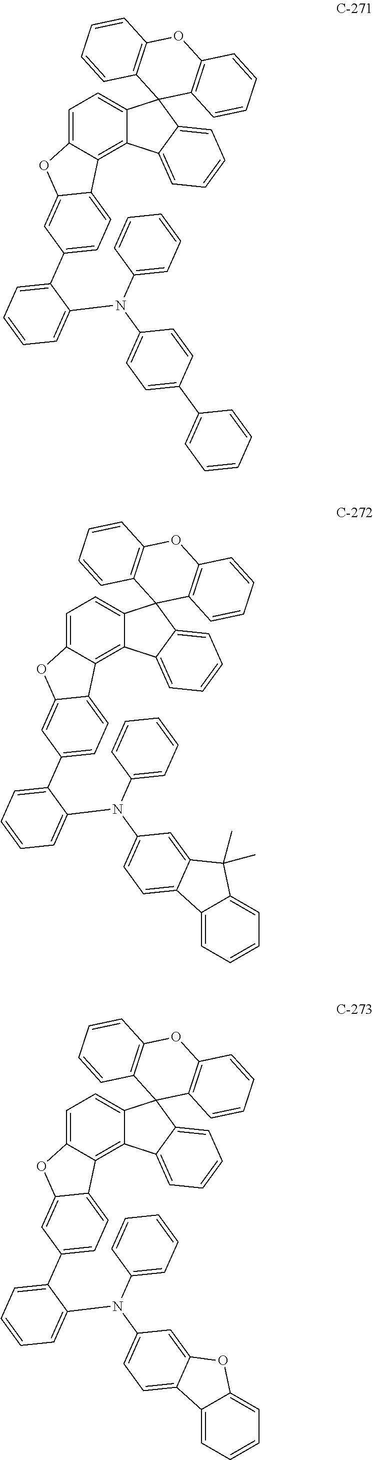 [SMILES: c1ccc(-c2ccc(N(c3ccccc3)c3ccccc3-c3ccc4c(c3)oc3ccc5c(c34)-c3ccccc3C53c4ccccc4Oc4ccccc43)cc2)cc1 ; CC1(C)c2ccccc2-c2ccc(N(c3ccccc3)c3ccccc3-c3ccc4c(c3)oc3ccc5c(c34)-c3ccccc3C53c4ccccc4Oc4ccccc43)cc21 ; c1ccc(N(c2ccc3c(c2)oc2ccccc23)c2ccccc2-c2ccc3c(c2)oc2ccc4c(c23)-c2ccccc2C42c3ccccc3Oc3ccccc32)cc1]

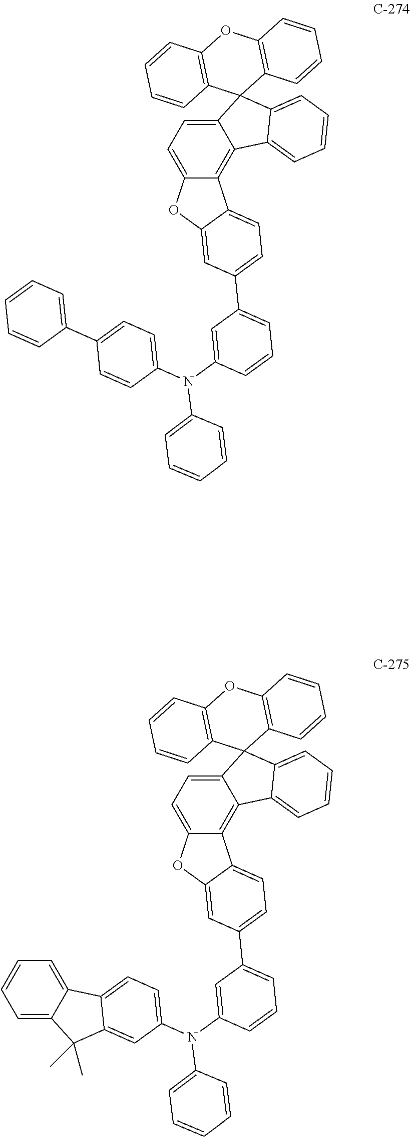 [SMILES: c1ccc(-c2ccc(N(c3ccccc3)c3cccc(-c4ccc5c(c4)oc4ccc6c(c45)-c4ccccc4C64c5ccccc5Oc5ccccc54)c3)cc2)cc1 ; CC1(C)c2ccccc2-c2ccc(N(c3ccccc3)c3cccc(-c4ccc5c(c4)oc4ccc6c(c45)-c4ccccc4C64c5ccccc5Oc5ccccc54)c3)cc21]

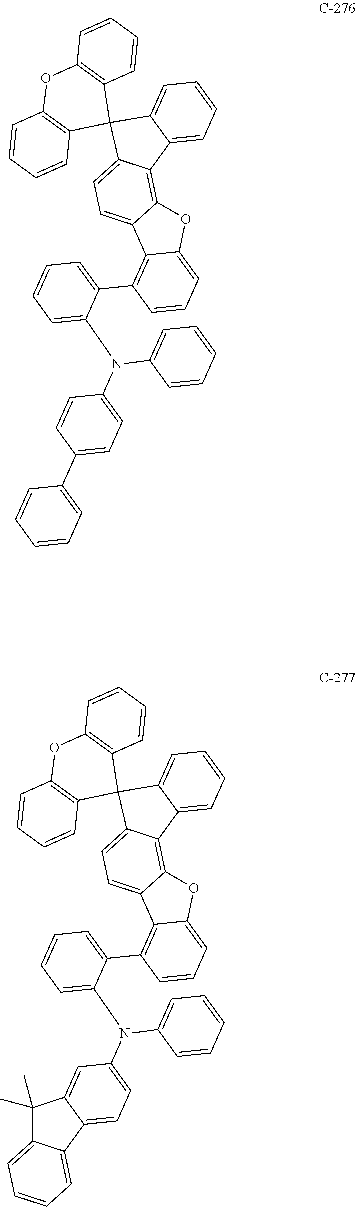 [SMILES: c1ccc(-c2ccc(N(c3ccccc3)c3ccccc3-c3cccc4oc5c6c(ccc5c34)C3(c4ccccc4Oc4ccccc43)c3ccccc3-6)cc2)cc1 ; CC1(C)c2ccccc2-c2ccc(N(c3ccccc3)c3ccccc3-c3cccc4oc5c6c(ccc5c34)C3(c4ccccc4Oc4ccccc43)c3ccccc3-6)cc21]

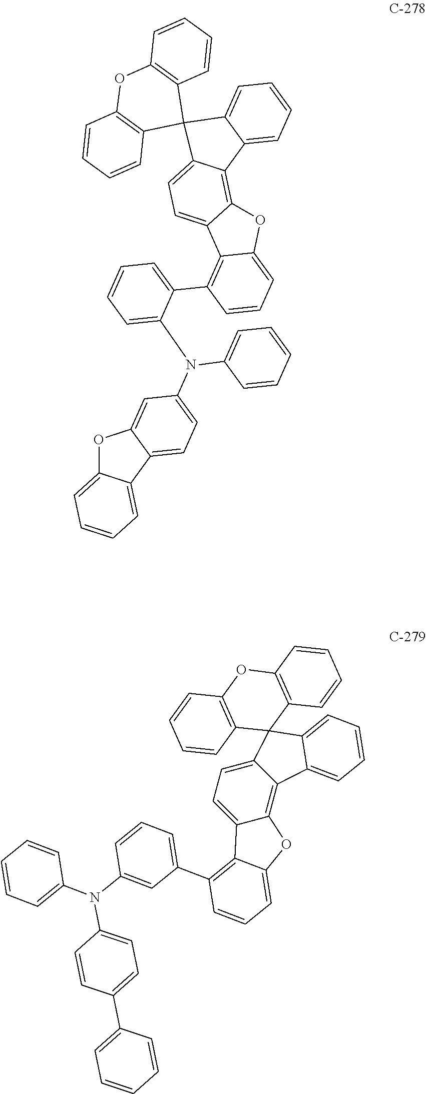 [SMILES: c1ccc(N(c2ccc3c(c2)oc2ccccc23)c2ccccc2-c2cccc3oc4c5c(ccc4c23)C2(c3ccccc3Oc3ccccc32)c2ccccc2-5)cc1 ; c1ccc(-c2ccc(N(c3ccccc3)c3cccc(-c4cccc5oc6c7c(ccc6c45)C4(c5ccccc5Oc5ccccc54)c4ccccc4-7)c3)cc2)cc1]

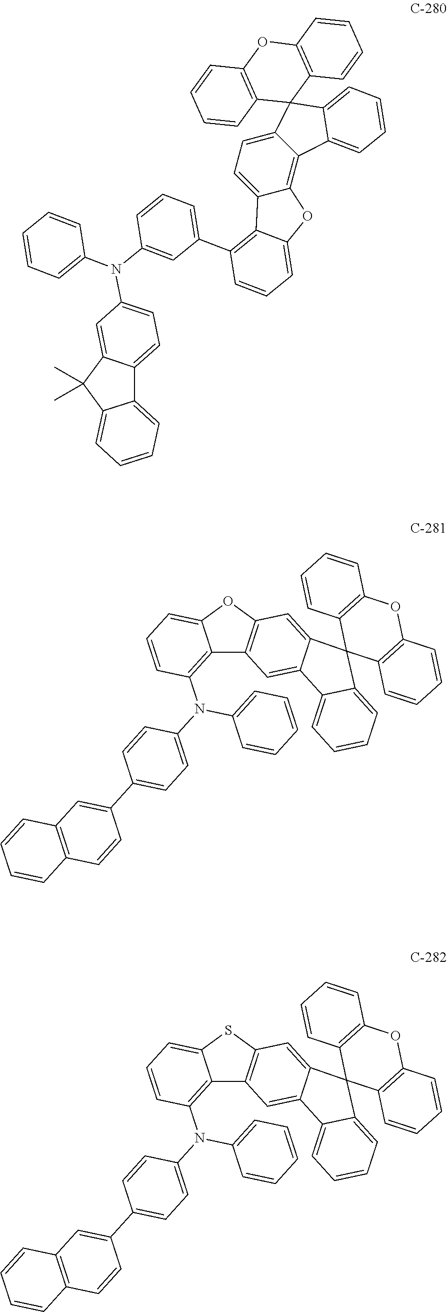 [SMILES: CC1(C)c2ccccc2-c2ccc(N(c3ccccc3)c3cccc(-c4cccc5oc6c7c(ccc6c45)C4(c5ccccc5Oc5ccccc54)c4ccccc4-7)c3)cc21 ; c1ccc(N(c2ccc(-c3ccc4ccccc4c3)cc2)c2cccc3oc4cc5c(cc4c23)-c2ccccc2C52c3ccccc3Oc3ccccc32)cc1 ; c1ccc(N(c2ccc(-c3ccc4ccccc4c3)cc2)c2cccc3sc4cc5c(cc4c23)-c2ccccc2C52c3ccccc3Oc3ccccc32)cc1]

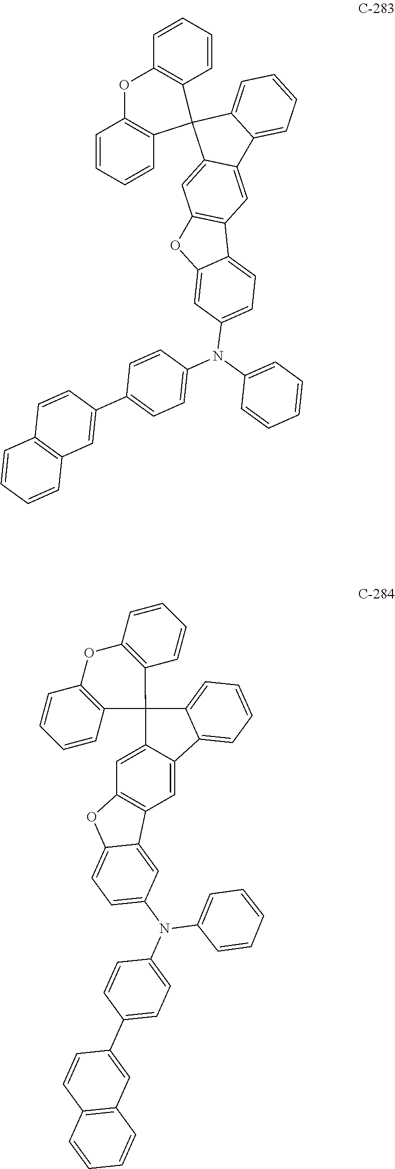 [SMILES: c1ccc(N(c2ccc(-c3ccc4ccccc4c3)cc2)c2ccc3c(c2)oc2cc4c(cc23)-c2ccccc2C42c3ccccc3Oc3ccccc32)cc1 ; c1ccc(N(c2ccc(-c3ccc4ccccc4c3)cc2)c2ccc3oc4cc5c(cc4c3c2)-c2ccccc2C52c3ccccc3Oc3ccccc32)cc1]

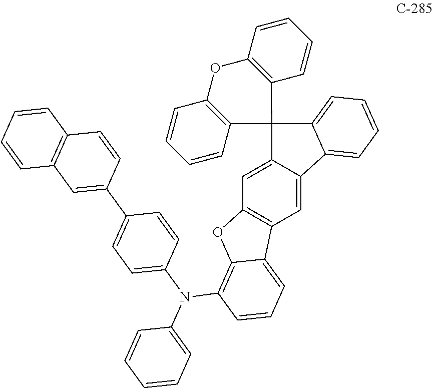 [SMILES: c1ccc(N(c2ccc(-c3ccc4ccccc4c3)cc2)c2cccc3c2oc2cc4c(cc23)-c2ccccc2C42c3ccccc3Oc3ccccc32)cc1]

The compound of formula 1 according to the present disclosure may be produced as represented by the following reaction schemes 1 to 3, but is not limited thereto. Further, it may be prepared by a synthetic method known to a person skilled in the art.

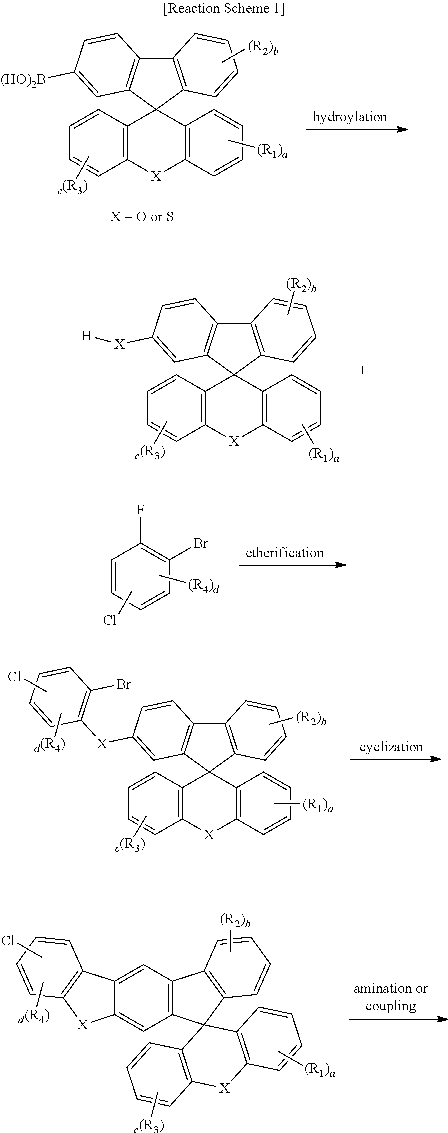

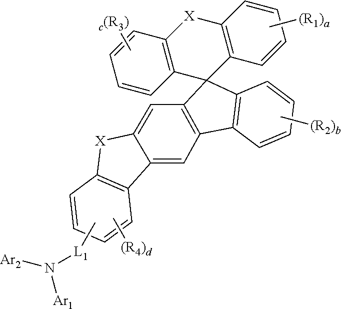

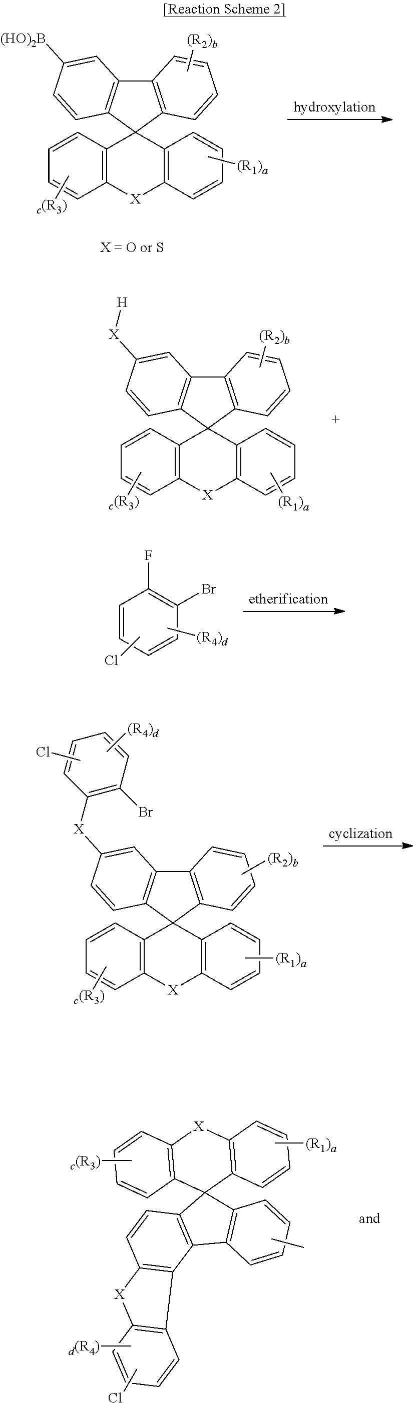

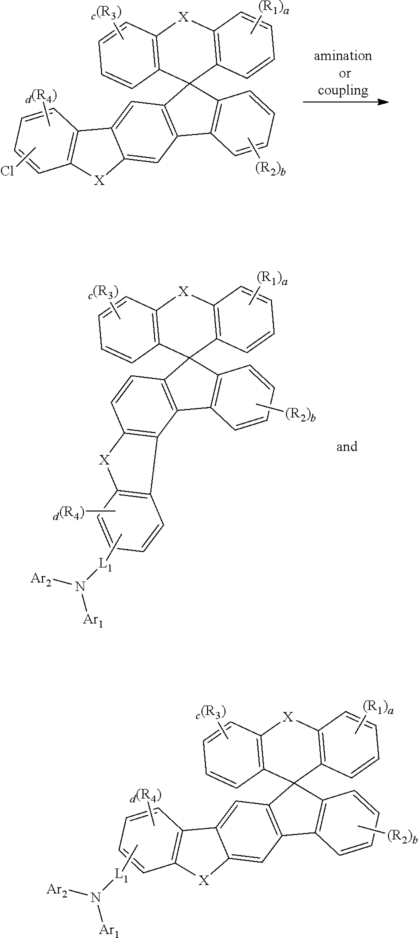

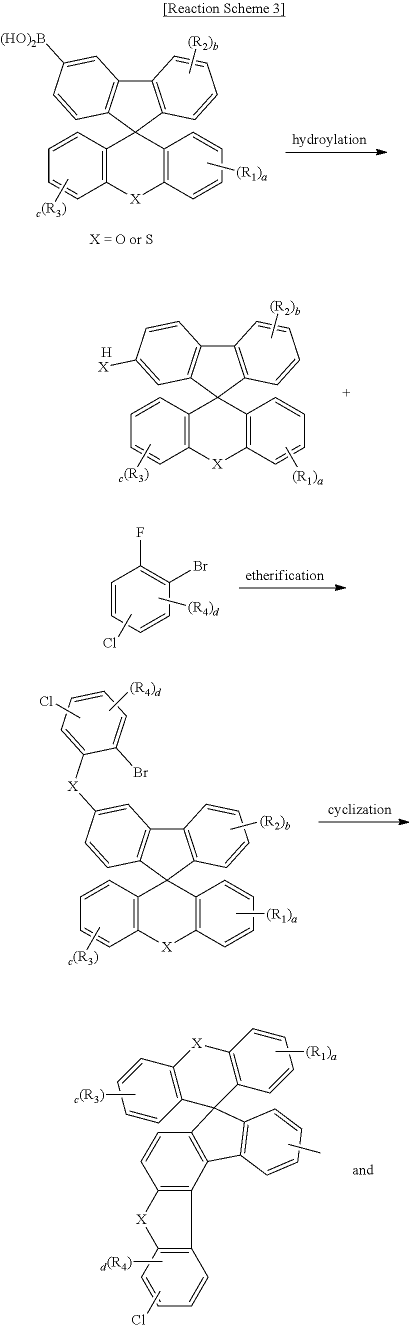

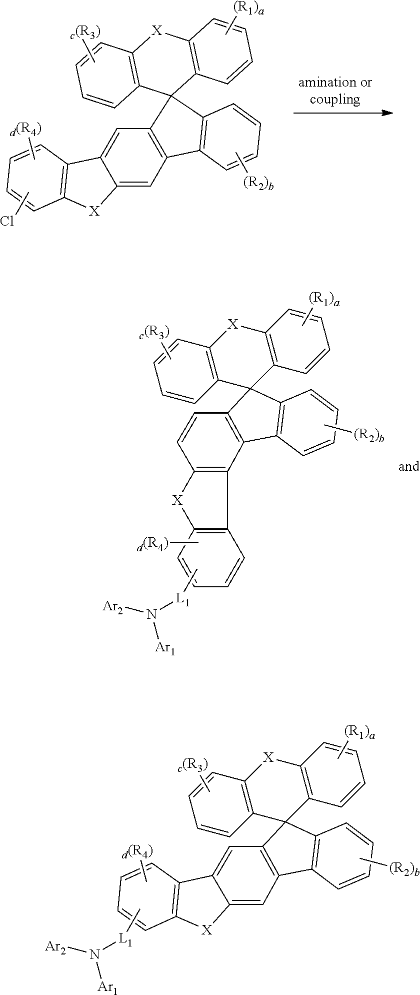

In reaction schemes 1 to 3 above, the definition of each substituents are as defined in formula 1 above.

As described above, exemplary synthesis examples of the compounds represented by formula 1 according to the present disclosure are described, but they are based on Buchwald-Hartwig cross coupling reaction, N-arylation reaction, H-mont-mediated etherification reaction, Miyaura borylation reaction, Suzuki cross-coupling reaction, Intramolecular acid-induced cyclization reaction, Pd(II)-catalyzed oxidative cyclization reaction. Grignard reaction, Heck reaction, Cyclic Dehydration reaction, SN₁ substitution reaction, SN₂ substitution reaction, and Phosphine-mediated reductive cyclization reaction etc. It will be understood by one skilled in the art that the above reaction proceeds even if other substituents defined in formula 1, other than the substituents described in the specific synthesis examples, are bonded,

According to one embodiment, the present disclosure can provide an organic electroluminescent material comprising an organic electroluminescent compound of formula 1, and an organic electroluminescent device comprising the organic electroluminescent material.

The organic electroluminescent material may be comprised solely of the organic electroluminescent compound of formula 1 of the present disclosure, or may further comprise conventional materials included in the organic electroluminescent material.

The organic electroluminescent material according to one embodiment may be comprised of at least one compound represented by the formula 1. The organic electroluminescent compound of formula 1 of the present disclosure may preferably be included as a hole transport material in the hole transport zone of the organic electroluminescent device. The hole transport zone includes a hole transport layer, In addition, the hole transport zone may further include at least one of a hole injection layer, a hole auxiliary layer, a light-emitting auxiliary layer, and an electron blocking layer in addition to the hole transport layer.

The organic electroluminescent material according to one embodiment may be a hole transport material, a hole injection material, a hole auxiliary material, a light-emitting auxiliary material, and an electron blocking material, preferably a hole transport material, a hole auxiliary material, or a light emission auxiliary material for a green light-emitting organic electroluminescent device, In the case where the hole transport layer is a plurality of layers, it may be a hole transport material (a hole auxiliary material) included in the hole transport layer adjacent to the light-emitting layer.

The organic electroluminescent material of the present disclosure may include at least one host compounds and at least one dopant compounds, in addition to the organic electroluminescent compound of formula 1 above.

Any known phosphorescent host may be used as the host included in the organic electroluminescent material of the present disclosure, and for example, a plurality of host compounds (co-host) may be used as the host materials. Wherein, it may be included in the organic electroluminescent material in a weight ratio of the first host material and the second host material of 1:9 to 9:1, for example, 2:8 to 8:2, 3:7 to 7:3, 4:6 to 6:4, and 5:5. When two or more of the materials are included in one layer, the materials may be mixed deposited to form a layer, or co-deposited separately at the same time to form a layer.

The dopant comprised in the organic electroluminescent material of the present disclosure may be at least one phosphorescent or fluorescent dopant, preferably a phosphorescent dopant. The phosphorescent dopant material applied to the organic electroluminescent device of the present disclosure is not particularly limited, but may be preferably a metallated complex compound(s) of a metal atom(s) selected from iridium (Ir), osmium (Os), copper (Cu), and platinum (Pt), as necessary; more preferably an ortho-metallated complex compound(s) of a metal atom(s) selected from iridium (Ir), osmium (Os), copper (Cu), and platinum (Pt), as necessary; and even more preferably ortho-metallated iridium complex compound(s), as necessary.

The dopant comprised in the organic electroluminescent device of the present disclosure may use the compound represented by the following formula 101 but is not limited thereto.

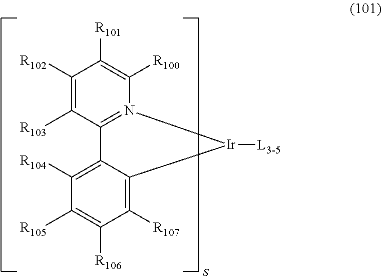

In formula 101,

L is selected from any one of the following structures 1 to 3;

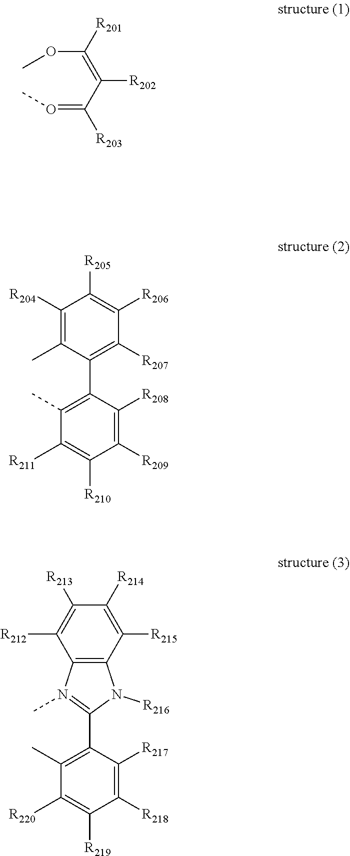

R₁₀₀ to R₁₀₃ each independently represent, hydrogen, deuterium, halogen, (C1-C30)alkyl unsubstituted or substituted with deuterium and/or halogen, a substituted or unsubstituted (C3-C30)cycloalkyl, a substituted or unsubstituted (C6-C30)aryl, cyano, a substituted or unsubstituted (3- to 30-membered)heteroaryl, or a substituted or unsubstituted (C1-C30)alkoxy; or may be linked to an adjacent substituent(s) to form a ring(s), for example, a substituted or unsubstituted quinoline, a substituted or unsubstituted benzofuropyridine, a substituted or unsubstituted benzothienopyridine, a substituted or unsubstituted indenopyridine, a substituted or unsubstituted benzofuroquinoline, a substituted or unsubstituted benzothienoquinoline, or a substituted or unsubstituted indenoquinoline, together with pyridine;

R₁₀₄ to R₁₀₇ each independently represent, hydrogen, deuterium, halogen, (C1-C30)alkyl unsubstituted or substituted with deuterium and/or halogen, a substituted or unsubstituted (C3-C30)cycloalkyl, a substituted or unsubstituted (C6-C30)aryl, a substituted or unsubstituted (3- to 30-membered)heteroaryl, cyano, or a substituted or unsubstituted (C1-C30)alkoxy; or may be linked to an adjacent substituent(s) to form a substituted or unsubstituted ring(s), for example, a substituted or unsubstituted naphthalene, a substituted or unsubstituted fluorene, a substituted or unsubstituted dibenzothiophene, a substituted or unsubstituted dibenzofuran, a substituted or unsubstituted indenopyridine, a substituted or unsubstituted benzofuropyridine, or a substituted or unsubstituted benzothienopyridine, together with benzene;

R₂₀₁ to R₂₂₀ each independently represent hydrogen, deuterium, halogen, (C1-C30)alkyl unsubstituted or substituted with deuterium and/or halogen, a substituted or unsubstituted (C3-C30)cycloalkyl, or a substituted or unsubstituted (C6-C30)aryl; or may be linked to an adjacent substituent(s) to form a substituted or unsubstituted ring(s); and

s represents an integer of 1 to 3.

Specifically, the specific examples of the dopant compound include the following, but are not limited thereto.

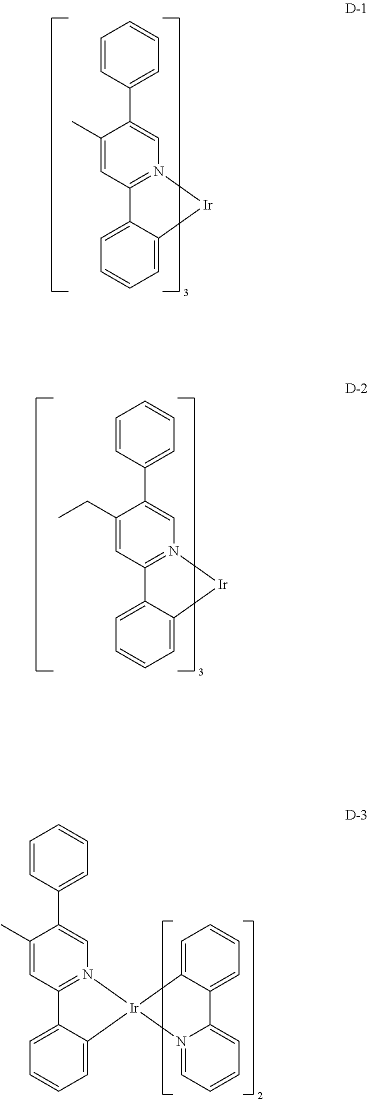

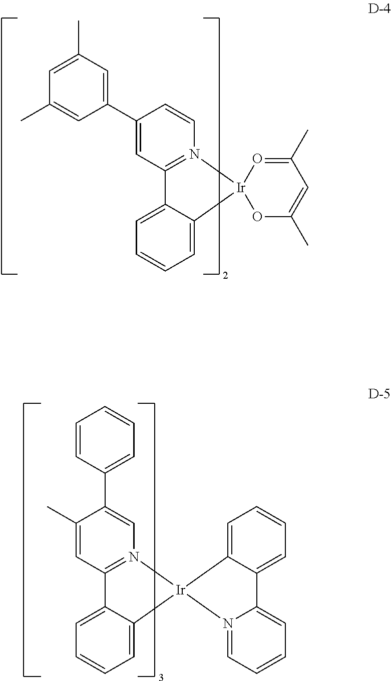

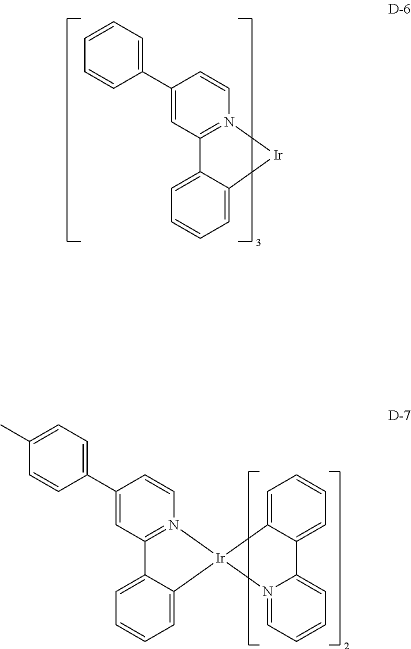

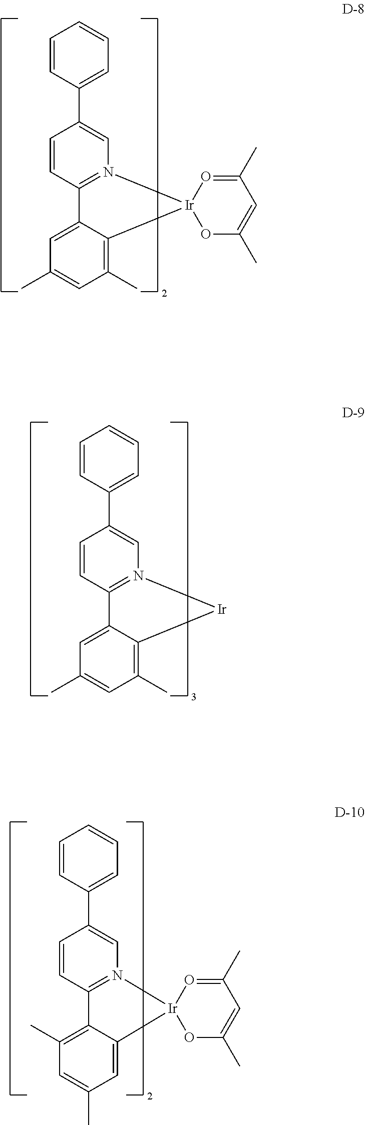

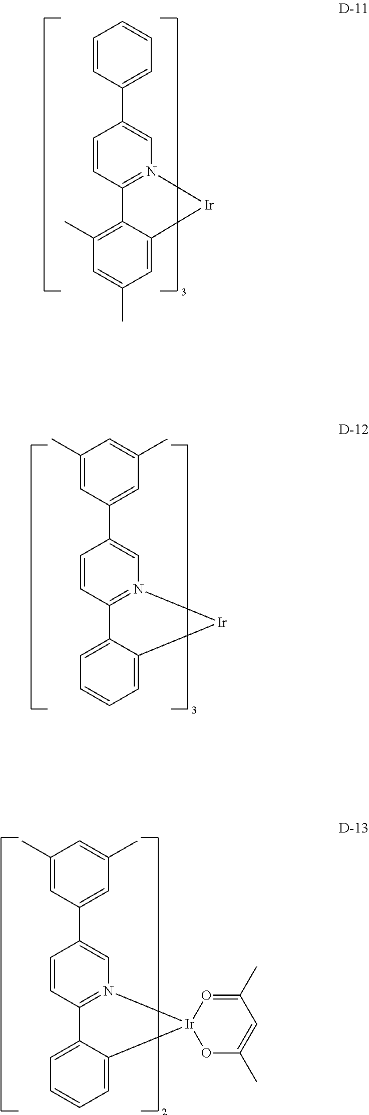

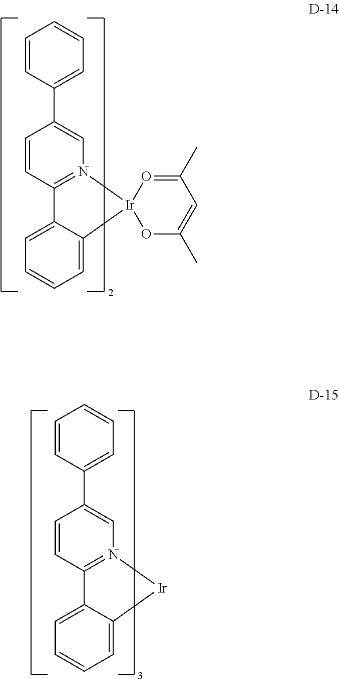

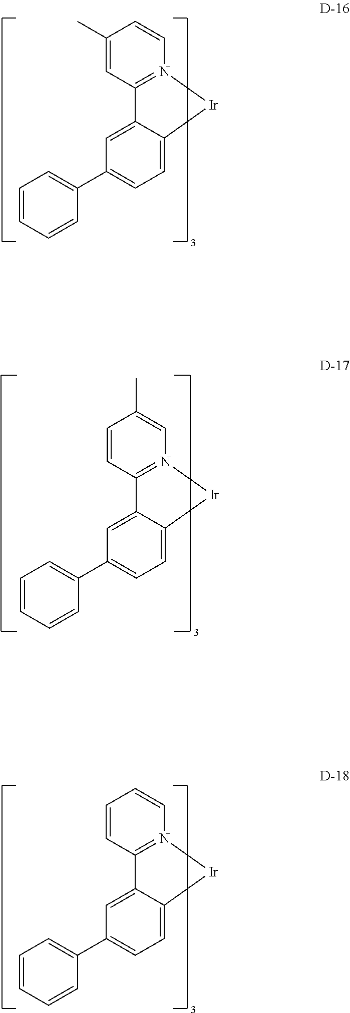

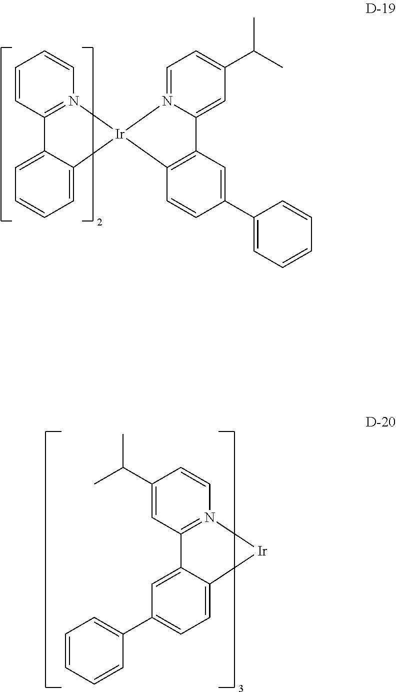

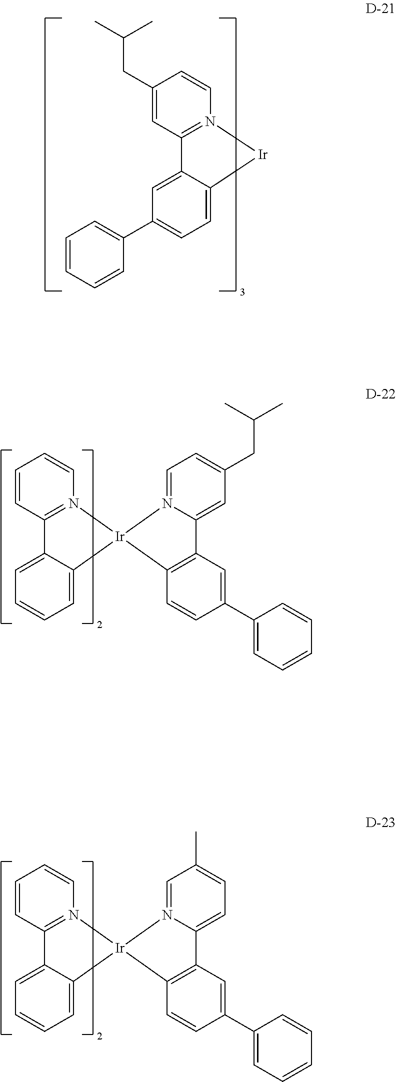

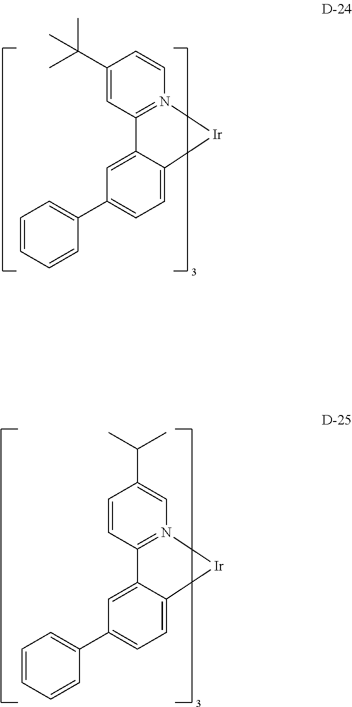

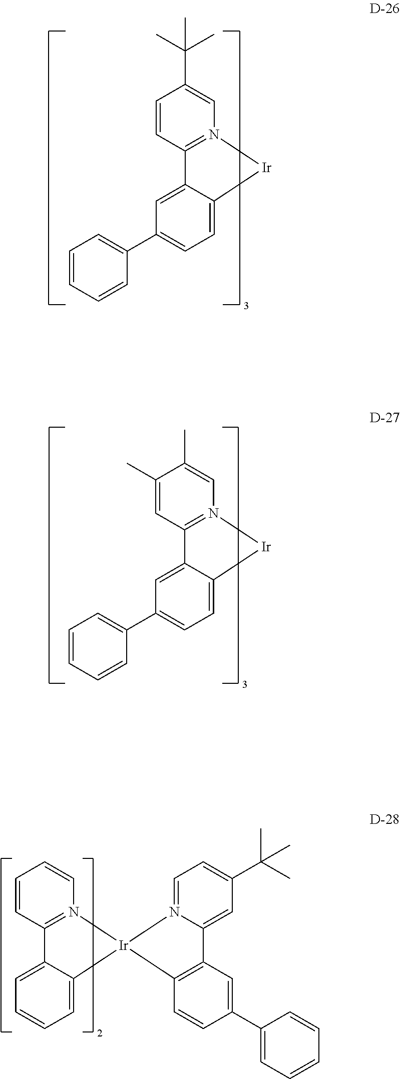

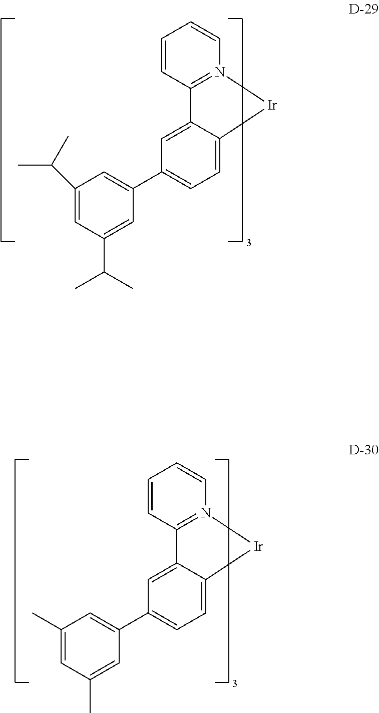

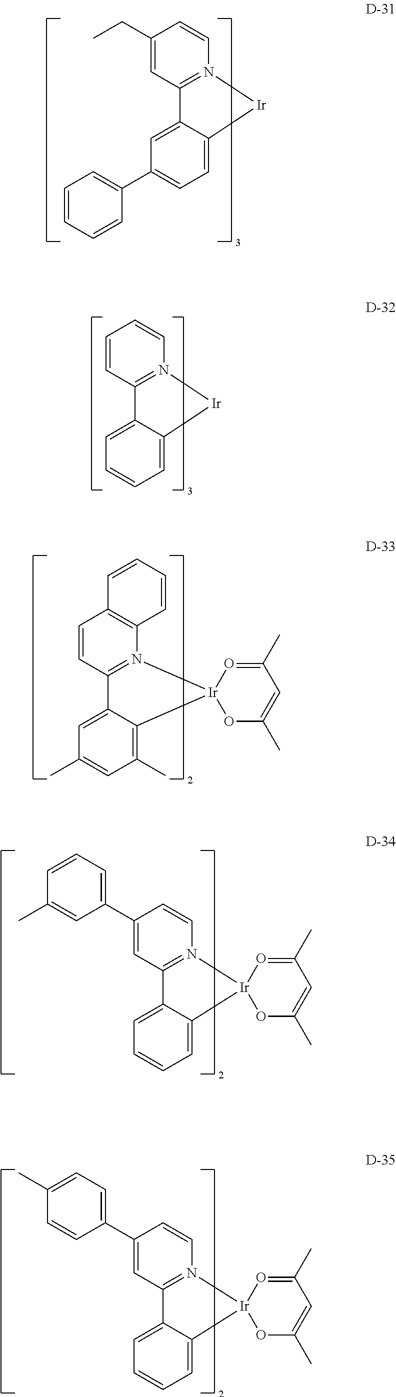

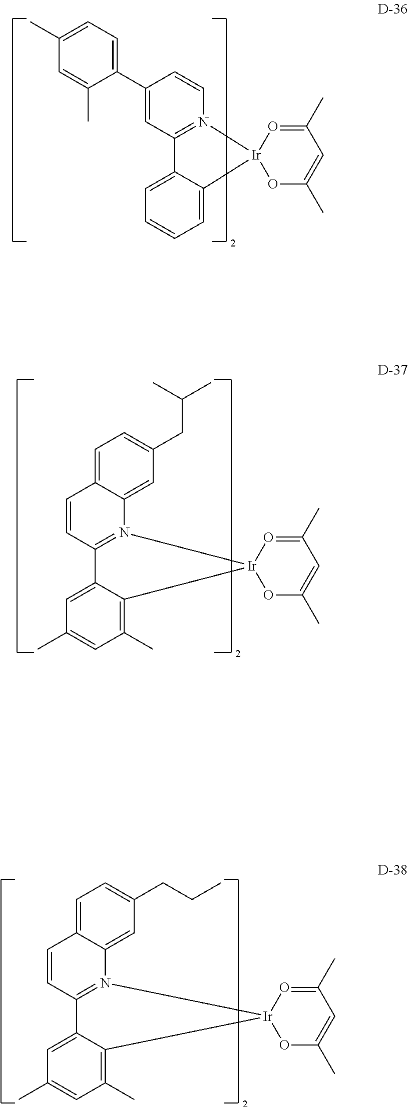

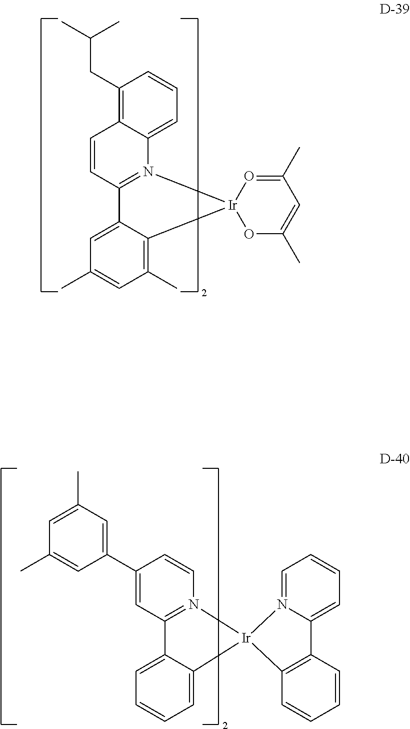

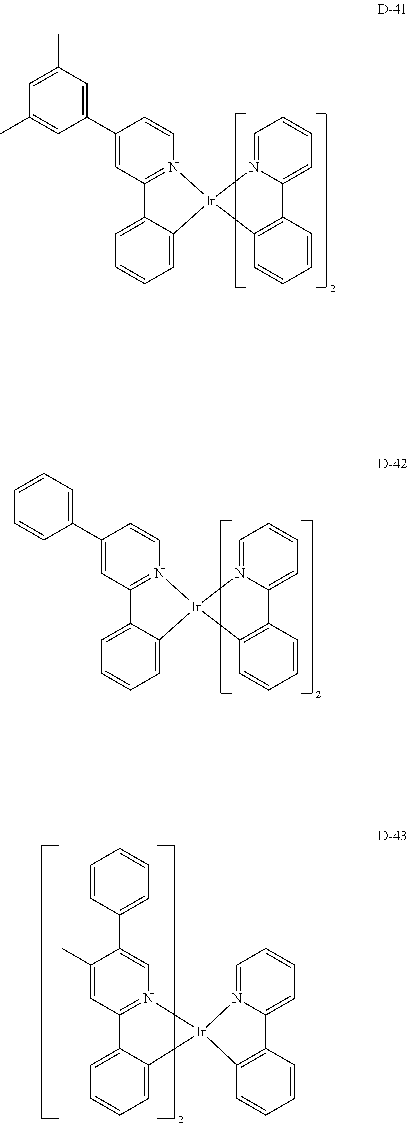

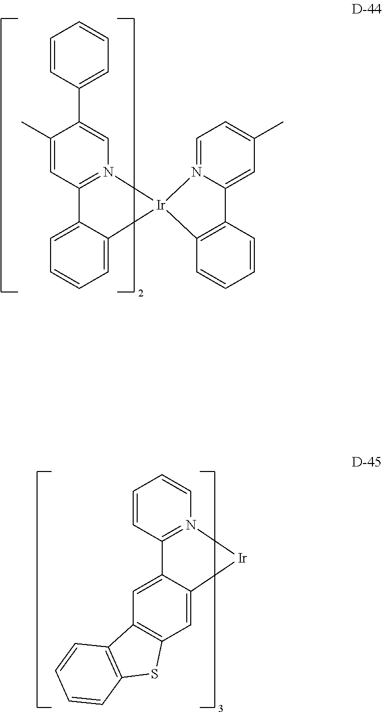

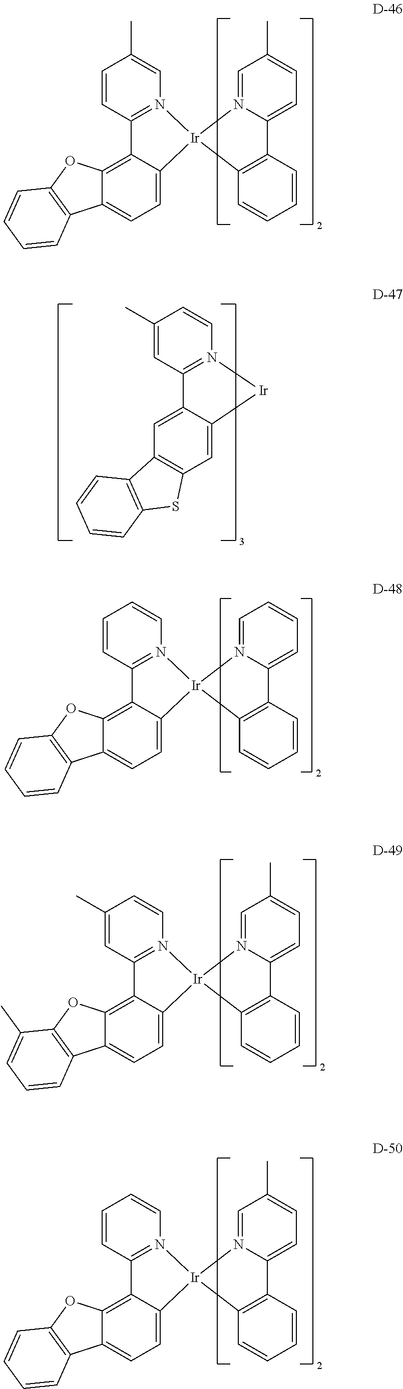

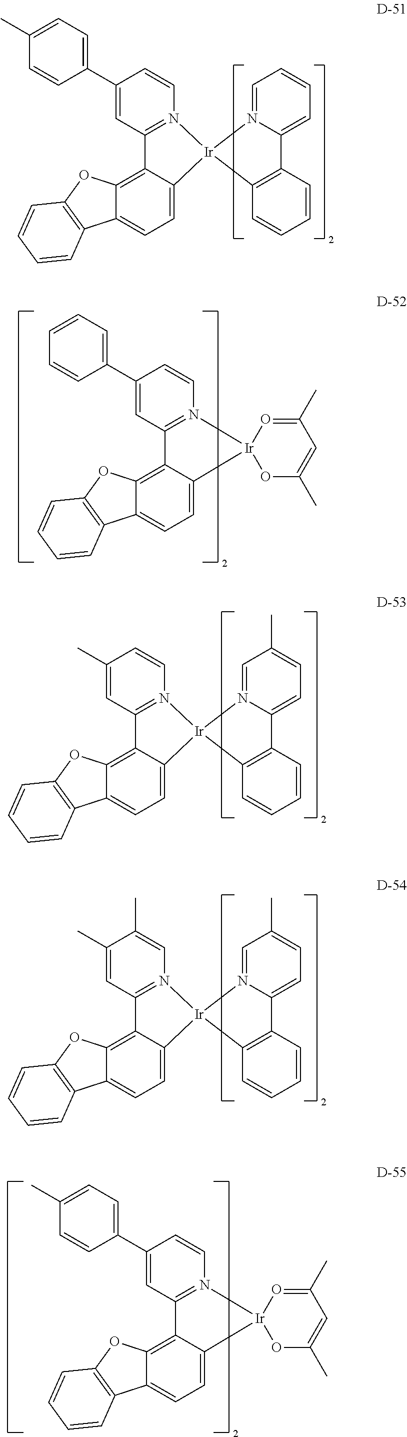

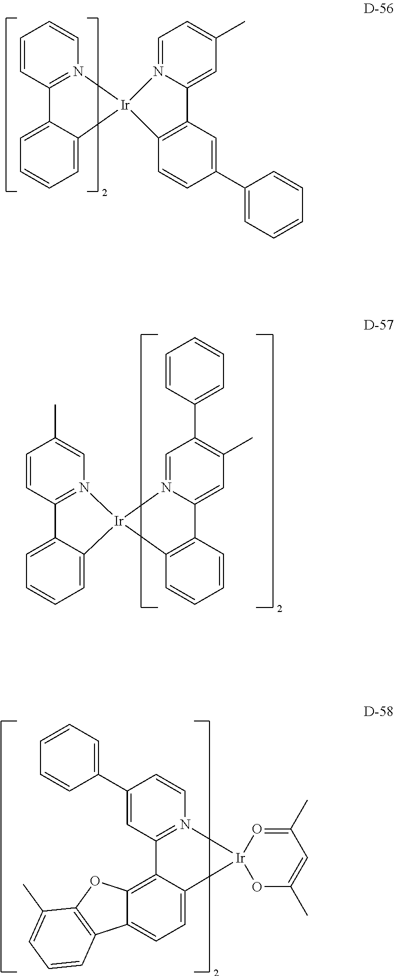

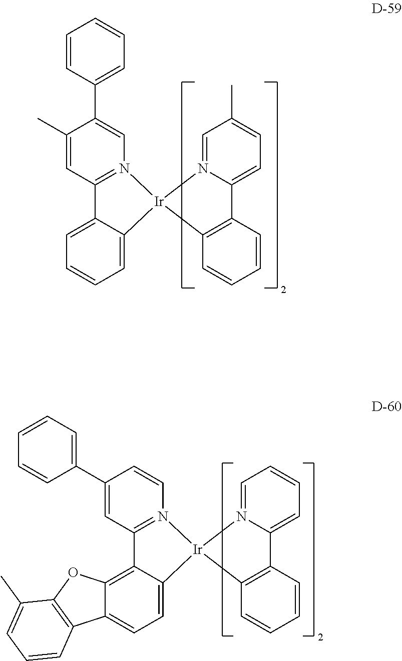

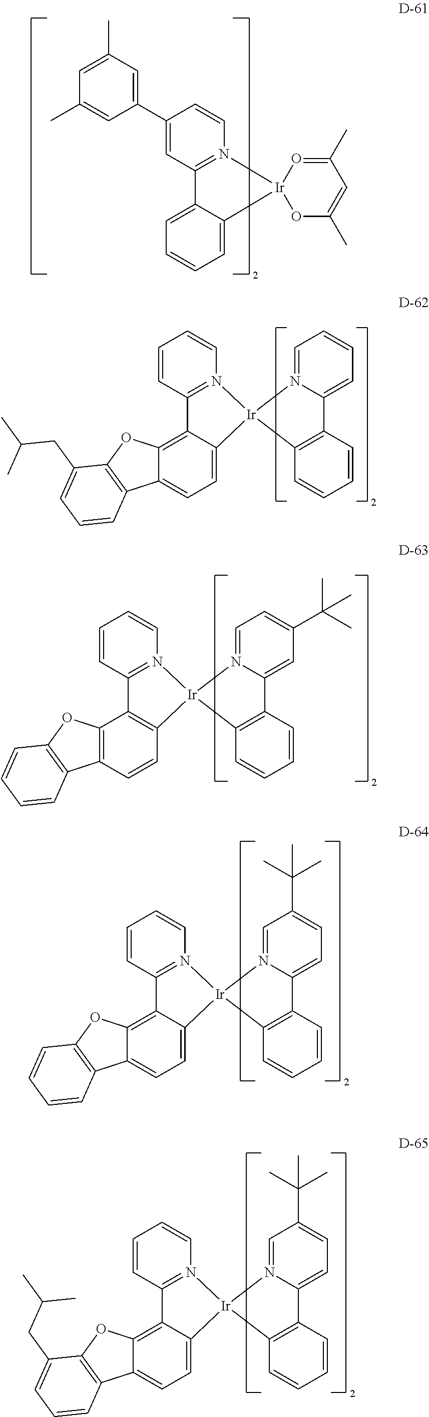

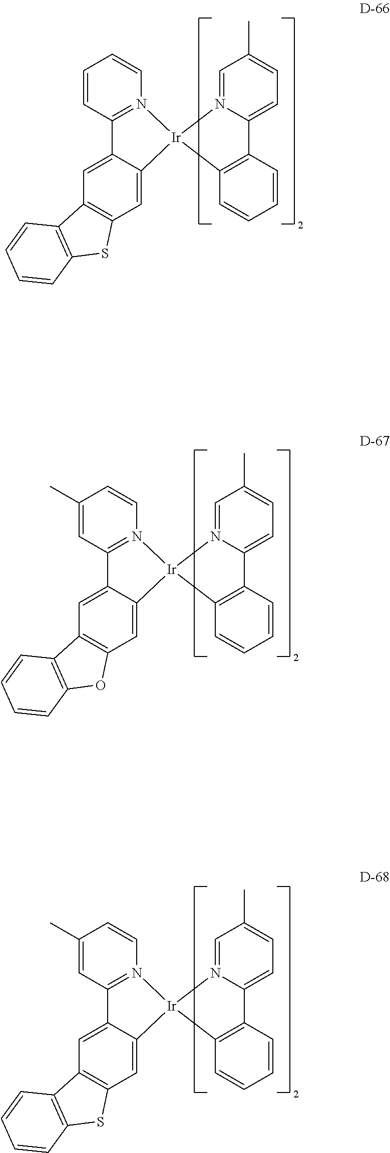

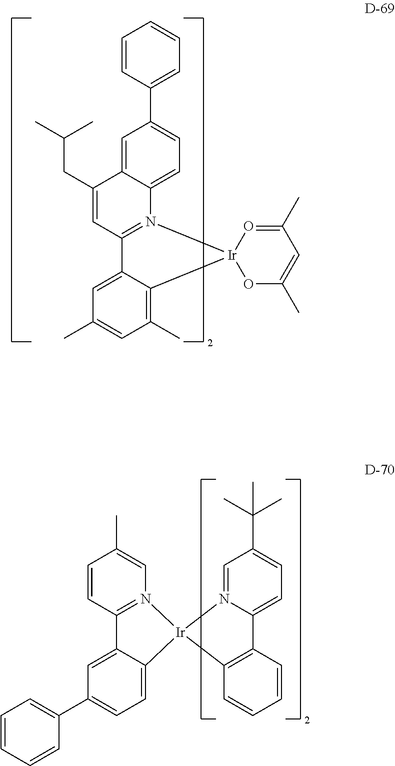

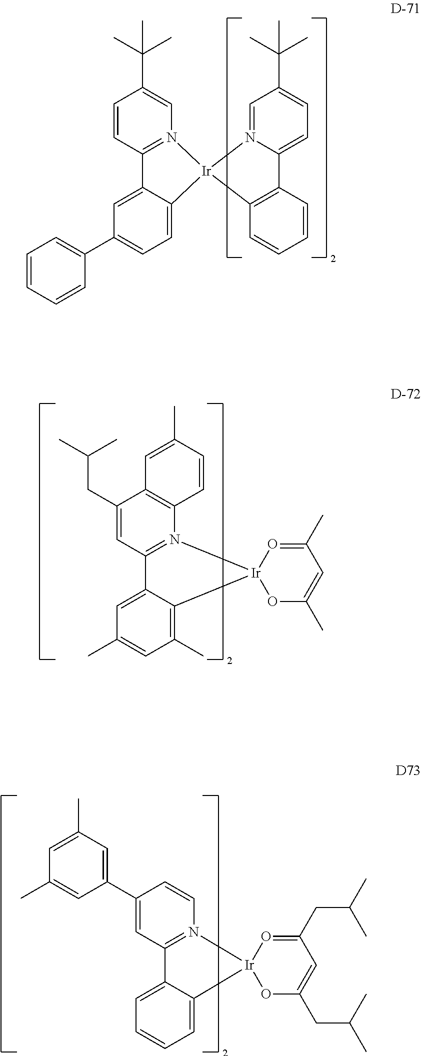

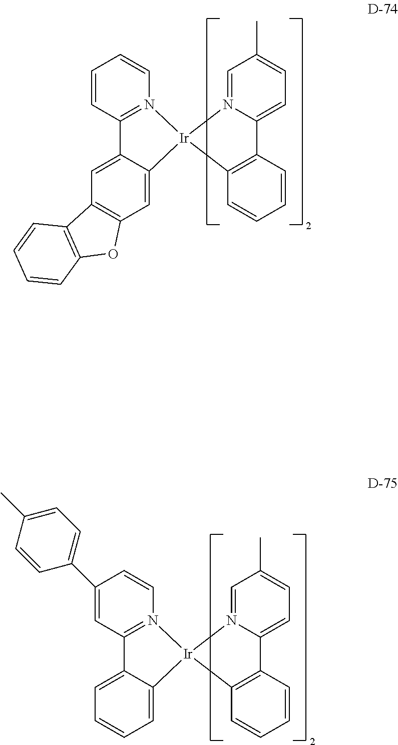

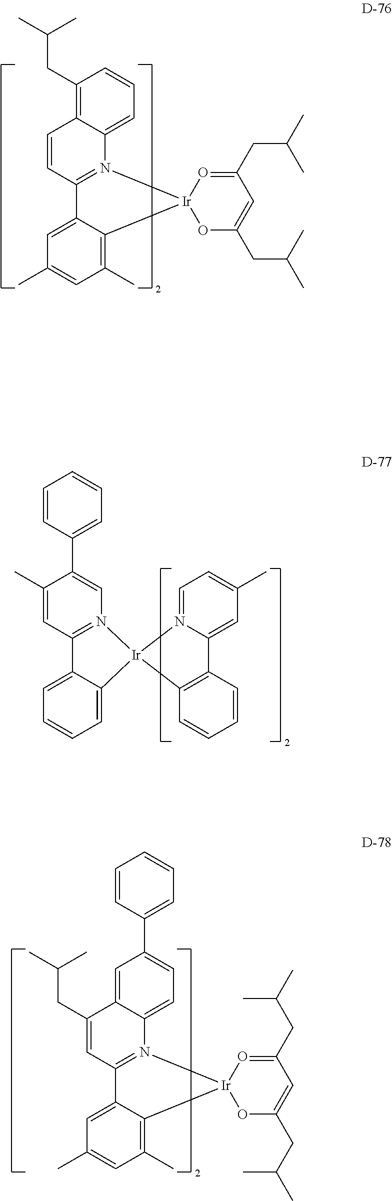

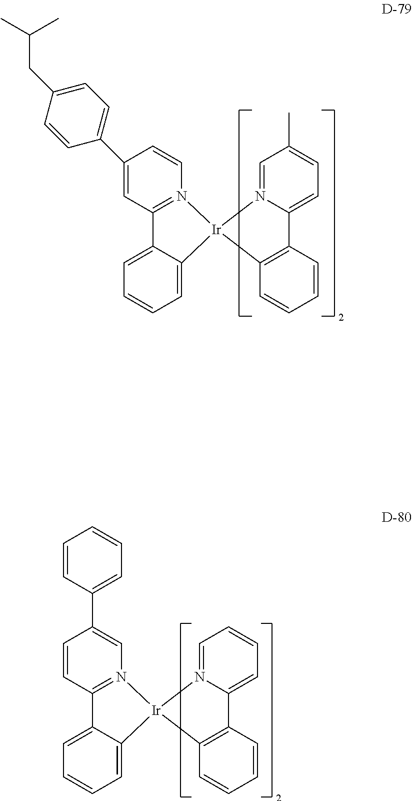

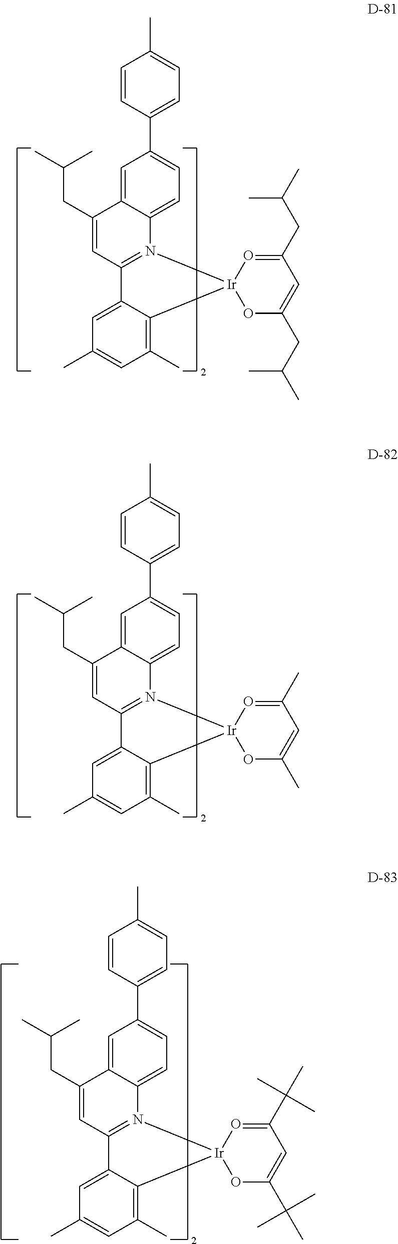

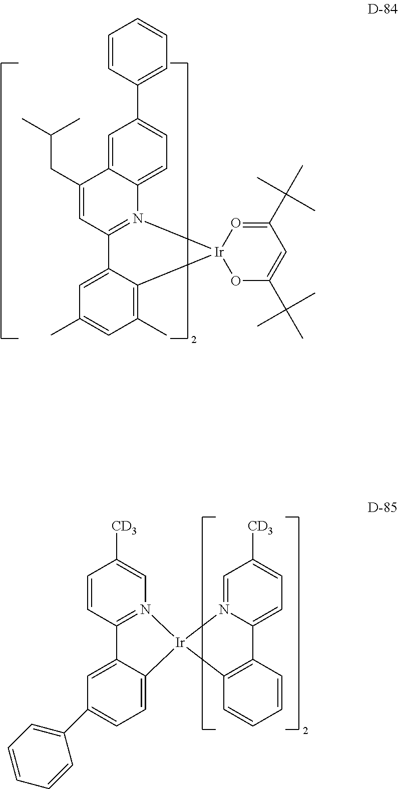

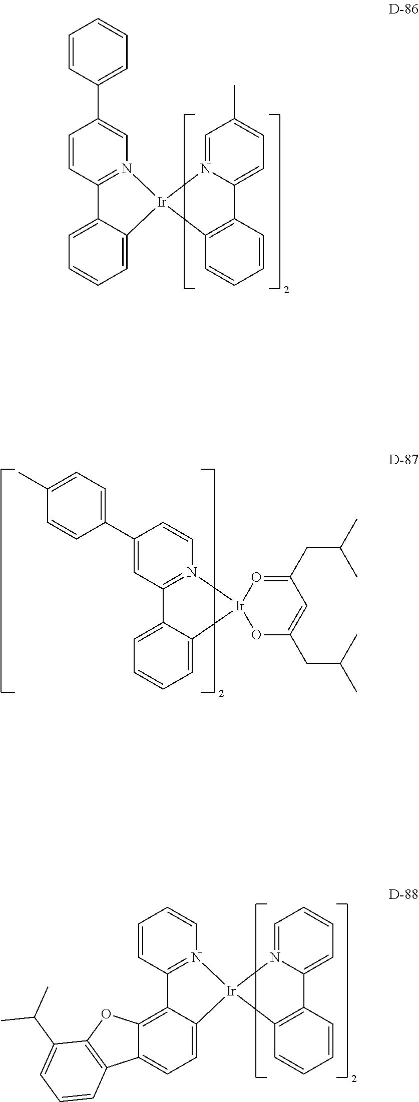

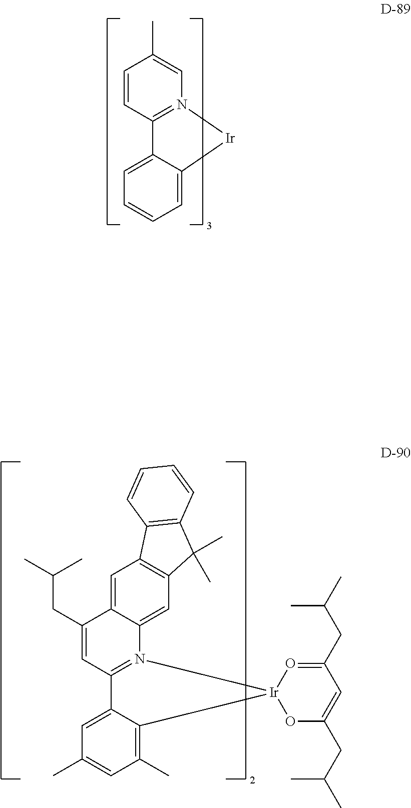

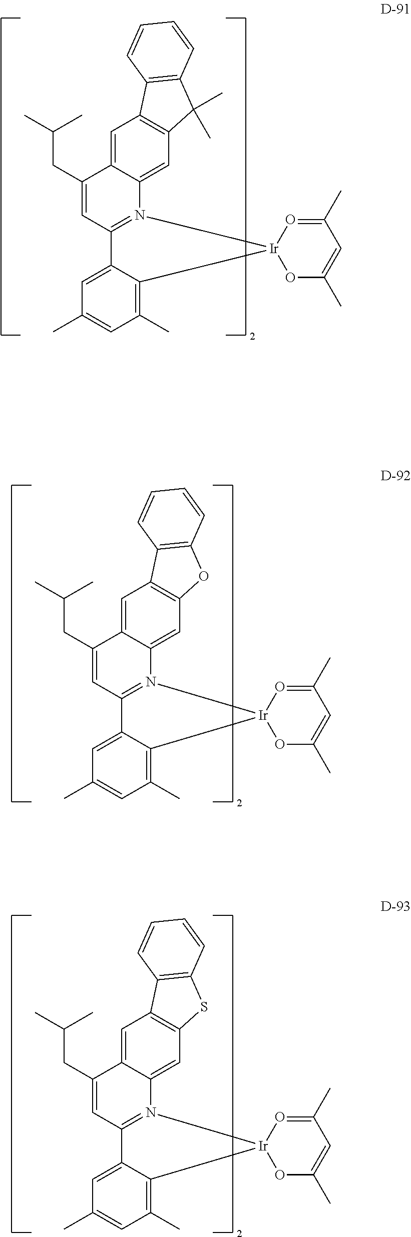

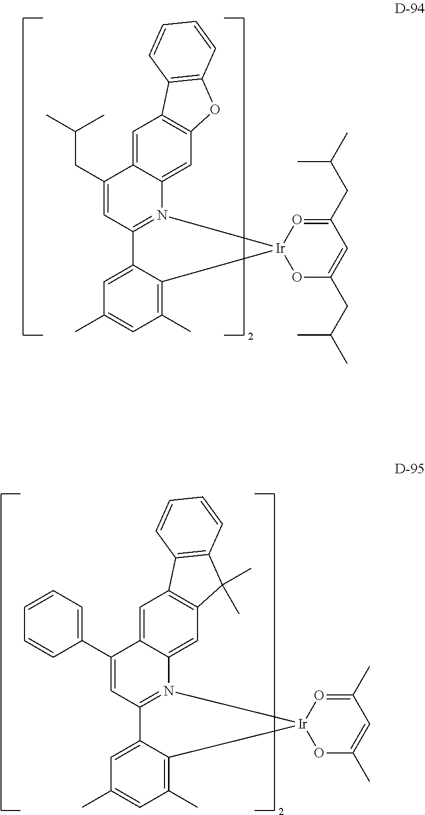

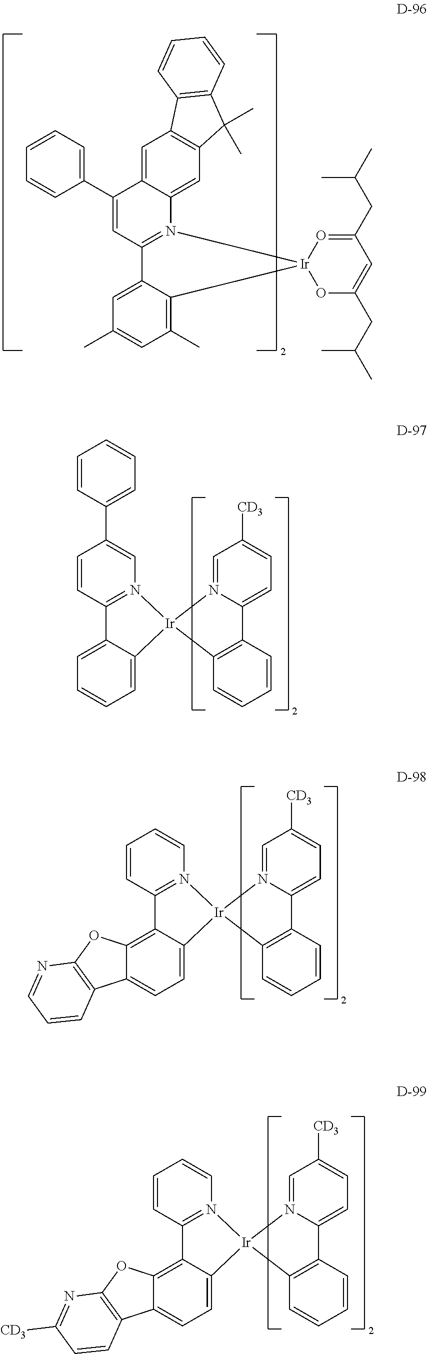

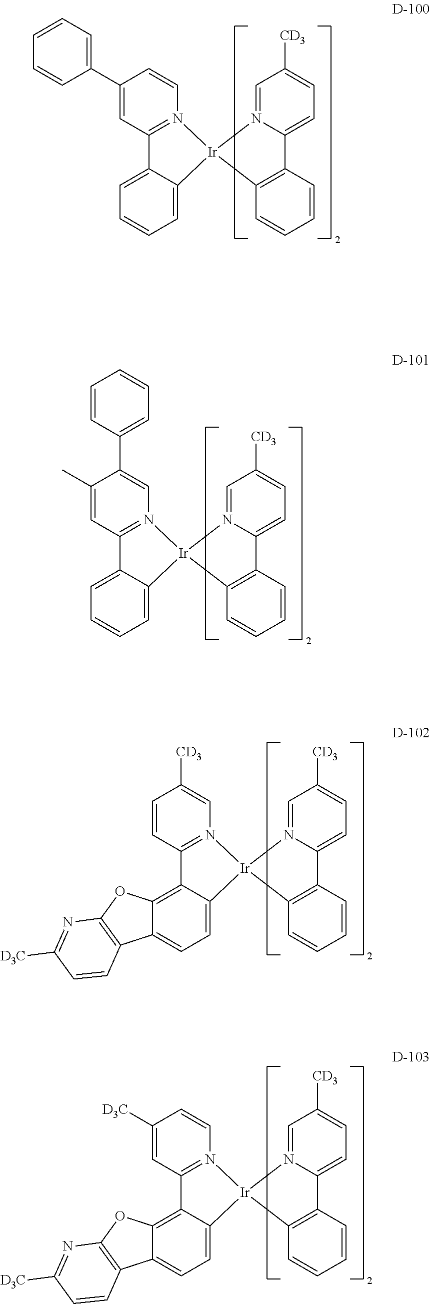

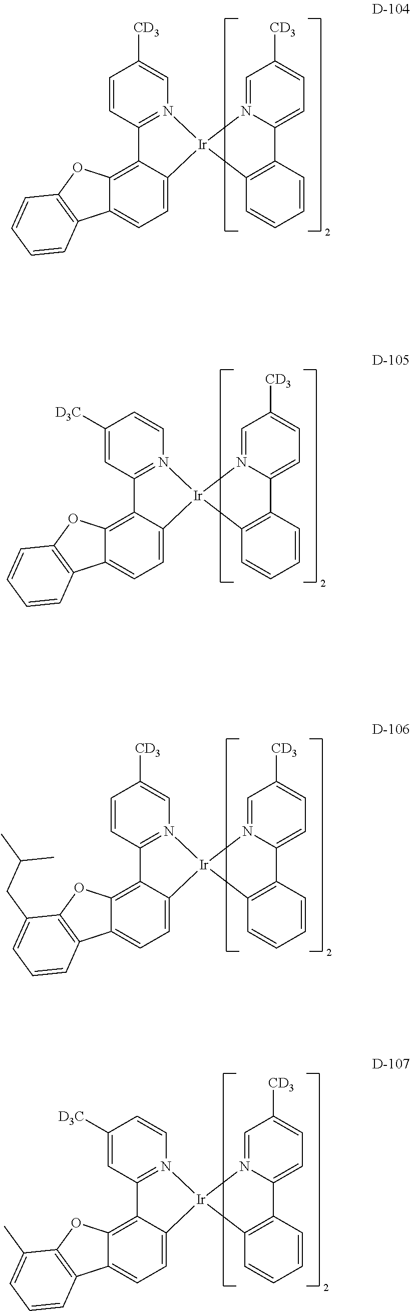

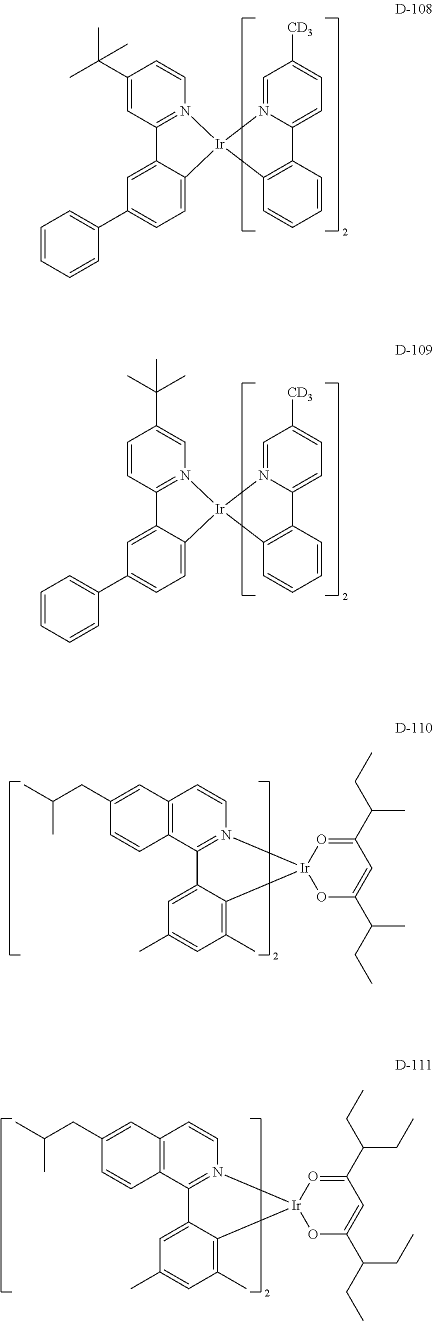

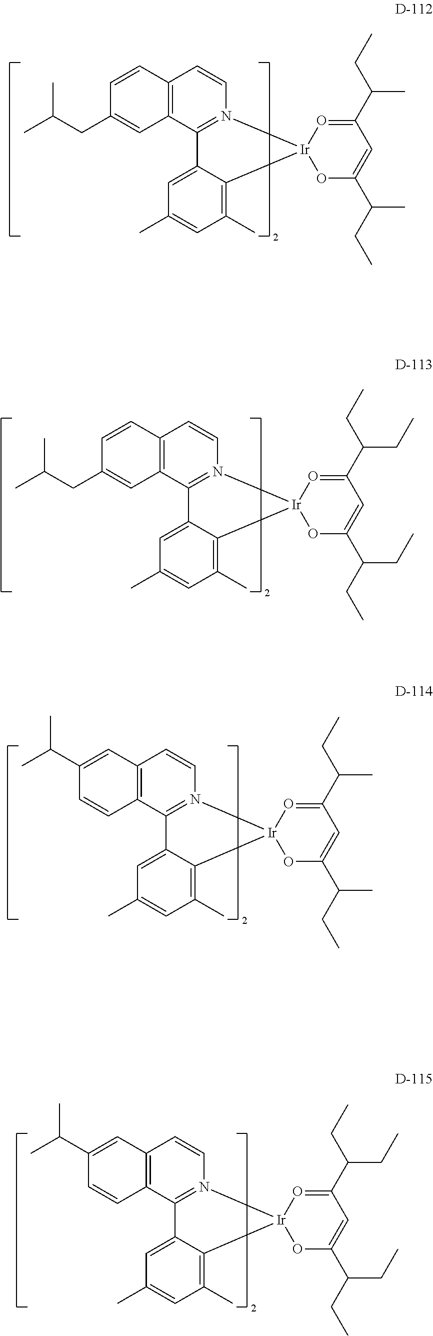

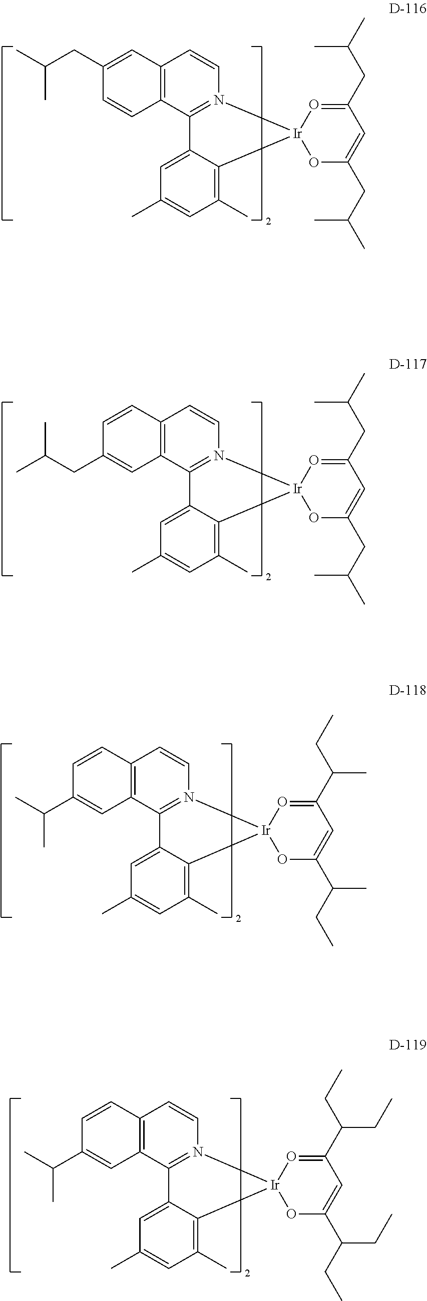

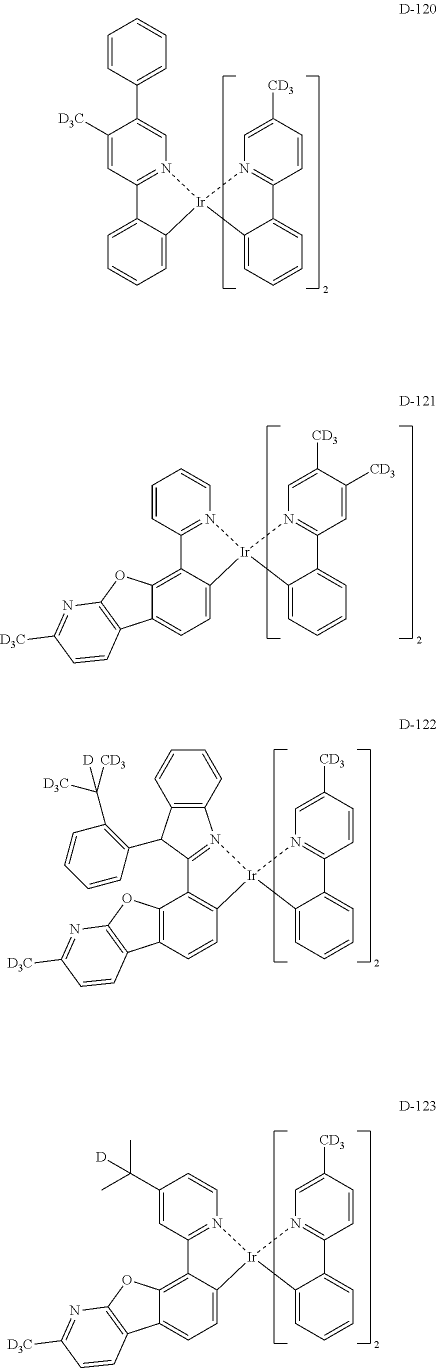

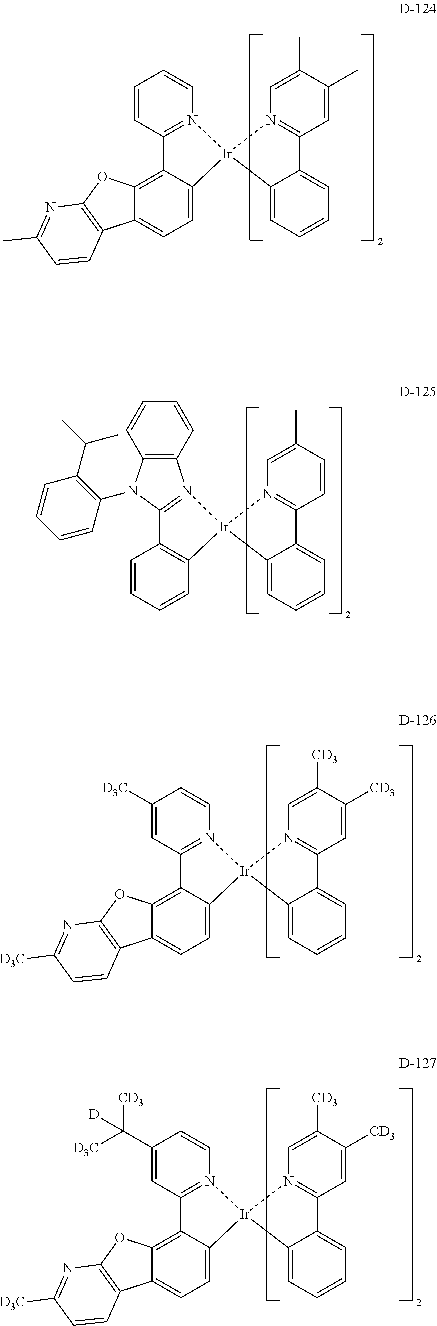

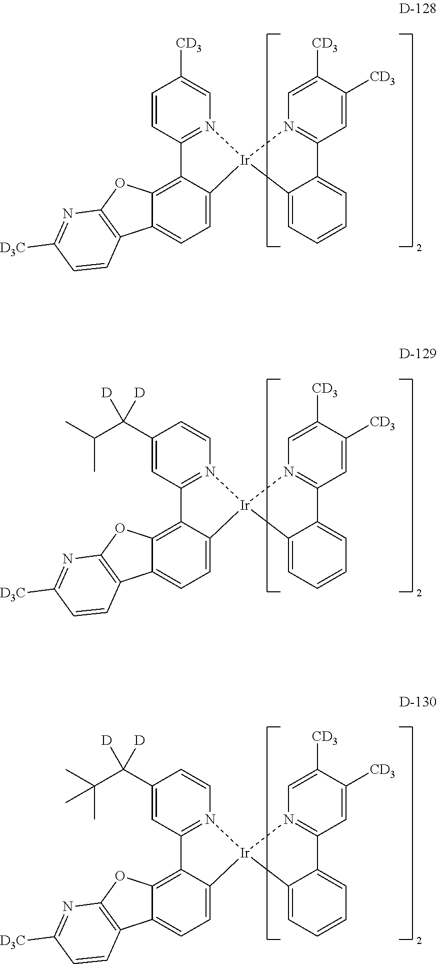

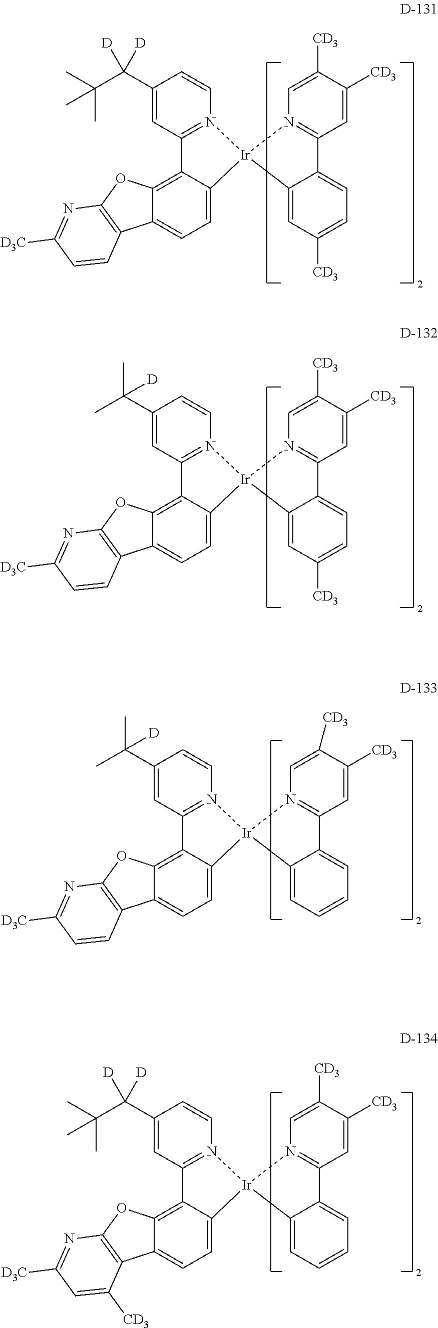

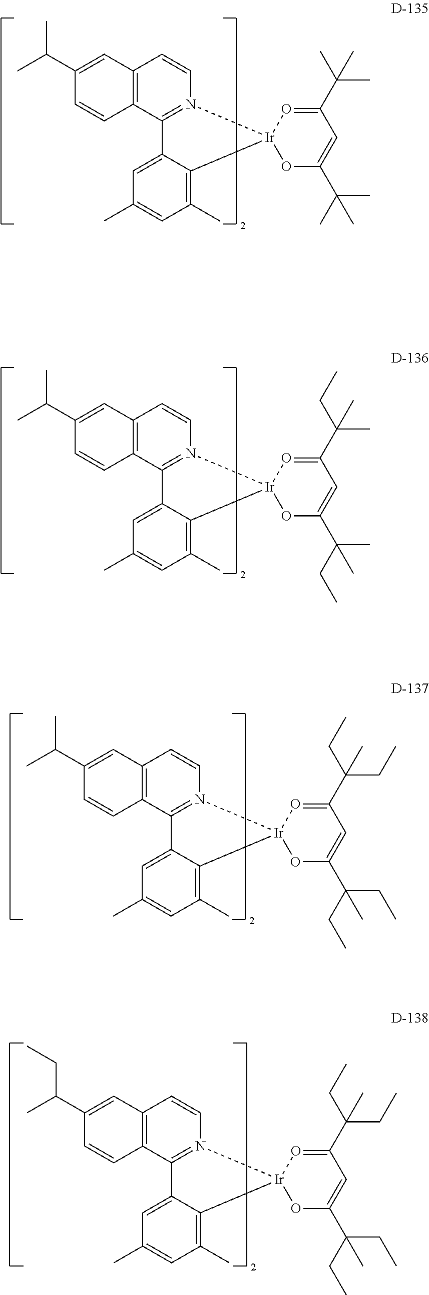

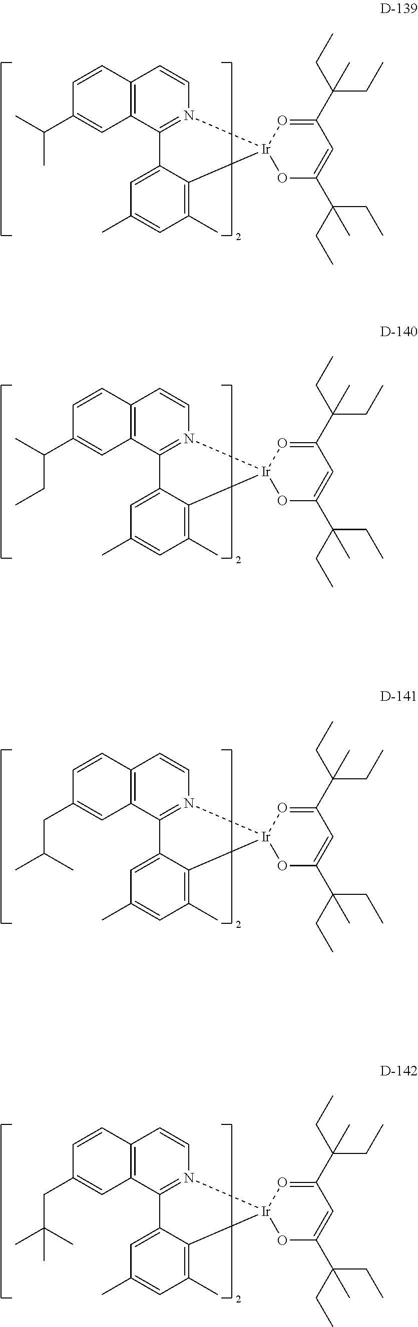

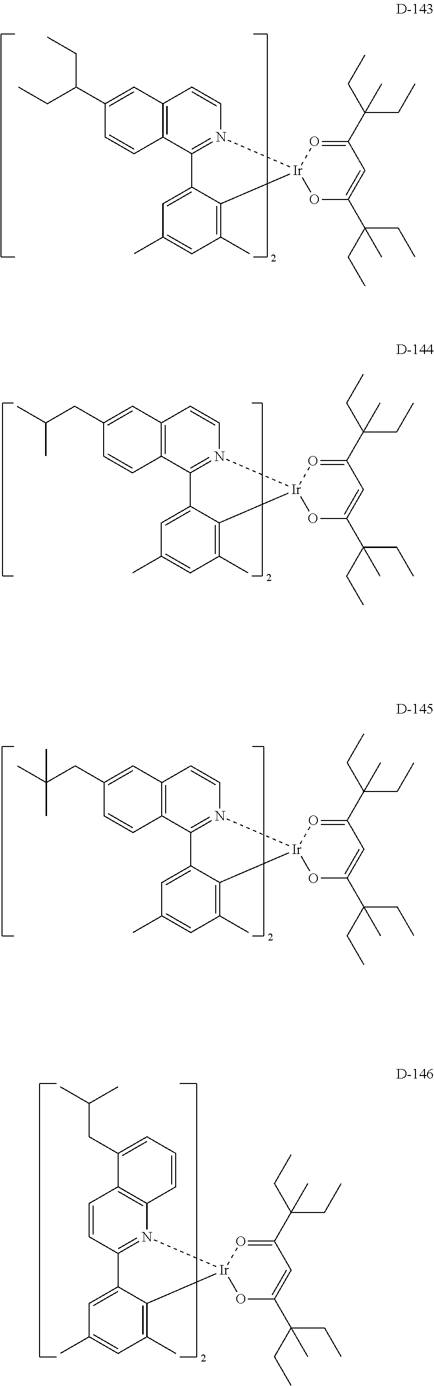

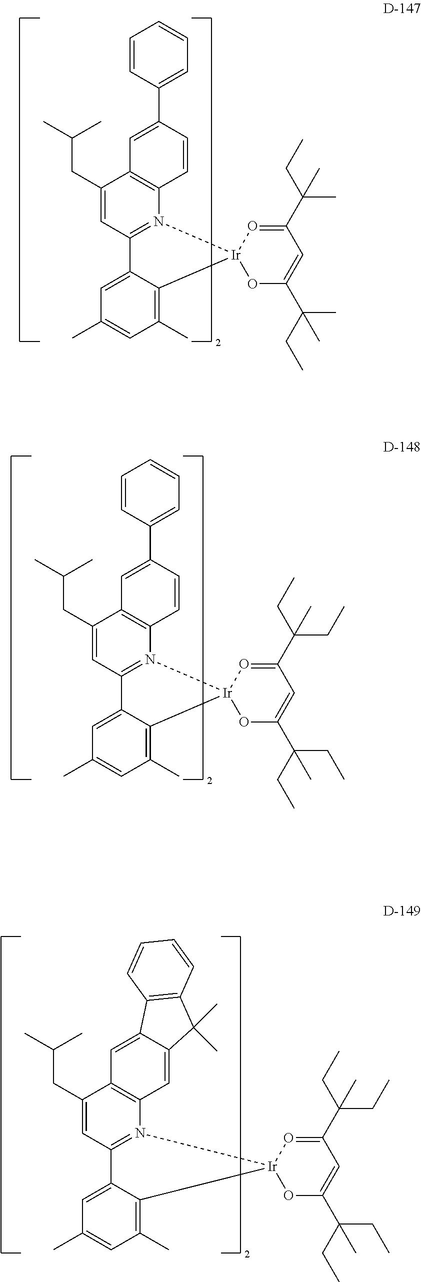

Hereinafter, an organic electroluminescent device to which the aforementioned organic electroluminescent compound or the organic electroluminescent material is applied, will be described.

The organic electroluminescent device according to one embodiment includes a first electrode; a second electrode; and at least one organic layer interposed between the first electrode and the second electrode.

In one embodiment, the organic layer includes a hole transport zone including the organic electroluminescent compound according to the present disclosure. The hole transport zone may include at least one layer selected from the group consisting of a hole injection layer, a hole transport layer, a hole auxiliary layer, a light-emitting auxiliary layer, and an electron blocking layer, for example, a hole transport layer, a hole injection layer, a hole auxiliary layer, and an electron blocking layer. For example, it may include the organic electroluminescent compound of the present disclosure alone, or a mixture of at least two of organic electroluminescent compounds and may further include conventional materials included in the organic electroluminescent material.

In addition, the organic layer may further include at least one layer selected from a light-emitting layer, an electron transport layer, an electron injection layer, an interlayer, a hole blocking layer, and an electron buffer layer, in addition to the hole transport zone, and each layer may be further comprised of a plurality of layers. In addition, the organic layer may further include at least one selected from an arylamine-based compound and a styrylarylamine-based compound. In addition, the organic layer further comprise at least one metal selected from the group consisting of metals of Group 1, metals of Group 2, transition metals of the 4^{th} period, transition metals of the 5^{th} period, lanthanides, and organic metals of the d-transition elements of the Periodic Table, or at least one complex compound comprising such a metal.

The organic electroluminescent material according to one embodiment may be used as light-emitting materials for a white organic light-emitting device. The white organic electroluminescent device has suggested various structures such as a parallel side-by-side arrangement method, a stacking arrangement method, or color conversion material (CCM) method, etc., according to the arrangement of R (Red), G (Green). YG (yellowish green), or B (Blue) light-emitting units. In addition, the organic electroluminescent material according to one embodiment may also be applied to the organic electroluminescent device comprising a QD (quantum dot).

One of the first electrode and the second electrode may be an anode and the other may be a cathode. Wherein, the first electrode and the second electrode may each be formed as a transmissive conductive material, a transflective conductive material, or a reflective conductive material. The organic electroluminescent device may be a top emission type, a bottom emission type, or a both-sides emission type according to the kinds of the material forming the first electrode and the second electrode.

A hole injection layer, a hole transport layer, an electron blocking layer, or a combination thereof can be used between the anode and the light-emitting layer. The hole injection layer may be multi-layers in order to lower the hole injection barrier (or hole injection voltage) from the anode to the hole transport layer or the electron blocking layer, wherein each of the multi-layers may use two compounds simultaneously. In addition, the hole injection layer may be doped as a p-dopant. Also, the electron blocking layer may be placed between the hole transport layer (or hole injection layer) and the light-emitting layer, and can confine the excitons within the light-emitting layer by blocking the overflow of electrons from the light-emitting layer to prevent a light-emitting leakage. The hole transport layer or the electron blocking layer may be multi-layers, and wherein each layer may use a plurality of compounds.

An electron buffer layer, a hole blocking layer, an electron transport layer, an electron injection layer, or a combination thereof can be used between the light-emitting layer and the cathode. The electron buffer layer may be multi-layers in order to control the injection of the electron and improve the interfacial properties between the light-emitting layer and the electron injection layer, wherein each of the multi-layers may use two compounds simultaneously. The hole blocking layer or the electron transport layer may also be multi-layers, wherein each layer may use a plurality of compounds. Also, the electron injection layer may be doped as an n-dopant.

The light-emitting auxiliary layer may be placed between the anode and the light-emitting layer, or between the cathode and the light-emitting layer. When the light-emitting auxiliary layer is placed between the anode and the light-emitting layer, it can be used for promoting the hole injection and/or the hole transport, or for preventing the overflow of electrons. When the light-emitting auxiliary layer is placed between the cathode and the light-emitting layer, it can be used for promoting the electron injection and/or the electron transport, or for preventing the overflow of holes. In addition, the hole auxiliary layer may be placed between the hole transport layer (or hole injection layer) and the light-emitting layer, and may be effective to promote or block the hole transport rate (or the hole injection rate), thereby enabling the charge balance to be controlled. When an organic electroluminescent device includes two or more hole transport layers, the hole transport layer, which is further included, may be used as the hole auxiliary layer or the electron blocking layer. The light-emitting auxiliary layer, the hole auxiliary layer, or the electron blocking layer may have an effect of improving the efficiency and/or the lifespan of the organic electroluminescent device.

In the organic electroluminescent device of the present disclosure, preferably, at least one layer (hereinafter, “a surface layer”) selected from a chalcogenide layer, a halogenated metal layer, and a metal oxide layer may be placed on an inner surface(s) of one or both electrode(s). Specifically, a chalcogenide (including oxides) layer of silicon and aluminum is preferably placed on an anode surface of an electroluminescent medium layer, and a halogenated metal layer or a metal oxide layer is preferably placed on a cathode surface of an electroluminescent medium layer. The operation stability for the organic electroluminescent device may be obtained by the surface layer. Preferably, the chalcogenide includes SiOₓ(1≤X≤2), AlOₓ(1≤X≤1.5), SiON, SiAlON, etc.; the halogenated metal includes LIF, MgF₂, CaF₂, a rare earth metal fluoride, etc.; and the metal oxide includes Cs₂O, Li₂O, MgO, SrO, BaO, CaO, etc.

Further, in the organic electroluminescent device of the present disclosure, preferably, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to an electroluminescent medium. Furthermore, the hole transport compound is oxidized to a cation, and thus it becomes easier to inject and transport holes from the mixed region to the electroluminescent medium. Preferably, the oxidative dopant includes various Lewis acids and acceptor compounds, and the reductive dopant includes alkali metals, alkali metal compounds, alkaline earth metals, rare-earth metals, and mixtures thereof. A reductive dopant layer may be employed as a charge generating layer to prepare an organic electroluminescent device having two or more light-emitting layers and emitting white light.

In order to form each layer of the organic electroluminescent device of the present disclosure, dry film-forming methods such as vacuum evaporation, sputtering, plasma, ion plating methods, etc., or wet film-forming methods such as ink jet printing, nozzle printing, slot coating, spin coating, dip coating, flow coating methods, etc., can be used.

When using a wet film-forming method, a thin film may be formed by dissolving or diffusing materials forming each layer into any suitable solvent such as ethanol, chloroform, tetrahydrofuran, dioxane, etc. The solvent may be any solvent where the materials forming each layer can be dissolved or diffused, and where there are no problems in film-formation capability.

When forming a layer by at least one of the organic electroluminescent compound according to one embodiment, the layer can be formed by co-deposition or mixed deposition, but is not limited thereto, The co-deposition is a mixed deposition method in which two or more materials are put into respective individual crucible sources and a current is applied to both cells simultaneously to evaporate the materials and to perform mixed deposition; and the mixed deposition is a mixed deposition method in which two or more materials are mixed in one crucible source before deposition, and then a current is applied to one cell to evaporate the materials.

According to one embodiment, by using the organic electroluminescent device of the present disclosure, display devices such as smartphones, tablets, notebooks, PCs, TVs, or display devices for vehicles, or lighting devices such as outdoor or indoor lighting can be prepared.

Hereinafter, the preparation method of compounds according to the present disclosure will be explained with reference to the synthesis method of a representative compound or intermediate compound in order to understand the present disclosure in detail.

### [EXAMPLE 1] PREPARATION OF COMPOUND C-1

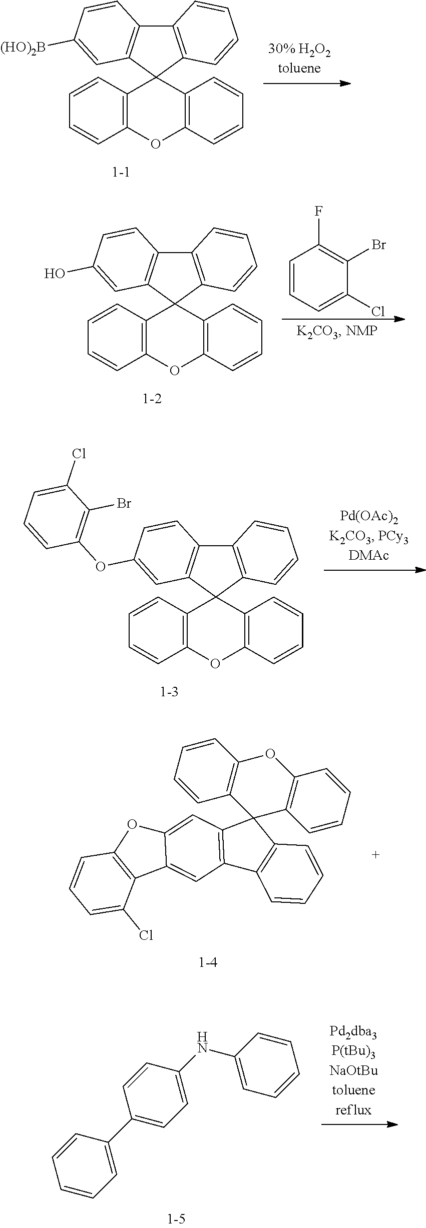

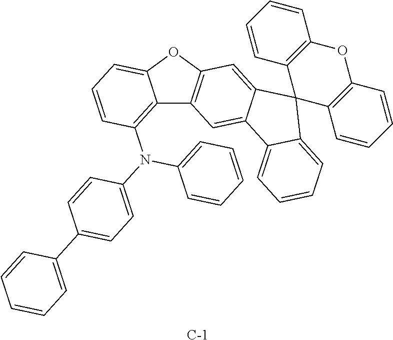

### 1) Synthesis of Compound 1-2

Compound 1-1 (141 g, 375 mmol) and 1.4 L of toluene were added to the flask and dissolved, and then 700 mL of 30% hydrogen peroxide (H₂O₂) was slowly added dropwise thereto at room temperature. Next, 200 mL of 35% sodium hydrogensulfite solution (aq) was slowly added dropwise thereto. After completion of the reaction, the organic layer was extracted with ethyl acetate, and the residual water was removed with magnesium sulfate followed by drying. Next, it was separated by column chromatography to obtain compound 1-2 (90 g, yield: 69%).

### 2) Synthesis of Compound 1-3

Compound 1-2 (90 g, 258 mmol), 2-bromo-1-chloro-3-fluorobenzene (54 g, 258 mmol), potassium carbonate (53 g, 387 mmol), and 900 mL of n-methylpyrrolidone (NMP) were added to the flask, and then refluxed at 160° C. for 4 hours. After completion of the reaction, the organic layer was extracted with ethyl acetate, and the residual water was removed with magnesium sulfate followed by drying. Next, it was separated by column chromatography to obtain compound 1-3 (97 g, yield: 70%).

### 3) Synthesis of Compound 1-4

Compound 1-3 (100 g, 186 mmol), palladium acetate (Pd(OAc)₂) (2 g, 9.3 mmol), tricyclohexyl phosphine (PCy₃) (5.2 g, 18 mmol), potassium carbonate (77 g, 558 mmol), and 1.5 L of dimethylacetaminde (DMAc) were added to the flask, and then reacted at 150° C. for 10 hours. After completion of the reaction, the organic layer was extracted with ethyl acetate, and the residual water was removed with magnesium sulfate followed by drying. Next, it was separated by column chromatography to obtain compound 1-4 (43 g, yield: 50%).

### 4) Synthesis of Compound C-1

Compound 1-4 (8.0 g, 17.5 mmol), compound 1-5 (5.2 g, 21.0 mmol), tris (dibenzylideneacetone)dipalladlum(0) (Pd₂dba₃) (0.80 g, 0.88 mmol), tri-tert-butylphosphine (P(tBu)₃) (0.86 mL, 1.75 mmol in 50% toluene solution), sodium t-butoxide (NaOtBu) (3.4 g, 35 mmol), and 90 mL of toluene were added to the flask, and then refluxed at 110° C. for 18 hours. After completion of the reaction, the reaction mixture was cooled to room temperature, and the solvent of the reaction mixture was removed with a rotary evaporator. Next, it was purified by column chromatography to obtain compound C-1 (8.8 g, yield: 76%) as a white solid.

| | HOMO | LUMO | Et | MP | Tg |
|---|---|---|---|---|---|
| C-1 | −5.043 | −1.140 | 2.802 | 253° C. | 147.5° C. |

### [EXAMPLE 2] PREPARATION OF COMPOUND C-2

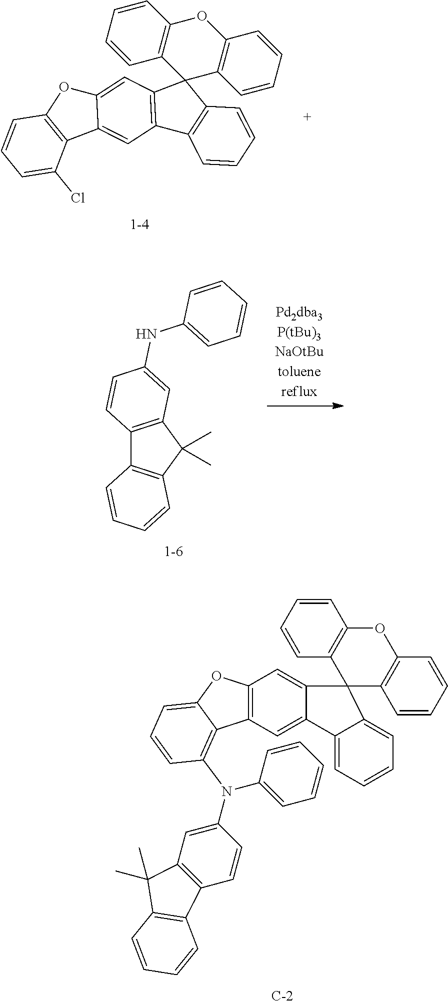

Compound 1 -4 (8.0 g, 17.5 mmol), compound 1 -6 (6.0 g, 21.0 mmol), Pd₂dba₃ (0.80 g, 0.88 mmol), P(tBu)₃ (0.86 mL, 1,75 mmol in 50% toluene solution), NaOtBu (3.4 g, 35 mmol), and 90 mL of toluene were added to the flask, and then refluxed at 110° C. for 18 hours. After completion of the reaction, the reaction mixture was cooled to room temperature, and the solvent of the reaction mixture was removed with a rotary evaporator. Next, it was purified by column chromatography to obtain compound C-2 (6.6 g, yield: 53%) as a white solid.

| | HOMO | LUMO | Et | MP | Tg |
|---|---|---|---|---|---|
| C-2 | −4.937 | −1.136 | 2.704 | 300° C. | 156.7° C. |

### [EXAMPLE 3] PREPARATION OF COMPOUND C-3

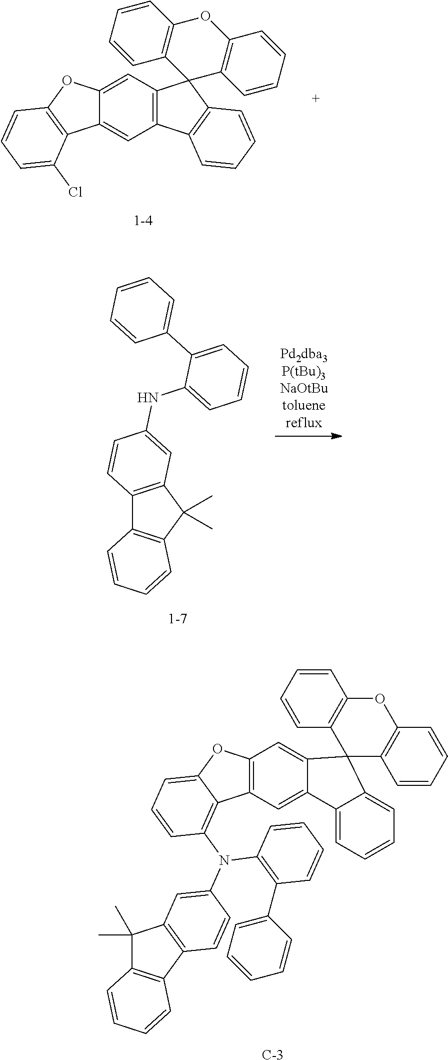

Compound 1-4 (10.0 g, 21.9 mmol), compound 1-7 (9.49 g, 26.3 mmol), Pd₂dba₃ (1.00 g, 1.09 mmol), P(tBu)₃ (1.08 mL, 2.19 mmol, in 50% toluene solution), NaOtBu (4.21 g, 43.8 mmol), and 90 mL of toluene were added to the flask, and then refluxed at 110° C. for 18 hours. After completion of the reaction, the reaction mixture was cooled to room temperature, and the solvent of the reaction mixture was removed with a rotary evaporator. Next, it was purified by column chromatography to obtain compound C-3 (8.7 g, yield: 51%) as a white solid.

| | HOMO | LUMO | Et | MP | Tg |
|---|---|---|---|---|---|
| C-3 | −4.949 | −1.067 | 2.715 | 298° C. | 160.7° C. |

### [EXAMPLE 4] SYNTHESIS OF COMPOUND C-6

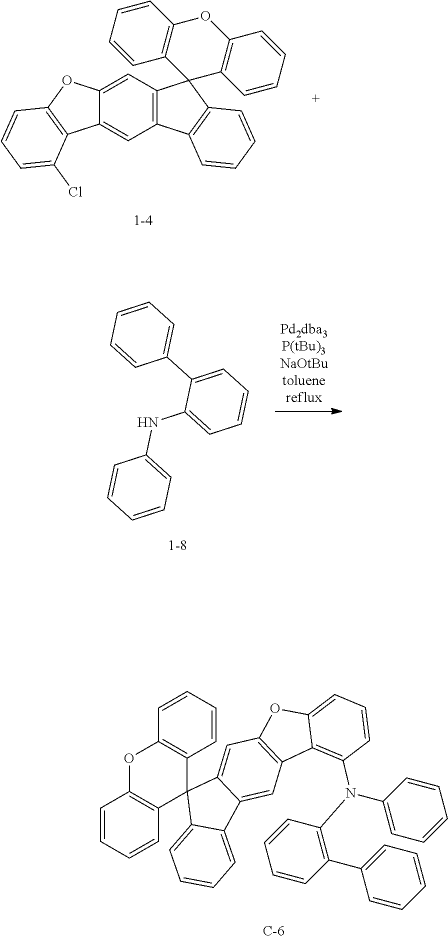

Compound 1-4 (5.0 g, 10.9 mmol), compound 1-8 (2.95 g, 12.0 mmol), Pd₂dba₃ (0.50 g, 0.55 mmol), P(tBu)₃ (0.54 mL, 1.09 mmol, 50% toluene solution), NaOtBu (2.10 g, 21.9 mmol), and 55 mL of toluene were added to the flask, and then refluxed for 18 hours. After completion of the reaction, the reaction mixture was cooled to room temperature, and the solvent of the reaction mixture was removed with a rotary evaporator. Next, it was purified by column chromatography to obtain compound C-6 (3.4 g, yield: 47%) as a white solid.

| | HOMO | LUMO | Et | MP | Tg |
|---|---|---|---|---|---|
| C-6 | −5.092 | −1.025 | 2.851 | 285° C. | 135.2° C. |

### [EXAMPLE 5] SYNTHESIS OF COMPOUND C-281

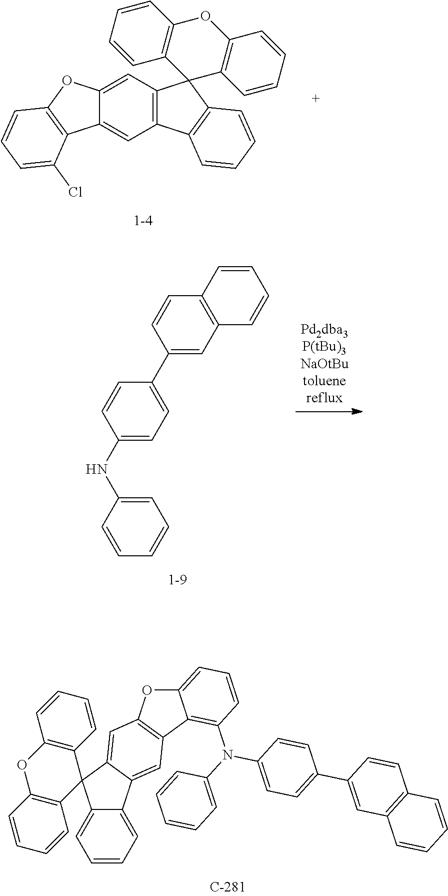 [SMILES: Clc1cccc2oc3cc4c(cc3c12)-c1ccccc1C41c2ccccc2Oc2ccccc21 ; c1ccc(Nc2ccc(-c3ccc4ccccc4c3)cc2)cc1 ; c1ccc(N(c2ccc(-c3ccc4ccccc4c3)cc2)c2cccc3oc4cc5c(cc4c23)-c2ccccc2C52c3ccccc3Oc3ccccc32)cc1]

Compound 1-4 (10.0 g, 21.9 mmol), compound 1-9 (6.46 g, 21.9 mmol), Pd₂dba₃ (1.00 g. 1.09 nmol), P(tBu)₃ (1.08 mL, 2.19 mmol, 50% toluene solution), NaOtBu (4.21 g, 43.8 mmol), and 110 mL of toluene were added to the flask, and then refluxed for 18 hours. After completion of the reaction, the reaction mixture was cooled to room temperature, and the solvent of the reaction mixture was removed with a rotary evaporator. Next, it was purified by column chromatography to obtain compound C-281 (9.0 g, yield: 57%) as a white solid.

| | HOMO | LUMO | Et | MP | Tg |
|---|---|---|---|---|---|
| C-281 | −5.045 | −1.245 | 2.548 | 288° C. | 155.9° C. |

Hereinafter, the light-emitting property of an organic electroluminescent device comprising the organic electroluminescent compound according to the present disclosure will be explained in order to understand the present disclosure in detail.

### [Device Example 1] Preparation of Green Light-Emitting OLED According to the Present Disclosure

An OLED according to the present disclosure was produced. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and thereafter was stored in isopropanol and then used. Thereafter, the ITO substrate was mounted on a substrate holder of a vacuum vapor deposition apparatus. Then, compound HI-1 was introduced into a cell of the vacuum vapor deposition apparatus, and compound HT-1 was introduced into another cell of the vacuum vapor deposition apparatus. The two materials were evaporated at different rates and compound HI-1 was deposited in a doping amount of 3 wt % based on the total amount of the two materials to form a first hole injection layer having a thickness of 10 nm. Next, compound HT-1 was deposited as a first hole transport layer having a thickness of 80 nm on the first hole injection layer. Next, compound C-1 was then introduced into another cell of the vacuum vapor deposition apparatus and was evaporated by applying an electric current to the cell, thereby forming a second hole transport layer having a thickness of 30 nm on the first hole transport layer. After forming the hole injection layer and the hole transport layers, a light-emitting layer was formed thereon as follows: Compounds H-1 and H-2 were introduced into two cells of the vacuum vapor deposition apparatus as hosts, respectively, and compound D-130 was introduced into another cell as a dopant. The two host materials were evaporated at a different rate of 2:1 and the dopant material was evaporated at a different rate, simultaneously, and was deposited in a doping amount of 10 wt % based on the total amount of the hosts and dopant to form a light-emitting layer having a thickness of 40 nm on the second hole transport layer. Next, compounds ETL-1 and EIL-1 as electron transport materials were deposited at a weight ratio of 40:60 to form an electron transport layer having a thickness of 35 nm on the light-emitting layer. After depositing compound EIL-1 as an electron injection layer having a thickness of 2 nm on the electron transport layer, an Al cathode having a thickness of 80 nm was deposited on the electron injection layer by another vacuum vapor deposition apparatus. Thus, an OLED was produced.

### [Device Example 2] Preparation of Green Light-Emitting OLED According to the Present Disclosure

An OLED was produced in the same manner as in Device Example 1, except that compound C-2 was used as a material of the second hole transport layer.

### [Device Example 3] Preparation of Green Light-Emitting OLED According to the Present Disclosure

An OLED was produced in the same manner as in Device Example 1, except that compound C-3 was used as a material of the second hole transport layer.

### [Device Example 4] Preparation of Green Light-Emitting OLED According to the Present Disclosure

An OLED was produced in the same manner as in Device Example 1, except that compound C-6 was used as a material of the second hole transport layer.

### [Comparative Example 1] Preparation of Green Light-Emitting OLED not According to the Present Disclosure

An OLED was produced in the same manner as in Device Example 1. except that compound HT-1 was used as a material of the second hole transport layer.

### [Comparative Example 2] Preparation of Green Light-Emitting OLED not According to the Present Disclosure

An OLED was produced in the same manner as in Device Example 1, except that compound HT-2 was used as a material of the second hole transport layer.

The driving voltage, the luminous efficiency. and the CIE color coordinates at a luminance of 1,000 nits of the OLEDs according to Device Examples and Comparative Examples produced as described above, are measured, and the results thereof are shown in Table 1 below:

TABLE 1

| | Material for | Driving | Luminous | CIE xy (1931) | |
|---|---|---|---|---|---|
| | Second Hole | Voltage | Efficiency | Color Coordinates | |
| | Transport Layer | (V) | (cd/A) | x | y |
| Device | C-1 | 3.5 | 100.8 | 0.321 | 0.643 |
| Example 1 | | | | | |
| Device | C-2 | 3.0 | 102.1 | 0.321 | 0.644 |
| Example 2 | | | | | |
| Device | C-3 | 3.0 | 102.0 | 0.326 | 0.641 |
| Example 3 | | | | | |
| Device | C-6 | 3.8 | 105.2 | 0.318 | 0.647 |
| Example4 | | | | | |
| Comparative | HT-1 | 3.4 | 87.4 | 0.323 | 0.642 |
| Example 1 | | | | | |
| Comparative | HT-2 | 2.9 | 89.2 | 0.326 | 0.641 |
| Example 2 | | | | | |

From Table 1 above, it can be seen that the organic electroluminescent device including the organic electroluminescent compound according to the present disclosure as a hole transport material exhibits low driving voltage and high luminous efficiency characteristics compared to the organic electroluminescent device including a conventional hole transport material.

The compounds used in Device Examples and Comparative Examples are specifically shown in the following Table 2:

TABLE 2

| | |
|---|---|
| Hole Injection Layer/ Hole Transport Layer | 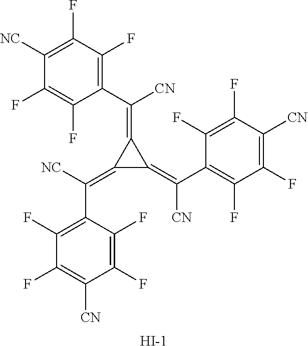 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; [I-]] |
| | 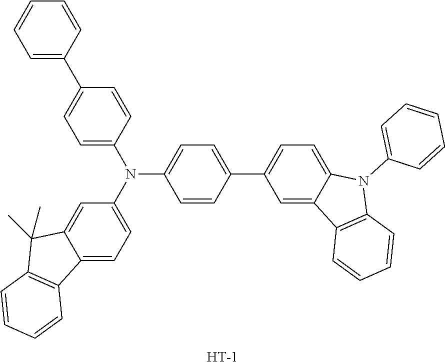 |
| | 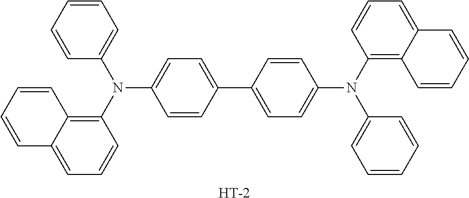 |
| | 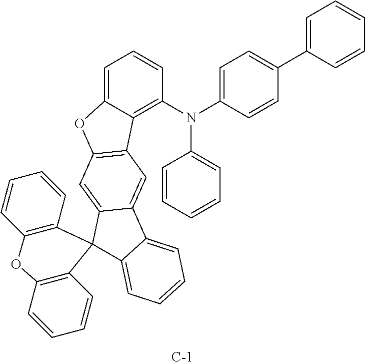 [SMILES: c1ccc(-c2ccc(N(c3ccccc3)c3cccc4oc5cc6c(cc5c34)-c3ccccc3C63c4ccccc4Oc4ccccc43)cc2)cc1 ; "C-1"] |
| | 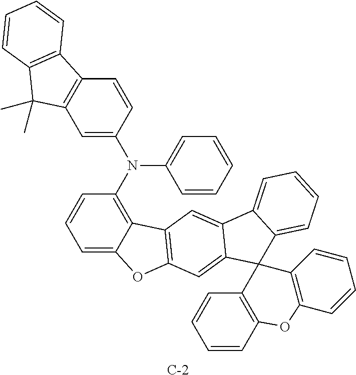 |
| | 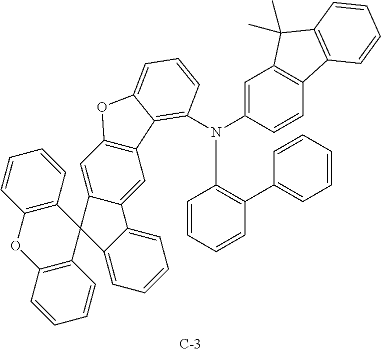 |
| | 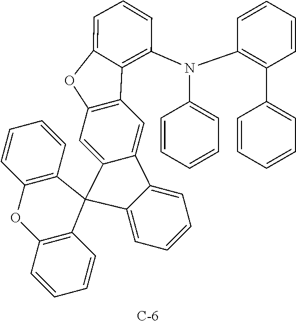 |
| Light-Emitting Layer | 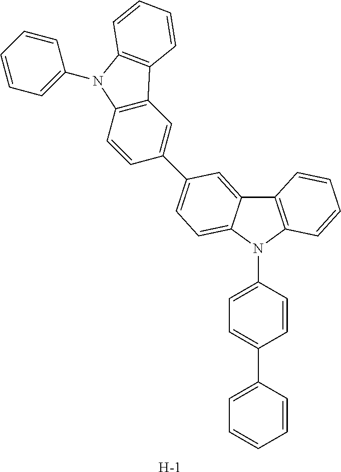 |
| | 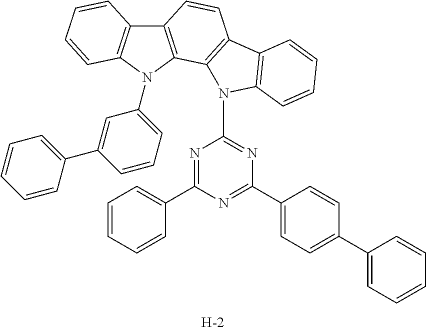 |
| | 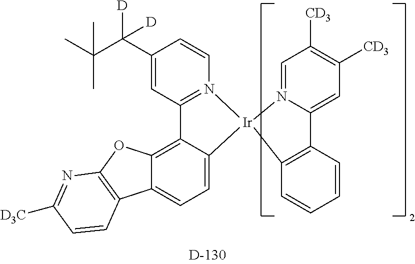 |
| Electron Transport Layer/ Electron Injection Layer | 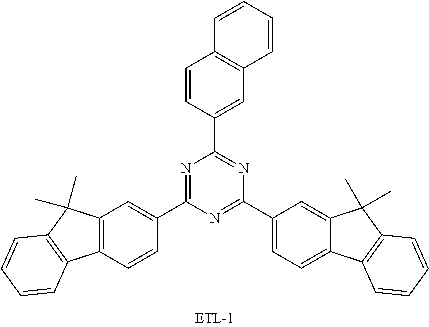 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | 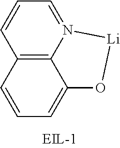 |

1. An organic electroluminescent compound represented by the following formula 1: 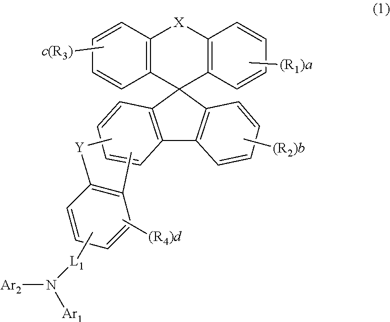
wherein
X and Y each independently represent, O or S;
L₁ represents a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene;
Ar₁ and Ar₂ each independently represent, a substituted or unsubstituted (CS-C30)aryl, a substituted or unsubstituted (3- to 30-membered)heteroaryl, a substituted or unsubstituted (C3-C30)cycloalkyl, a substituted or unsubstituted fused ring of (C3-C30) aliphatic ring and (C6-C30) aromatic ring, or -L₂-N-(Ar₃)(Ar₄); or may be linked to an adjacent substituent(s) to form a ring(s);
R₁ to R₄ each independently represent, hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or -L₃-N-(Ar₅)(Ar₆); or may be linked to an adjacent substituent(s) to form a ring(s);
L₂ and L₃ each independently represent, a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene;
Ar₃ to Ar₆ each independently represent, a substituted or unsubstituted (C1-C30)alkyl, a substituted or unsubstituted (C2-C30)alkenyl, a substituted or unsubstituted (C6-C30)aryl, a substituted or unsubstituted fused ring of (C3-C30) aliphatic ring and (C6-C30) aromatic ring, or a substituted or unsubstituted (3- to 30-membered)heteroaryl;
a to c each independently represent, an integer of 1 to 4, and d represents an integer of 1 to 3; and
when a to d each independently are an integer of 2 or more, each of R₁ to R₄ may be the same or different.

2. The organic electroluminescent compound according to claim 1, wherein the formula 1 is represented by any one of the following formulas 1-1 to 1-4: 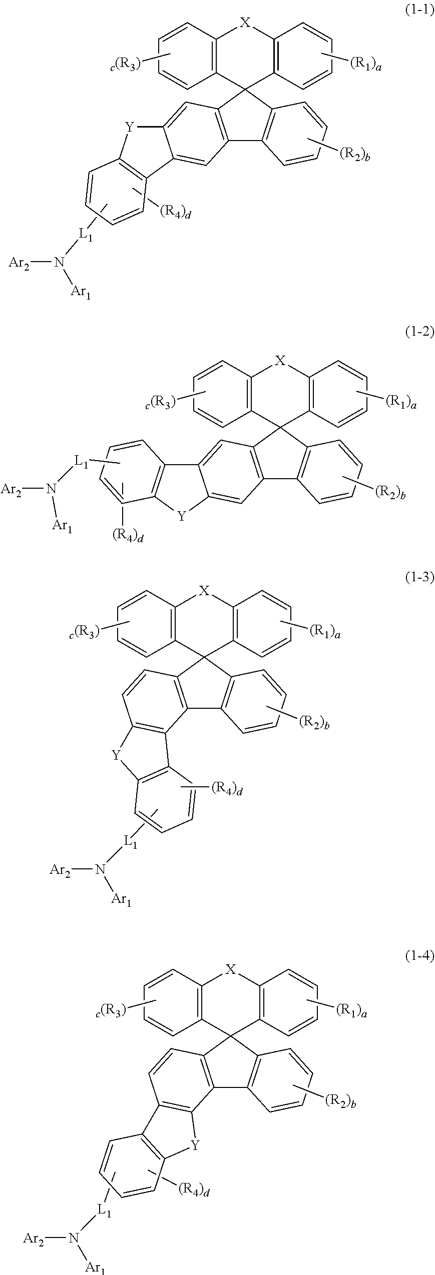
wherein
R₁ to R₄, X, Y, L₁, Ar₁, Ar₂, and a to d each independently are as defined in claim 1,

3. The organic electroluminescent compound according to claim 2, wherein
L₁ represents a single bond or a substituted or unsubstituted (C6-C30)arylene;
Ar₁ and Ar₂ each independently represent, a substituted or unsubstituted (C6-C30)aryl, a substituted or unsubstituted (3- to 30-membered)heteroaryl, or a substituted or unsubstituted fused ring of (C3-C30) aliphatic ring and (C6-C30) aromatic ring.

4. The organic electroluminescent compound according to claim 1, wherein the substituents of the substituted alkyl, the substituted alkylene, the substituted alkenyl, the substituted aryl, the substituted arylene, the substituted heteroaryl, the substituted heteroarylene, the substituted cycloalkyl, the substituted cycloalkylene, the substituted alkoxy, the substituted trialkylsilyl, the substituted dialkylarylsilyl, the substituted alkyldiarylsilyl, the substituted triarylsilyl and the substituted fused ring of aliphatic ring and aromatic ring, each independently are at least one selected from the group consisting of deuterium; halogen; cyano; carboxyl; nitro; hydroxy; phosphine oxide; (C1-C30)alkyl; halo(C1-C30)alkyl; (C2-C30)alkenyl; (C2-C30)alkynyl: (C1-C30)alkoxy; (C1-C30)alkylthio: (C3-C30)cycloalkyl; (C3-C30)cycloalkenyl; (3- to 7-membered) heterocycloalkyl; (C6-C30)aryloxy; (C6-C30)arylthio: (5- to 50-membered)heteroaryl unsubstituted or substituted with at least one of (C1-C30)alkyl, (C6-C30)aryl and di(C6-C30)arylamino; (C6-C30)aryl unsubstituted or substituted with at least one of (C1-C30)alkyl, (3- to 50-membered)heteroaryl, and mono- or di- (C6-C30)arylamino: tri(C1-C30)alkylsilyl; tri(C6-C30)arylsilyl; di(C1-C30)alkyl(C6-C30)arylsilyl; (C1-C30)alkyldi(C6-C30)arylsilyl; amino; mono- or di- (C1-C30)alkylamino; mono- or di- (C2-C30)alkenylamino; mono- or di- (C6-C30)arylamino unsubstituted or substituted with at least one of (C1-C30)alkyl, (5- to 30-membered)heteroaryl and di(C6-C30)arylamino; mono- or di- (3- to 30-membered)heteroarylamino; (C1-C30)alkyl(C2-C30)alkenylamino; (C1-C30)alkyl(C6-C30)arylamino: (C1-C30)alkyl(3- to 30-membered)heteroarylamino; (C2-C30)alkenyl(C6-C30)arylamino; (C2-C30)alkenyl(3- to 30-membered)heteroarylamino; (C6-C30)aryl(3- to 30-membered)heteroarylamino; (C1-C30)alkylcarbonyl; (C1 -C30)alkoxycarbonyl; (C6-C30)arylcarbonyl; (C6-C30)arylphosphinyl; di(C6-C30)arylboronyl; di(C1-C30)alkylboronyl; (C1 -C30)alkyl(C6-C30)alylboronyl; (C6-C30)ar(C1 -C30)alkyl; and (C1 -C30)alkyl(C6-C30)aryl.

5. The organic electroluminescent compound according to claim 1, wherein the compound represented by the formula 1 is selected from the following compounds:

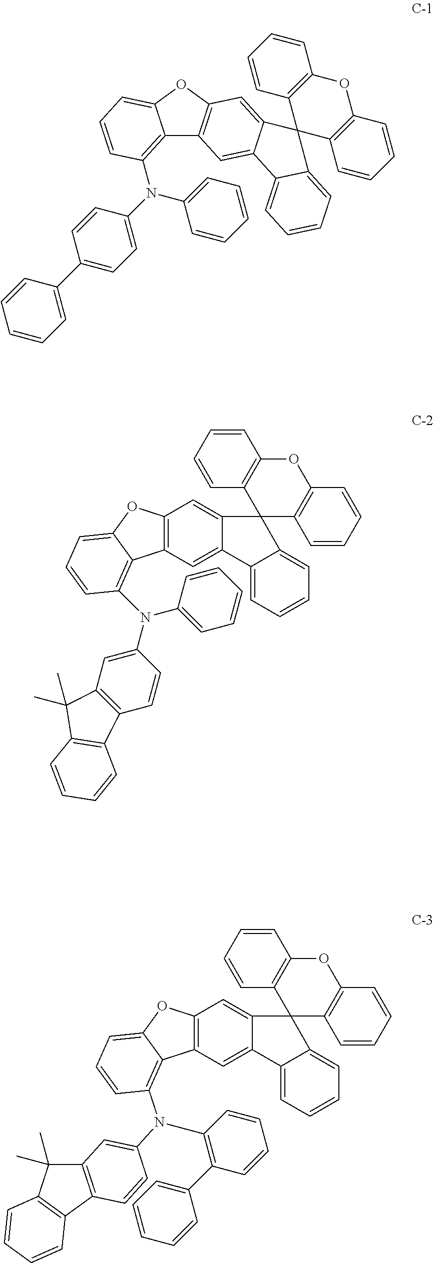 [SMILES: c1ccc(-c2ccc(N(c3ccccc3)c3cccc4oc5cc6c(cc5c34)-c3ccccc3C63c4ccccc4Oc4ccccc43)cc2)cc1 ; CC1(C)c2ccccc2-c2ccc(N(c3ccccc3)c3cccc4oc5cc6c(cc5c34)-c3ccccc3C63c4ccccc4Oc4ccccc43)cc21 ; CC1(C)c2ccccc2-c2ccc(N(c3ccccc3-c3ccccc3)c3cccc4oc5cc6c(cc5c34)-c3ccccc3C63c4ccccc4Oc4ccccc43)cc21]

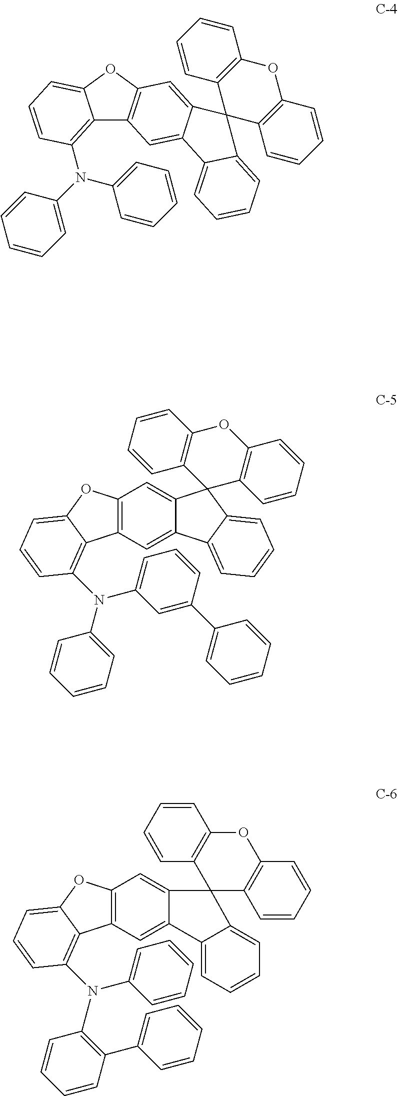 [SMILES: c1ccc(N(c2ccccc2)c2cccc3oc4cc5c(cc4c23)-c2ccccc2C52c3ccccc3Oc3ccccc32)cc1 ; c1ccc(-c2cccc(N(c3ccccc3)c3cccc4oc5cc6c(cc5c34)-c3ccccc3C63c4ccccc4Oc4ccccc43)c2)cc1 ; c1ccc(-c2ccccc2N(c2ccccc2)c2cccc3oc4cc5c(cc4c23)-c2ccccc2C52c3ccccc3Oc3ccccc32)cc1]

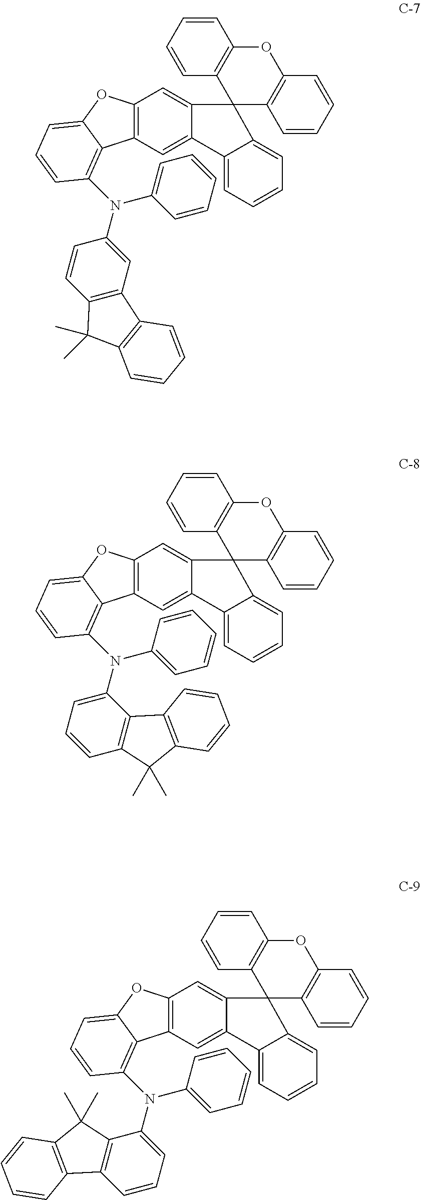 [SMILES: CC1(C)c2ccccc2-c2cc(N(c3ccccc3)c3cccc4oc5cc6c(cc5c34)-c3ccccc3C63c4ccccc4Oc4ccccc43)ccc21 ; CC1(C)c2ccccc2-c2c(N(c3ccccc3)c3cccc4oc5cc6c(cc5c34)-c3ccccc3C63c4ccccc4Oc4ccccc43)cccc21 ; CC1(C)c2ccccc2-c2cccc(N(c3ccccc3)c3cccc4oc5cc6c(cc5c34)-c3ccccc3C63c4ccccc4Oc4ccccc43)c21]

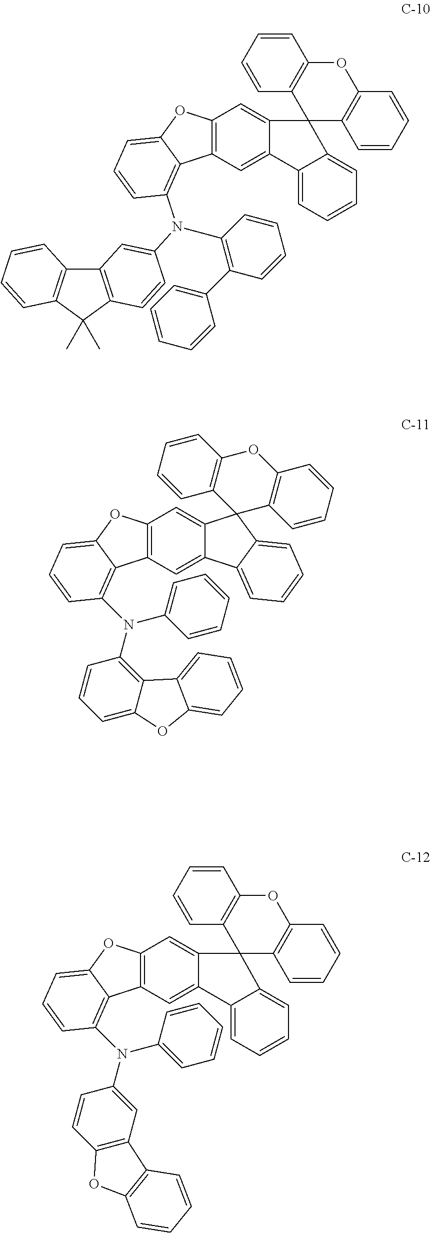 [SMILES: CC1(C)c2ccccc2-c2cc(N(c3ccccc3-c3ccccc3)c3cccc4oc5cc6c(cc5c34)-c3ccccc3C63c4ccccc4Oc4ccccc43)ccc21 ; c1ccc(N(c2cccc3oc4ccccc4c23)c2cccc3oc4cc5c(cc4c23)-c2ccccc2C52c3ccccc3Oc3ccccc32)cc1 ; c1ccc(N(c2ccc3oc4ccccc4c3c2)c2cccc3oc4cc5c(cc4c23)-c2ccccc2C52c3ccccc3Oc3ccccc32)cc1]

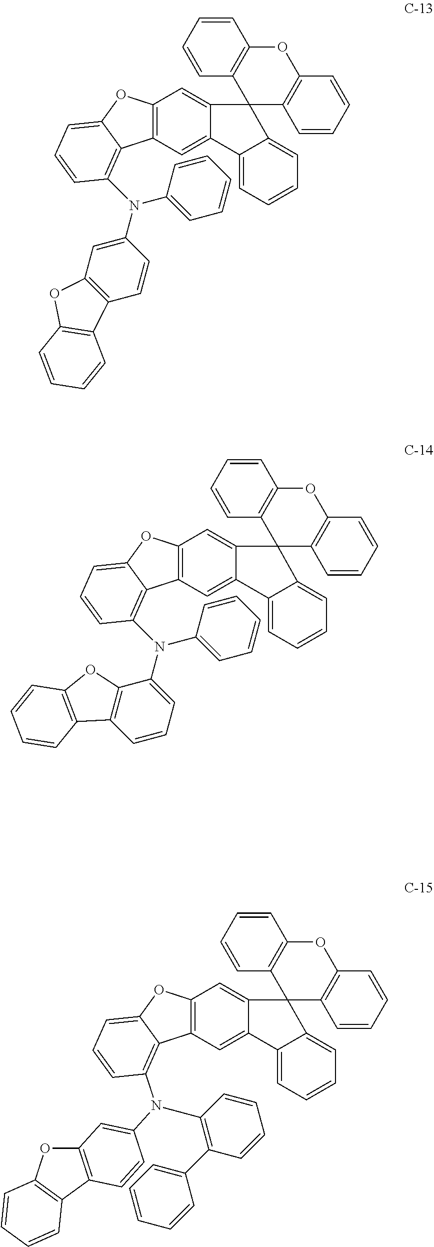 [SMILES: c1ccc(N(c2ccc3c(c2)oc2ccccc23)c2cccc3oc4cc5c(cc4c23)-c2ccccc2C52c3ccccc3Oc3ccccc32)cc1 ; c1ccc(N(c2cccc3c2oc2ccccc23)c2cccc3oc4cc5c(cc4c23)-c2ccccc2C52c3ccccc3Oc3ccccc32)cc1 ; c1ccc(-c2ccccc2N(c2ccc3c(c2)oc2ccccc23)c2cccc3oc4cc5c(cc4c23)-c2ccccc2C52c3ccccc3Oc3ccccc32)cc1]

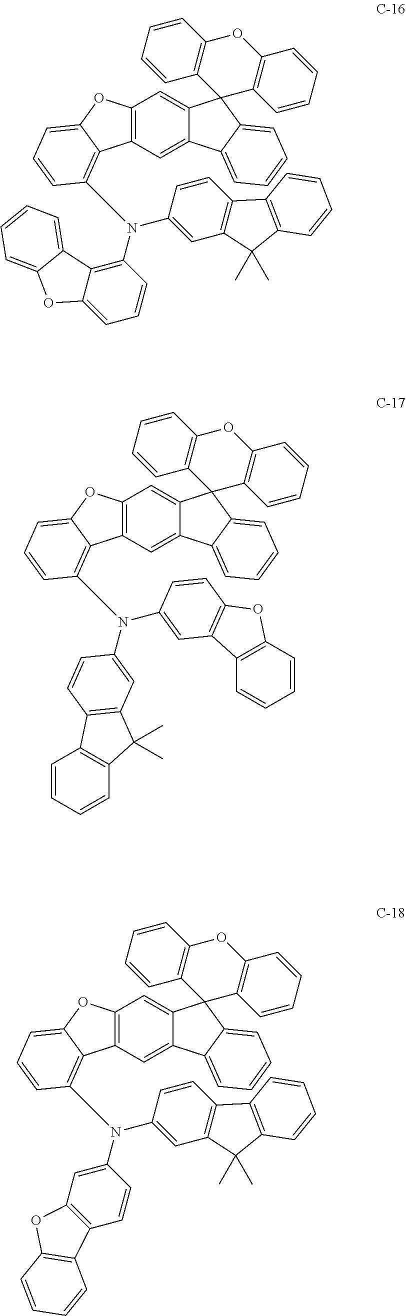 [SMILES: CC1(C)c2ccccc2-c2ccc(N(c3cccc4oc5ccccc5c34)c3cccc4oc5cc6c(cc5c34)-c3ccccc3C63c4ccccc4Oc4ccccc43)cc21 ; CC1(C)c2ccccc2-c2ccc(N(c3ccc4oc5ccccc5c4c3)c3cccc4oc5cc6c(cc5c34)-c3ccccc3C63c4ccccc4Oc4ccccc43)cc21 ; CC1(C)c2ccccc2-c2ccc(N(c3ccc4c(c3)oc3ccccc34)c3cccc4oc5cc6c(cc5c34)-c3ccccc3C63c4ccccc4Oc4ccccc43)cc21]

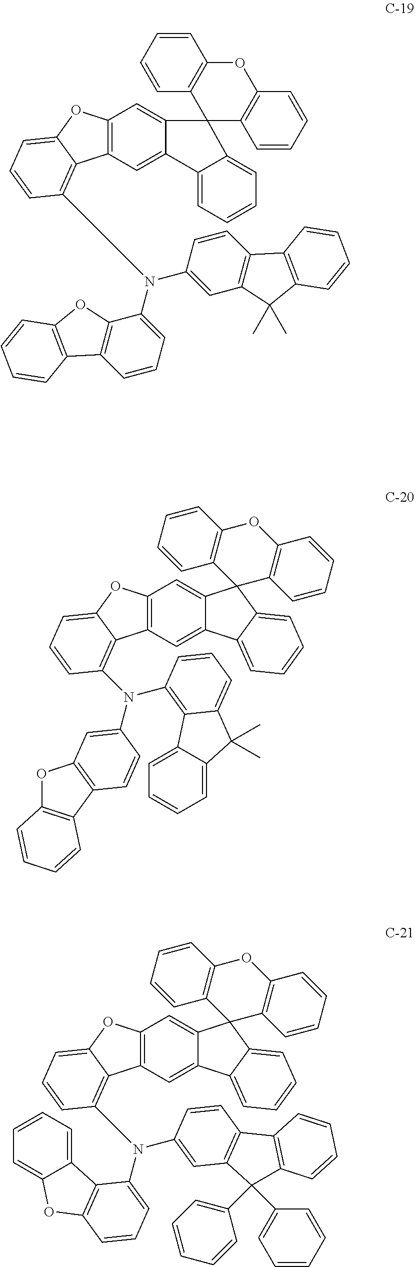 [SMILES: CC1(C)c2ccccc2-c2ccc(N(c3cccc4c3oc3ccccc34)c3cccc4oc5cc6c(cc5c34)-c3ccccc3C63c4ccccc4Oc4ccccc43)cc21 ; CC1(C)c2ccccc2-c2c(N(c3ccc4c(c3)oc3ccccc34)c3cccc4oc5cc6c(cc5c34)-c3ccccc3C63c4ccccc4Oc4ccccc43)cccc21 ; c1ccc(C2(c3ccccc3)c3ccccc3-c3ccc(N(c4cccc5oc6ccccc6c45)c4cccc5oc6cc7c(cc6c45)-c4ccccc4C74c5ccccc5Oc5ccccc54)cc32)cc1]

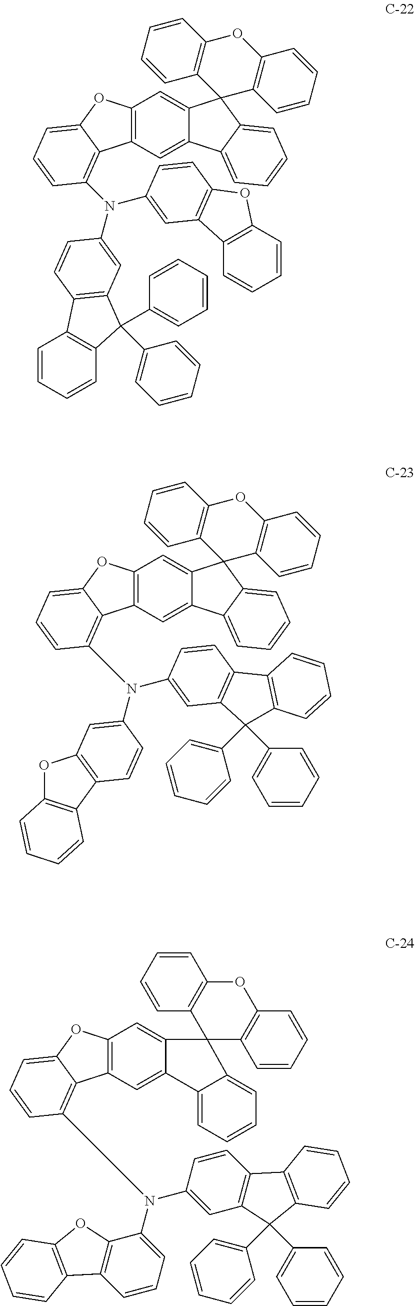 [SMILES: c1ccc(C2(c3ccccc3)c3ccccc3-c3ccc(N(c4ccc5oc6ccccc6c5c4)c4cccc5oc6cc7c(cc6c45)-c4ccccc4C74c5ccccc5Oc5ccccc54)cc32)cc1 ; c1ccc(C2(c3ccccc3)c3ccccc3-c3ccc(N(c4ccc5c(c4)oc4ccccc45)c4cccc5oc6cc7c(cc6c45)-c4ccccc4C74c5ccccc5Oc5ccccc54)cc32)cc1 ; c1ccc(C2(c3ccccc3)c3ccccc3-c3ccc(N(c4cccc5c4oc4ccccc45)c4cccc5oc6cc7c(cc6c45)-c4ccccc4C74c5ccccc5Oc5ccccc54)cc32)cc1]

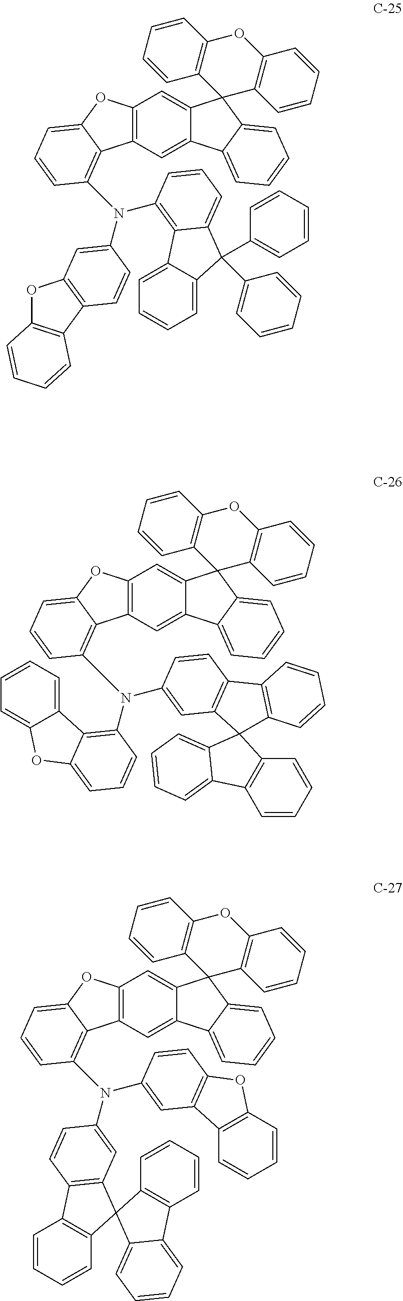 [SMILES: c1ccc(C2(c3ccccc3)c3ccccc3-c3c(N(c4ccc5c(c4)oc4ccccc45)c4cccc5oc6cc7c(cc6c45)-c4ccccc4C74c5ccccc5Oc5ccccc54)cccc32)cc1 ; c1ccc2c(c1)Oc1ccccc1C21c2ccccc2-c2cc3c(cc21)oc1cccc(N(c2ccc4c(c2)C2(c5ccccc5-c5ccccc52)c2ccccc2-4)c2cccc4oc5ccccc5c24)c13 ; c1ccc2c(c1)Oc1ccccc1C21c2ccccc2-c2cc3c(cc21)oc1cccc(N(c2ccc4c(c2)C2(c5ccccc5-c5ccccc52)c2ccccc2-4)c2ccc4oc5ccccc5c4c2)c13]

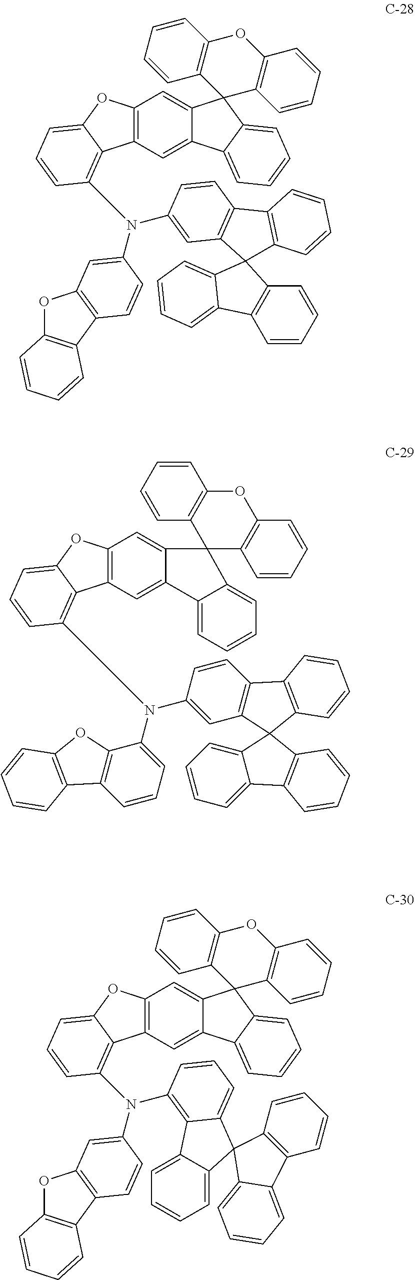 [SMILES: c1ccc2c(c1)Oc1ccccc1C21c2ccccc2-c2cc3c(cc21)oc1cccc(N(c2ccc4c(c2)C2(c5ccccc5-c5ccccc52)c2ccccc2-4)c2ccc4c(c2)oc2ccccc24)c13 ; c1ccc2c(c1)Oc1ccccc1C21c2ccccc2-c2cc3c(cc21)oc1cccc(N(c2ccc4c(c2)C2(c5ccccc5-c5ccccc52)c2ccccc2-4)c2cccc4c2oc2ccccc24)c13 ; c1ccc2c(c1)Oc1ccccc1C21c2ccccc2-c2cc3c(cc21)oc1cccc(N(c2ccc4c(c2)oc2ccccc24)c2cccc4c2-c2ccccc2C42c4ccccc4-c4ccccc42)c13]

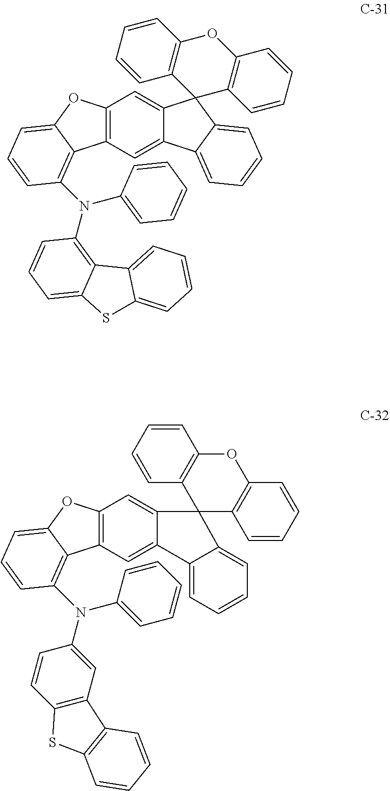 [SMILES: c1ccc(N(c2cccc3oc4cc5c(cc4c23)-c2ccccc2C52c3ccccc3Oc3ccccc32)c2cccc3sc4ccccc4c23)cc1 ; c1ccc(N(c2ccc3sc4ccccc4c3c2)c2cccc3oc4cc5c(cc4c23)-c2ccccc2C52c3ccccc3Oc3ccccc32)cc1]

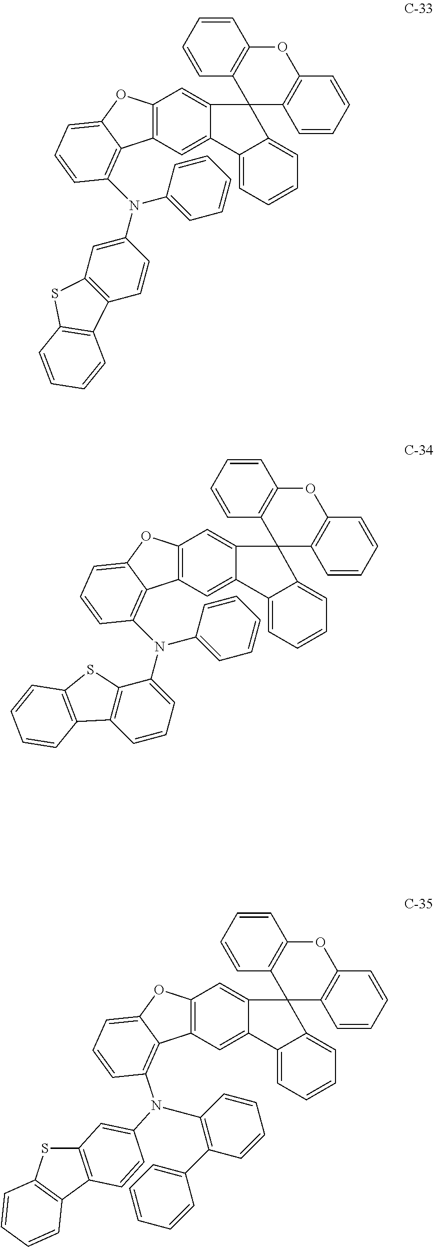 [SMILES: c1ccc(N(c2ccc3c(c2)sc2ccccc23)c2cccc3oc4cc5c(cc4c23)-c2ccccc2C52c3ccccc3Oc3ccccc32)cc1 ; c1ccc(N(c2cccc3c2sc2ccccc23)c2cccc3oc4cc5c(cc4c23)-c2ccccc2C52c3ccccc3Oc3ccccc32)cc1 ; c1ccc(-c2ccccc2N(c2ccc3c(c2)sc2ccccc23)c2cccc3oc4cc5c(cc4c23)-c2ccccc2C52c3ccccc3Oc3ccccc32)cc1]

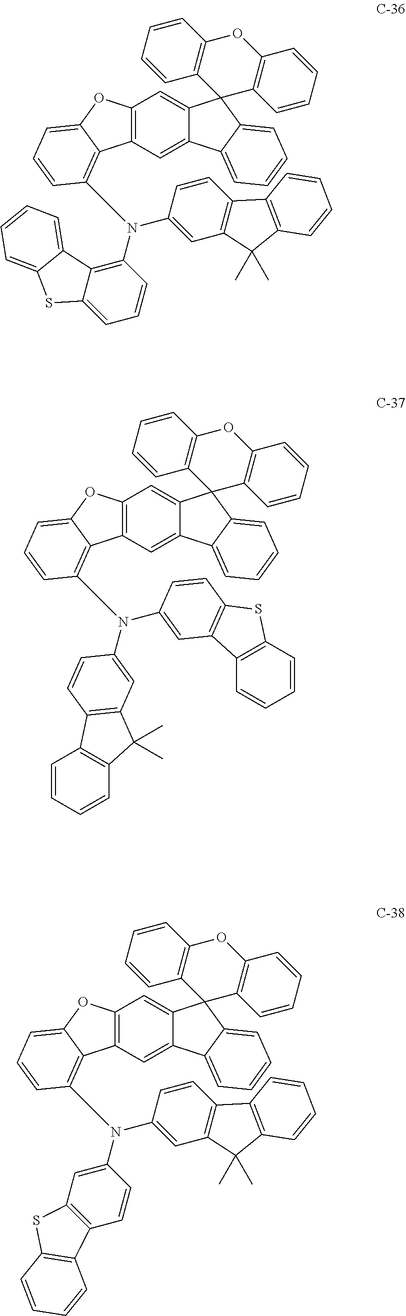 [SMILES: CC1(C)c2ccccc2-c2ccc(N(c3cccc4oc5cc6c(cc5c34)-c3ccccc3C63c4ccccc4Oc4ccccc43)c3cccc4sc5ccccc5c34)cc21 ; CC1(C)c2ccccc2-c2ccc(N(c3ccc4sc5ccccc5c4c3)c3cccc4oc5cc6c(cc5c34)-c3ccccc3C63c4ccccc4Oc4ccccc43)cc21 ; CC1(C)c2ccccc2-c2ccc(N(c3ccc4c(c3)sc3ccccc34)c3cccc4oc5cc6c(cc5c34)-c3ccccc3C63c4ccccc4Oc4ccccc43)cc21]

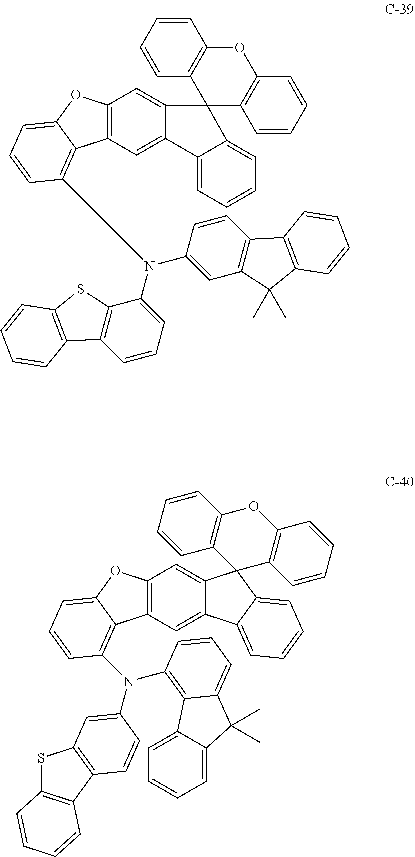 [SMILES: CC1(C)c2ccccc2-c2ccc(N(c3cccc4c3sc3ccccc34)c3cccc4oc5cc6c(cc5c34)-c3ccccc3C63c4ccccc4Oc4ccccc43)cc21 ; CC1(C)c2ccccc2-c2c(N(c3ccc4c(c3)sc3ccccc34)c3cccc4oc5cc6c(cc5c34)-c3ccccc3C63c4ccccc4Oc4ccccc43)cccc21]

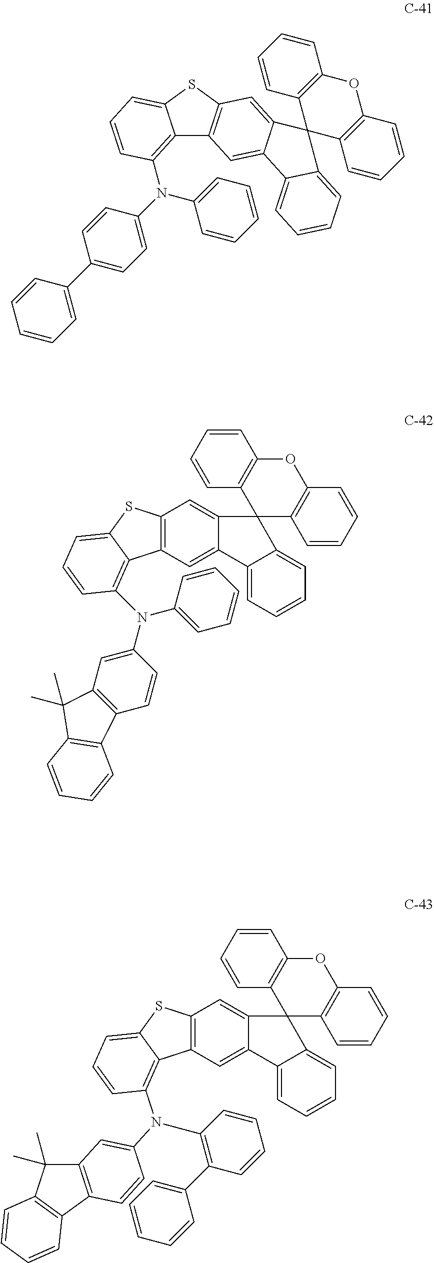 [SMILES: c1ccc(-c2ccc(N(c3ccccc3)c3cccc4sc5cc6c(cc5c34)-c3ccccc3C63c4ccccc4Oc4ccccc43)cc2)cc1 ; CC1(C)c2ccccc2-c2ccc(N(c3ccccc3)c3cccc4sc5cc6c(cc5c34)-c3ccccc3C63c4ccccc4Oc4ccccc43)cc21 ; CC1(C)c2ccccc2-c2ccc(N(c3ccccc3-c3ccccc3)c3cccc4sc5cc6c(cc5c34)-c3ccccc3C63c4ccccc4Oc4ccccc43)cc21]

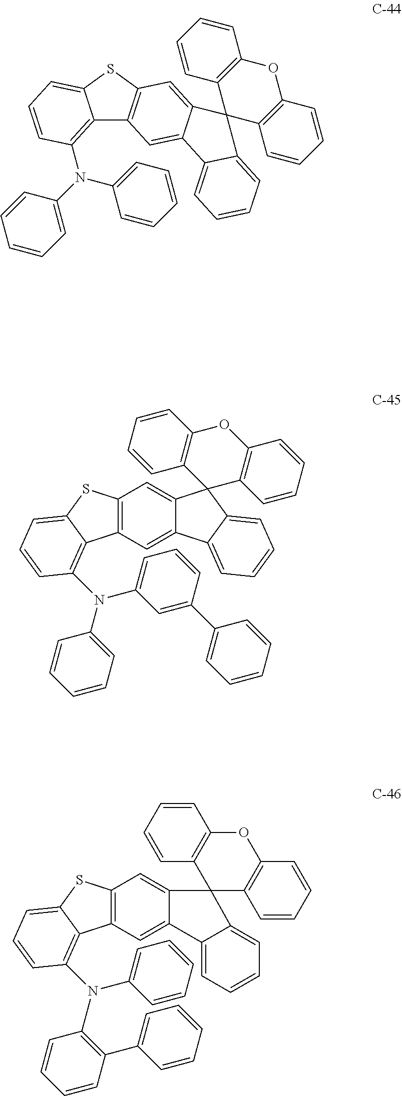 [SMILES: c1ccc(N(c2ccccc2)c2cccc3sc4cc5c(cc4c23)-c2ccccc2C52c3ccccc3Oc3ccccc32)cc1 ; c1ccc(-c2cccc(N(c3ccccc3)c3cccc4sc5cc6c(cc5c34)-c3ccccc3C63c4ccccc4Oc4ccccc43)c2)cc1 ; c1ccc(-c2ccccc2N(c2ccccc2)c2cccc3sc4cc5c(cc4c23)-c2ccccc2C52c3ccccc3Oc3ccccc32)cc1]

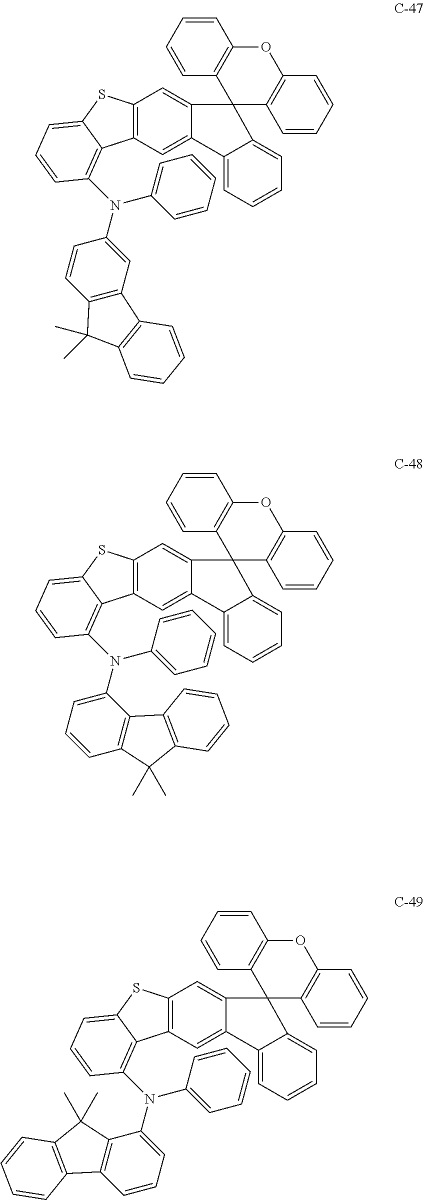 [SMILES: CC1(C)c2ccccc2-c2cc(N(c3ccccc3)c3cccc4sc5cc6c(cc5c34)-c3ccccc3C63c4ccccc4Oc4ccccc43)ccc21 ; CC1(C)c2ccccc2-c2c(N(c3ccccc3)c3cccc4sc5cc6c(cc5c34)-c3ccccc3C63c4ccccc4Oc4ccccc43)cccc21 ; CC1(C)c2ccccc2-c2cccc(N(c3ccccc3)c3cccc4sc5cc6c(cc5c34)-c3ccccc3C63c4ccccc4Oc4ccccc43)c21]

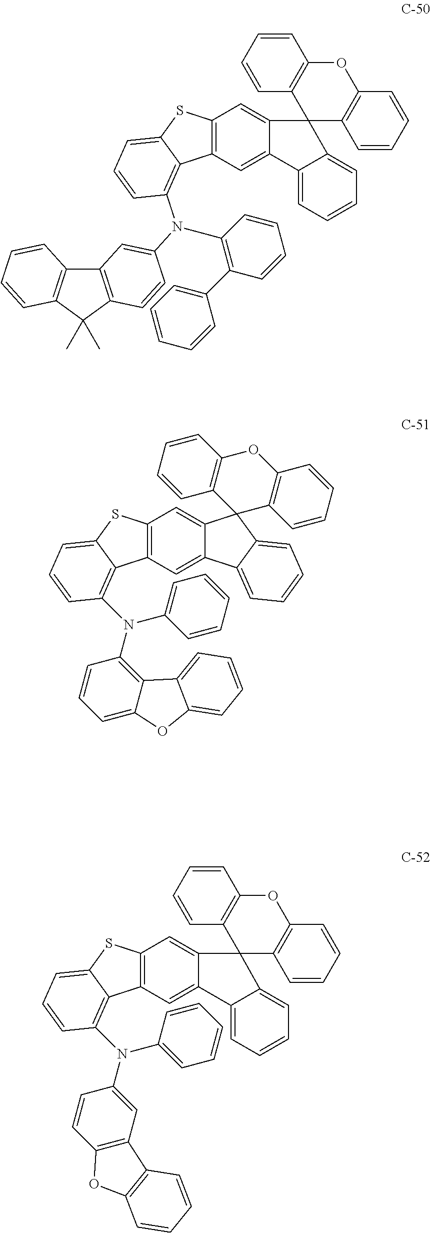 [SMILES: CC1(C)c2ccccc2-c2cc(N(c3ccccc3-c3ccccc3)c3cccc4sc5cc6c(cc5c34)-c3ccccc3C63c4ccccc4Oc4ccccc43)ccc21 ; c1ccc(N(c2cccc3oc4ccccc4c23)c2cccc3sc4cc5c(cc4c23)-c2ccccc2C52c3ccccc3Oc3ccccc32)cc1 ; c1ccc(N(c2ccc3oc4ccccc4c3c2)c2cccc3sc4cc5c(cc4c23)-c2ccccc2C52c3ccccc3Oc3ccccc32)cc1]

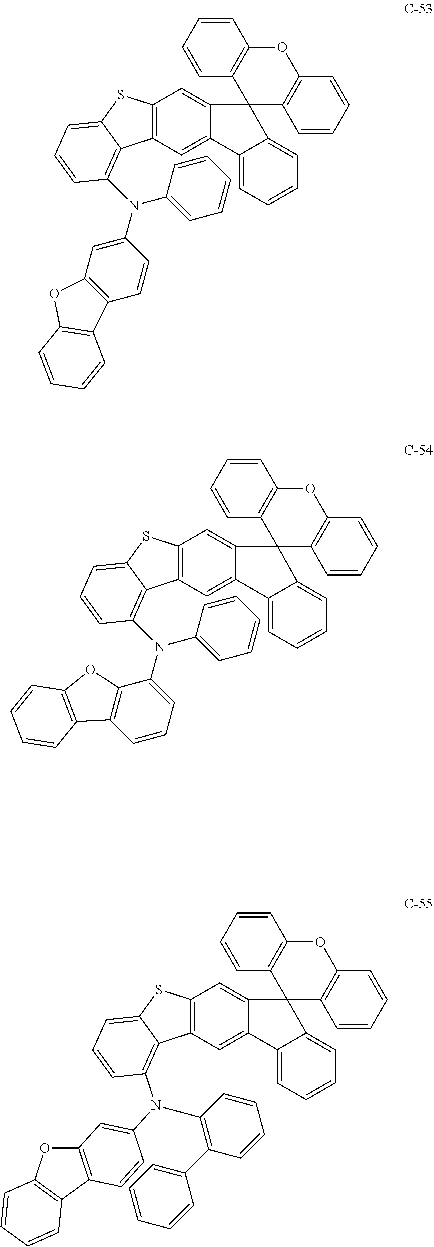 [SMILES: c1ccc(N(c2ccc3c(c2)oc2ccccc23)c2cccc3sc4cc5c(cc4c23)-c2ccccc2C52c3ccccc3Oc3ccccc32)cc1 ; c1ccc(N(c2cccc3c2oc2ccccc23)c2cccc3sc4cc5c(cc4c23)-c2ccccc2C52c3ccccc3Oc3ccccc32)cc1 ; c1ccc(-c2ccccc2N(c2ccc3c(c2)oc2ccccc23)c2cccc3sc4cc5c(cc4c23)-c2ccccc2C52c3ccccc3Oc3ccccc32)cc1]

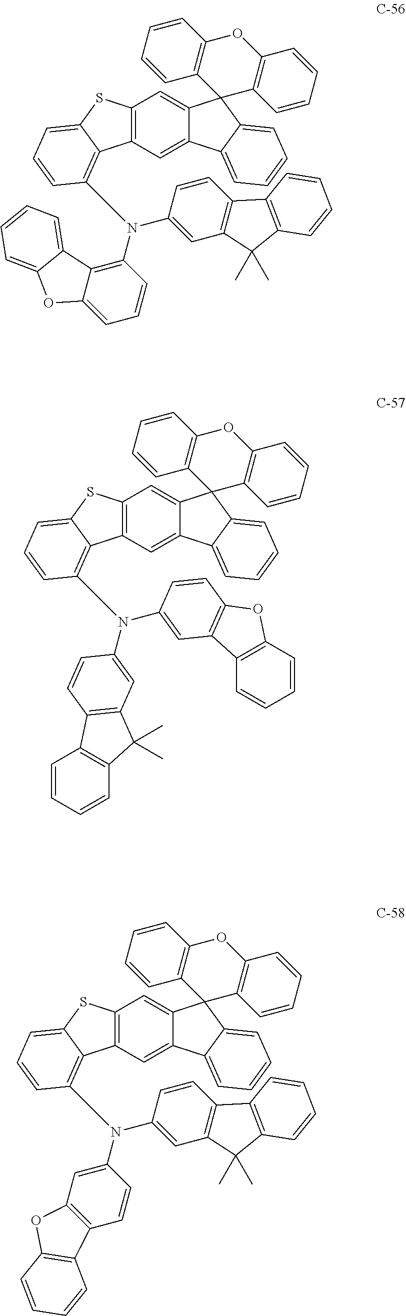 [SMILES: CC1(C)c2ccccc2-c2ccc(N(c3cccc4oc5ccccc5c34)c3cccc4sc5cc6c(cc5c34)-c3ccccc3C63c4ccccc4Oc4ccccc43)cc21 ; CC1(C)c2ccccc2-c2ccc(N(c3ccc4oc5ccccc5c4c3)c3cccc4sc5cc6c(cc5c34)-c3ccccc3C63c4ccccc4Oc4ccccc43)cc21 ; CC1(C)c2ccccc2-c2ccc(N(c3ccc4c(c3)oc3ccccc34)c3cccc4sc5cc6c(cc5c34)-c3ccccc3C63c4ccccc4Oc4ccccc43)cc21]

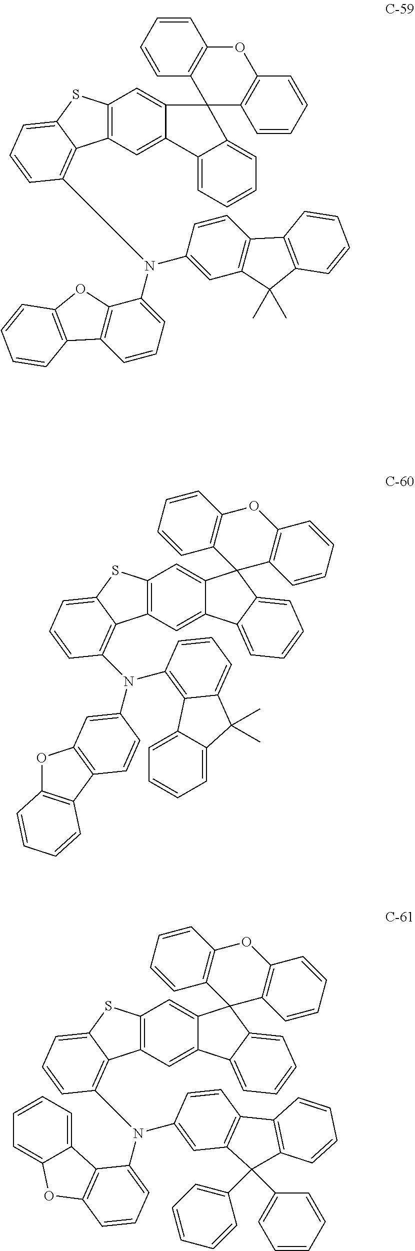 [SMILES: CC1(C)c2ccccc2-c2ccc(N(c3cccc4c3oc3ccccc34)c3cccc4sc5cc6c(cc5c34)-c3ccccc3C63c4ccccc4Oc4ccccc43)cc21 ; CC1(C)c2ccccc2-c2c(N(c3ccc4c(c3)oc3ccccc34)c3cccc4sc5cc6c(cc5c34)-c3ccccc3C63c4ccccc4Oc4ccccc43)cccc21 ; c1ccc(C2(c3ccccc3)c3ccccc3-c3ccc(N(c4cccc5oc6ccccc6c45)c4cccc5sc6cc7c(cc6c45)-c4ccccc4C74c5ccccc5Oc5ccccc54)cc32)cc1]

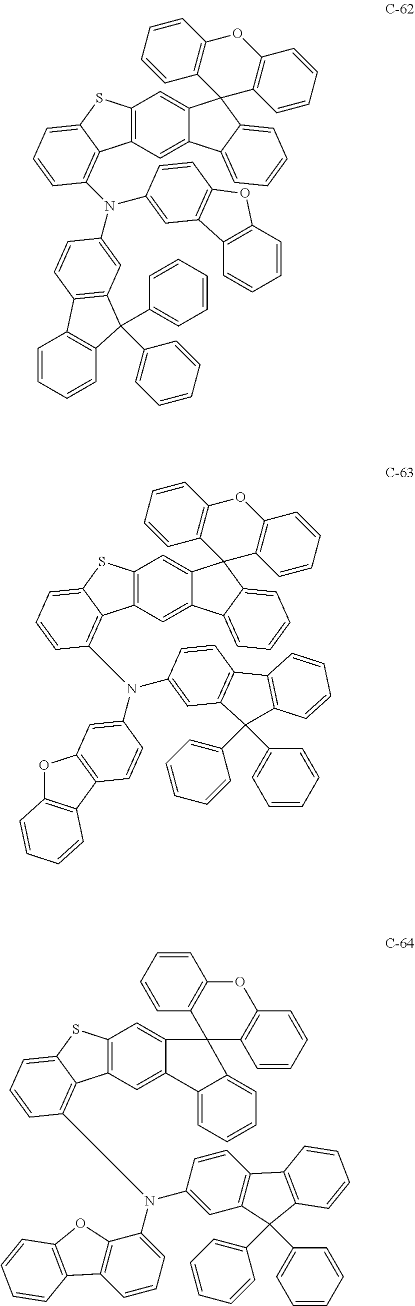 [SMILES: c1ccc(C2(c3ccccc3)c3ccccc3-c3ccc(N(c4ccc5oc6ccccc6c5c4)c4cccc5sc6cc7c(cc6c45)-c4ccccc4C74c5ccccc5Oc5ccccc54)cc32)cc1 ; c1ccc(C2(c3ccccc3)c3ccccc3-c3ccc(N(c4ccc5c(c4)oc4ccccc45)c4cccc5sc6cc7c(cc6c45)-c4ccccc4C74c5ccccc5Oc5ccccc54)cc32)cc1 ; c1ccc(C2(c3ccccc3)c3ccccc3-c3ccc(N(c4cccc5c4oc4ccccc45)c4cccc5sc6cc7c(cc6c45)-c4ccccc4C74c5ccccc5Oc5ccccc54)cc32)cc1]

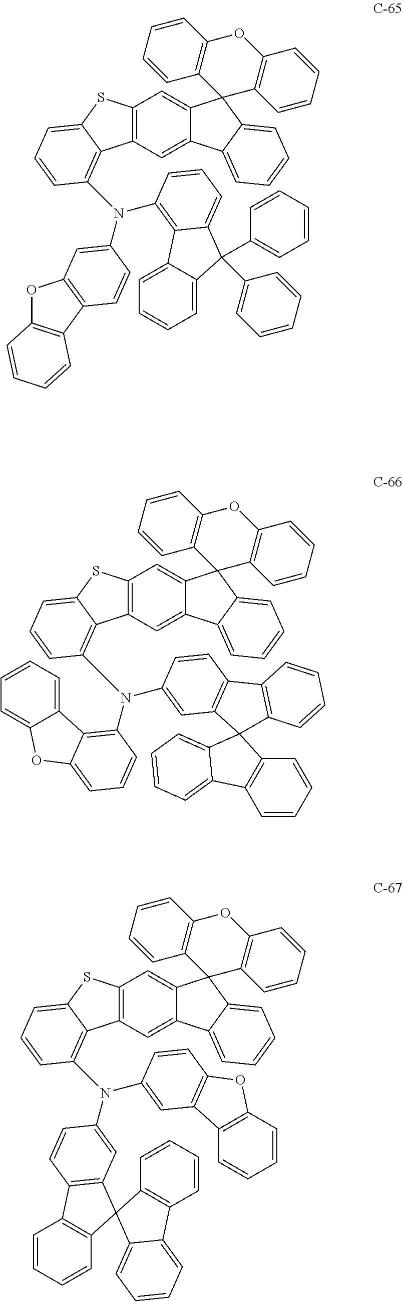 [SMILES: c1ccc(C2(c3ccccc3)c3ccccc3-c3c(N(c4ccc5c(c4)oc4ccccc45)c4cccc5sc6cc7c(cc6c45)-c4ccccc4C74c5ccccc5Oc5ccccc54)cccc32)cc1 ; c1ccc2c(c1)Oc1ccccc1C21c2ccccc2-c2cc3c(cc21)sc1cccc(N(c2ccc4c(c2)C2(c5ccccc5-c5ccccc52)c2ccccc2-4)c2cccc4oc5ccccc5c24)c13 ; c1ccc2c(c1)Oc1ccccc1C21c2ccccc2-c2cc3c(cc21)sc1cccc(N(c2ccc4c(c2)C2(c5ccccc5-c5ccccc52)c2ccccc2-4)c2ccc4oc5ccccc5c4c2)c13]

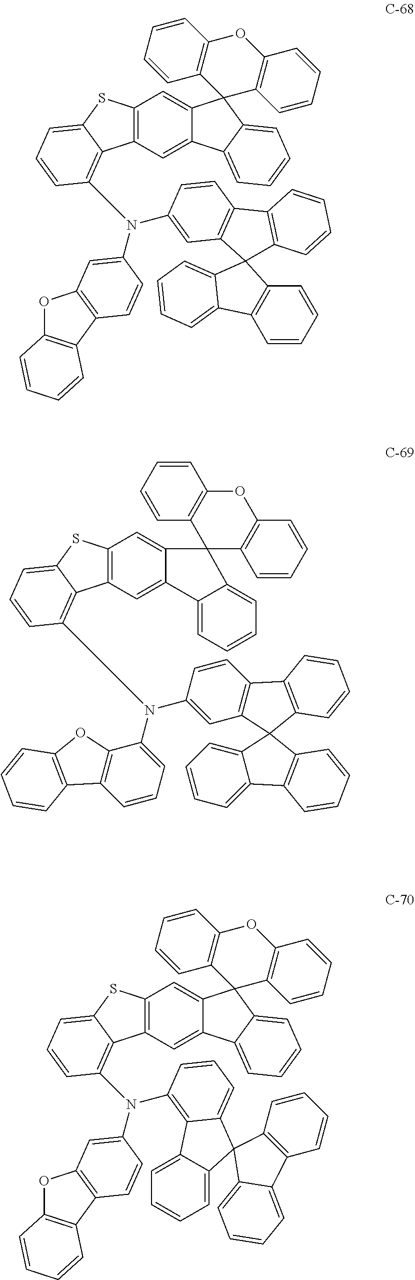 [SMILES: c1ccc2c(c1)Oc1ccccc1C21c2ccccc2-c2cc3c(cc21)sc1cccc(N(c2ccc4c(c2)C2(c5ccccc5-c5ccccc52)c2ccccc2-4)c2ccc4c(c2)oc2ccccc24)c13 ; c1ccc2c(c1)Oc1ccccc1C21c2ccccc2-c2cc3c(cc21)sc1cccc(N(c2ccc4c(c2)C2(c5ccccc5-c5ccccc52)c2ccccc2-4)c2cccc4c2oc2ccccc24)c13 ; c1ccc2c(c1)Oc1ccccc1C21c2ccccc2-c2cc3c(cc21)sc1cccc(N(c2ccc4c(c2)oc2ccccc24)c2cccc4c2-c2ccccc2C42c4ccccc4-c4ccccc42)c13]

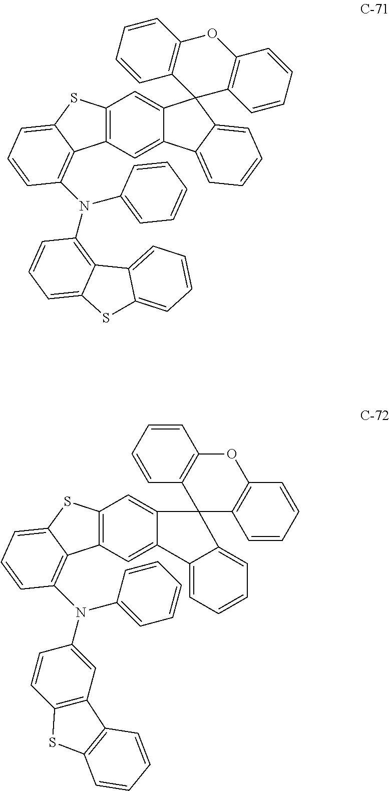 [SMILES: c1ccc(N(c2cccc3sc4ccccc4c23)c2cccc3sc4cc5c(cc4c23)-c2ccccc2C52c3ccccc3Oc3ccccc32)cc1 ; c1ccc(N(c2ccc3sc4ccccc4c3c2)c2cccc3sc4cc5c(cc4c23)-c2ccccc2C52c3ccccc3Oc3ccccc32)cc1]

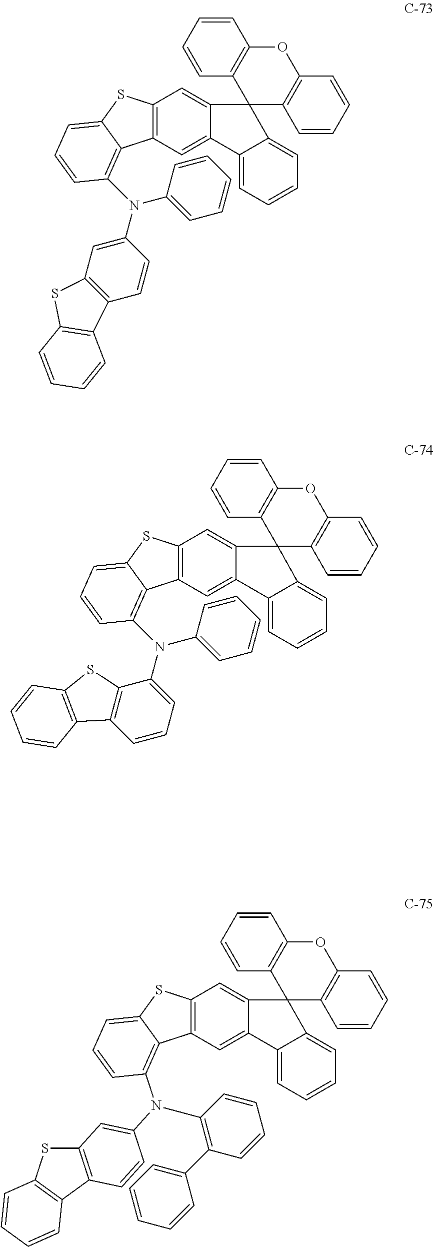 [SMILES: c1ccc(N(c2ccc3c(c2)sc2ccccc23)c2cccc3sc4cc5c(cc4c23)-c2ccccc2C52c3ccccc3Oc3ccccc32)cc1 ; c1ccc(N(c2cccc3c2sc2ccccc23)c2cccc3sc4cc5c(cc4c23)-c2ccccc2C52c3ccccc3Oc3ccccc32)cc1 ; c1ccc(-c2ccccc2N(c2ccc3c(c2)sc2ccccc23)c2cccc3sc4cc5c(cc4c23)-c2ccccc2C52c3ccccc3Oc3ccccc32)cc1]

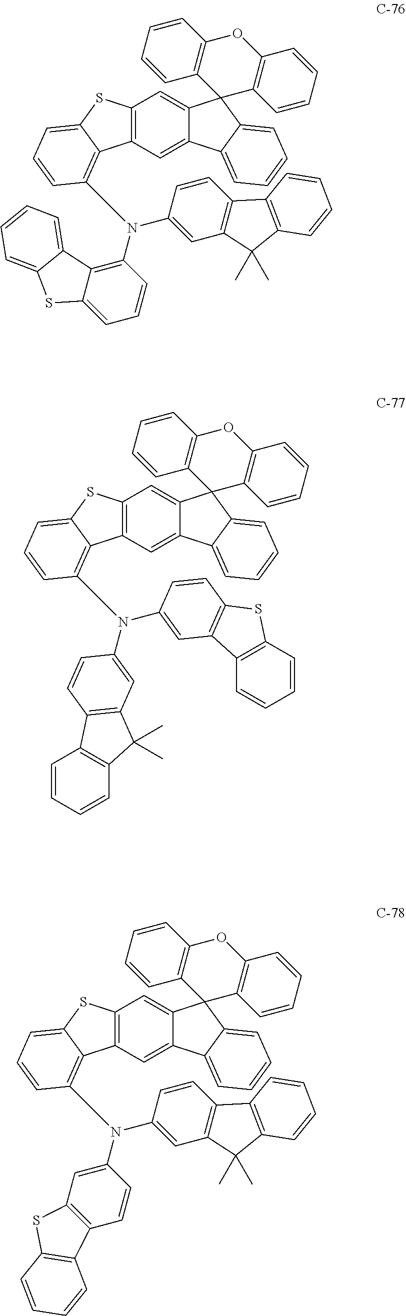 [SMILES: CC1(C)c2ccccc2-c2ccc(N(c3cccc4sc5ccccc5c34)c3cccc4sc5cc6c(cc5c34)-c3ccccc3C63c4ccccc4Oc4ccccc43)cc21 ; CC1(C)c2ccccc2-c2ccc(N(c3ccc4sc5ccccc5c4c3)c3cccc4sc5cc6c(cc5c34)-c3ccccc3C63c4ccccc4Oc4ccccc43)cc21 ; CC1(C)c2ccccc2-c2ccc(N(c3ccc4c(c3)sc3ccccc34)c3cccc4sc5cc6c(cc5c34)-c3ccccc3C63c4ccccc4Oc4ccccc43)cc21]

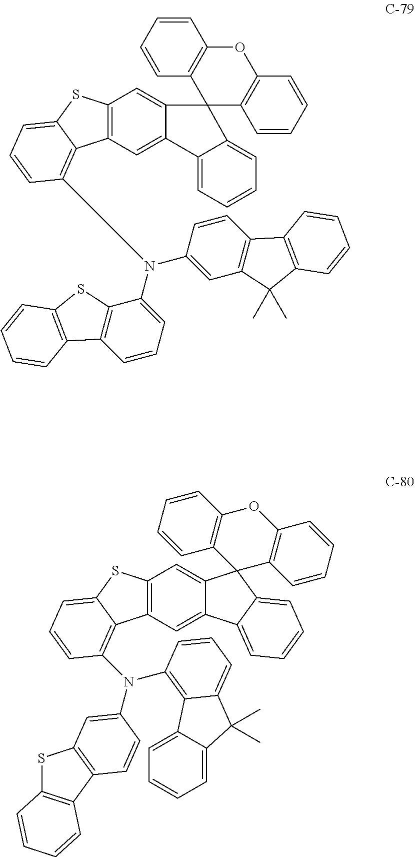 [SMILES: CC1(C)c2ccccc2-c2ccc(N(c3cccc4c3sc3ccccc34)c3cccc4sc5cc6c(cc5c34)-c3ccccc3C63c4ccccc4Oc4ccccc43)cc21 ; CC1(C)c2ccccc2-c2c(N(c3ccc4c(c3)sc3ccccc34)c3cccc4sc5cc6c(cc5c34)-c3ccccc3C63c4ccccc4Oc4ccccc43)cccc21]

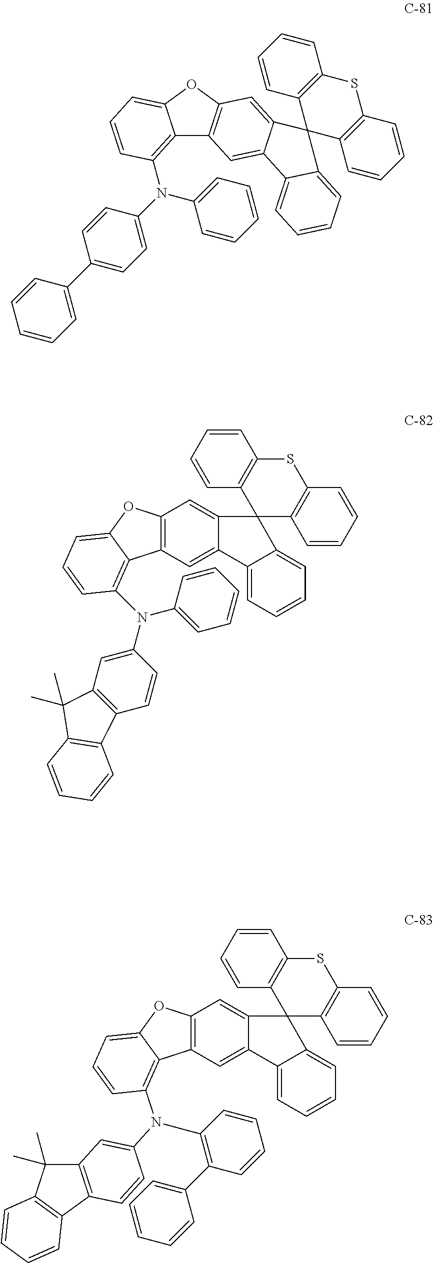 [SMILES: c1ccc(-c2ccc(N(c3ccccc3)c3cccc4oc5cc6c(cc5c34)-c3ccccc3C63c4ccccc4Sc4ccccc43)cc2)cc1 ; CC1(C)c2ccccc2-c2ccc(N(c3ccccc3)c3cccc4oc5cc6c(cc5c34)-c3ccccc3C63c4ccccc4Sc4ccccc43)cc21 ; CC1(C)c2ccccc2-c2ccc(N(c3ccccc3-c3ccccc3)c3cccc4oc5cc6c(cc5c34)-c3ccccc3C63c4ccccc4Sc4ccccc43)cc21]

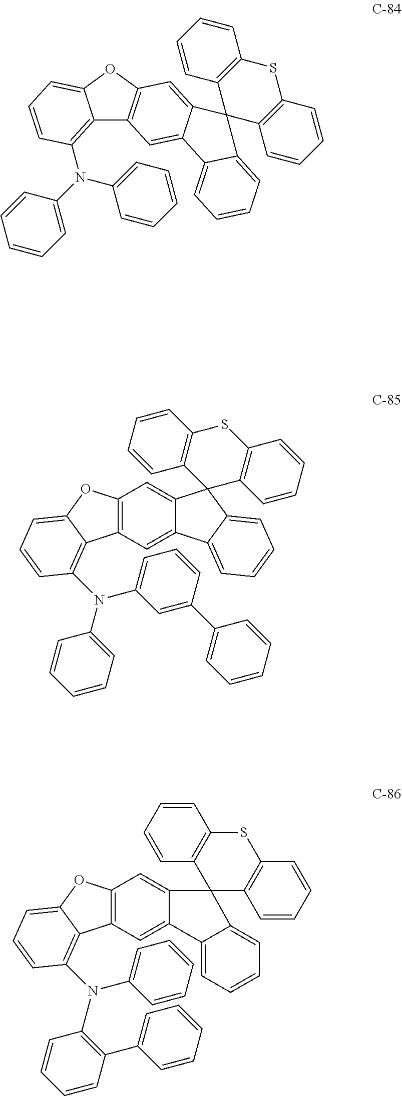 [SMILES: c1ccc(N(c2ccccc2)c2cccc3oc4cc5c(cc4c23)-c2ccccc2C52c3ccccc3Sc3ccccc32)cc1 ; c1ccc(-c2cccc(N(c3ccccc3)c3cccc4oc5cc6c(cc5c34)-c3ccccc3C63c4ccccc4Sc4ccccc43)c2)cc1 ; c1ccc(-c2ccccc2N(c2ccccc2)c2cccc3oc4cc5c(cc4c23)-c2ccccc2C52c3ccccc3Sc3ccccc32)cc1]

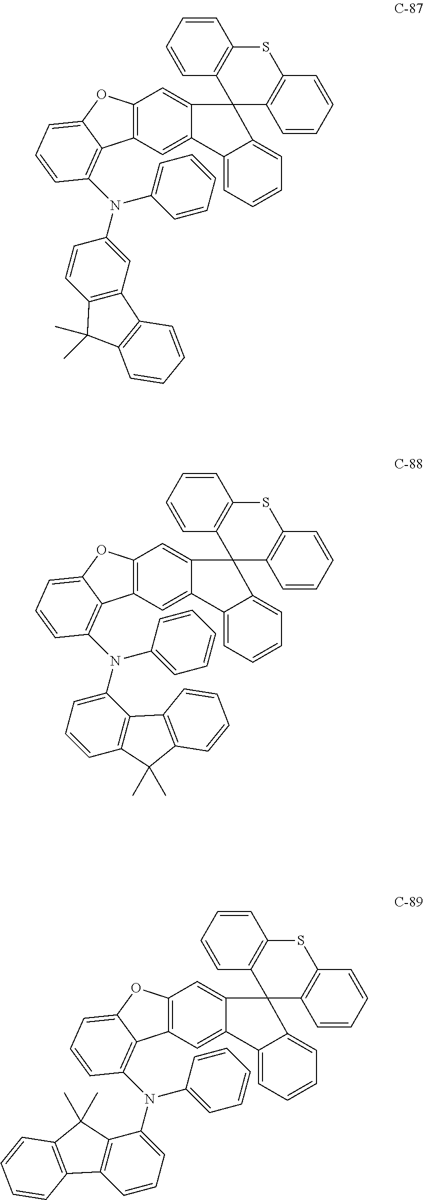 [SMILES: CC1(C)c2ccccc2-c2cc(N(c3ccccc3)c3cccc4oc5cc6c(cc5c34)-c3ccccc3C63c4ccccc4Sc4ccccc43)ccc21 ; CC1(C)c2ccccc2-c2c(N(c3ccccc3)c3cccc4oc5cc6c(cc5c34)-c3ccccc3C63c4ccccc4Sc4ccccc43)cccc21 ; CC1(C)c2ccccc2-c2cccc(N(c3ccccc3)c3cccc4oc5cc6c(cc5c34)-c3ccccc3C63c4ccccc4Sc4ccccc43)c21]

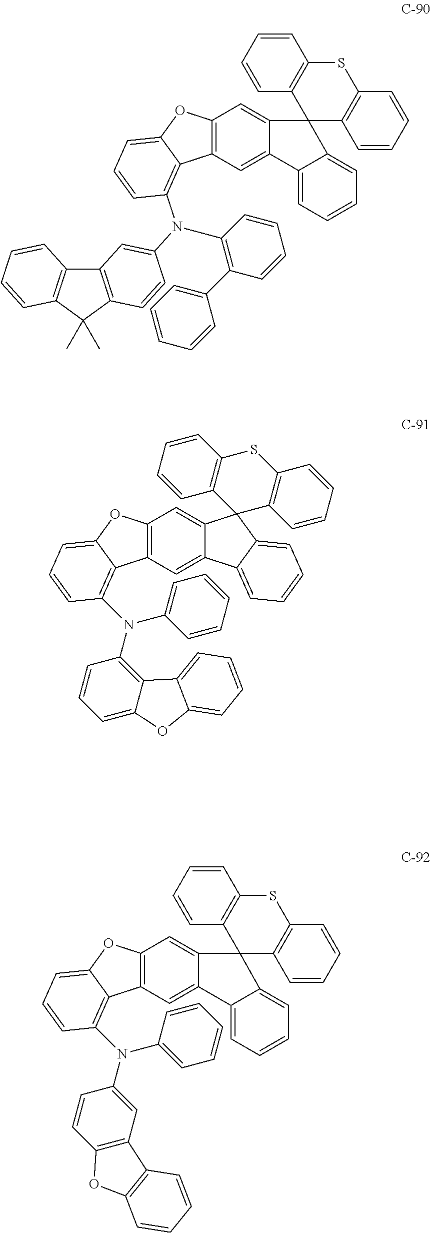 [SMILES: CC1(C)c2ccccc2-c2cc(N(c3ccccc3-c3ccccc3)c3cccc4oc5cc6c(cc5c34)-c3ccccc3C63c4ccccc4Sc4ccccc43)ccc21 ; c1ccc(N(c2cccc3oc4ccccc4c23)c2cccc3oc4cc5c(cc4c23)-c2ccccc2C52c3ccccc3Sc3ccccc32)cc1 ; c1ccc(N(c2ccc3oc4ccccc4c3c2)c2cccc3oc4cc5c(cc4c23)-c2ccccc2C52c3ccccc3Sc3ccccc32)cc1]

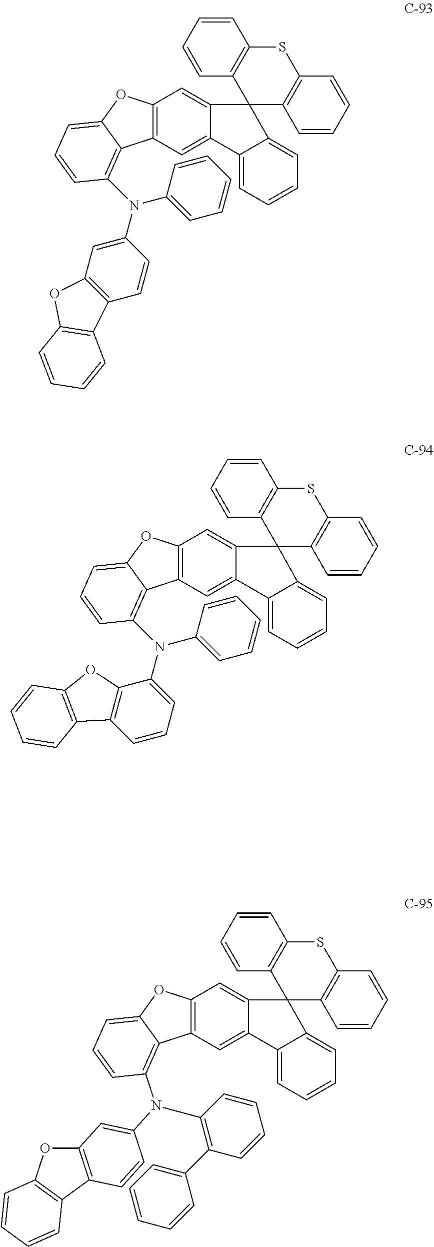 [SMILES: c1ccc(N(c2ccc3c(c2)oc2ccccc23)c2cccc3oc4cc5c(cc4c23)-c2ccccc2C52c3ccccc3Sc3ccccc32)cc1 ; c1ccc(N(c2cccc3c2oc2ccccc23)c2cccc3oc4cc5c(cc4c23)-c2ccccc2C52c3ccccc3Sc3ccccc32)cc1 ; c1ccc(-c2ccccc2N(c2ccc3c(c2)oc2ccccc23)c2cccc3oc4cc5c(cc4c23)-c2ccccc2C52c3ccccc3Sc3ccccc32)cc1]

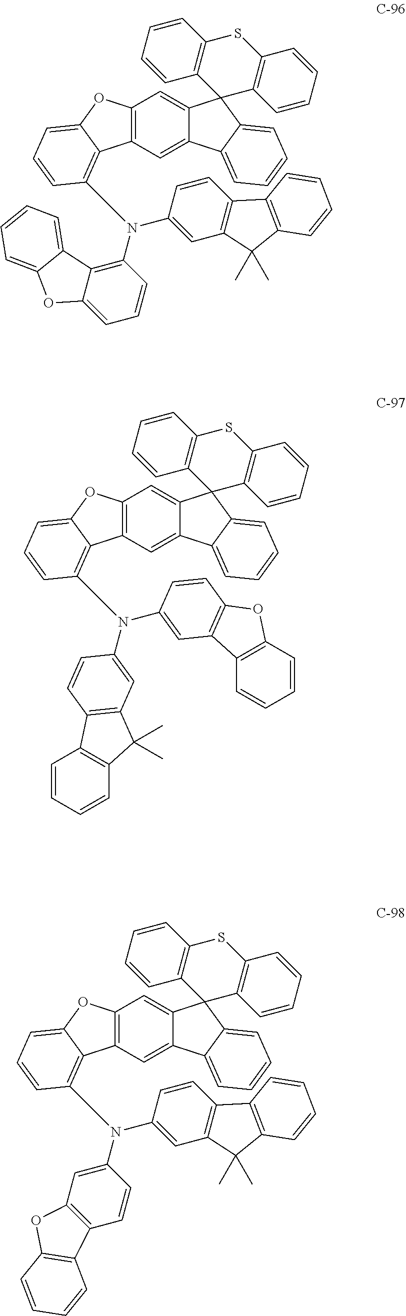 [SMILES: CC1(C)c2ccccc2-c2ccc(N(c3cccc4oc5ccccc5c34)c3cccc4oc5cc6c(cc5c34)-c3ccccc3C63c4ccccc4Sc4ccccc43)cc21 ; CC1(C)c2ccccc2-c2ccc(N(c3ccc4oc5ccccc5c4c3)c3cccc4oc5cc6c(cc5c34)-c3ccccc3C63c4ccccc4Sc4ccccc43)cc21 ; CC1(C)c2ccccc2-c2ccc(N(c3ccc4c(c3)oc3ccccc34)c3cccc4oc5cc6c(cc5c34)-c3ccccc3C63c4ccccc4Sc4ccccc43)cc21]

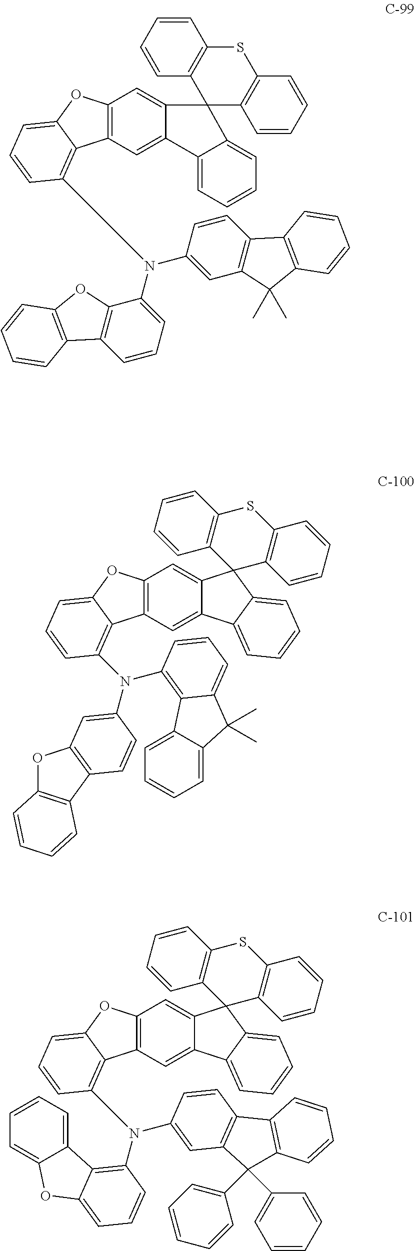 [SMILES: CC1(C)c2ccccc2-c2ccc(N(c3cccc4c3oc3ccccc34)c3cccc4oc5cc6c(cc5c34)-c3ccccc3C63c4ccccc4Sc4ccccc43)cc21 ; CC1(C)c2ccccc2-c2c(N(c3ccc4c(c3)oc3ccccc34)c3cccc4oc5cc6c(cc5c34)-c3ccccc3C63c4ccccc4Sc4ccccc43)cccc21 ; c1ccc(C2(c3ccccc3)c3ccccc3-c3ccc(N(c4cccc5oc6ccccc6c45)c4cccc5oc6cc7c(cc6c45)-c4ccccc4C74c5ccccc5Sc5ccccc54)cc32)cc1]

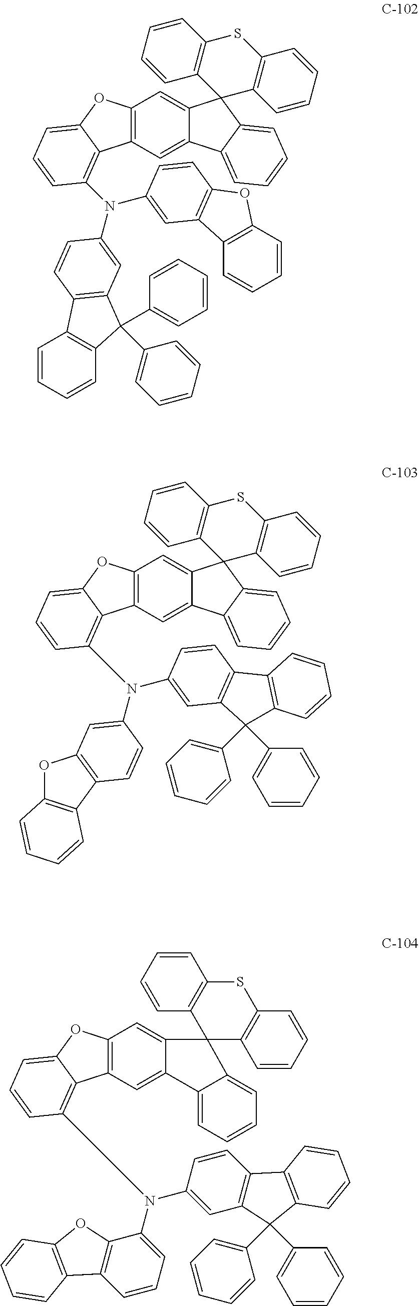 [SMILES: c1ccc(C2(c3ccccc3)c3ccccc3-c3ccc(N(c4ccc5oc6ccccc6c5c4)c4cccc5oc6cc7c(cc6c45)-c4ccccc4C74c5ccccc5Sc5ccccc54)cc32)cc1 ; c1ccc(C2(c3ccccc3)c3ccccc3-c3ccc(N(c4ccc5c(c4)oc4ccccc45)c4cccc5oc6cc7c(cc6c45)-c4ccccc4C74c5ccccc5Sc5ccccc54)cc32)cc1 ; c1ccc(C2(c3ccccc3)c3ccccc3-c3ccc(N(c4cccc5c4oc4ccccc45)c4cccc5oc6cc7c(cc6c45)-c4ccccc4C74c5ccccc5Sc5ccccc54)cc32)cc1]

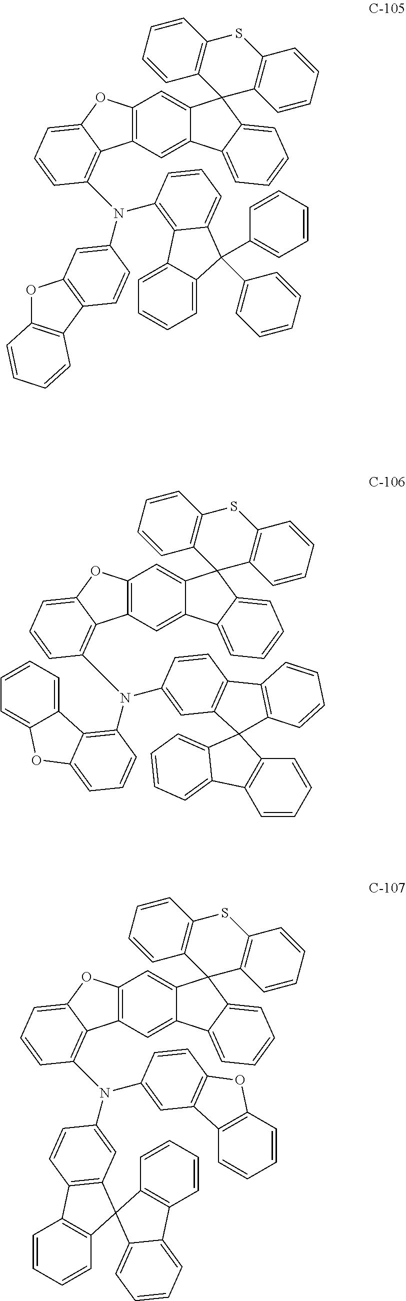 [SMILES: c1ccc(C2(c3ccccc3)c3ccccc3-c3c(N(c4ccc5c(c4)oc4ccccc45)c4cccc5oc6cc7c(cc6c45)-c4ccccc4C74c5ccccc5Sc5ccccc54)cccc32)cc1 ; c1ccc2c(c1)Sc1ccccc1C21c2ccccc2-c2cc3c(cc21)oc1cccc(N(c2ccc4c(c2)C2(c5ccccc5-c5ccccc52)c2ccccc2-4)c2cccc4oc5ccccc5c24)c13 ; c1ccc2c(c1)Sc1ccccc1C21c2ccccc2-c2cc3c(cc21)oc1cccc(N(c2ccc4c(c2)C2(c5ccccc5-c5ccccc52)c2ccccc2-4)c2ccc4oc5ccccc5c4c2)c13]

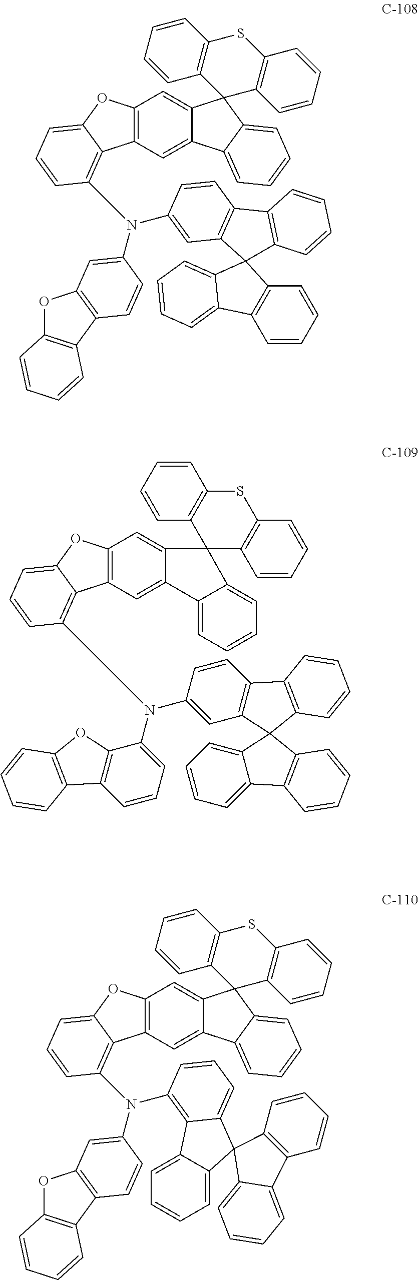 [SMILES: c1ccc2c(c1)Sc1ccccc1C21c2ccccc2-c2cc3c(cc21)oc1cccc(N(c2ccc4c(c2)C2(c5ccccc5-c5ccccc52)c2ccccc2-4)c2ccc4c(c2)oc2ccccc24)c13 ; c1ccc2c(c1)Sc1ccccc1C21c2ccccc2-c2cc3c(cc21)oc1cccc(N(c2ccc4c(c2)C2(c5ccccc5-c5ccccc52)c2ccccc2-4)c2cccc4c2oc2ccccc24)c13 ; c1ccc2c(c1)Sc1ccccc1C21c2ccccc2-c2cc3c(cc21)oc1cccc(N(c2ccc4c(c2)oc2ccccc24)c2cccc4c2-c2ccccc2C42c4ccccc4-c4ccccc42)c13]

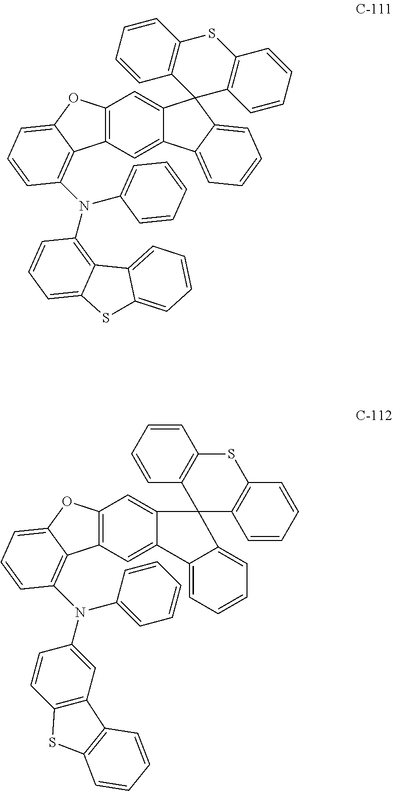 [SMILES: c1ccc(N(c2cccc3oc4cc5c(cc4c23)-c2ccccc2C52c3ccccc3Sc3ccccc32)c2cccc3sc4ccccc4c23)cc1 ; c1ccc(N(c2ccc3sc4ccccc4c3c2)c2cccc3oc4cc5c(cc4c23)-c2ccccc2C52c3ccccc3Sc3ccccc32)cc1]

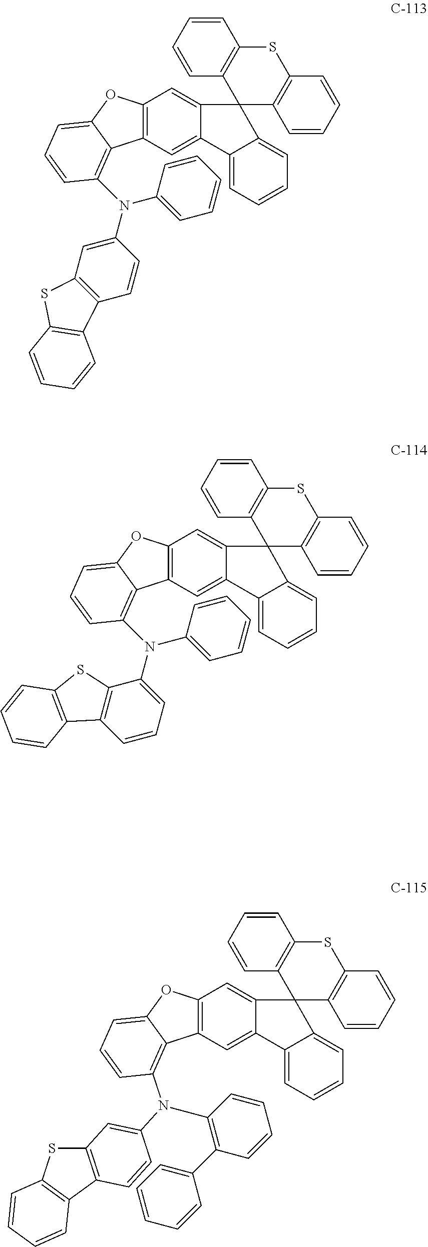 [SMILES: c1ccc(N(c2ccc3c(c2)sc2ccccc23)c2cccc3oc4cc5c(cc4c23)-c2ccccc2C52c3ccccc3Sc3ccccc32)cc1 ; c1ccc(N(c2cccc3c2sc2ccccc23)c2cccc3oc4cc5c(cc4c23)-c2ccccc2C52c3ccccc3Sc3ccccc32)cc1 ; c1ccc(-c2ccccc2N(c2ccc3c(c2)sc2ccccc23)c2cccc3oc4cc5c(cc4c23)-c2ccccc2C52c3ccccc3Sc3ccccc32)cc1]

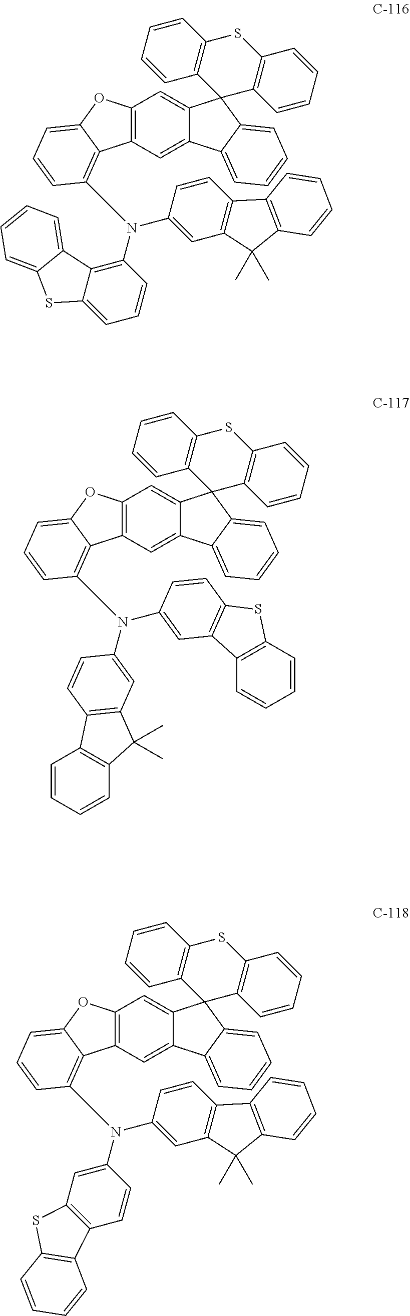 [SMILES: CC1(C)c2ccccc2-c2ccc(N(c3cccc4oc5cc6c(cc5c34)-c3ccccc3C63c4ccccc4Sc4ccccc43)c3cccc4sc5ccccc5c34)cc21 ; CC1(C)c2ccccc2-c2ccc(N(c3ccc4sc5ccccc5c4c3)c3cccc4oc5cc6c(cc5c34)-c3ccccc3C63c4ccccc4Sc4ccccc43)cc21 ; CC1(C)c2ccccc2-c2ccc(N(c3ccc4c(c3)sc3ccccc34)c3cccc4oc5cc6c(cc5c34)-c3ccccc3C63c4ccccc4Sc4ccccc43)cc21]

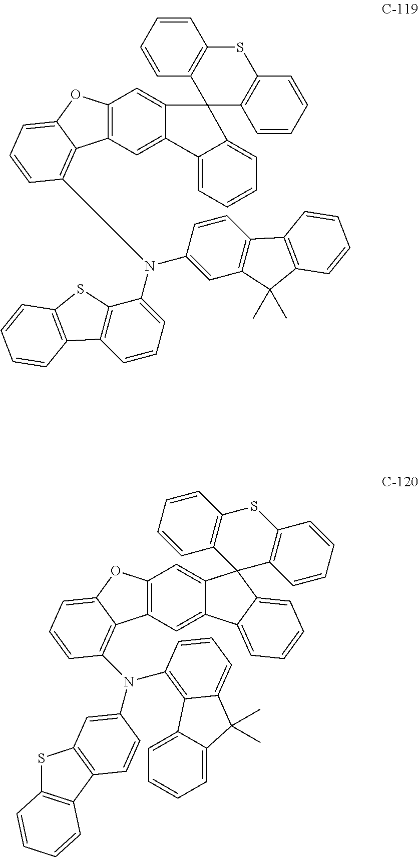 [SMILES: CC1(C)c2ccccc2-c2ccc(N(c3cccc4c3sc3ccccc34)c3cccc4oc5cc6c(cc5c34)-c3ccccc3C63c4ccccc4Sc4ccccc43)cc21 ; CC1(C)c2ccccc2-c2c(N(c3ccc4c(c3)sc3ccccc34)c3cccc4oc5cc6c(cc5c34)-c3ccccc3C63c4ccccc4Sc4ccccc43)cccc21]

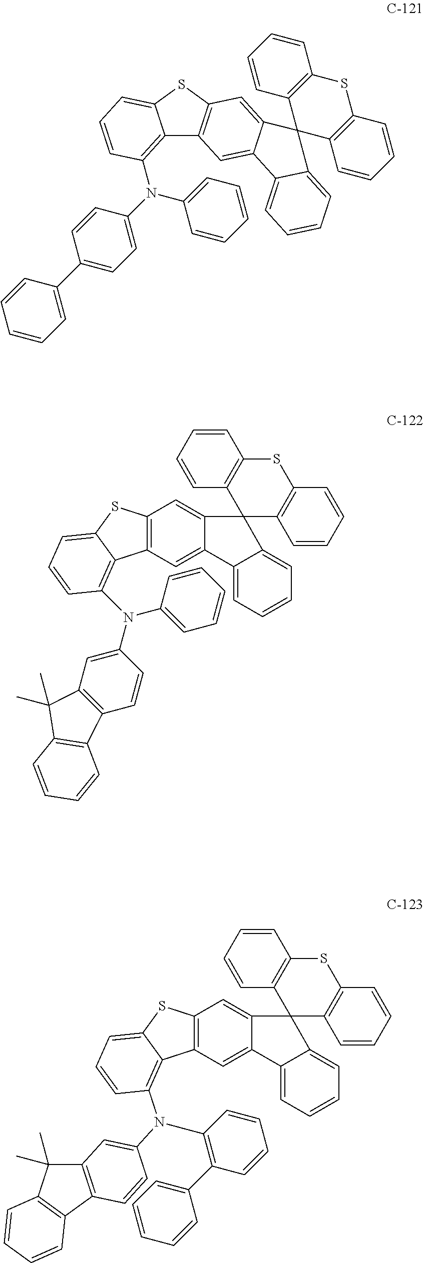 [SMILES: c1ccc(-c2ccc(N(c3ccccc3)c3cccc4sc5cc6c(cc5c34)-c3ccccc3C63c4ccccc4Sc4ccccc43)cc2)cc1 ; CC1(C)c2ccccc2-c2ccc(N(c3ccccc3)c3cccc4sc5cc6c(cc5c34)-c3ccccc3C63c4ccccc4Sc4ccccc43)cc21 ; CC1(C)c2ccccc2-c2ccc(N(c3ccccc3-c3ccccc3)c3cccc4sc5cc6c(cc5c34)-c3ccccc3C63c4ccccc4Sc4ccccc43)cc21]

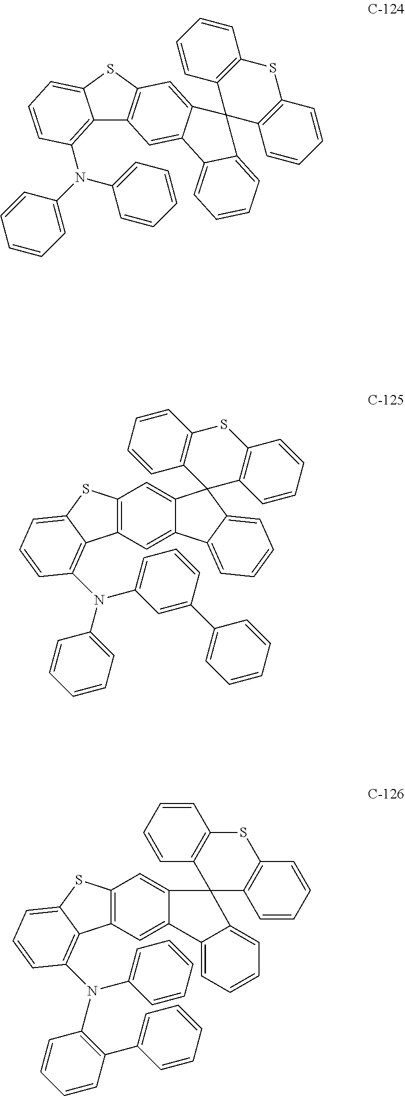 [SMILES: c1ccc(N(c2ccccc2)c2cccc3sc4cc5c(cc4c23)-c2ccccc2C52c3ccccc3Sc3ccccc32)cc1 ; c1ccc(-c2cccc(N(c3ccccc3)c3cccc4sc5cc6c(cc5c34)-c3ccccc3C63c4ccccc4Sc4ccccc43)c2)cc1 ; c1ccc(-c2ccccc2N(c2ccccc2)c2cccc3sc4cc5c(cc4c23)-c2ccccc2C52c3ccccc3Sc3ccccc32)cc1]

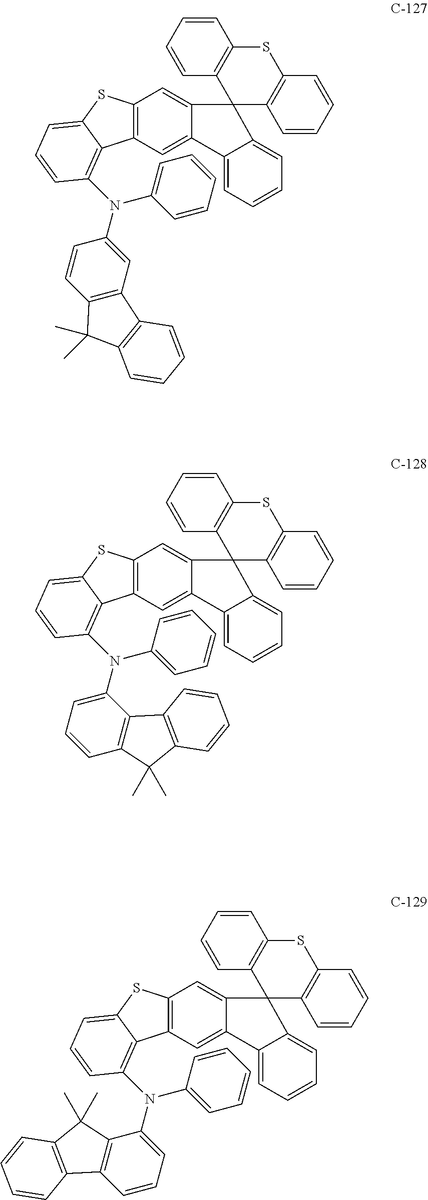 [SMILES: CC1(C)c2ccccc2-c2cc(N(c3ccccc3)c3cccc4sc5cc6c(cc5c34)-c3ccccc3C63c4ccccc4Sc4ccccc43)ccc21 ; CC1(C)c2ccccc2-c2c(N(c3ccccc3)c3cccc4sc5cc6c(cc5c34)-c3ccccc3C63c4ccccc4Sc4ccccc43)cccc21 ; CC1(C)c2ccccc2-c2cccc(N(c3ccccc3)c3cccc4sc5cc6c(cc5c34)-c3ccccc3C63c4ccccc4Sc4ccccc43)c21]

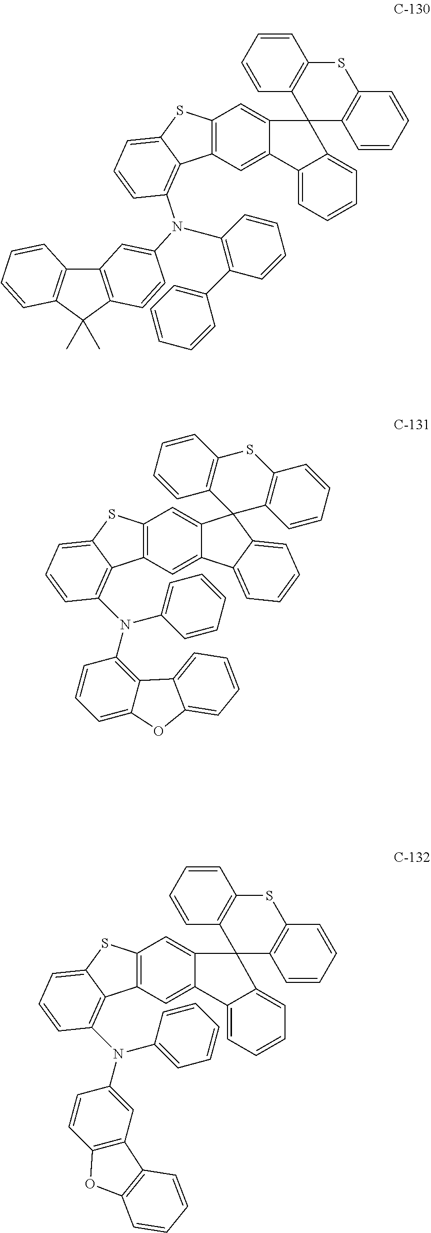 [SMILES: CC1(C)c2ccccc2-c2cc(N(c3ccccc3-c3ccccc3)c3cccc4sc5cc6c(cc5c34)-c3ccccc3C63c4ccccc4Sc4ccccc43)ccc21 ; c1ccc(N(c2cccc3oc4ccccc4c23)c2cccc3sc4cc5c(cc4c23)-c2ccccc2C52c3ccccc3Sc3ccccc32)cc1 ; c1ccc(N(c2ccc3oc4ccccc4c3c2)c2cccc3sc4cc5c(cc4c23)-c2ccccc2C52c3ccccc3Sc3ccccc32)cc1]

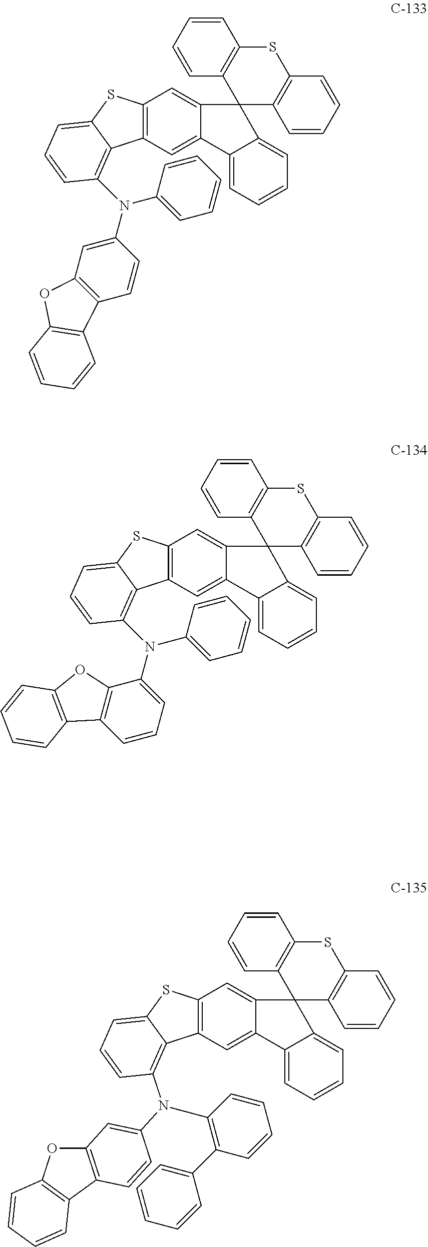 [SMILES: c1ccc(N(c2ccc3c(c2)oc2ccccc23)c2cccc3sc4cc5c(cc4c23)-c2ccccc2C52c3ccccc3Sc3ccccc32)cc1 ; c1ccc(N(c2cccc3c2oc2ccccc23)c2cccc3sc4cc5c(cc4c23)-c2ccccc2C52c3ccccc3Sc3ccccc32)cc1 ; c1ccc(-c2ccccc2N(c2ccc3c(c2)oc2ccccc23)c2cccc3sc4cc5c(cc4c23)-c2ccccc2C52c3ccccc3Sc3ccccc32)cc1]

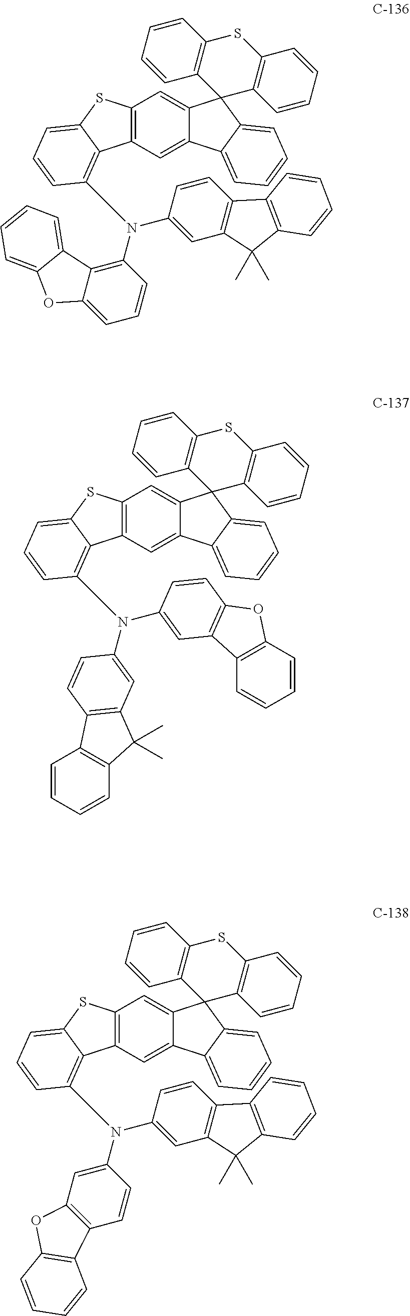 [SMILES: CC1(C)c2ccccc2-c2ccc(N(c3cccc4oc5ccccc5c34)c3cccc4sc5cc6c(cc5c34)-c3ccccc3C63c4ccccc4Sc4ccccc43)cc21 ; CC1(C)c2ccccc2-c2ccc(N(c3ccc4oc5ccccc5c4c3)c3cccc4sc5cc6c(cc5c34)-c3ccccc3C63c4ccccc4Sc4ccccc43)cc21 ; CC1(C)c2ccccc2-c2ccc(N(c3ccc4c(c3)oc3ccccc34)c3cccc4sc5cc6c(cc5c34)-c3ccccc3C63c4ccccc4Sc4ccccc43)cc21]

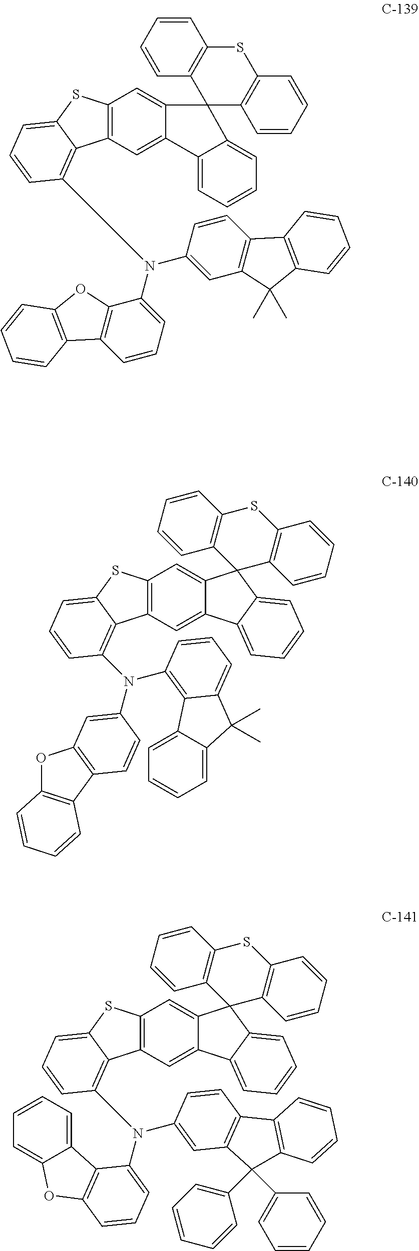 [SMILES: CC1(C)c2ccccc2-c2ccc(N(c3cccc4c3oc3ccccc34)c3cccc4sc5cc6c(cc5c34)-c3ccccc3C63c4ccccc4Sc4ccccc43)cc21 ; CC1(C)c2ccccc2-c2c(N(c3ccc4c(c3)oc3ccccc34)c3cccc4sc5cc6c(cc5c34)-c3ccccc3C63c4ccccc4Sc4ccccc43)cccc21 ; c1ccc(C2(c3ccccc3)c3ccccc3-c3ccc(N(c4cccc5oc6ccccc6c45)c4cccc5sc6cc7c(cc6c45)-c4ccccc4C74c5ccccc5Sc5ccccc54)cc32)cc1]

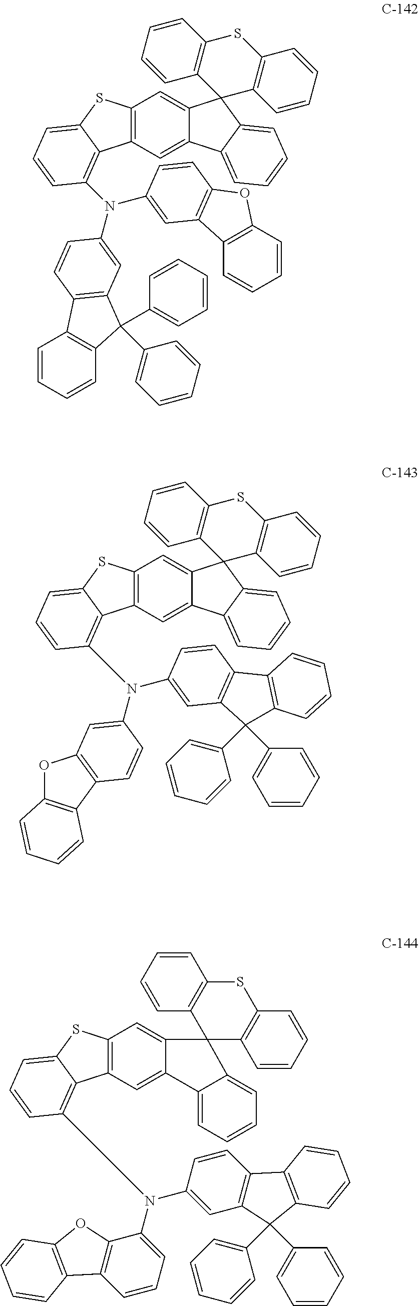 [SMILES: c1ccc(C2(c3ccccc3)c3ccccc3-c3ccc(N(c4ccc5oc6ccccc6c5c4)c4cccc5sc6cc7c(cc6c45)-c4ccccc4C74c5ccccc5Sc5ccccc54)cc32)cc1 ; c1ccc(C2(c3ccccc3)c3ccccc3-c3ccc(N(c4ccc5c(c4)oc4ccccc45)c4cccc5sc6cc7c(cc6c45)-c4ccccc4C74c5ccccc5Sc5ccccc54)cc32)cc1 ; c1ccc(C2(c3ccccc3)c3ccccc3-c3ccc(N(c4cccc5c4oc4ccccc45)c4cccc5sc6cc7c(cc6c45)-c4ccccc4C74c5ccccc5Sc5ccccc54)cc32)cc1]

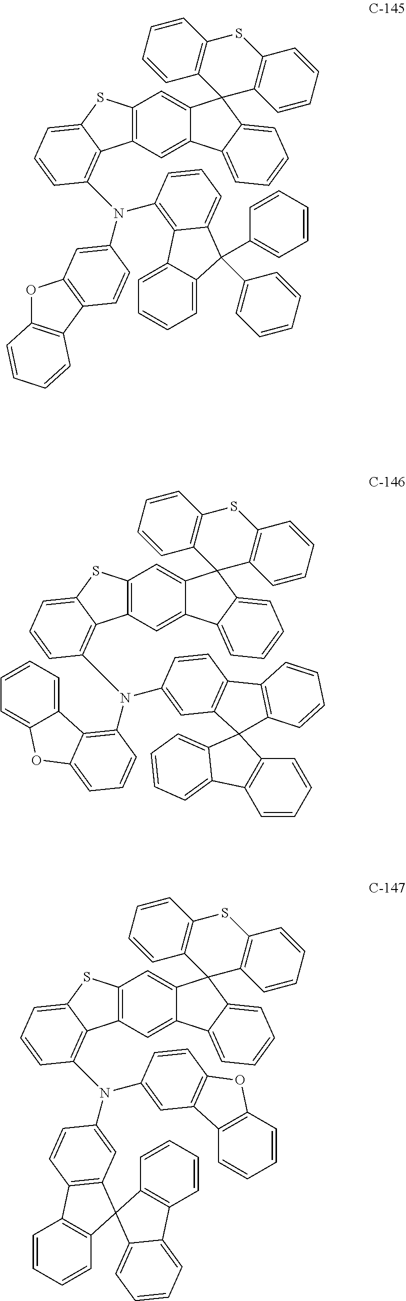 [SMILES: c1ccc(C2(c3ccccc3)c3ccccc3-c3c(N(c4ccc5c(c4)oc4ccccc45)c4cccc5sc6cc7c(cc6c45)-c4ccccc4C74c5ccccc5Sc5ccccc54)cccc32)cc1 ; c1ccc2c(c1)Sc1ccccc1C21c2ccccc2-c2cc3c(cc21)sc1cccc(N(c2ccc4c(c2)C2(c5ccccc5-c5ccccc52)c2ccccc2-4)c2cccc4oc5ccccc5c24)c13 ; c1ccc2c(c1)Sc1ccccc1C21c2ccccc2-c2cc3c(cc21)sc1cccc(N(c2ccc4c(c2)C2(c5ccccc5-c5ccccc52)c2ccccc2-4)c2ccc4oc5ccccc5c4c2)c13]

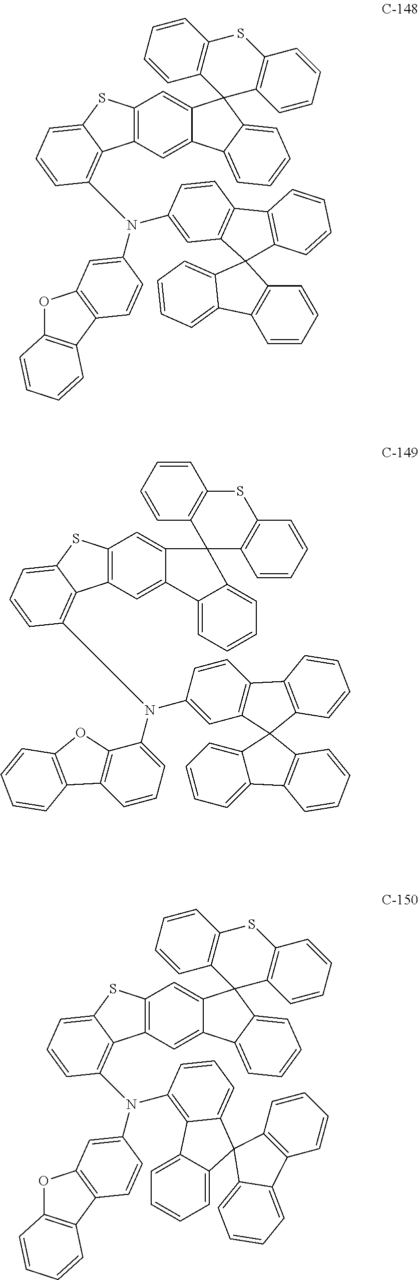 [SMILES: c1ccc2c(c1)Sc1ccccc1C21c2ccccc2-c2cc3c(cc21)sc1cccc(N(c2ccc4c(c2)C2(c5ccccc5-c5ccccc52)c2ccccc2-4)c2ccc4c(c2)oc2ccccc24)c13 ; c1ccc2c(c1)Sc1ccccc1C21c2ccccc2-c2cc3c(cc21)sc1cccc(N(c2ccc4c(c2)C2(c5ccccc5-c5ccccc52)c2ccccc2-4)c2cccc4c2oc2ccccc24)c13 ; c1ccc2c(c1)Sc1ccccc1C21c2ccccc2-c2cc3c(cc21)sc1cccc(N(c2ccc4c(c2)oc2ccccc24)c2cccc4c2-c2ccccc2C42c4ccccc4-c4ccccc42)c13]

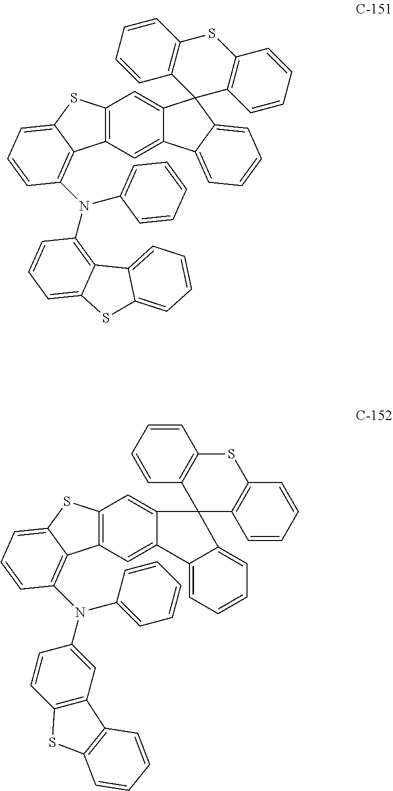 [SMILES: c1ccc(N(c2cccc3sc4ccccc4c23)c2cccc3sc4cc5c(cc4c23)-c2ccccc2C52c3ccccc3Sc3ccccc32)cc1 ; c1ccc(N(c2ccc3sc4ccccc4c3c2)c2cccc3sc4cc5c(cc4c23)-c2ccccc2C52c3ccccc3Sc3ccccc32)cc1]

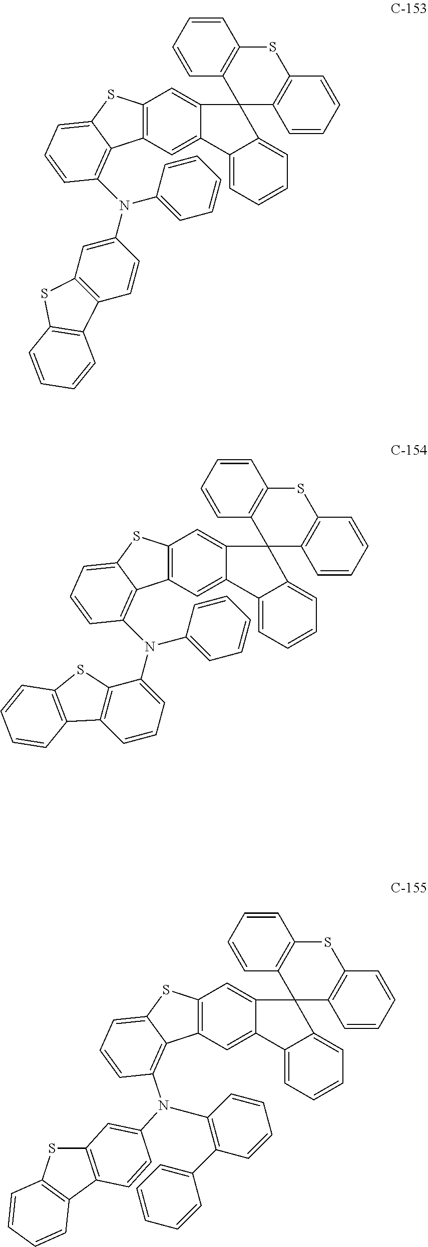 [SMILES: c1ccc(N(c2ccc3c(c2)sc2ccccc23)c2cccc3sc4cc5c(cc4c23)-c2ccccc2C52c3ccccc3Sc3ccccc32)cc1 ; c1ccc(N(c2cccc3c2sc2ccccc23)c2cccc3sc4cc5c(cc4c23)-c2ccccc2C52c3ccccc3Sc3ccccc32)cc1 ; c1ccc(-c2ccccc2N(c2ccc3c(c2)sc2ccccc23)c2cccc3sc4cc5c(cc4c23)-c2ccccc2C52c3ccccc3Sc3ccccc32)cc1]

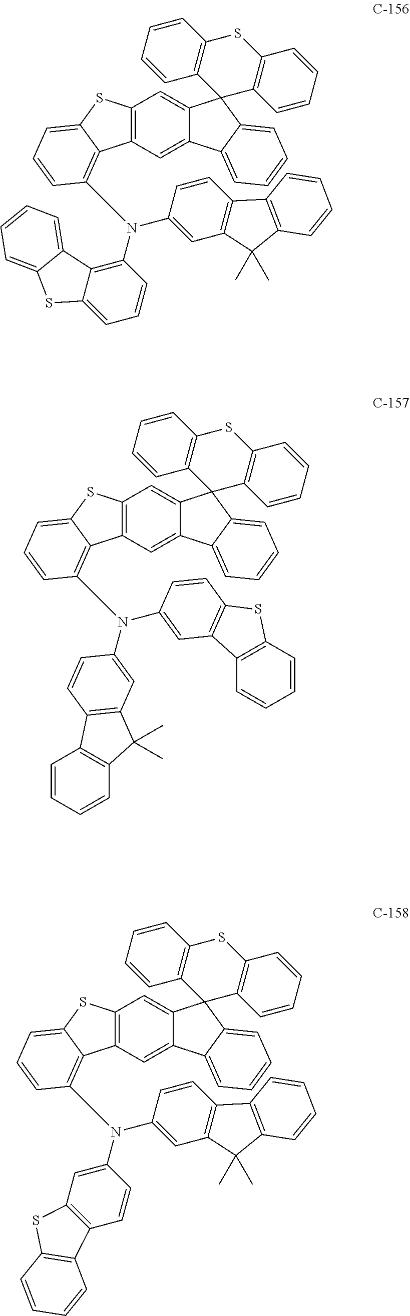 [SMILES: CC1(C)c2ccccc2-c2ccc(N(c3cccc4sc5ccccc5c34)c3cccc4sc5cc6c(cc5c34)-c3ccccc3C63c4ccccc4Sc4ccccc43)cc21 ; CC1(C)c2ccccc2-c2ccc(N(c3ccc4sc5ccccc5c4c3)c3cccc4sc5cc6c(cc5c34)-c3ccccc3C63c4ccccc4Sc4ccccc43)cc21 ; CC1(C)c2ccccc2-c2ccc(N(c3ccc4c(c3)sc3ccccc34)c3cccc4sc5cc6c(cc5c34)-c3ccccc3C63c4ccccc4Sc4ccccc43)cc21]

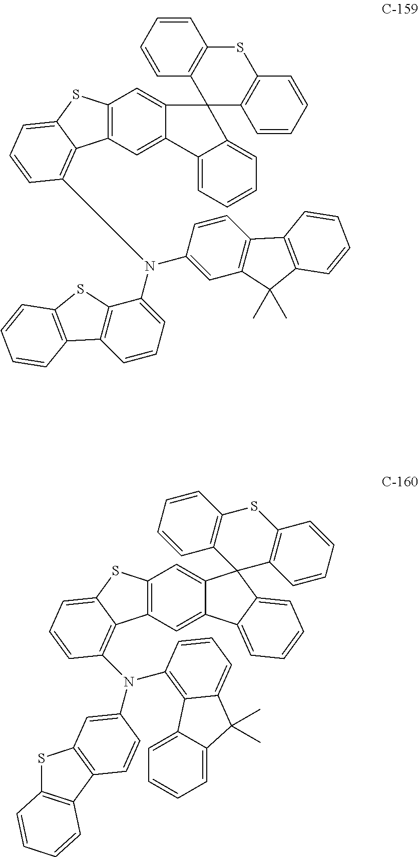 [SMILES: CC1(C)c2ccccc2-c2ccc(N(c3cccc4c3sc3ccccc34)c3cccc4sc5cc6c(cc5c34)-c3ccccc3C63c4ccccc4Sc4ccccc43)cc21 ; CC1(C)c2ccccc2-c2c(N(c3ccc4c(c3)sc3ccccc34)c3cccc4sc5cc6c(cc5c34)-c3ccccc3C63c4ccccc4Sc4ccccc43)cccc21]

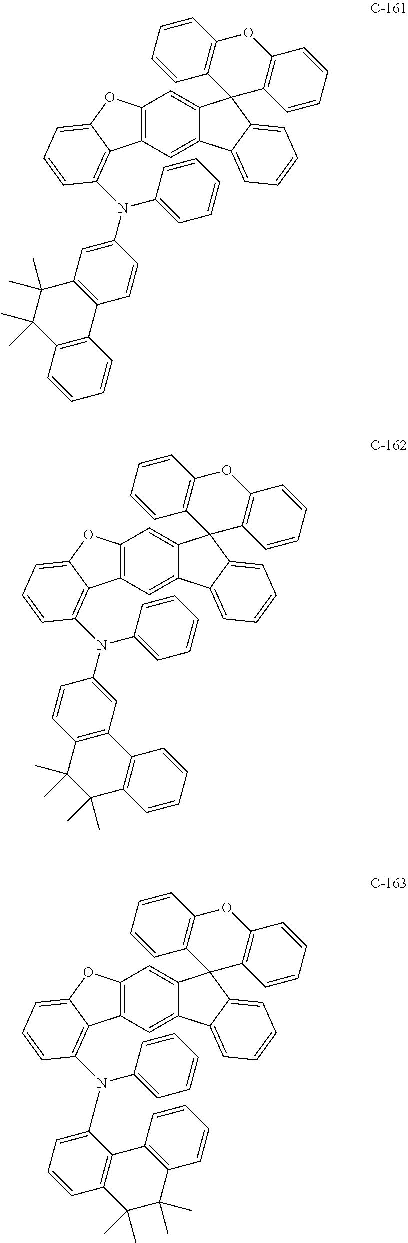 [SMILES: CC1(C)c2ccccc2-c2ccc(N(c3ccccc3)c3cccc4oc5cc6c(cc5c34)-c3ccccc3C63c4ccccc4Oc4ccccc43)cc2C1(C)C ; CC1(C)c2ccccc2-c2cc(N(c3ccccc3)c3cccc4oc5cc6c(cc5c34)-c3ccccc3C63c4ccccc4Oc4ccccc43)ccc2C1(C)C ; CC1(C)c2ccccc2-c2c(N(c3ccccc3)c3cccc4oc5cc6c(cc5c34)-c3ccccc3C63c4ccccc4Oc4ccccc43)cccc2C1(C)C]

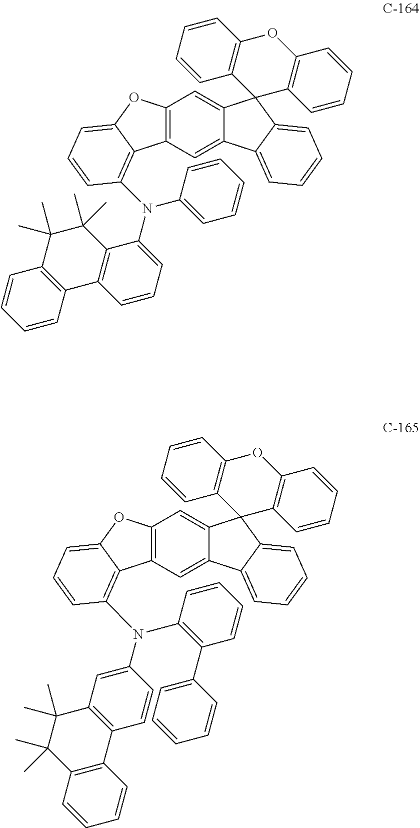 [SMILES: CC1(C)c2ccccc2-c2cccc(N(c3ccccc3)c3cccc4oc5cc6c(cc5c34)-c3ccccc3C63c4ccccc4Oc4ccccc43)c2C1(C)C ; CC1(C)c2ccccc2-c2ccc(N(c3ccccc3-c3ccccc3)c3cccc4oc5cc6c(cc5c34)-c3ccccc3C63c4ccccc4Oc4ccccc43)cc2C1(C)C]

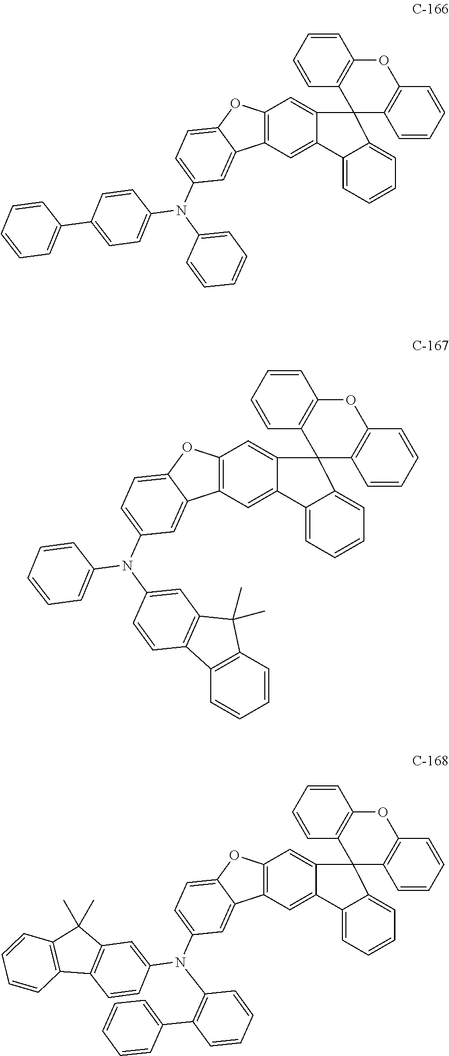 [SMILES: c1ccc(-c2ccc(N(c3ccccc3)c3ccc4oc5cc6c(cc5c4c3)-c3ccccc3C63c4ccccc4Oc4ccccc43)cc2)cc1 ; CC1(C)c2ccccc2-c2ccc(N(c3ccccc3)c3ccc4oc5cc6c(cc5c4c3)-c3ccccc3C63c4ccccc4Oc4ccccc43)cc21 ; CC1(C)c2ccccc2-c2ccc(N(c3ccc4oc5cc6c(cc5c4c3)-c3ccccc3C63c4ccccc4Oc4ccccc43)c3ccccc3-c3ccccc3)cc21]

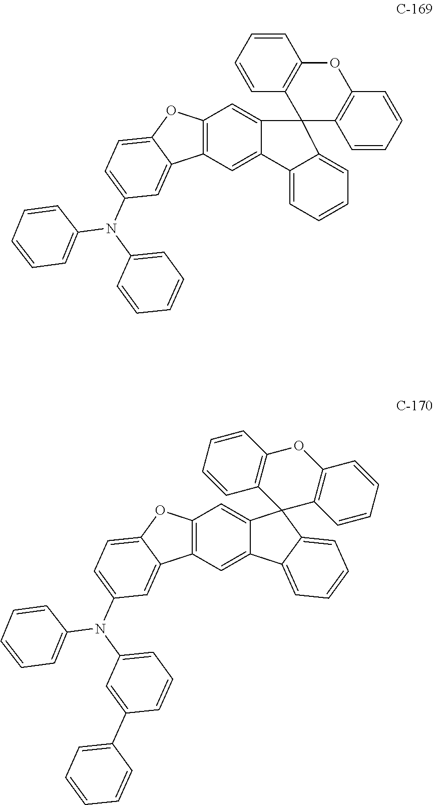 [SMILES: c1ccc(N(c2ccccc2)c2ccc3oc4cc5c(cc4c3c2)-c2ccccc2C52c3ccccc3Oc3ccccc32)cc1 ; c1ccc(-c2cccc(N(c3ccccc3)c3ccc4oc5cc6c(cc5c4c3)-c3ccccc3C63c4ccccc4Oc4ccccc43)c2)cc1]

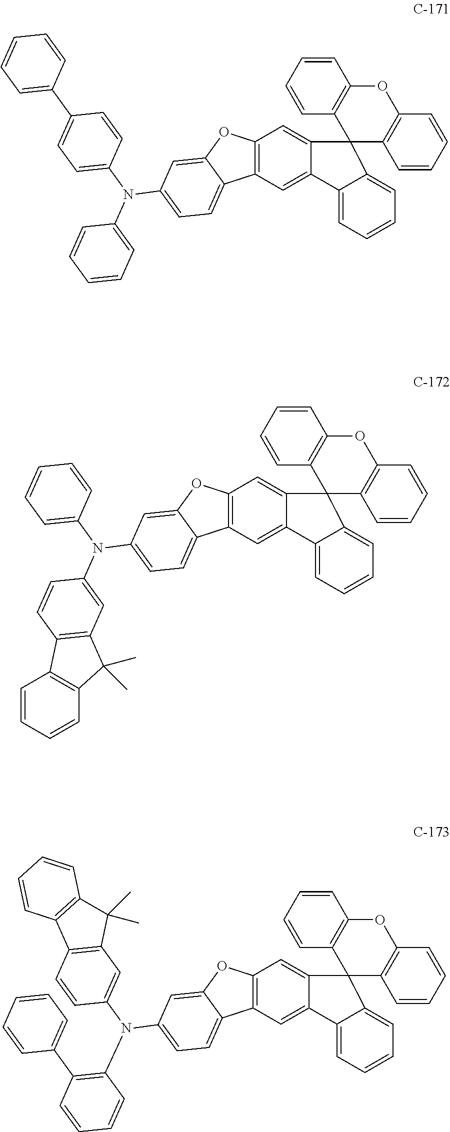 [SMILES: c1ccc(-c2ccc(N(c3ccccc3)c3ccc4c(c3)oc3cc5c(cc34)-c3ccccc3C53c4ccccc4Oc4ccccc43)cc2)cc1 ; CC1(C)c2ccccc2-c2ccc(N(c3ccccc3)c3ccc4c(c3)oc3cc5c(cc34)-c3ccccc3C53c4ccccc4Oc4ccccc43)cc21 ; CC1(C)c2ccccc2-c2ccc(N(c3ccc4c(c3)oc3cc5c(cc34)-c3ccccc3C53c4ccccc4Oc4ccccc43)c3ccccc3-c3ccccc3)cc21]

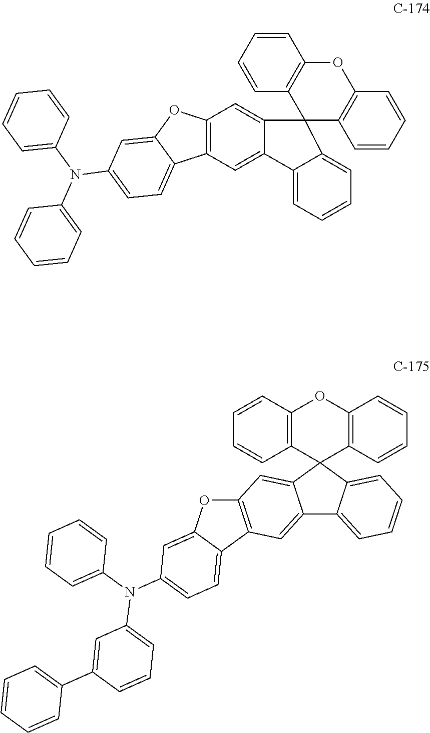 [SMILES: c1ccc(N(c2ccccc2)c2ccc3c(c2)oc2cc4c(cc23)-c2ccccc2C42c3ccccc3Oc3ccccc32)cc1 ; c1ccc(-c2cccc(N(c3ccccc3)c3ccc4c(c3)oc3cc5c(cc34)-c3ccccc3C53c4ccccc4Oc4ccccc43)c2)cc1]

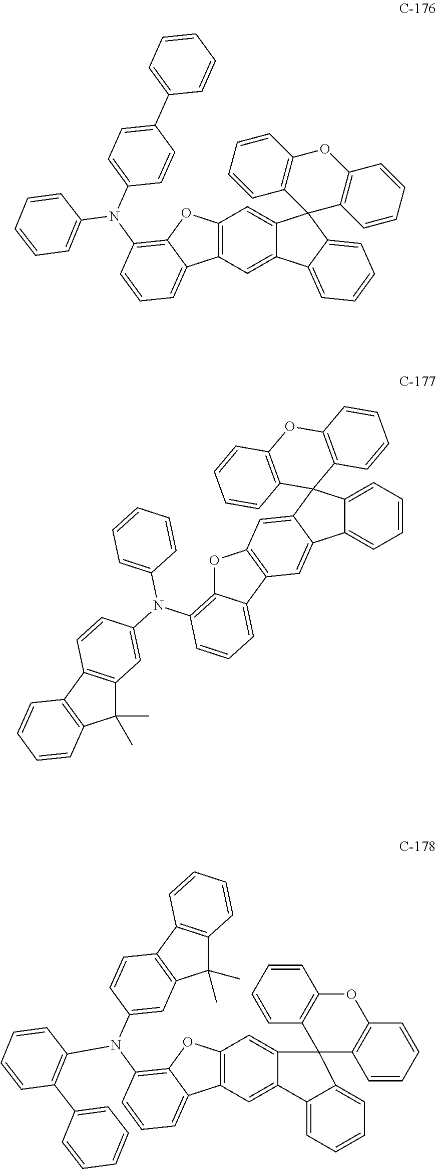 [SMILES: c1ccc(-c2ccc(N(c3ccccc3)c3cccc4c3oc3cc5c(cc34)-c3ccccc3C53c4ccccc4Oc4ccccc43)cc2)cc1 ; CC1(C)c2ccccc2-c2ccc(N(c3ccccc3)c3cccc4c3oc3cc5c(cc34)-c3ccccc3C53c4ccccc4Oc4ccccc43)cc21 ; CC1(C)c2ccccc2-c2ccc(N(c3ccccc3-c3ccccc3)c3cccc4c3oc3cc5c(cc34)-c3ccccc3C53c4ccccc4Oc4ccccc43)cc21]

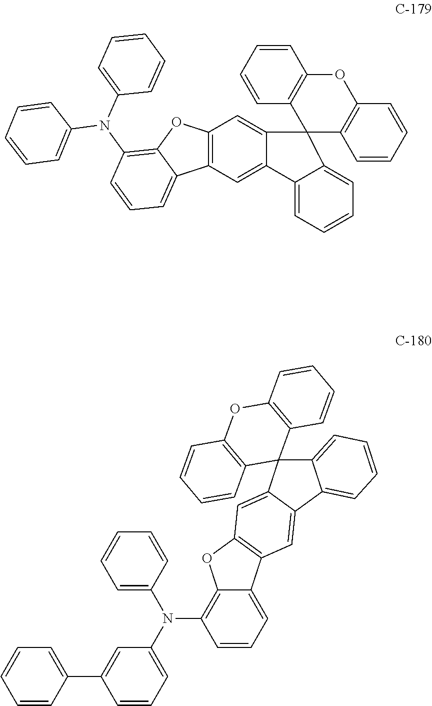 [SMILES: c1ccc(N(c2ccccc2)c2cccc3c2oc2cc4c(cc23)-c2ccccc2C42c3ccccc3Oc3ccccc32)cc1 ; c1ccc(-c2cccc(N(c3ccccc3)c3cccc4c3oc3cc5c(cc34)-c3ccccc3C53c4ccccc4Oc4ccccc43)c2)cc1]

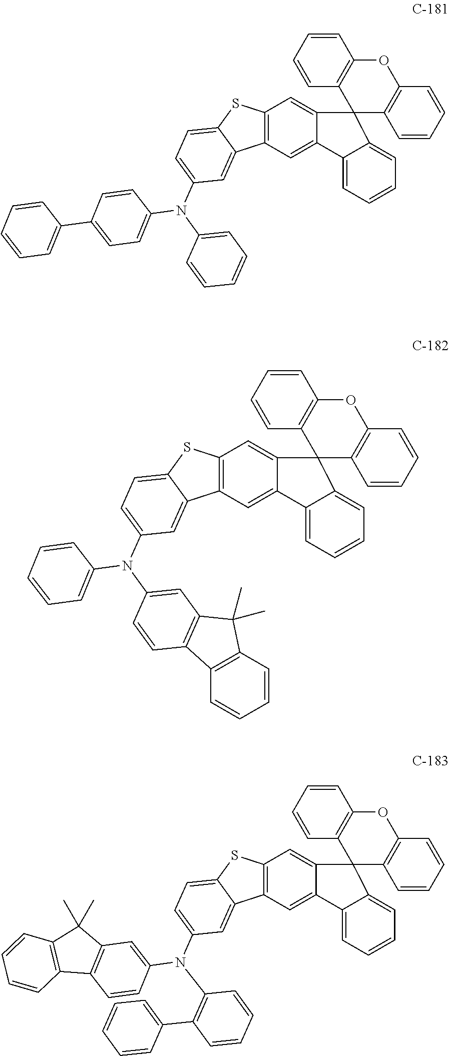 [SMILES: c1ccc(-c2ccc(N(c3ccccc3)c3ccc4sc5cc6c(cc5c4c3)-c3ccccc3C63c4ccccc4Oc4ccccc43)cc2)cc1 ; CC1(C)c2ccccc2-c2ccc(N(c3ccccc3)c3ccc4sc5cc6c(cc5c4c3)-c3ccccc3C63c4ccccc4Oc4ccccc43)cc21 ; CC1(C)c2ccccc2-c2ccc(N(c3ccc4sc5cc6c(cc5c4c3)-c3ccccc3C63c4ccccc4Oc4ccccc43)c3ccccc3-c3ccccc3)cc21]

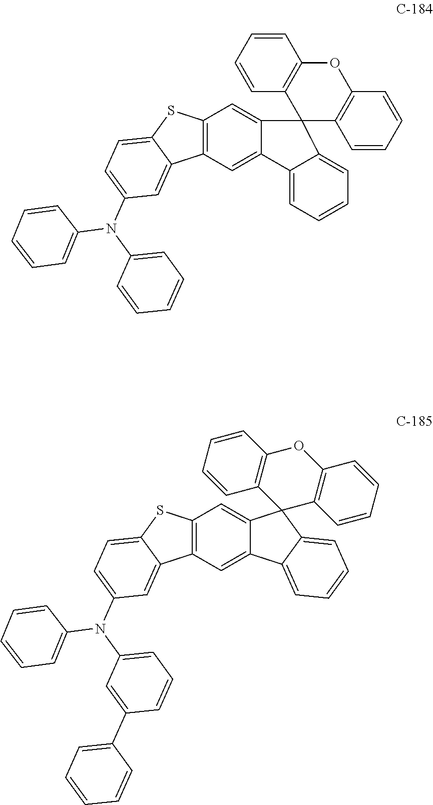 [SMILES: c1ccc(N(c2ccccc2)c2ccc3sc4cc5c(cc4c3c2)-c2ccccc2C52c3ccccc3Oc3ccccc32)cc1 ; c1ccc(-c2cccc(N(c3ccccc3)c3ccc4sc5cc6c(cc5c4c3)-c3ccccc3C63c4ccccc4Oc4ccccc43)c2)cc1]

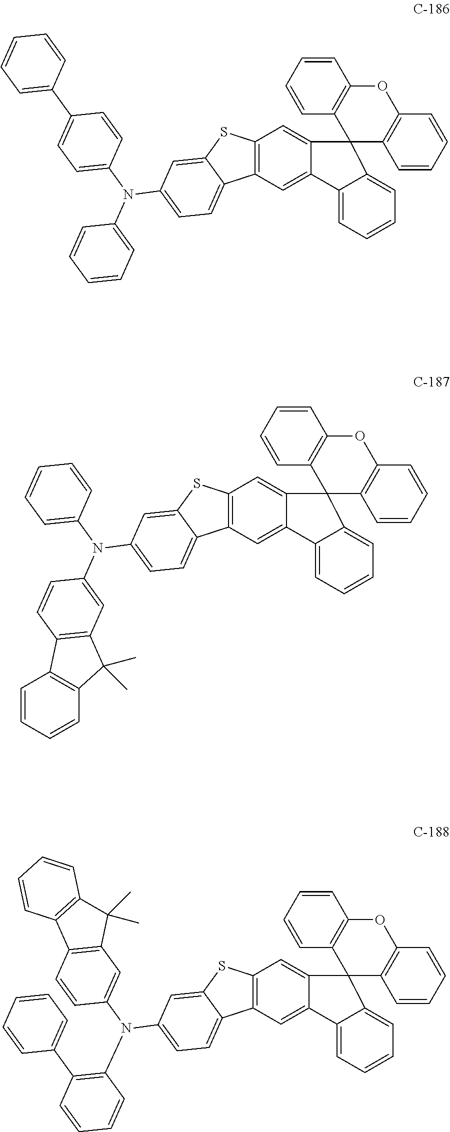 [SMILES: c1ccc(-c2ccc(N(c3ccccc3)c3ccc4c(c3)sc3cc5c(cc34)-c3ccccc3C53c4ccccc4Oc4ccccc43)cc2)cc1 ; CC1(C)c2ccccc2-c2ccc(N(c3ccccc3)c3ccc4c(c3)sc3cc5c(cc34)-c3ccccc3C53c4ccccc4Oc4ccccc43)cc21 ; CC1(C)c2ccccc2-c2ccc(N(c3ccc4c(c3)sc3cc5c(cc34)-c3ccccc3C53c4ccccc4Oc4ccccc43)c3ccccc3-c3ccccc3)cc21]

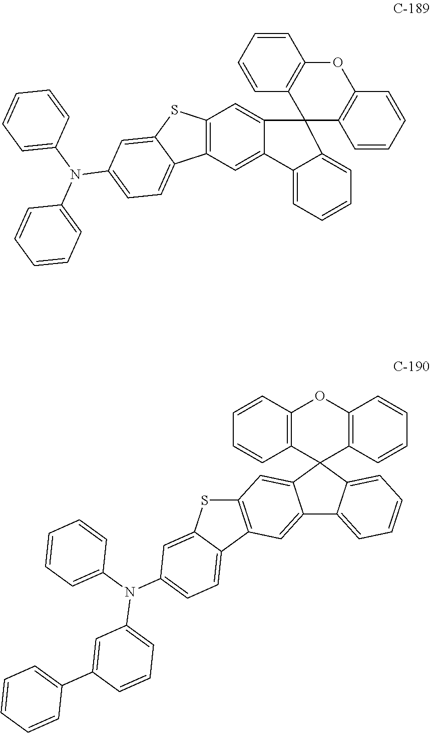 [SMILES: c1ccc(N(c2ccccc2)c2ccc3c(c2)sc2cc4c(cc23)-c2ccccc2C42c3ccccc3Oc3ccccc32)cc1 ; c1ccc(-c2cccc(N(c3ccccc3)c3ccc4c(c3)sc3cc5c(cc34)-c3ccccc3C53c4ccccc4Oc4ccccc43)c2)cc1]

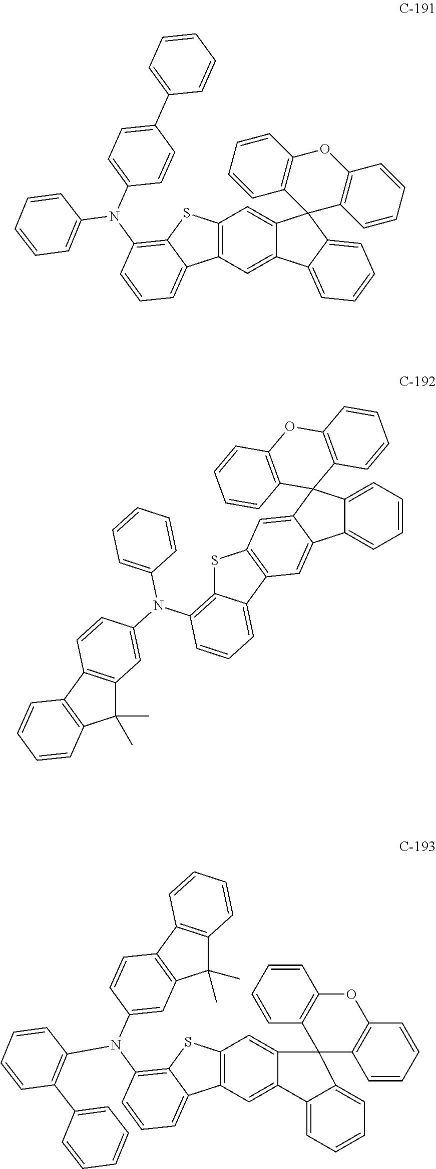 [SMILES: c1ccc(-c2ccc(N(c3ccccc3)c3cccc4c3sc3cc5c(cc34)-c3ccccc3C53c4ccccc4Oc4ccccc43)cc2)cc1 ; CC1(C)c2ccccc2-c2ccc(N(c3ccccc3)c3cccc4c3sc3cc5c(cc34)-c3ccccc3C53c4ccccc4Oc4ccccc43)cc21 ; CC1(C)c2ccccc2-c2ccc(N(c3ccccc3-c3ccccc3)c3cccc4c3sc3cc5c(cc34)-c3ccccc3C53c4ccccc4Oc4ccccc43)cc21]

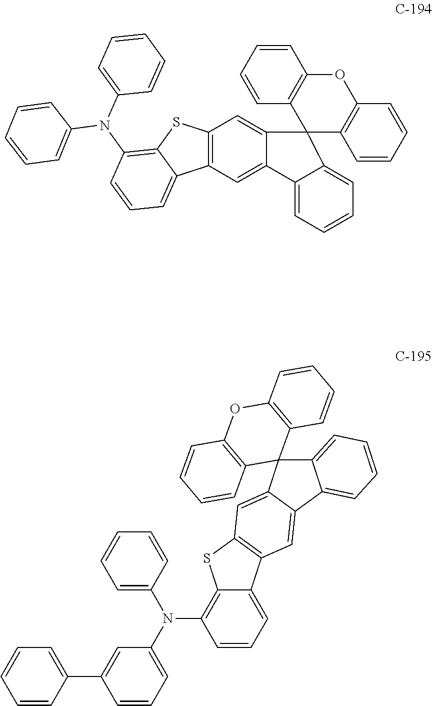 [SMILES: c1ccc(N(c2ccccc2)c2cccc3c2sc2cc4c(cc23)-c2ccccc2C42c3ccccc3Oc3ccccc32)cc1 ; c1ccc(-c2cccc(N(c3ccccc3)c3cccc4c3sc3cc5c(cc34)-c3ccccc3C53c4ccccc4Oc4ccccc43)c2)cc1]

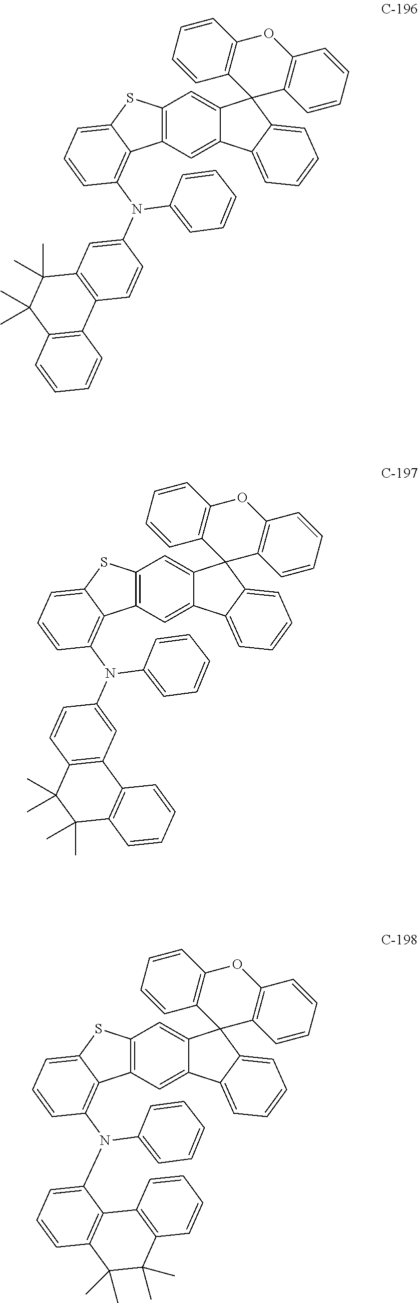 [SMILES: CC1(C)c2ccccc2-c2ccc(N(c3ccccc3)c3cccc4sc5cc6c(cc5c34)-c3ccccc3C63c4ccccc4Oc4ccccc43)cc2C1(C)C ; CC1(C)c2ccccc2-c2cc(N(c3ccccc3)c3cccc4sc5cc6c(cc5c34)-c3ccccc3C63c4ccccc4Oc4ccccc43)ccc2C1(C)C ; CC1(C)c2ccccc2-c2c(N(c3ccccc3)c3cccc4sc5cc6c(cc5c34)-c3ccccc3C63c4ccccc4Oc4ccccc43)cccc2C1(C)C]

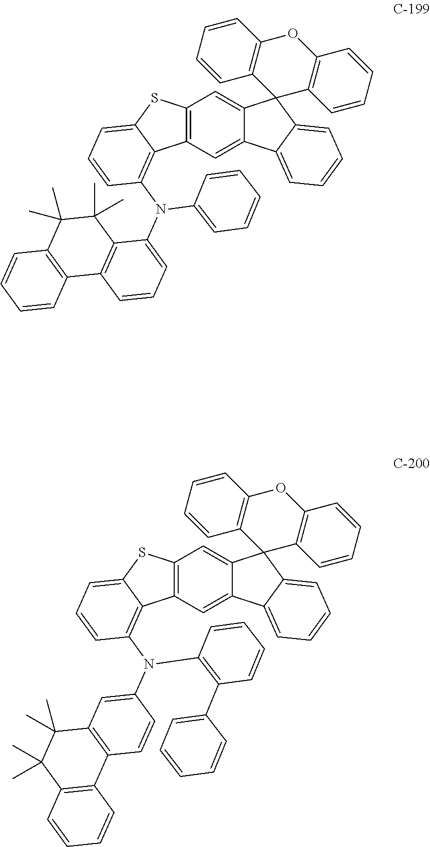 [SMILES: CC1(C)c2ccccc2-c2cccc(N(c3ccccc3)c3cccc4sc5cc6c(cc5c34)-c3ccccc3C63c4ccccc4Oc4ccccc43)c2C1(C)C ; CC1(C)c2ccccc2-c2ccc(N(c3ccccc3-c3ccccc3)c3cccc4sc5cc6c(cc5c34)-c3ccccc3C63c4ccccc4Oc4ccccc43)cc2C1(C)C]

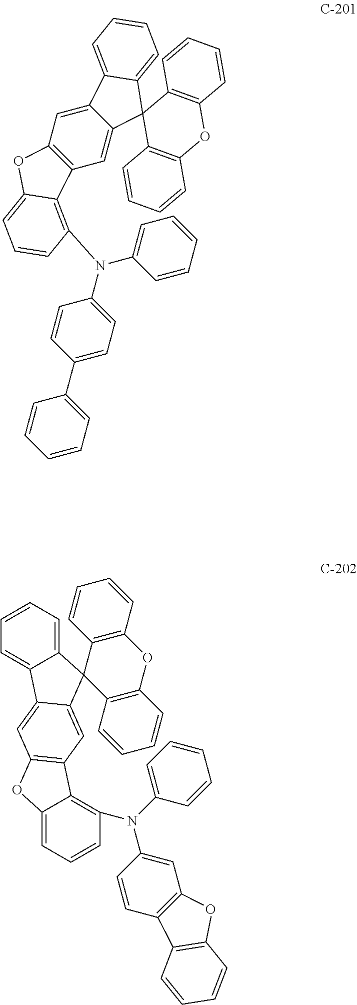 [SMILES: c1ccc(-c2ccc(N(c3ccccc3)c3cccc4oc5cc6c(cc5c34)C3(c4ccccc4Oc4ccccc43)c3ccccc3-6)cc2)cc1 ; c1ccc(N(c2ccc3c(c2)oc2ccccc23)c2cccc3oc4cc5c(cc4c23)C2(c3ccccc3Oc3ccccc32)c2ccccc2-5)cc1]

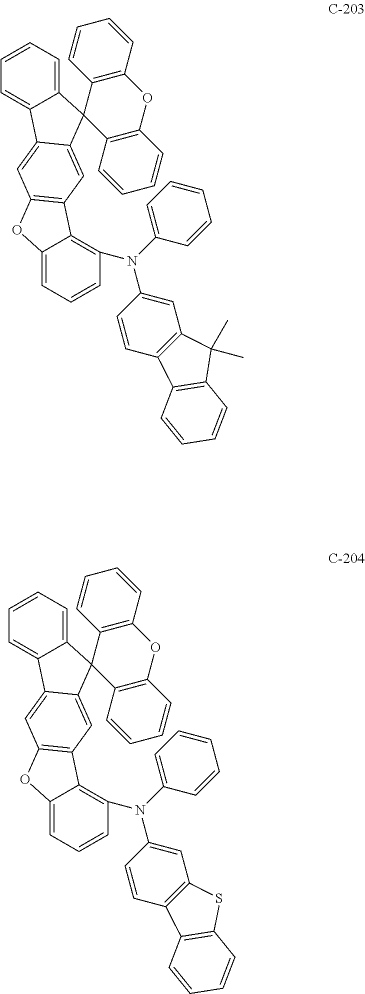 [SMILES: CC1(C)c2ccccc2-c2ccc(N(c3ccccc3)c3cccc4oc5cc6c(cc5c34)C3(c4ccccc4Oc4ccccc43)c3ccccc3-6)cc21 ; c1ccc(N(c2ccc3c(c2)sc2ccccc23)c2cccc3oc4cc5c(cc4c23)C2(c3ccccc3Oc3ccccc32)c2ccccc2-5)cc1]

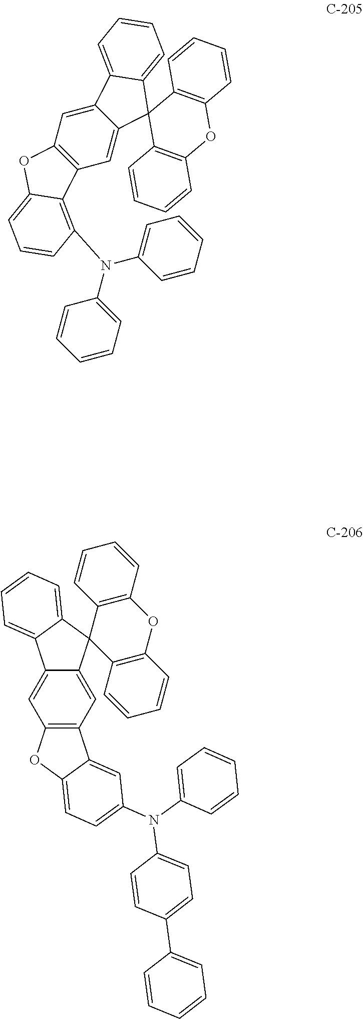 [SMILES: c1ccc(N(c2ccccc2)c2cccc3oc4cc5c(cc4c23)C2(c3ccccc3Oc3ccccc32)c2ccccc2-5)cc1 ; c1ccc(-c2ccc(N(c3ccccc3)c3ccc4oc5cc6c(cc5c4c3)C3(c4ccccc4Oc4ccccc43)c3ccccc3-6)cc2)cc1]

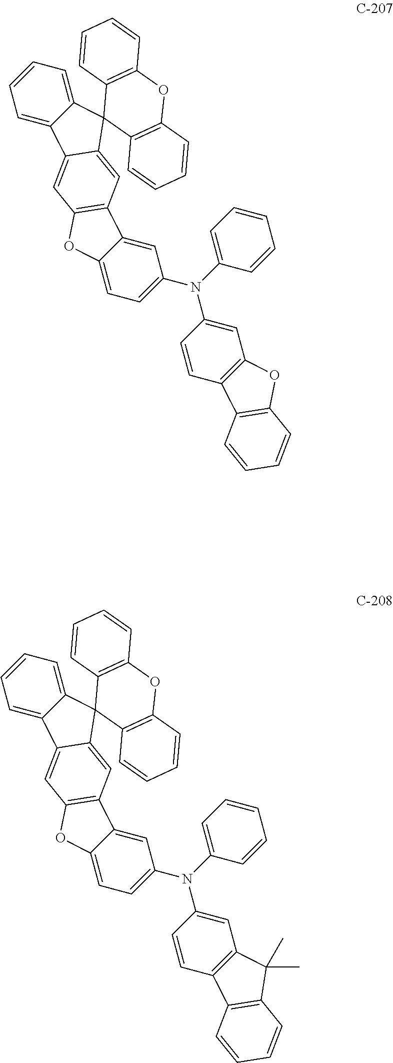 [SMILES: c1ccc(N(c2ccc3c(c2)oc2ccccc23)c2ccc3oc4cc5c(cc4c3c2)C2(c3ccccc3Oc3ccccc32)c2ccccc2-5)cc1 ; CC1(C)c2ccccc2-c2ccc(N(c3ccccc3)c3ccc4oc5cc6c(cc5c4c3)C3(c4ccccc4Oc4ccccc43)c3ccccc3-6)cc21]

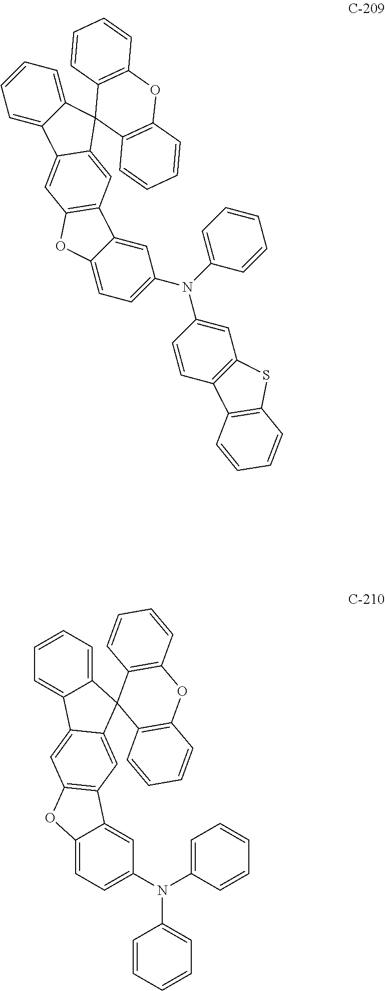 [SMILES: c1ccc(N(c2ccc3c(c2)sc2ccccc23)c2ccc3oc4cc5c(cc4c3c2)C2(c3ccccc3Oc3ccccc32)c2ccccc2-5)cc1 ; c1ccc(N(c2ccccc2)c2ccc3oc4cc5c(cc4c3c2)C2(c3ccccc3Oc3ccccc32)c2ccccc2-5)cc1]

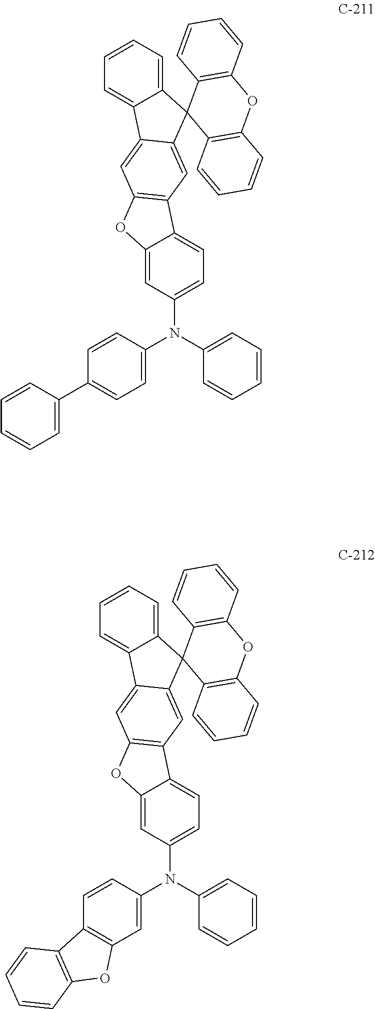 [SMILES: c1ccc(-c2ccc(N(c3ccccc3)c3ccc4c(c3)oc3cc5c(cc34)C3(c4ccccc4Oc4ccccc43)c3ccccc3-5)cc2)cc1 ; c1ccc(N(c2ccc3c(c2)oc2ccccc23)c2ccc3c(c2)oc2cc4c(cc23)C2(c3ccccc3Oc3ccccc32)c2ccccc2-4)cc1]

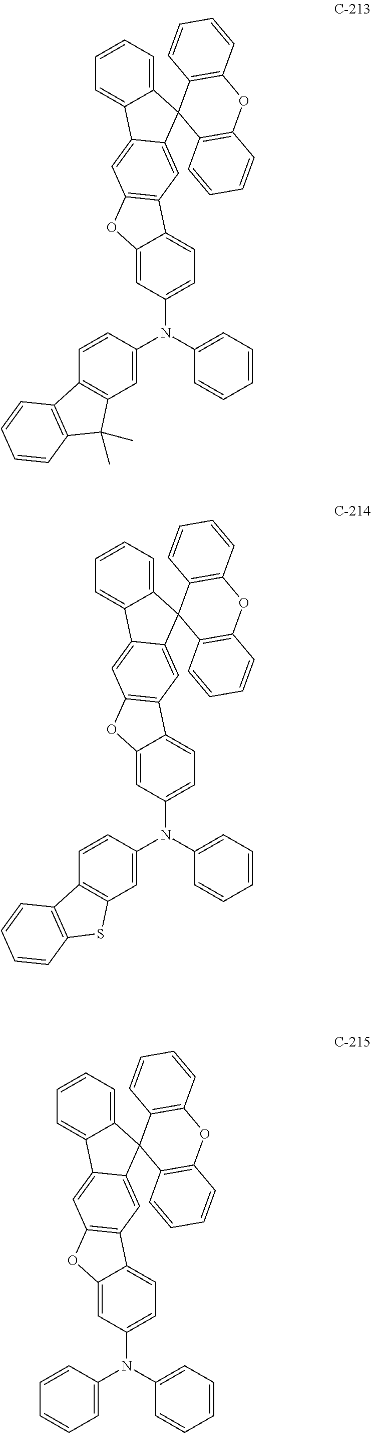 [SMILES: CC1(C)c2ccccc2-c2ccc(N(c3ccccc3)c3ccc4c(c3)oc3cc5c(cc34)C3(c4ccccc4Oc4ccccc43)c3ccccc3-5)cc21 ; c1ccc(N(c2ccc3c(c2)oc2cc4c(cc23)C2(c3ccccc3Oc3ccccc32)c2ccccc2-4)c2ccc3c(c2)sc2ccccc23)cc1 ; c1ccc(N(c2ccccc2)c2ccc3c(c2)oc2cc4c(cc23)C2(c3ccccc3Oc3ccccc32)c2ccccc2-4)cc1]

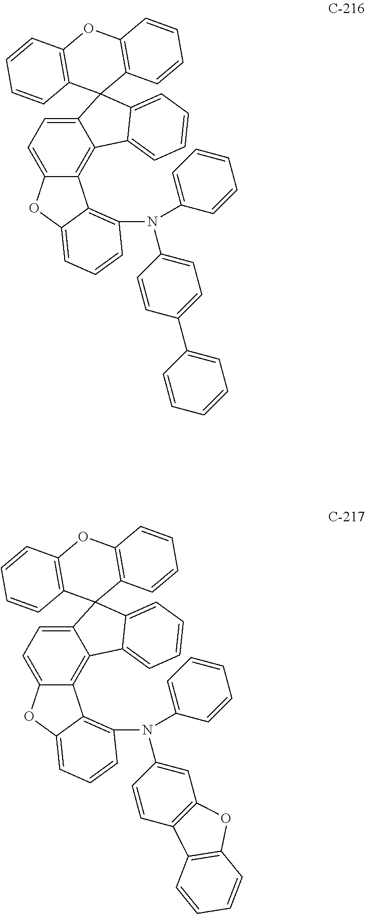 [SMILES: c1ccc(-c2ccc(N(c3ccccc3)c3cccc4oc5ccc6c(c5c34)-c3ccccc3C63c4ccccc4Oc4ccccc43)cc2)cc1 ; c1ccc(N(c2ccc3c(c2)oc2ccccc23)c2cccc3oc4ccc5c(c4c23)-c2ccccc2C52c3ccccc3Oc3ccccc32)cc1]

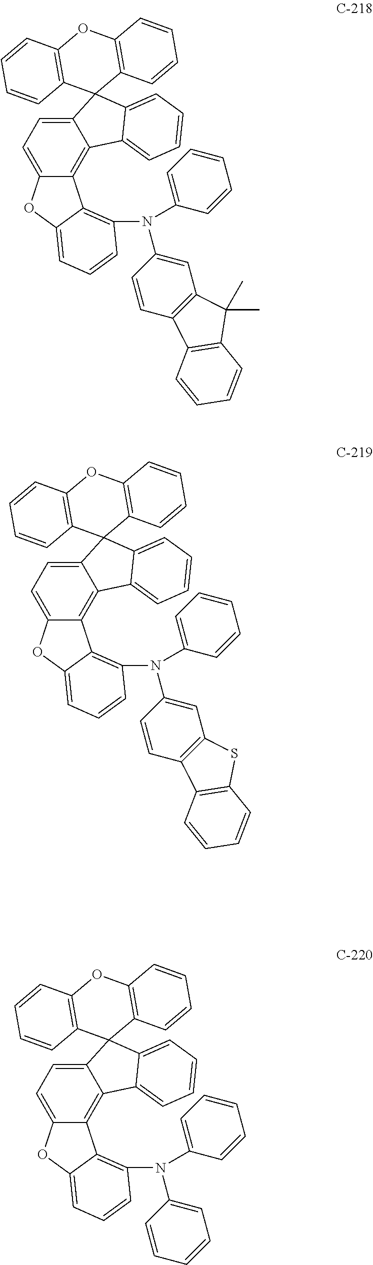 [SMILES: CC1(C)c2ccccc2-c2ccc(N(c3ccccc3)c3cccc4oc5ccc6c(c5c34)-c3ccccc3C63c4ccccc4Oc4ccccc43)cc21 ; c1ccc(N(c2ccc3c(c2)sc2ccccc23)c2cccc3oc4ccc5c(c4c23)-c2ccccc2C52c3ccccc3Oc3ccccc32)cc1 ; c1ccc(N(c2ccccc2)c2cccc3oc4ccc5c(c4c23)-c2ccccc2C52c3ccccc3Oc3ccccc32)cc1]

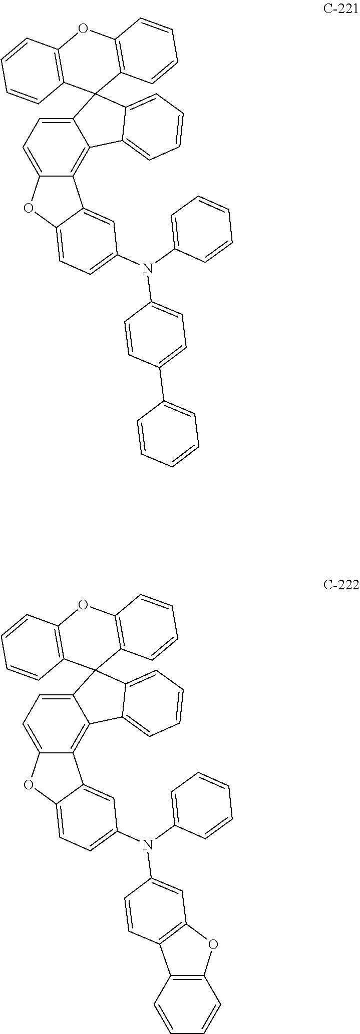 [SMILES: c1ccc(-c2ccc(N(c3ccccc3)c3ccc4oc5ccc6c(c5c4c3)-c3ccccc3C63c4ccccc4Oc4ccccc43)cc2)cc1 ; c1ccc(N(c2ccc3c(c2)oc2ccccc23)c2ccc3oc4ccc5c(c4c3c2)-c2ccccc2C52c3ccccc3Oc3ccccc32)cc1]

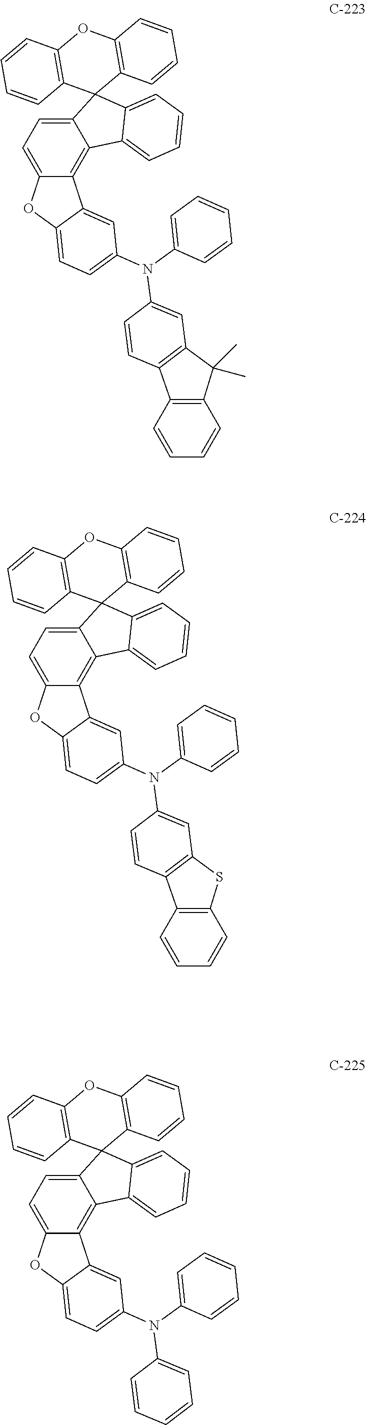 [SMILES: CC1(C)c2ccccc2-c2ccc(N(c3ccccc3)c3ccc4oc5ccc6c(c5c4c3)-c3ccccc3C63c4ccccc4Oc4ccccc43)cc21 ; c1ccc(N(c2ccc3c(c2)sc2ccccc23)c2ccc3oc4ccc5c(c4c3c2)-c2ccccc2C52c3ccccc3Oc3ccccc32)cc1 ; c1ccc(N(c2ccccc2)c2ccc3oc4ccc5c(c4c3c2)-c2ccccc2C52c3ccccc3Oc3ccccc32)cc1]

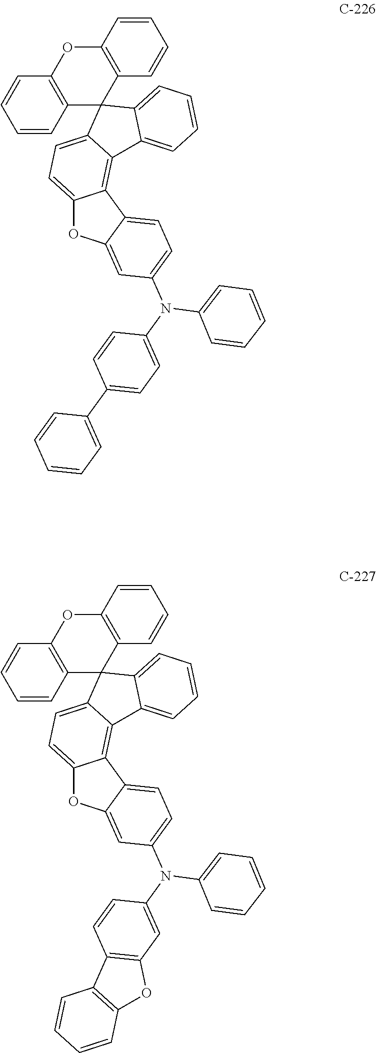 [SMILES: c1ccc(-c2ccc(N(c3ccccc3)c3ccc4c(c3)oc3ccc5c(c34)-c3ccccc3C53c4ccccc4Oc4ccccc43)cc2)cc1 ; c1ccc(N(c2ccc3c(c2)oc2ccccc23)c2ccc3c(c2)oc2ccc4c(c23)-c2ccccc2C42c3ccccc3Oc3ccccc32)cc1]

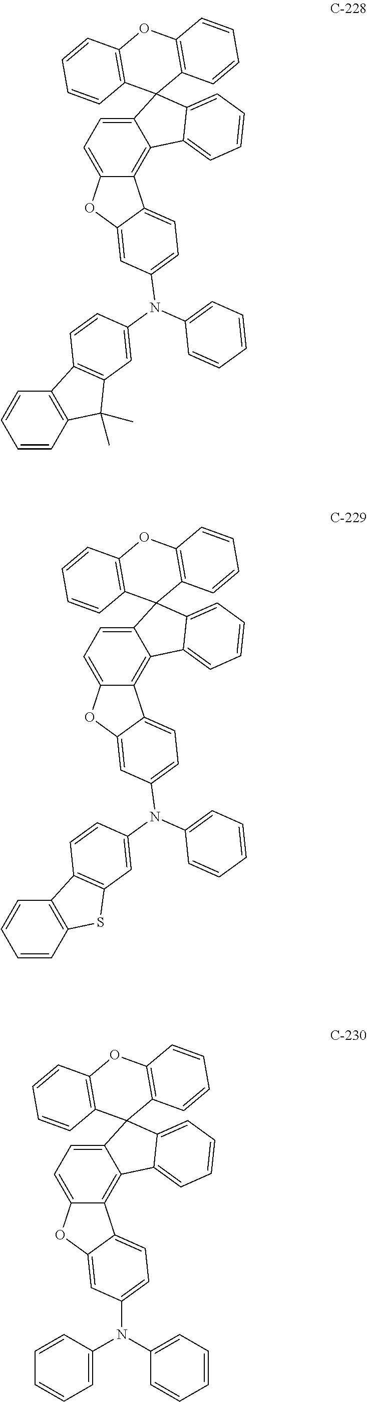 [SMILES: CC1(C)c2ccccc2-c2ccc(N(c3ccccc3)c3ccc4c(c3)oc3ccc5c(c34)-c3ccccc3C53c4ccccc4Oc4ccccc43)cc21 ; c1ccc(N(c2ccc3c(c2)oc2ccc4c(c23)-c2ccccc2C42c3ccccc3Oc3ccccc32)c2ccc3c(c2)sc2ccccc23)cc1 ; c1ccc(N(c2ccccc2)c2ccc3c(c2)oc2ccc4c(c23)-c2ccccc2C42c3ccccc3Oc3ccccc32)cc1]

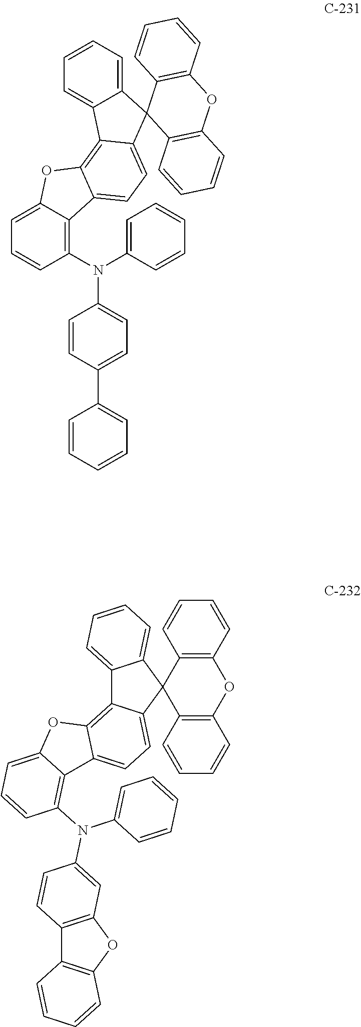 [SMILES: c1ccc(-c2ccc(N(c3ccccc3)c3cccc4oc5c6c(ccc5c34)C3(c4ccccc4Oc4ccccc43)c3ccccc3-6)cc2)cc1 ; c1ccc(N(c2ccc3c(c2)oc2ccccc23)c2cccc3oc4c5c(ccc4c23)C2(c3ccccc3Oc3ccccc32)c2ccccc2-5)cc1]

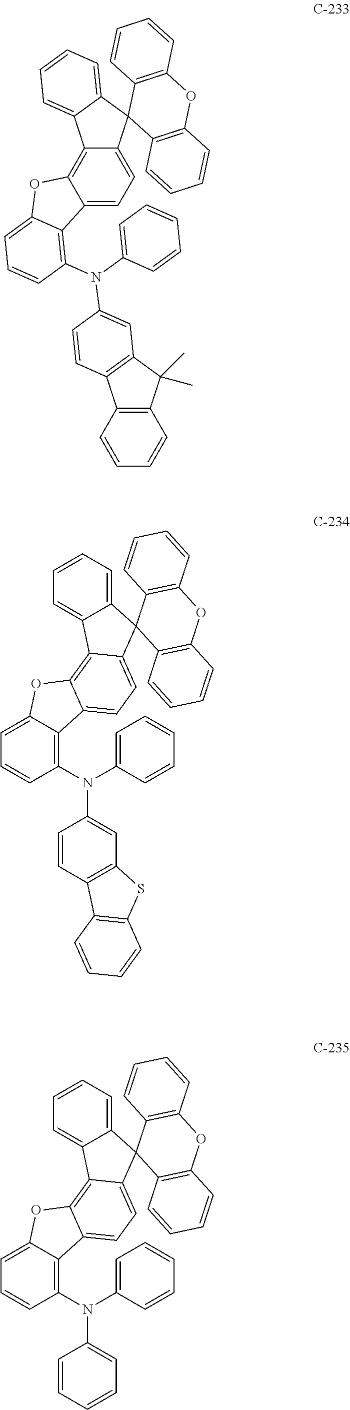 [SMILES: CC1(C)c2ccccc2-c2ccc(N(c3ccccc3)c3cccc4oc5c6c(ccc5c34)C3(c4ccccc4Oc4ccccc43)c3ccccc3-6)cc21 ; c1ccc(N(c2ccc3c(c2)sc2ccccc23)c2cccc3oc4c5c(ccc4c23)C2(c3ccccc3Oc3ccccc32)c2ccccc2-5)cc1 ; c1ccc(N(c2ccccc2)c2cccc3oc4c5c(ccc4c23)C2(c3ccccc3Oc3ccccc32)c2ccccc2-5)cc1]

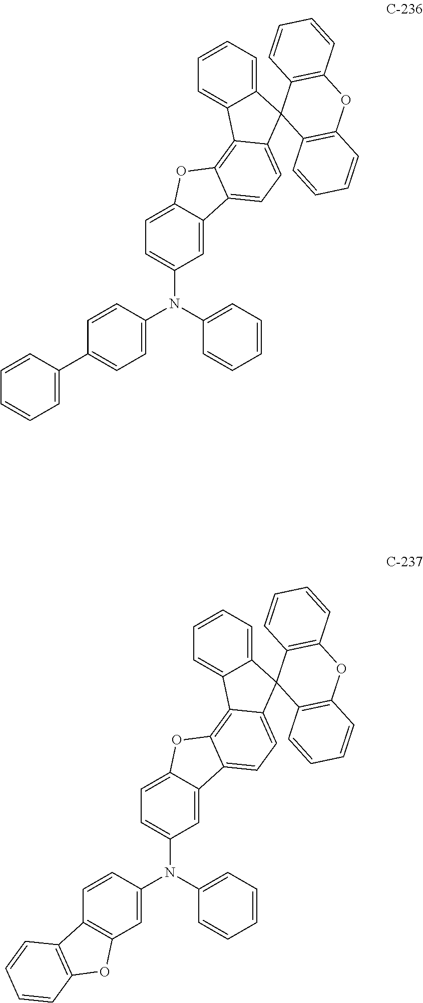 [SMILES: c1ccc(-c2ccc(N(c3ccccc3)c3ccc4oc5c6c(ccc5c4c3)C3(c4ccccc4Oc4ccccc43)c3ccccc3-6)cc2)cc1 ; c1ccc(N(c2ccc3c(c2)oc2ccccc23)c2ccc3oc4c5c(ccc4c3c2)C2(c3ccccc3Oc3ccccc32)c2ccccc2-5)cc1]

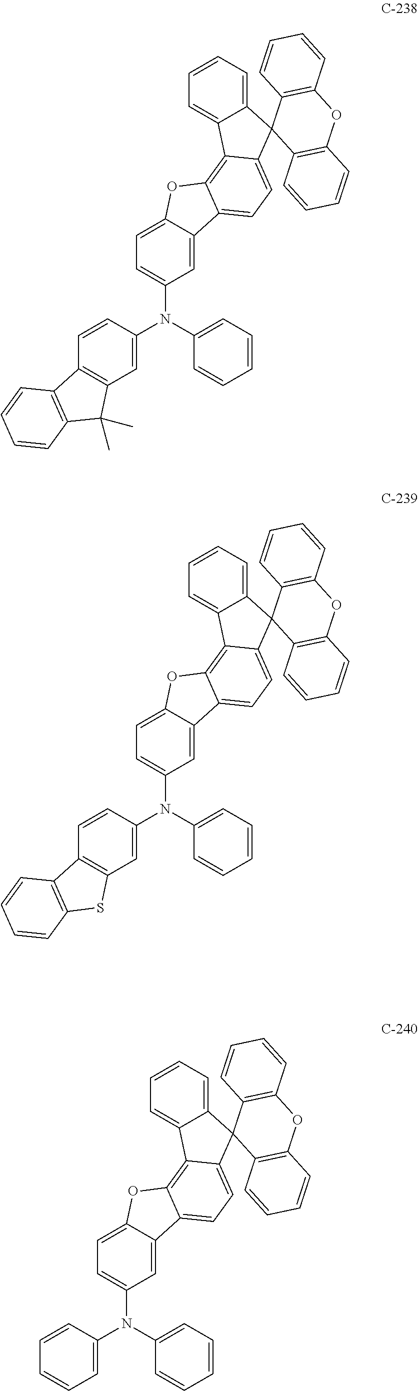 [SMILES: CC1(C)c2ccccc2-c2ccc(N(c3ccccc3)c3ccc4oc5c6c(ccc5c4c3)C3(c4ccccc4Oc4ccccc43)c3ccccc3-6)cc21 ; c1ccc(N(c2ccc3c(c2)sc2ccccc23)c2ccc3oc4c5c(ccc4c3c2)C2(c3ccccc3Oc3ccccc32)c2ccccc2-5)cc1 ; c1ccc(N(c2ccccc2)c2ccc3oc4c5c(ccc4c3c2)C2(c3ccccc3Oc3ccccc32)c2ccccc2-5)cc1]

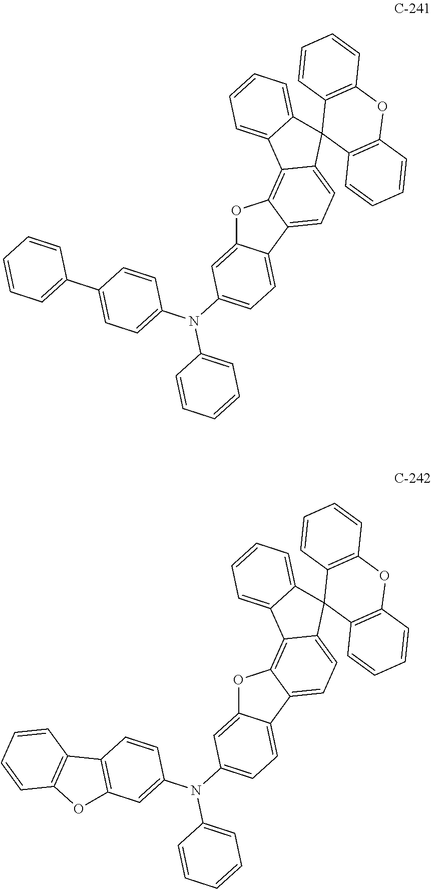 [SMILES: c1ccc(-c2ccc(N(c3ccccc3)c3ccc4c(c3)oc3c5c(ccc34)C3(c4ccccc4Oc4ccccc43)c3ccccc3-5)cc2)cc1 ; c1ccc(N(c2ccc3c(c2)oc2ccccc23)c2ccc3c(c2)oc2c4c(ccc23)C2(c3ccccc3Oc3ccccc32)c2ccccc2-4)cc1]

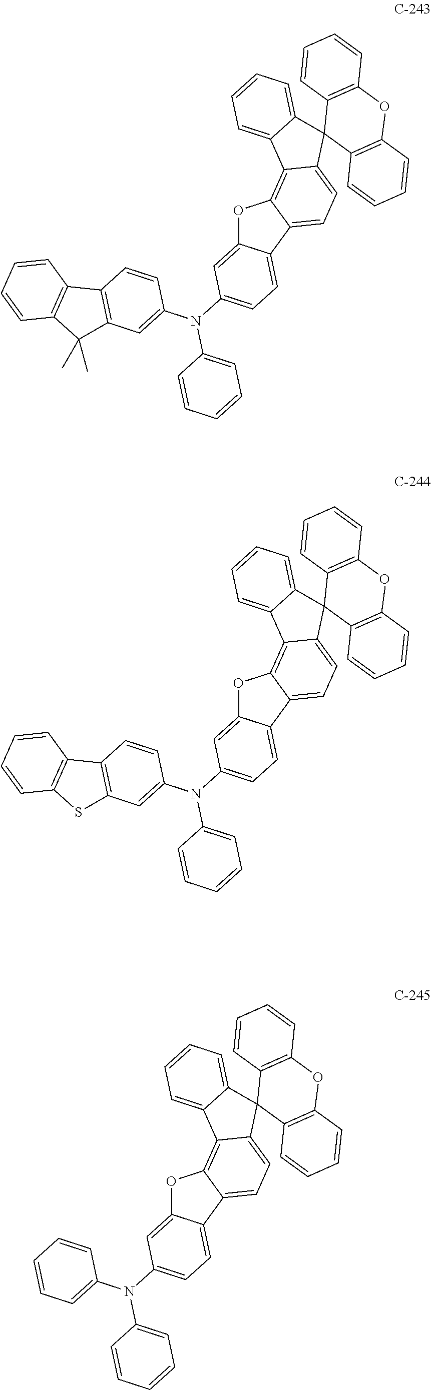 [SMILES: CC1(C)c2ccccc2-c2ccc(N(c3ccccc3)c3ccc4c(c3)oc3c5c(ccc34)C3(c4ccccc4Oc4ccccc43)c3ccccc3-5)cc21 ; c1ccc(N(c2ccc3c(c2)oc2c4c(ccc23)C2(c3ccccc3Oc3ccccc32)c2ccccc2-4)c2ccc3c(c2)sc2ccccc23)cc1 ; c1ccc(N(c2ccccc2)c2ccc3c(c2)oc2c4c(ccc23)C2(c3ccccc3Oc3ccccc32)c2ccccc2-4)cc1]

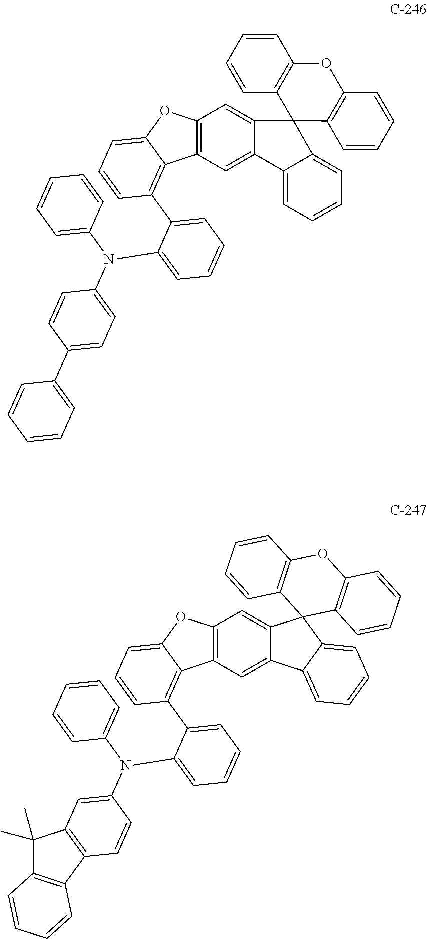 [SMILES: c1ccc(-c2ccc(N(c3ccccc3)c3ccccc3-c3cccc4oc5cc6c(cc5c34)-c3ccccc3C63c4ccccc4Oc4ccccc43)cc2)cc1 ; CC1(C)c2ccccc2-c2ccc(N(c3ccccc3)c3ccccc3-c3cccc4oc5cc6c(cc5c34)-c3ccccc3C63c4ccccc4Oc4ccccc43)cc21]

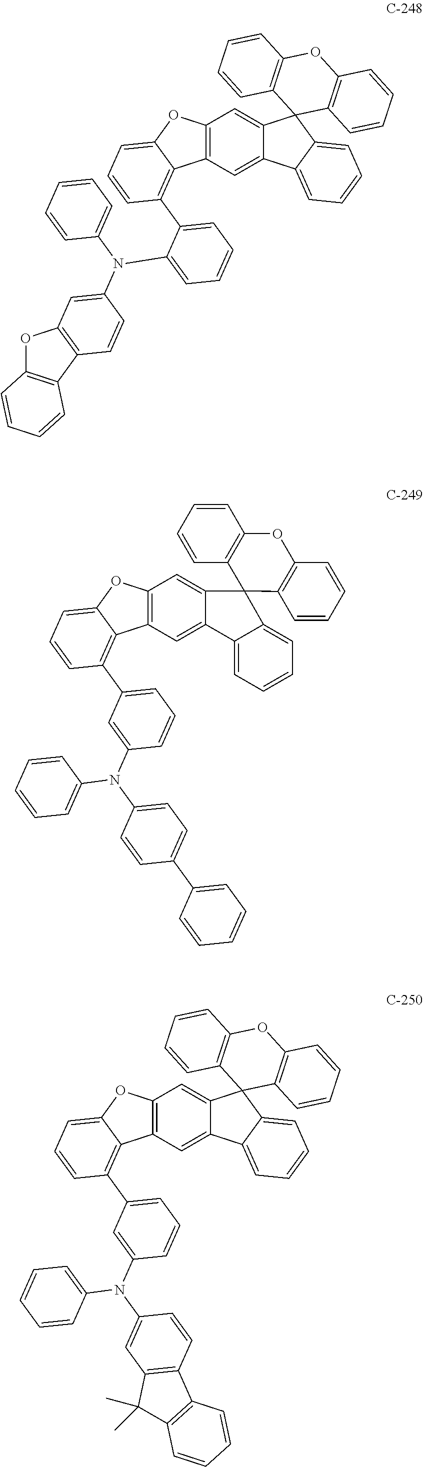 [SMILES: c1ccc(N(c2ccc3c(c2)oc2ccccc23)c2ccccc2-c2cccc3oc4cc5c(cc4c23)-c2ccccc2C52c3ccccc3Oc3ccccc32)cc1 ; c1ccc(-c2ccc(N(c3ccccc3)c3cccc(-c4cccc5oc6cc7c(cc6c45)-c4ccccc4C74c5ccccc5Oc5ccccc54)c3)cc2)cc1 ; CC1(C)c2ccccc2-c2ccc(N(c3ccccc3)c3cccc(-c4cccc5oc6cc7c(cc6c45)-c4ccccc4C74c5ccccc5Oc5ccccc54)c3)cc21]

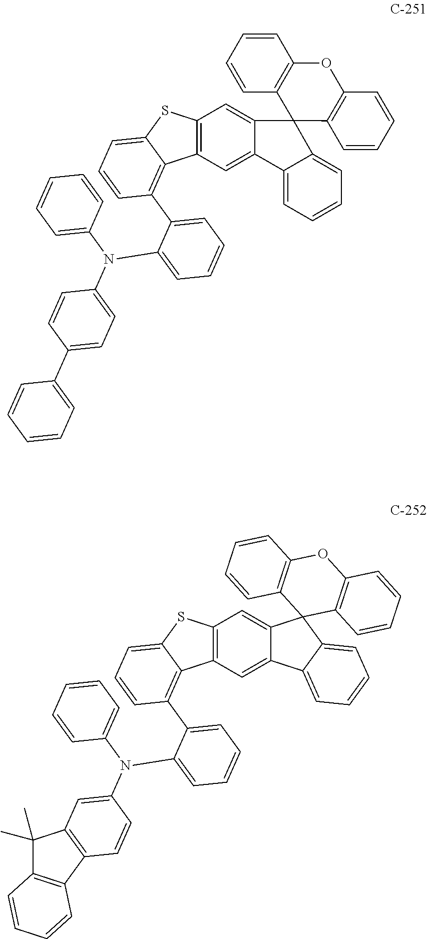 [SMILES: c1ccc(-c2ccc(N(c3ccccc3)c3ccccc3-c3cccc4sc5cc6c(cc5c34)-c3ccccc3C63c4ccccc4Oc4ccccc43)cc2)cc1 ; CC1(C)c2ccccc2-c2ccc(N(c3ccccc3)c3ccccc3-c3cccc4sc5cc6c(cc5c34)-c3ccccc3C63c4ccccc4Oc4ccccc43)cc21]

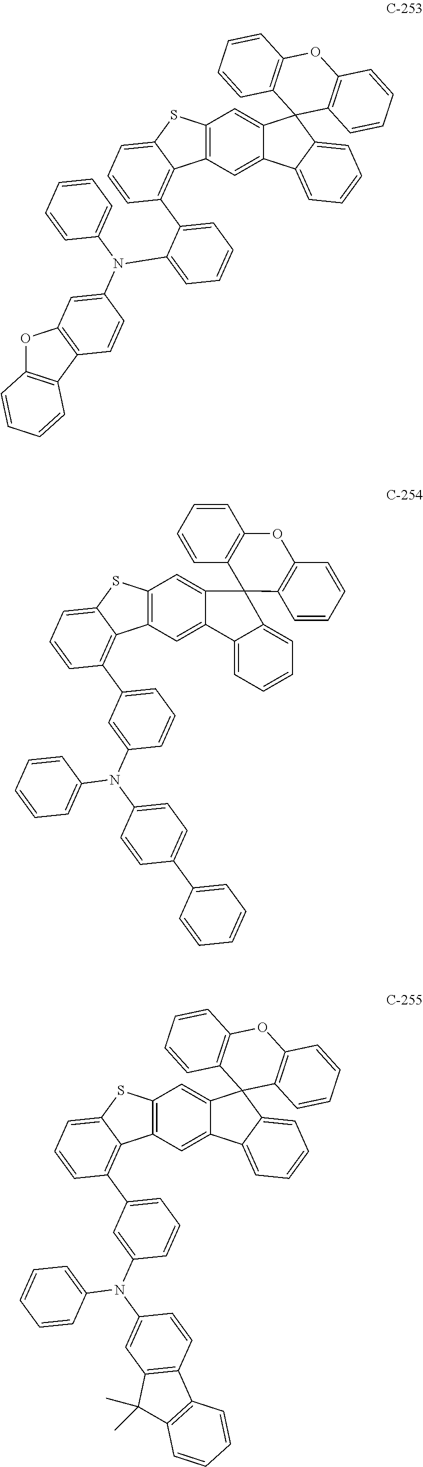 [SMILES: c1ccc(N(c2ccc3c(c2)oc2ccccc23)c2ccccc2-c2cccc3sc4cc5c(cc4c23)-c2ccccc2C52c3ccccc3Oc3ccccc32)cc1 ; c1ccc(-c2ccc(N(c3ccccc3)c3cccc(-c4cccc5sc6cc7c(cc6c45)-c4ccccc4C74c5ccccc5Oc5ccccc54)c3)cc2)cc1 ; CC1(C)c2ccccc2-c2ccc(N(c3ccccc3)c3cccc(-c4cccc5sc6cc7c(cc6c45)-c4ccccc4C74c5ccccc5Oc5ccccc54)c3)cc21]

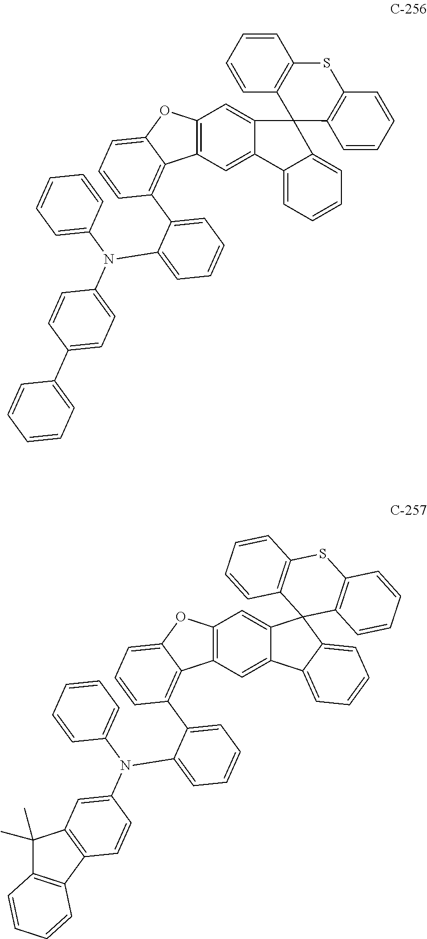 [SMILES: c1ccc(-c2ccc(N(c3ccccc3)c3ccccc3-c3cccc4oc5cc6c(cc5c34)-c3ccccc3C63c4ccccc4Sc4ccccc43)cc2)cc1 ; CC1(C)c2ccccc2-c2ccc(N(c3ccccc3)c3ccccc3-c3cccc4oc5cc6c(cc5c34)-c3ccccc3C63c4ccccc4Sc4ccccc43)cc21]

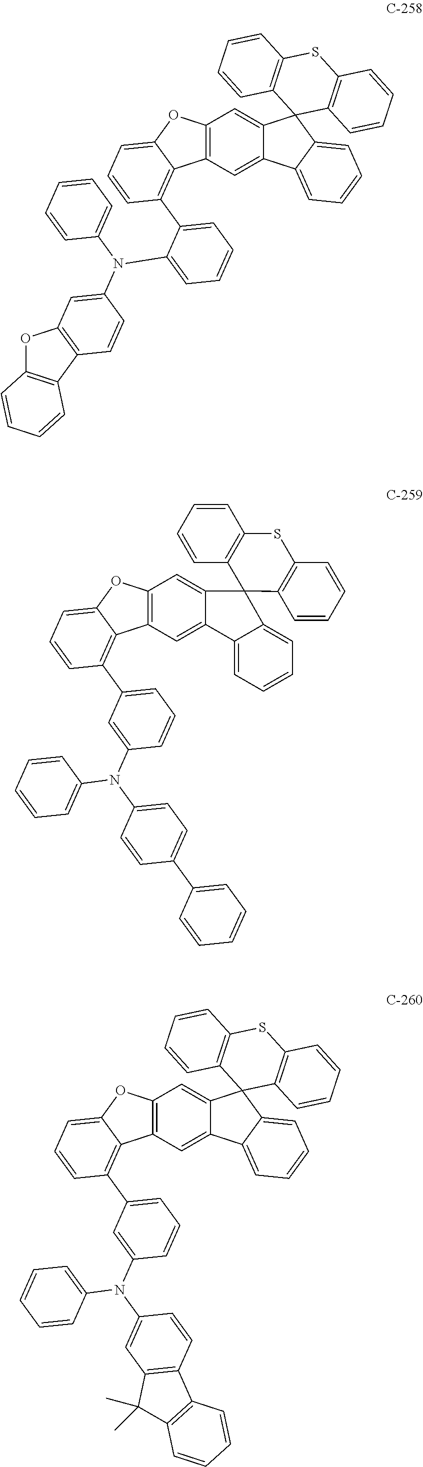 [SMILES: c1ccc(N(c2ccc3c(c2)oc2ccccc23)c2ccccc2-c2cccc3oc4cc5c(cc4c23)-c2ccccc2C52c3ccccc3Sc3ccccc32)cc1 ; c1ccc(-c2ccc(N(c3ccccc3)c3cccc(-c4cccc5oc6cc7c(cc6c45)-c4ccccc4C74c5ccccc5Sc5ccccc54)c3)cc2)cc1 ; CC1(C)c2ccccc2-c2ccc(N(c3ccccc3)c3cccc(-c4cccc5oc6cc7c(cc6c45)-c4ccccc4C74c5ccccc5Sc5ccccc54)c3)cc21]

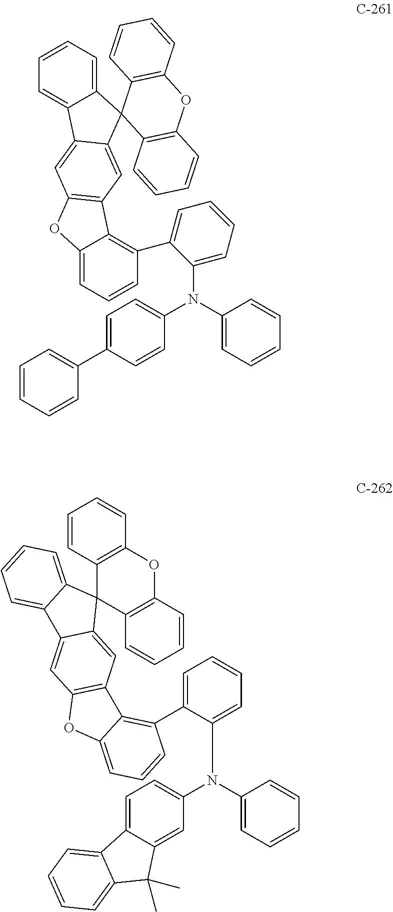 [SMILES: c1ccc(-c2ccc(N(c3ccccc3)c3ccccc3-c3cccc4oc5cc6c(cc5c34)C3(c4ccccc4Oc4ccccc43)c3ccccc3-6)cc2)cc1 ; CC1(C)c2ccccc2-c2ccc(N(c3ccccc3)c3ccccc3-c3cccc4oc5cc6c(cc5c34)C3(c4ccccc4Oc4ccccc43)c3ccccc3-6)cc21]

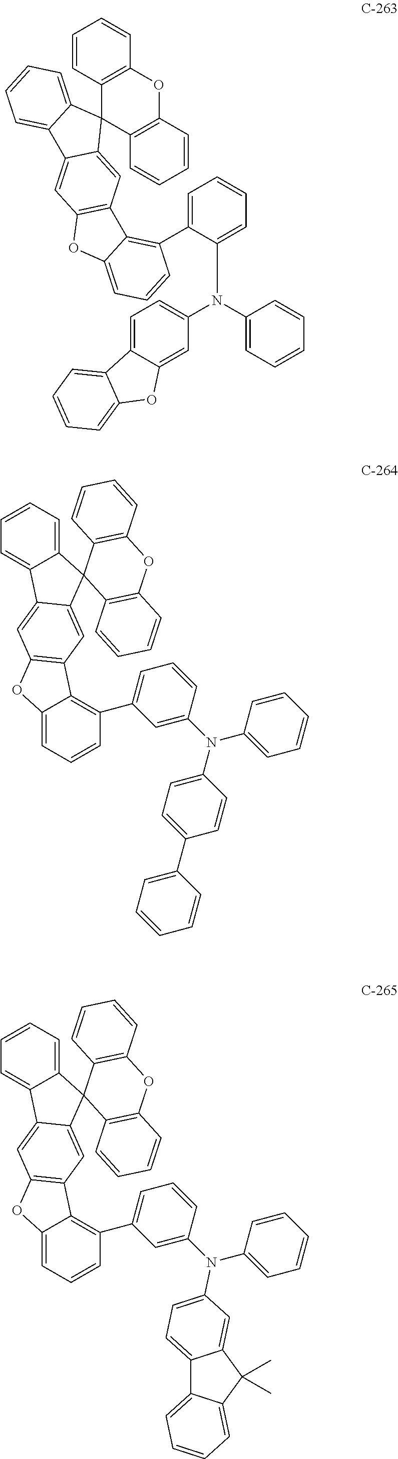 [SMILES: c1ccc(N(c2ccc3c(c2)oc2ccccc23)c2ccccc2-c2cccc3oc4cc5c(cc4c23)C2(c3ccccc3Oc3ccccc32)c2ccccc2-5)cc1 ; c1ccc(-c2ccc(N(c3ccccc3)c3cccc(-c4cccc5oc6cc7c(cc6c45)C4(c5ccccc5Oc5ccccc54)c4ccccc4-7)c3)cc2)cc1 ; CC1(C)c2ccccc2-c2ccc(N(c3ccccc3)c3cccc(-c4cccc5oc6cc7c(cc6c45)C4(c5ccccc5Oc5ccccc54)c4ccccc4-7)c3)cc21]

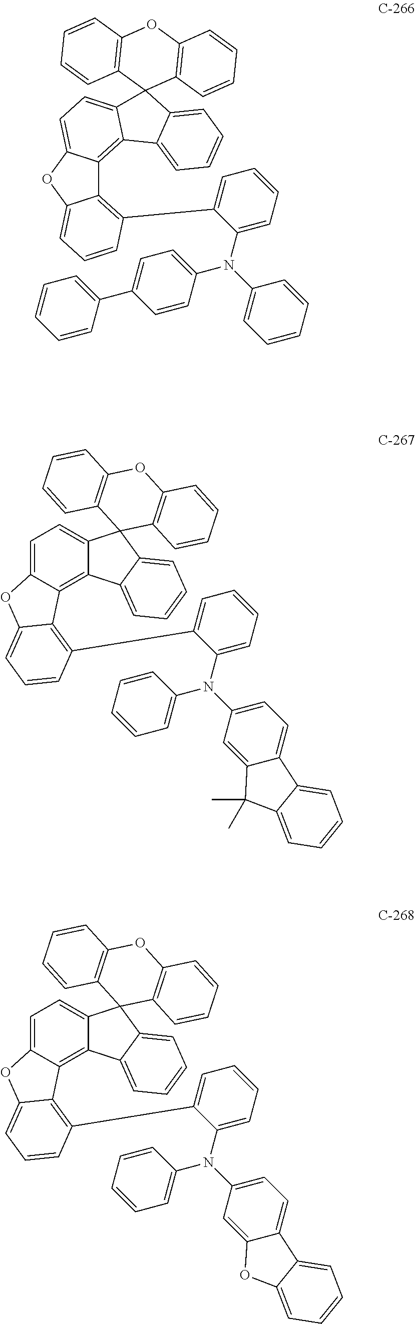 [SMILES: c1ccc(-c2ccc(N(c3ccccc3)c3ccccc3-c3cccc4oc5ccc6c(c5c34)-c3ccccc3C63c4ccccc4Oc4ccccc43)cc2)cc1 ; CC1(C)c2ccccc2-c2ccc(N(c3ccccc3)c3ccccc3-c3cccc4oc5ccc6c(c5c34)-c3ccccc3C63c4ccccc4Oc4ccccc43)cc21 ; c1ccc(N(c2ccc3c(c2)oc2ccccc23)c2ccccc2-c2cccc3oc4ccc5c(c4c23)-c2ccccc2C52c3ccccc3Oc3ccccc32)cc1]

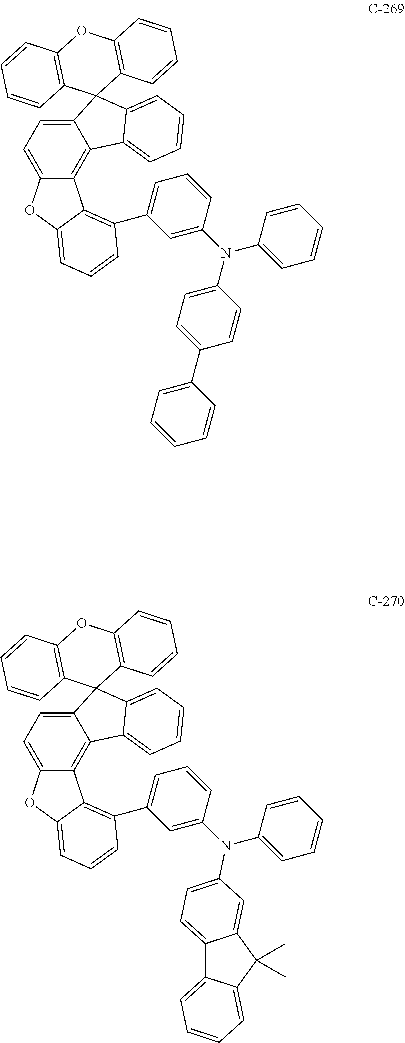 [SMILES: c1ccc(-c2ccc(N(c3ccccc3)c3cccc(-c4cccc5oc6ccc7c(c6c45)-c4ccccc4C74c5ccccc5Oc5ccccc54)c3)cc2)cc1 ; CC1(C)c2ccccc2-c2ccc(N(c3ccccc3)c3cccc(-c4cccc5oc6ccc7c(c6c45)-c4ccccc4C74c5ccccc5Oc5ccccc54)c3)cc21]

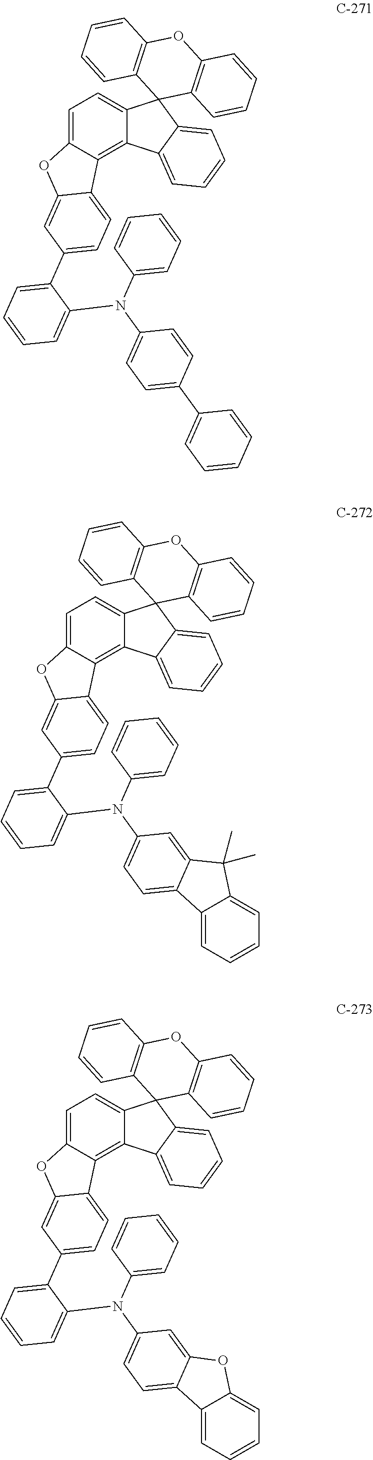 [SMILES: c1ccc(-c2ccc(N(c3ccccc3)c3ccccc3-c3ccc4c(c3)oc3ccc5c(c34)-c3ccccc3C53c4ccccc4Oc4ccccc43)cc2)cc1 ; CC1(C)c2ccccc2-c2ccc(N(c3ccccc3)c3ccccc3-c3ccc4c(c3)oc3ccc5c(c34)-c3ccccc3C53c4ccccc4Oc4ccccc43)cc21 ; c1ccc(N(c2ccc3c(c2)oc2ccccc23)c2ccccc2-c2ccc3c(c2)oc2ccc4c(c23)-c2ccccc2C42c3ccccc3Oc3ccccc32)cc1]

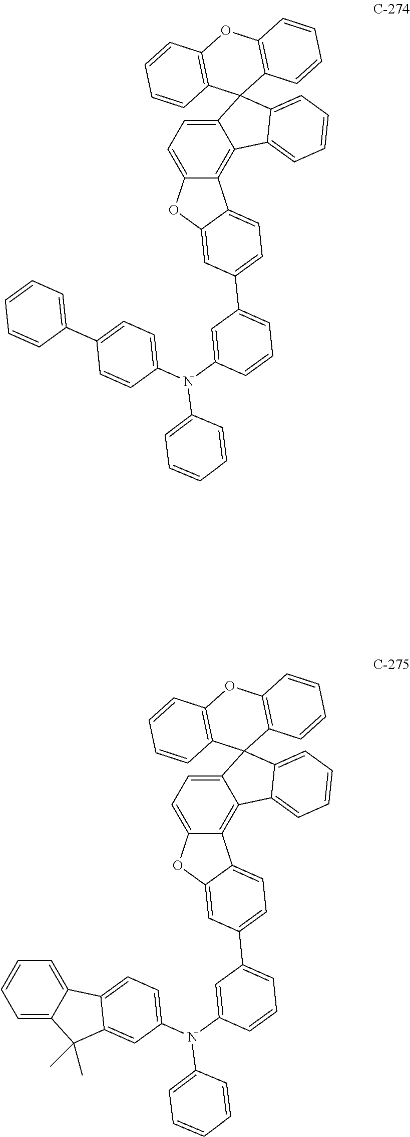 [SMILES: c1ccc(-c2ccc(N(c3ccccc3)c3cccc(-c4ccc5c(c4)oc4ccc6c(c45)-c4ccccc4C64c5ccccc5Oc5ccccc54)c3)cc2)cc1 ; CC1(C)c2ccccc2-c2ccc(N(c3ccccc3)c3cccc(-c4ccc5c(c4)oc4ccc6c(c45)-c4ccccc4C64c5ccccc5Oc5ccccc54)c3)cc21]

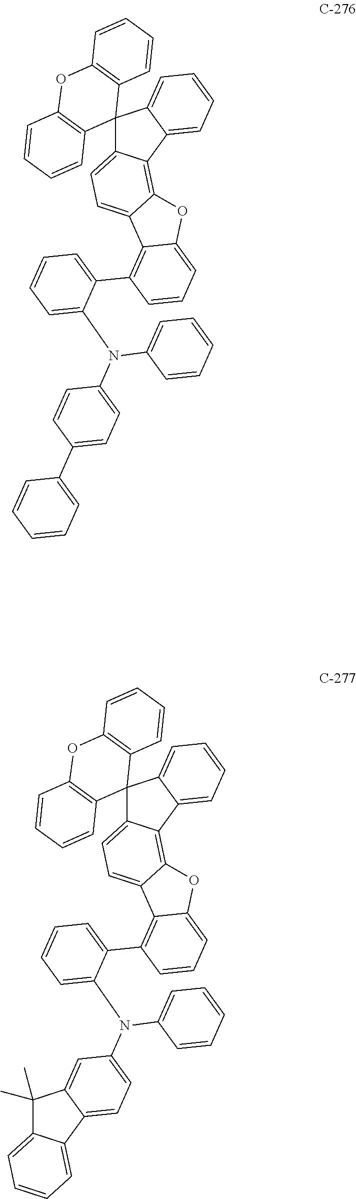 [SMILES: c1ccc(-c2ccc(N(c3ccccc3)c3ccccc3-c3cccc4oc5c6c(ccc5c34)C3(c4ccccc4Oc4ccccc43)c3ccccc3-6)cc2)cc1 ; CC1(C)c2ccccc2-c2ccc(N(c3ccccc3)c3ccccc3-c3cccc4oc5c6c(ccc5c34)C3(c4ccccc4Oc4ccccc43)c3ccccc3-6)cc21]

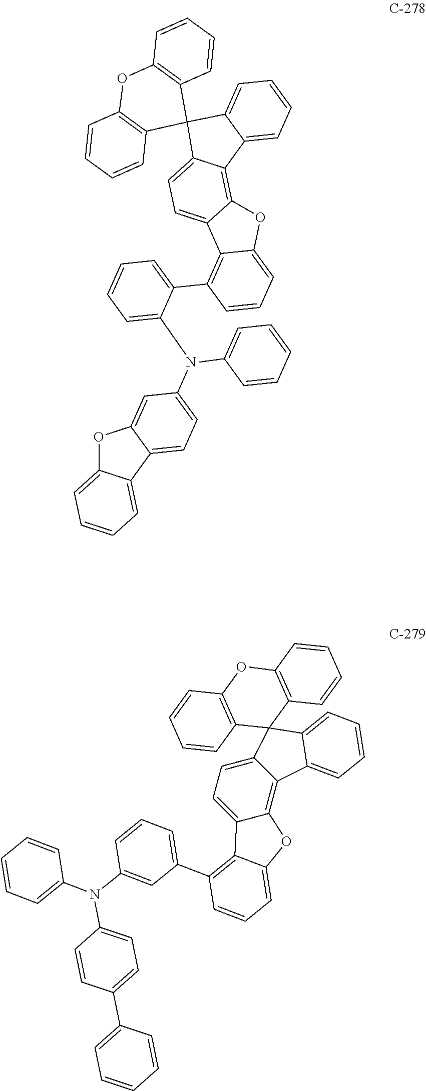 [SMILES: c1ccc(N(c2ccc3c(c2)oc2ccccc23)c2ccccc2-c2cccc3oc4c5c(ccc4c23)C2(c3ccccc3Oc3ccccc32)c2ccccc2-5)cc1 ; c1ccc(-c2ccc(N(c3ccccc3)c3cccc(-c4cccc5oc6c7c(ccc6c45)C4(c5ccccc5Oc5ccccc54)c4ccccc4-7)c3)cc2)cc1]

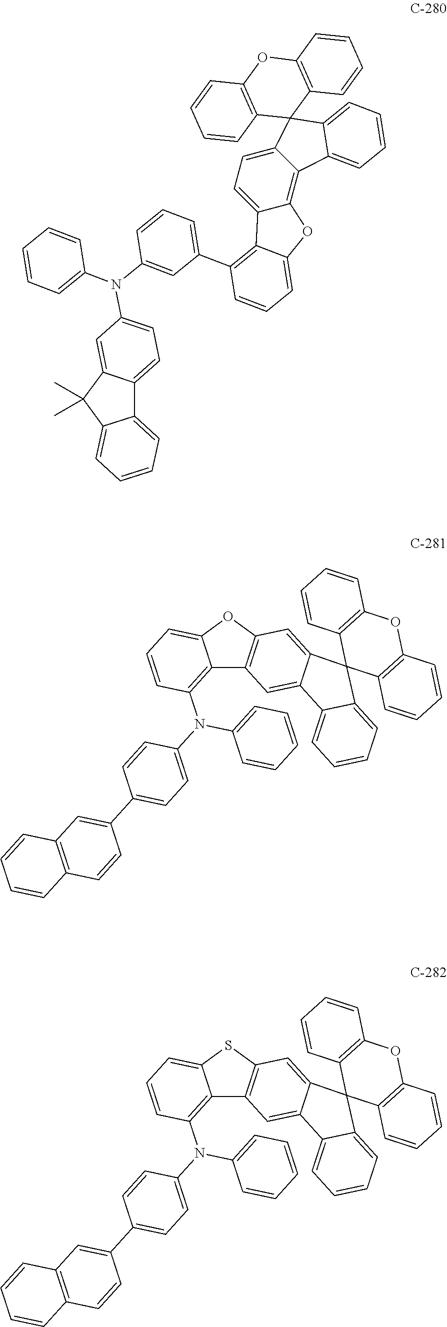 [SMILES: CC1(C)c2ccccc2-c2ccc(N(c3ccccc3)c3cccc(-c4cccc5oc6c7c(ccc6c45)C4(c5ccccc5Oc5ccccc54)c4ccccc4-7)c3)cc21 ; c1ccc(N(c2ccc(-c3ccc4ccccc4c3)cc2)c2cccc3oc4cc5c(cc4c23)-c2ccccc2C52c3ccccc3Oc3ccccc32)cc1 ; c1ccc(N(c2ccc(-c3ccc4ccccc4c3)cc2)c2cccc3sc4cc5c(cc4c23)-c2ccccc2C52c3ccccc3Oc3ccccc32)cc1]

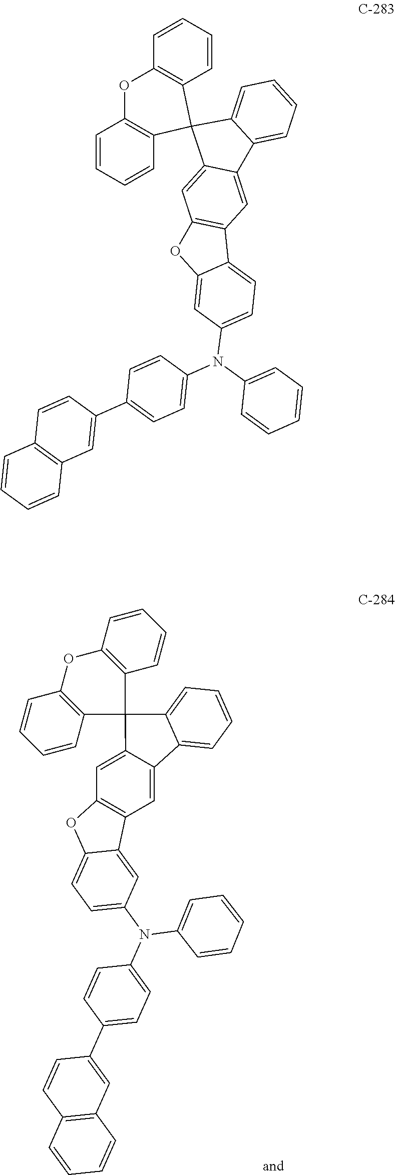 [SMILES: c1ccc(N(c2ccc(-c3ccc4ccccc4c3)cc2)c2ccc3c(c2)oc2cc4c(cc23)-c2ccccc2C42c3ccccc3Oc3ccccc32)cc1 ; c1ccc(N(c2ccc(-c3ccc4ccccc4c3)cc2)c2ccc3oc4cc5c(cc4c3c2)-c2ccccc2C52c3ccccc3Oc3ccccc32)cc1]

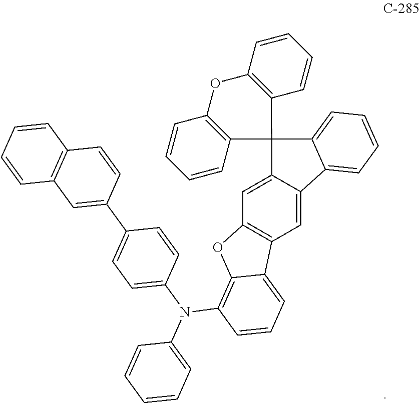 [SMILES: c1ccc(N(c2ccc(-c3ccc4ccccc4c3)cc2)c2cccc3c2oc2cc4c(cc23)-c2ccccc2C42c3ccccc3Oc3ccccc32)cc1]

6. An organic electroluminescent device comprising an organic electroluminescent compound according to claim 1.

7. The organic electroluminescent device according to claim 6, wherein the organic electroluminescent compound is included in a hole transport zone.
